(19) United States
(12) Patent Application Publication
(10) Pub. No.: US 2011/0082098 A1
(43) Pub. Date: Apr. 7, 2011

(54) NOVOBIOCIN ANALOGUES AND TREATMENT OF POLYCYSTIC KIDNEY DISEASE

(75) Inventors: James P. Calvet, Lenexa, KS (US); Brian S.J. Blagg, Lawrence, KS (US); Shirin V. Sundar, Overland Park, KS (US); Brenda S. Magenheimer, Kansas City, MO (US)

(73) Assignee: UNIVERSITY OF KANSAS, Lawrence, KS (US)

(21) Appl. No.: 12/894,654

(22) Filed: Sep. 30, 2010

Related U.S. Application Data
(60) Provisional application No. 61/247,490, filed on Sep. 30, 2009.

(51) Int. Cl. A61K 31/7048; A61K 31/706; A61K 31/7056; A61K 31/685; A61K 31/353; A61K 31/4433; A61K 31/453; A61K 31/404; A61P 13/12

(52) U.S. Cl. 514/27; 514/100; 514/457; 514/337; 514/320; 514/414

(57) ABSTRACT

Novobiocin analogues are useful in methods of treating, inhibiting, and/or preventing cyst formation in autosomal dominant polycystic kidney disease (ADPKD) in a subject. The disclosure provides methods of treating ADPKD comprising administering a therapeutically effective amount of a coumarin-3-carboxamide novobiocin analogue. Accordingly, the method can include administering a novobiocin analogue in a therapeutically effective amount for reducing levels of mTOR pathway phosphoproteins P-mTOR, P-Akt and P-S6K, or combinations thereof. Further, the method can include administering a novobiocin analogue in a therapeutically effective amount for reducing levels of Hsp-90 client proteins CFTR, ErbB2, c-Raf and Cdk4, or combinations thereof.

### CROSS-REFERENCE TO RELATED APPLICATIONS

This application claims the benefit of U.S. provisional application Ser. No. 61/247,490, filed Sep. 30, 2009, which is incorporated herein by specific reference in its entirety.

### STATEMENT REGARDING FEDERALLY SPONSORED RESEARCH OR DEVELOPMENT

This invention was made with government support under grant number P50 DK05301 awarded by the National Institutes of Health. The government has certain rights in the invention.

### FIELD OF THE INVENTION

Novobiocin analogues are useful in methods of treating, inhibiting, and/or preventing cyst formation in autosomal dominant polycystic kidney disease (ADPKD) in a subject. The disclosure provides methods of treating ADPKD comprising administering a therapeutically effective amount of a coumarin-3-carboxamide novobiocin analogue.

### BACKGROUND OF THE INVENTION

Polycystic kidney disease (PKD or PCKD, also known as polycystic kidney syndrome) is a genetic disorder of the kidneys. PKD is characterized by cyst formation and progressive enlargement of both kidneys, leading to end-stage renal disease (ESRD). In PKD, clusters of cysts develop primarily within the kidneys. Cysts are non-cancerous round sacs containing water-like fluid. Cysts vary in size as they accumulate fluid and can grow extremely large.

There are two types of PKD; autosomal dominant PKD and autosomal recessive PKD. Autosomal dominant polycystic kidney disease (ADPKD) is the most common life-threatening hereditary disorder affecting approximately one in every 400-1000 live births. ADPKD is typically a late-onset (adult onset) disorder. In a small percentage of patients with no family history, ADPKD is thought to result from a spontaneous gene mutation.

Autosomal recessive polycystic kidney disease (ARPKD) occurs in about one of every 20,000 live births. ARPKD, also known as congenital hepatic fibrosis (CHF), is commonly diagnosed early in life; about 50% of cases are diagnosed prenatally. Neonatal death occurs in about 50% of cases diagnosed prenatally, typically due to pulmonary hyperplasia; underdeveloped lungs.

PKD is not limited to the kidneys. Liver damage is the second most common manifestation of PKD. Cyst development also occurs in the pancreas, in the membranes surrounding the brain and central nervous system, and in seminal vesicles. Hypertension is common in patients with PKD, and as patients progress to end-stage renal disease, almost all will develop hypertension. Abdominal hernias are common in patients with ADPKD. Therefore, numerous complications are associated with PKD including high blood pressure, loss of kidney function, pregnancy complications, growth of cysts in the liver, frequent urinary tract infection, intracranial and aortic aneurysms, heart valve abnormalities, colon problems, and chronic pain. An additional complication of ARPKD is hepatic fibrosis. There is currently no PKD therapy that slows or prevents cyst formation and kidney enlargement in humans. Belibi and Edelstein 2010, Novel targets for treatment of autosomal dominant polycystic kidney disease, Expert Opin. Investig. Drugs 19(3):315-328.

ADPKD is a genetic disorder resulting from mutations in either the PKD1 or PKD2 gene. Polycystins are the protein products of PKD1 and PKD2, which encode polycystin-1 (PCI, 460 kDa) and polycystin-2 (PC2, 110 kDa), respectively. PC1 is a transmembrane receptor-like protein, and PC2 is a calcium channel. PC1 and PC2 can interact to form functional polycystin complexes that are widely expressed in various tissue types. Polycystins have a heterogenous distribution with localization to the primary cilia expressed in epithelial cells of the kidney, liver, pancreas, and breast, the smooth muscle and endothelial cells in the vasculature and astrocytes of the brain. Polycystins also have non-ciliary distribution. Both PC1 and PC2 appear to play roles in kidney development, and mutations in either gene can ultimately lead to cyst formation in ADPKD. Gene therapy has shown promise in diseases with single mutations such as cystic fibrosis, but it is not a favored approach because of the high genetic heterogeneity of ADPKD. The numerous ADPKD mutations in PKD1 and PKD2 genes are also highly variable.

Clinical trials are currently evaluating new therapies to reduce cyst formation and/or growth in ADPKD. Therapeutic approaches to interfere with the molecular pathways of cystogenesis are the focus of current drug development. Some strategies have targeted cAMP-mediated processes of transepithelial fluid secretion and epithelial cell proliferation, or mammalian target of rapamycin (mTOR)-mediated processes of cell proliferation and apoptotic cell death. It has been determined that certain inhibitors of cAMP, mTOR, CDK, TNF-alpha, the RAAS (renin angiotensin aldosterone system) and HMG-CoA reductase pathways reduce cyst formation and improve renal function in rat and mouse models of PKD. For example, Tolvaptan (vasopressin V2 receptor antagonist), sirolimus and everolimus (mTOR inhibitors), angiotensin converting enzyme (ACE) inhibitors (e.g., captopril), and angiotensin receptor blockers (ARBs, e.g. losartan), and statins that reduce cyst formation and improve renal function in animal models of PKD are being tested in clinical trials in humans. However, due to unknown clinical efficacy and side-effect profiles of current clinical candidates, there is a need for additional therapeutic approaches for the treatment of ADPKD.

### SUMMARY OF THE INVENTION

The disclosure is directed to methods of treating polycystic kidney disease by administration of coumarin-3-carboxamide novobiocin analogues either possessing or lacking a noviose sugar substituent. In one aspect, administration of the coumarin-3-carboxamide novobiocin analogue results in reduction of cyst formation in the kidneys. In one aspect, the PKD is autosomal dominant polycystic kidney disease.

The disclosure provides a method for treating, inhibiting, and/or preventing one or more symptoms of polycystic kidney disease (PKD) in a subject in need thereof, the method comprising administering to the subject a therapeutically effective amount of a compound according to Formula I:

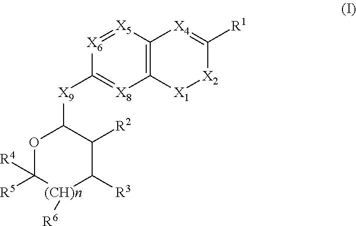

wherein R¹ is an amido which is NR′COR″, and R′ is hydrogen and R″ is C₁-C₄ alkyl, aryl or heterocycle, each optionally substituted with one or more hydroxy, nitro, amino, alkyl, alkenyl, aryl, alkoxy or halo groups; wherein R² is hydrogen, hydroxy, or —R⁸—OR⁹, wherein R⁸ is a covalent bond or alkyl, and R⁹ is C-amido or acyl; or R² together with R³ and the atoms to which they are attached form a heterocyclic ring having 4 to 8 ring members with at least one heteroatom selected from oxygen or nitrogen; wherein R³ is hydrogen, hydroxy, or —R¹⁰—O—R¹¹, wherein R¹⁰ is a covalent bond or alkyl, and R¹¹ is C-amido or acyl; or R³ together with R² and the atoms to which they are attached form a heterocyclic ring having 4 to 8 ring members with at least one heteroatom selected from oxygen or nitrogen; wherein R⁴ is hydrogen, hydroxy, alkyl, carboxyl, —R¹²—O—R¹³, or —R¹²-R¹⁴, and wherein R¹² is a covalent bond or alkyl, and R¹³ is C-amido or acyl, and R¹⁴ is N-amido, —POR¹⁵R¹⁶, —SO₂R¹⁷, or sulfonamido and wherein R¹⁵, R¹⁶, R¹⁷ are independently alkoxy; wherein R⁵ is hydrogen, alkyl, alkenyl, alkynyl, aryl, or aralkyl; wherein R⁶ is hydrogen, alkyl, alkenyl, alkynyl, aryl, aralkyl, alkoxy, aryloxy, or aralkoxy; wherein X₁ is —O—; wherein X₂ is —CO—; wherein X₄ is —CR²⁰—, wherein R²⁰ is hydrogen, alkyl, alkenyl, or alkynyl; wherein X₅ is —CR²¹, wherein R²¹ is hydrogen, alky, alkenyl, alkynyl, or alkoxy; wherein X₆, is —CR²² wherein R²² is hydrogen, alkyl, alkenyl, alkynyl, alkoxy, aryl, aralkyl, halogen, or nitro; wherein X₈, is —CR²³, wherein R²³ is hydrogen, alkyl, alkenyl, alkynyl, aryl, or alkoxy; wherein X₉ is alkyl, alkenyl, alkynyl, ether, secondary or tertiary amino, or sulfanyl; and wherein n is 1; or a pharmaceutically acceptable salt thereof.

The disclosure further provides a method for the manufacture of a medicament useful for treating, inhibiting, and/or preventing one or more symptoms of polycystic kidney disease (PKD) in a subject in need thereof, the method comprising preparation of a pharmaceutical composition useful for administering to the subject a therapeutically effective amount of a compound according to Formula I shown above.

In one aspect, the method of treating, inhibiting, and/or preventing a symptom of PKD refers to wherein the PKD is autosomal dominant polycystic kidney disease (ADPKD). In another aspect, the one or more symptoms are selected from the group consisting of cyst formation in the kidneys, increase in cyst size in the kidneys, increase in number of cysts in the kidneys, increase in kidney size, and end stage renal disease. In one aspect, the symptom is cyst formation in the kidneys.

In one aspect, the disclosure provides methods of treating, inhibiting, and/or preventing a symptom of PKD comprising administration of a compound of Formula I; and also provides methods for the manufacture of a medicament useful for treating, inhibiting, and/or preventing one or more symptoms of polycystic kidney disease (PKD) in a subject in need thereof, the method comprising preparation of a pharmaceutical composition useful for administering to the subject a therapeutically effective amount of a compound according to Formula I; wherein the compound is according to Formula I with the following substituents:

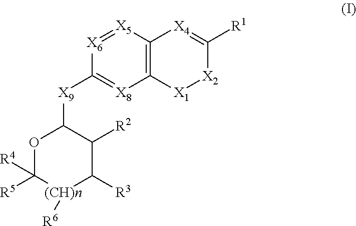

wherein R¹ is an amido which is NR′COR″, and R′ is hydrogen and R″ is C₁-C₄ alkyl, aryl or heterocycle, each optionally substituted with one or more hydroxy, nitro, amino, alkyl, alkenyl, aryl, alkoxy or halo groups; wherein R² is hydrogen, hydroxy, or —R⁸—OR⁹, wherein R⁸ is a covalent bond or alkyl, and R⁹ is C-amido or acyl; or R² together with R³ and the atoms to which they are attached form a heterocyclic ring having 4 to 8 ring members with at least one heteroatom selected from oxygen or nitrogen; wherein R³ is hydrogen, hydroxy, or —R¹⁰—O—R¹¹, wherein R¹⁰ is a covalent bond or alkyl, and R¹¹ is C-amido or acyl; or R³ together with R² and the atoms to which they are attached form a heterocyclic ring having 4 to 8 ring members with at least one heteroatom selected from oxygen or nitrogen; wherein R⁴ is hydrogen, hydroxy, alkyl, carboxyl, —R¹²—O—R¹³, or —R¹²-R¹⁴, and wherein R¹² is a covalent bond or alkyl, and R¹³ is C-amido or acyl, and R¹⁴ is N-amido, —POR¹⁵R¹⁶, —SO₂R¹⁷, or sulfonamido and wherein R¹⁵, R¹⁶, R¹⁷ are independently alkoxy; wherein R⁵ is hydrogen, alkyl, alkenyl, alkynyl, aryl, or aralkyl; wherein R⁶ is hydrogen, alkyl, alkenyl, alkynyl, aryl, aralkyl, alkoxy, aryloxy, or aralkoxy; wherein X₁ is —O—; wherein X₂ is —CO—; wherein X₄ is —CR²⁰—, wherein R²⁰ is hydrogen, alkyl, alkenyl, or alkynyl; wherein X₅ is —CR²¹, wherein R²¹ is hydrogen, alky, alkenyl, alkynyl, or alkoxy; wherein X₆, is —CR²² wherein R²² is hydrogen, alkyl, alkenyl, alkynyl, alkoxy, aryl, aralkyl, halogen, or nitro; wherein X₈, is —CR²³, wherein R²³ is hydrogen, alkyl, alkenyl, alkynyl, aryl, or alkoxy; wherein X₉ is alkyl, alkenyl, alkynyl, ether, secondary or tertiary amino, or sulfanyl; and wherein n is 1; or a pharmaceutically acceptable salt thereof.

In certain aspects, the disclosure provides methods of treating, inhibiting, and/or preventing a symptom of PKD comprising administration of a compound of Formula I; and also provides methods for the manufacture of a medicament useful for treating, inhibiting, and/or preventing one or more symptoms of polycystic kidney disease (PKD) in a subject in need thereof, the method comprising preparation of a pharmaceutical composition useful for administering to the subject a therapeutically effective amount of a compound according to Formula I; wherein the compound is according to Formula I with the following substituents:

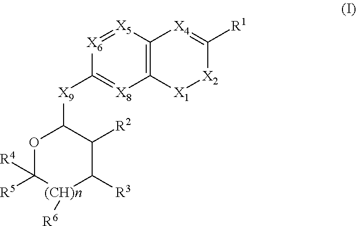

wherein R¹ is an amido which is NR′COR″, and R′ is hydrogen and R″ is aryl or heterocycle; wherein R² is hydrogen or hydroxy; wherein R³ is hydrogen or hydroxy; wherein R⁴ is hydrogen or methyl; wherein R⁵ is hydrogen, or alkyl; wherein R⁶ is alkoxy; wherein X₄ is —CR²⁰—, wherein R²⁰ is hydrogen; wherein X₅, is —CR²¹, wherein R²¹ is hydrogen; wherein X₆, is —CR²², wherein R²² is hydrogen, alkyl, or alkoxy; wherein X₈, is —CR²³, wherein R²³ is hydrogen, alkyl, or alkoxy; and wherein X₉ is ether.

In a particular aspect, the compound of formula I comprises certain substituents wherein R¹ is an amido which is NR′COR″, and R′ is hydrogen and R″ is aryl according to:

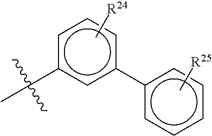

wherein R²⁴ and R²⁵ are independently hydrogen, alkyl, amino, halo, hydroxy, or alkoxy.

In a another aspect, the compound of formula I comprises certain substituents wherein R¹ is an amido which is NR′COR″, and R′ is hydrogen and R″ is aryl according to:

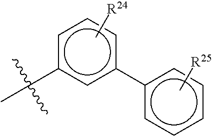

wherein R²⁴ and R²⁵ are hydrogen, alkyl or alkoxy.

In a another aspect, the compound of formula I comprises certain substituents wherein R¹ is an amido which is NR′COR″, and wherein R′ is hydrogen and R″ is aryl according to:

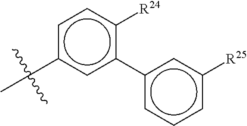

wherein R²⁴ and R²⁵ are alkoxy.

In another aspect, the disclosure provides methods of treating, inhibiting, and/or preventing a symptom of PKD comprising administration of a compound of Formula I; and also provides methods for the manufacture of a medicament useful for treating, inhibiting, and/or preventing one or more symptoms of polycystic kidney disease (PKD) in a subject in need thereof, the method comprising preparation of a pharmaceutical composition useful for administering to the subject a therapeutically effective amount of a compound according to Formula I; wherein the compound is according to Formula I is selected from N-(7-((2R,3R,4S,5R)-3,4-dihydroxy-5-methoxy-6,6-dimethyltetrahydro-2H-pyran-2-yloxy)-8-methyl-2-oxo-2H-chromen-3-yl)-6-methoxybiphenyl-3-carboxamide (28); N-(7-((2R,3R,4S,5R)-3,4-dihydroxy-5-methoxy-6,6-dimethyltetrahydro-2H-pyran-2-yloxy)-8-methyl-2-oxo-2H-chromen-3-yl)-6-methoxy-2′-methylbiphenyl-3-carboxamide (29); N-(7-((2R,3R,4S,5R)-3,4-dihydroxy-5-methoxy-6,6-dimethyltetrahydro-2H-pyran-2-yloxy)-8-methyl-2-oxo-2H-chromen-3-yl)-6-methoxy-3′-methylbiphenyl-3-carboxamide (30); N-(7-((2R,3R,4S,5R)-3,4-dihydroxy-5-methoxy-6,6-dimethyltetrahydro-2H-pyran-2-yloxy)-8-methyl-2-oxo-2H-chromen-3-yl)-6-methoxy-4′-methylbiphenyl-3-carboxamide (31); N-(7-((2R,3R,4S,5R)-3,4-dihydroxy-5-methoxy-6,6-dimethyltetrahydro-2H-pyran-2-yloxy)-8-methyl-2-oxo-2H-chromen-3-yl)-2′,6-dimethoxybiphenyl-3′-carboxamide (32); N-(7-((2R,3R,4S,5R)-3,4-dihydroxy-5-methoxy-6,6-dimethyltetrahydro-2H-pyran-2-yloxy)-8-methyl-2-oxo-2H-chromen-3-yl)-3′,6-dimethoxybiphenyl-3-carboxamide (33); N-(7-((2R,3R,4S,5R)-3,4-dihydroxy-5-methoxy-6,6-dimethyltetrahydro-2H-pyran-2-yloxy)-8-methyl-2-oxo-2H-chromen-3-yl)-4′,6-dimethoxybiphenyl-3′-carboxamide (34); N-(7-((2R,3R,4S,5R)-3,4-dihydroxy-5-methoxy-6,6-dimethyltetrahydro-2H-pyran-2-yloxy)-8-methyl-2-oxo-2H-chromen-3-yl)-2′-hydroxy-6-methoxybiphenyl-3-carboxamide (35); N-(7-((2R,3R,4S,5R)-3,4-dihydroxy-5-methoxy-6,6-dimethyltetrahydro-2H-pyran-2-yloxy)-8-methyl-2-oxo-2H-chromen-3-yl)-3′-hydroxy-6-methoxybiphenyl-3-carboxamide (36); and N-(7-((2R,3R,4S,5R)-3,4-dihydroxy-5-methoxy-6,6-dimethyltetrahydro-2H-pyran-2-yloxy)-8-methyl-2-oxo-2H-chromen-3-yl)-4′-hydroxy-6-methoxybiphenyl-3-carboxamide (37).

In another aspect, the compound according to Formula I comprises an R¹ which is an amido group which is NR′COR″, and wherein R′ is hydrogen and R″ is aryl according to:

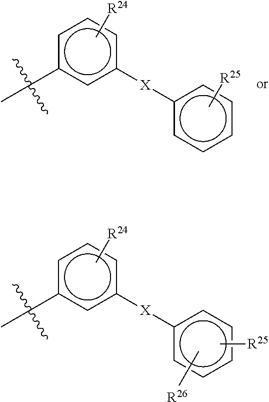

wherein X is ether or amino; wherein R²⁴ is alkoxy; wherein R²⁵ is hydrogen, hydroxy, alkoxy, or aryloxy; and wherein R²⁶ is hydrogen, alkoxy, aryloxy, or amino.

In another aspect, the compound according to Formula I comprises an R¹ amido which is NR′COR″, and wherein R′ is hydrogen and R″ is an indole according to:

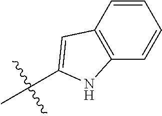

or a R″ is a pendant aryl according to:

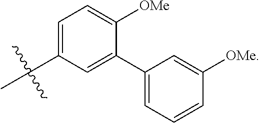

In another aspect the methods of the disclosure utilize a compound selected from the group consisting of N-(7-((2S,3R,4S,5R)-3,4-dihydroxy-5-methoxy-6,6-dimethyltetrahydro-2H-pyran-2-yloxy)-6-methoxy-8-methyl-2-oxo-2H-chromen-3-yl)-3′,6-dimethoxybiphenyl-3-carboxamide (26a); N-(7-((2S,3R,4S,5R)-3,4-dihydroxy-5-methoxy-6,6-dimethyltetrahydro-2H-pyran-2-yloxy)-8-methyl-2-oxo-6-propoxy-2H-chromen-3-yl)-3′,6-dimethoxybiphenyl-3-carboxamide (26b); N-(7-((2S,3R,4S,5R)₇3,4-dihydroxy-5-methoxy-6,6-dimethyltetrahydro-2H-pyran-2-yloxy)-6-isopropoxy-8-methyl-2-oxo-2H-chromen-3-yl)-3′,6-dimeth-oxybiphenyl-3-carboxamide (26c); N-(7-((2R,3R,4S,5R)-3,4-dihydroxy-5-methoxy-6,6-dimethyltetrahydro-2H-pyran-2-yloxy)-5-methoxy-8-methyl-2-oxo-2H-chromen-3-yl)-3′,6-dimethoxybiphenyl-3-carboxamide (26d); N-(8-benzyl-7-((2R,3R,4S,5R)-3,4-dihydroxy-5-methoxy-6,6-dimethyltetrahydro-2H-pyran-2-yloxy)-2-oxo-2H-chromen-3-yl)-3′,6-dimethoxybiphenyl-3-carboxamide (26e); N-(7-((2R,3R,4S,5R)-3,4-dihydroxy-5-methoxy-6,6-dimethyltetrahydro-2H-pyran-2-yloxy)-2-oxo-8-phenyl-2H-chromen-3-yl)-3′,6-dimethoxybiphenyl-3-carboxamide (26f); N-(7-((2S,3R,4S,5R)-3,4-dihydroxy-5-methoxy-6,6-dimethyltetrahydro-2H-pyran-2-yloxy)-8-methoxy-2-oxo-2H-chromen-3-yl)-3′,6-dimethoxybiphenyl-3-carboxamide (26g, KU-174); N-(7-((2R,3R,4S,5R)-3,4-dihydroxy-5-methoxy-6,6-dimethyltetrahydro-2H-pyran-2-yloxy)-8-ethyl-2-oxo-2H-chromen-3-yl)-3′,6-dimethoxybiphenyl-3-carboxamide (26h).

In one specific aspect, the disclosure provides a method of treating, inhibiting, and/or preventing a symptom of PKD comprising administration of a compound of Formula I; or a method for the manufacture of a medicament useful for treating, inhibiting, and/or preventing one or more symptoms of polycystic kidney disease (PKD) in a subject in need thereof, the method comprising preparation of a pharmaceutical composition useful for administering to the subject a therapeutically effective amount of a compound according to Formula I; wherein the compound is according to Formula I is N-(7-((2S,3R,4S,5R)-3,4-dihydroxy-5-methoxy-6,6-dimethyltetrahydro-2H-pyran-2-yloxy)-8-methoxy-2-oxo-2H-chromen-3-yl)-3′,6-dimethoxybiphenyl-3-carboxamide (26g, KU-174).

In other aspects, the methods of the disclosure utilize a compound selected from the group consisting of: N-(7-((2S,3R,4S,5R)-3,4-dihydroxy-5-methoxy-6,6-dimethyltetrahydro-2H-pyran-2-yloxy)-6-methoxy-8-methyl-2-oxo-2H-chromen-3-yl)-1H-indole-2-carboxamide (26i); N-(7-((2S,3R,4S,5R)-3,4-dihydroxy-5-methoxy-6,6-dimethyltetrahydro-2H-pyran-2-yloxy)-8-methyl-2-oxo-6-propoxy-2H-chromen-3-yl)-1H-indole-2-carboxamide (26j); N-(7-((2S,3R,4S,5R)-3,4-dihydroxy-5-methoxy-6,6-dimethyltetrahydro-2H-pyran-2-yloxy)-6-isopropoxy-8-methyl-2-oxo-2H-chromen-3-yl)-1H-indole-2-carboxamide (26k); N-(7-((2R,3R,4S,5R)-3,4-dihydroxy-5-methoxy-6,6-dimethyltetrahydro-2H-pyran-2-yloxy)-5-methoxy-8-methyl-2-oxo-2H-chromen-3-yl)-1H-indole-2-carboxamide (261); N-(8-benzyl-7-((2R,3R,4S,5R)-3,4-dihydroxy-5-methoxy-6,6-dimethyltetrahydro-2H-pyran-2-yloxy)-2-oxo-2H-chromen-3-yl)-1H-indole-2-carboxamide (26m); N-(7-((2R,3R,4S,5R)-3,4-dihydroxy-5-methoxy-6,6-dimethyltetrahydro-2H-pyran-2-yloxy)-2-oxo-8-phenyl-2H-chromen-3-yl)-1H-indole-2-carboxamide (26n); N-(7-((2S,3R,4S,5R)-3,4-dihydroxy-5-methoxy-6,6-dimethyltetrahydro-2H-pyran-2-yloxy)-8-methoxy-2-oxo-2H-chromen-3-yl)-1H-indole-2-carboxamide (26o); and N-(7-((2R,3R,4S,5R)-3,4-dihydroxy-5-methoxy-6,6-dimethyltetrahydro-2H-pyran-2-yloxy)-8-ethyl-2-oxo-2H-chromen-3-yl)-1H-indole-2-carboxamide (26p).

In other aspects, the methods of the disclosure provide for use of a compound of Formula I wherein R¹ is an amido which is NR′COR″, and R′ is hydrogen and R″ is a heterocycle selected from the group consisting of pyridine, benzofuran, indole, and oxazole.

In further aspects, the methods of the disclosure provide for use of a compound of Formula I wherein R¹ is an amido which is NR′COR″, and R′ is hydrogen and R″ is aryl or heterocycle according to:

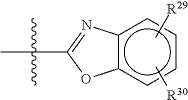

wherein R²⁹ is hydrogen, alkoxy, or amino; and wherein R³⁰ is hydrogen, alkoxy, or aryloxy.

In other aspects, the methods of the disclosure provide for use of a compound of Formula I wherein R¹ is an amido which is NR′COR″, and R′ is hydrogen and R″ is a heterocycle according to:

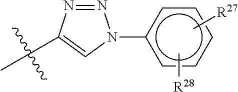

wherein R²⁷ is hydrogen, hydroxy, alkoxy, or aryloxy; and wherein R²⁸ is hydrogen, alkoxy, aryloxy, or amino. In further aspects, the methods of the disclosure provide for use of a compound of Formula I wherein R¹ is an amido which is NR′COR″, and R′ is hydrogen and R″ is a heterocyle according to:

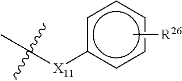

wherein X₁₁ is a covalent bond, alkyl, alkenyl, alkynyl, or —OCH₂— wherein R²⁶ is hydrogen, aryl, amino, or hydroxy.

In further aspects, the compound of Formula I is selected wherein R¹ is an amido which is NR′COR″, and W is hydrogen and R″ is aryl according to one of the following:

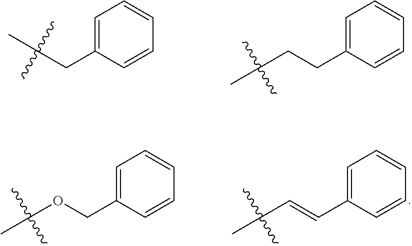

In further aspects, the compound of Formula I is selected from the group consisting of N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)-2-phenylacetamide (22); N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)-3-phenylpropanamide (23); Benzyl 7-((2R,3R,4S,5R)-3,4-dihydroxy-5-methoxy-6,6-dimethyltetrahydro-2H-pyran-2-yloxy)-8-methyl-2-oxo-2H-chromen-3-ylcarbamate (24); and N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)cinnamamide (25).

In further aspects, the compound of Formula I is selected wherein R¹ is an amido which is NR′COR″, and R′ is hydrogen and R″ is a heterocycle according to one of the following:

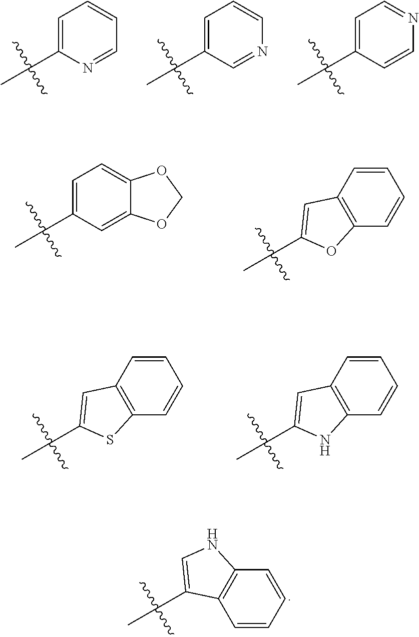

In further aspects, the compound of Formula I is selected from the group consisting of N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)picolinamide (40); N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)nicotinamide (41); N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)isonicotinamide (42); N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)benzofuran-2-carboxamide (43); N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)-1H-indole-2-carboxamide (46); and N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)-1H-indole-3-carboxamide (47). In one specific aspect, the compound of formula I is N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)-1H-indole-2-carboxamide (46).

In further aspects, the compound of Formula I is selected from the group consisting of N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)benzamide (8); N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)biphenyl-2-carboxamide (12); N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)biphenyl-3-carboxamide (13); 2-Amino-N-(7-((2R,3R,4S,5R)-3,4-dihydroxy-5-methoxy-6,6-dimethyltetrahydro-2H-pyran-2-yloxy)-8-methyl-2-oxo-2H-chromen-3-yl)benzamide (18); 3-Amino-N-(7-((2R,3R,4S,5R)-3,4-dihydroxy-5-methoxy-6,6-dimethyltetrahydro-2H-pyran-2-yloxy)-8-methyl-2-oxo-2H-chromen-3-yl)benzamide (19); 4-Amino-N-(7-((2R,3R,4S,5R)-3,4-dihydroxy-5-methoxy-6,6-dimethyltetrahydro-2H-pyran-2-yloxy)-8-methyl-2-oxo-2H-chromen-3-yl)benzamide (20); N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)-2-methoxybenzamide (9); N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)-3-methoxybenzamide (10); and N-(7-((2R,3R4S,5R)-3,4-Dihydroxy-5-methoxy-6,6-dimethyltetrahydro-2H-pyran-2-yloxy)-8-methyl-2-oxo-2H-chromen-3-yl)-4-methoxybenzamide (11).

In another specific aspect, the compound of Formula I is KU-32:

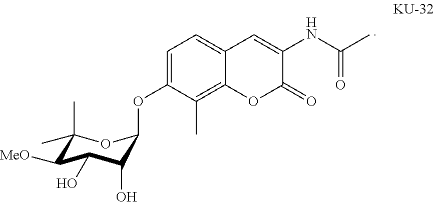

In another embodiment, the disclosure provides methods of treating, inhibiting, and/or preventing a symptom of PKD comprising administration of a compound of Formula II; and also provides methods for the manufacture of a medicament useful for treating, inhibiting, and/or preventing one or more symptoms of polycystic kidney disease (PKD) in a subject in need thereof, the method comprising preparation of a pharmaceutical composition useful for administering to the subject a therapeutically effective amount of a compound according to Formula II:

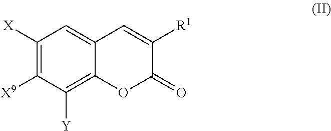

Wherein R¹ is —NHCOR″, where R″ is a C₁-C₄ alkyl, aryl or heterocyclic group, each optionally substituted with one or more hydroxy, nitro, amino, alkyl, alkenyl, aryl, alkoxy or halo groups; X⁹ is —O-alkyl, —O-alkylamino, —O-cycloalkyl, —O—(CO)-alkyl, —O—(CO)-cycloalkyl, —O—(CH₂)ₙ-pyridinyl, —O—(CH₂)ₙ-piperidinyl, —O—(CH₂)ₙ-pyrrolino, or —O—(CH₂)ₙ-pyrrolidinyl, each substituted with one or more amino, amido, alkyl, alkoxy, halo, pyrrolidinyl, or hydroxyl groups; and where n is 0, 1 2 or 3; or —O-mono-hydroxylated furanose, —O-dihydroxylated furanose, —O-mono-hydroxylated pyranose, —O-dihydroxylated pyranose, —O-trihydroxylated pyranose, —O-mono-hydroxylatedoxepinose, —O-dihydroxylated oxepinose, —O-azasugar, —O-acyl, ester, amino, amido, carbamate, phosphate ester, tosylate, mesylate or —OH; X is H, nitrile, halo, amino, amido, C₁-C₄ alkyl or alkoxy; and Y is H, amido, ester, amino, C₁-C₄ alkyl or alkoxy; or a pharmaceutically acceptable salt thereof.

In one aspect, the compound of Formula II is selected wherein X₉ is —O-alkyl, —O-alkylamino, —O-cycloalkyl, —O—(CO)-alkyl, —O—(CO)-cycloalkyl, —O—(CH₂)ₙ-pyridinyl, —O—(CH₂)ₙ-piperidinyl, —O—(CH₂)ₙ-pyrrolino, or —O—(CH₂)ₙ-pyrrolidinyl, each optionally substituted with one or more amino, amido alkyl, halo, alkoxy, or hydroxyl groups.

In another aspect, the compound of Formula II is selected wherein X₉ is

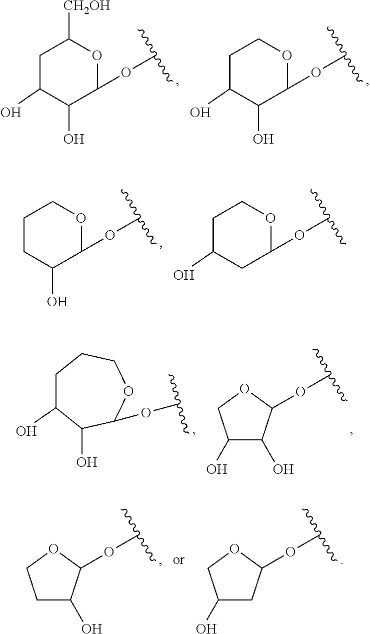

In another aspect, the compound of Formula II is selected wherein is —NHCOCH₃.

In another aspect, the compound of Formula II is selected wherein R″ is an aryl group selected from:

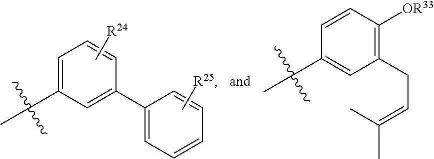

wherein R²⁴ and R²⁵ are independently H, C₁-C₄ alkyl, hydroxy or alkoxy; and R³³ is H, C₁-C₄ alkyl, C₁-C₄ alkylamino, —(CO)—C₁-C₄ alkyl, or piperidinyl, each optionally substituted with C₁-C₄ alkyl; or R″ is a heterocyclic group:

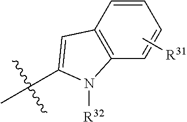

wherein R³¹ is H, halo, C₁-C₄ alkyl, hydroxy or alkoxy; and R³² is H or C₁-C₄ alkyl.

In another aspect, the compound of Formula II is selected wherein R″ is an aryl group:

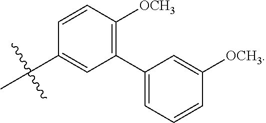

In a further aspect, the compound of Formula II is selected from the group consisting of:

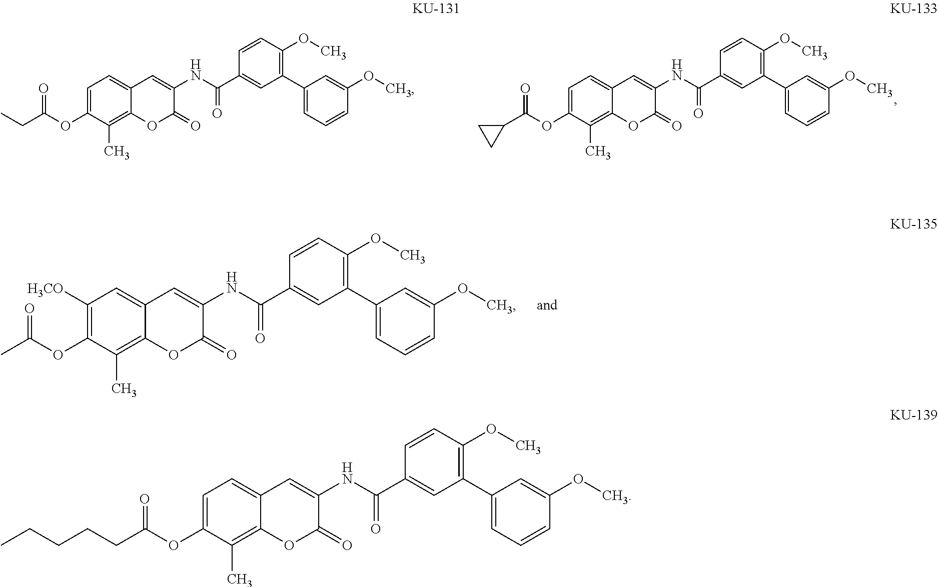

In another aspect, the compound of Formula II is selected wherein R″ is aryl according to:

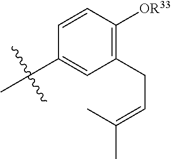

wherein R³³ is H, —CH₃, —COCH₃, —CH₂CH₂N(CH₃)₂, —CH₂CH₂CH₂N(CH₃)₂, or

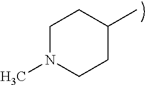

In a further aspect, the compound of Formula II is selected from the group consisting of 4-(8-Methyl-7-(1-methylpiperidin-4-yloxy)-2-oxo-2H-chromen-3-ylcarbamoyl)-2-(3-methylbut-2-enyl)phenyl acetate (29a, KU-397); 4-(8-Methyl-7-(1-methylpiperidin-3-yloxy)-2-oxo-2H-chromen-3-ylcarbamoyl)-2-(3-methylbut-2-enyl)phenyl acetate (29c, KU-417); 44742-(Dimethylamino)ethoxy)-8-methyl-2-oxo-2H-chromen-3-ylcarbamoyl)-2-(3-methylbut-2-enyl)phenyl acetate (29e, KU-421); 4-(7-(3-(Dimethylamino)propoxy)-8-methyl-2-oxo-2H-chromen-3-ylcarbamoyl)-2-(3-methylbut-2-enyl)phenyl acetate (29f, KU-406); 4-(8-methyl-2-oxo-7-(piperidin-4-yloxy)-2H-chromen-3-ylcarbamoyl)-2-(3-methylbut-2-enyl)phenyl acetate (30b, KU-415); 4-(8-methyl-2-oxo-7-(piperidin-3-yloxy)-2H-chromen-3-ylcarbamoyl)-2-(3-methylbut-2-enyl)phenyl acetate (30d, KU-419); 4-(8-methyl-7-(2-(methylamino)ethoxy)-2-oxo-2H-chromen-3-ylcarbamoyl)-2-(3-methylbut-2-enyl)phenyl acetate (30g, KU-423); 4-Hydroxy-N-(8-methyl-7-(1-methylpiperidin-4-yloxy)-2-oxo-2H-chromen-3-yl)-3-(3-methylbut-2-enyl)benzamide (31a, KU-398); 4-Hydroxy-N-(8-methyl-2-oxo-7-(piperidin-4-yloxy)-2H-chromen-3-yl)-3-(3-methylbut-2-enyl)benzamide (31b, KU-416); 4-Hydroxy-N-(8-methyl-7-(1-methylpiperidin-3-yloxy)-2-oxo-2H-chromen-3-yl)-3-(3-methylbut-2-enyl)benzamide (31c, KU-418); 4-Hydroxy-N-(8-methyl-2-oxo-7-(piperidin-3-yloxy)-2H-chromen-3-yl)-3-(3-methylbut-2-enyl)benzamide (31d, KU-420); N-(7-(2-(Dimethylamino)ethoxy)-8-methyl-2-oxo-2H-chromen-3-yl)-4-hydroxy-3-(3-methylbut-2-enyl)benzamide (31e, KU-422); N-(7-(3-(Dimethylamino)propoxy)-8-methyl-2-oxo-2H-chromen-3-yl)-4-hydroxy-3-(3-methylbut-2-enyl)benzamide (31f, KU-407); 4-Hydroxy-N-(8-methyl-7-(2-(methylamino)ethoxy)-2-oxo-2H-chromen-3-yl)-3-(3-methylbut-2-enyl)benzamide (31g, KU-424); N-(7-((2R,3R,4R)-3,4-dihydroxytetrahydro-2H-pyran-2-yloxy)-8-methyl-2-oxo-2H-chromen-3-yl)-4-hydroxy-3-(3-methylbut-2-enyl)benzamide (16a, KU-425); N-(7-((2S,3R,4R)-3,4-dihydroxytetrahydro-2H-pyran-2-yloxy)-8-methyl-2-oxo-2H-chromen-3-yl)-4-hydroxy-3-(3-methylbut-2-enyl)benzamide (16b, KU-426); 4-Hydroxy-N-(7-((2R,3R)-3-hydroxytetrahydro-2H-pyran-2-yloxy)-8-methyl-2-oxo-2H-chromen-3-yl)-3-(3-methylbut-2-enyl)benzamide (17a, KU-247); 4-Hydroxy-N-(7-((2S,3R)-3-hydroxytetrahydro-2H-pyran-2-yloxy)-8-methyl-2-oxo-2H-chromen-3-yl)-3-(3-methylbut-2-enyl)benzamide (17b, KU-428); 4-Hydroxy-N-(7-((2S,4R)-4-hydroxytetrahydro-2H-pyran-2-yloxy)-8-methyl-2-oxo-2H-chromen-3-yl)-3-(3-methylbut-2-enyl)benzamide (18a, KU-429); 4-Hydroxy-N-(7-((2R,4R)-4-hydroxytetrahydro-2H-pyran-2-yloxy)-8-methyl-2-oxo-2H-chromen-3-yl)-3-(3-methylbut-2-enyl)benzamide (18b, KU-430); 4-(7-((2S,3S,4S)-3,4-Dihydroxytetrahydrofuran-2-yloxy)-8-methyl-2-oxo-2H-chromen-3-ylcarbamoyl)-2-(3-methylbut-2-enyl)phenyl acetate (19, KU-431); 4-(7-((2S,4R)-4-Hydroxytetrahydrofuran-2-yloxy)-8-methyl-2-oxo-2H-chromen-3-ylcarbamoyl)-2-(3-methylbut-2-enyl)phenyl acetate (20a, KU-432); and 4-(7-((2R,4R)-4-Hydroxytetrahydrofuran-2-yloxy)-8-methyl-2-oxo-2H-chromen-3-ylcarbamoyl)-2-(3-methylbut-2-enyl)phenyl acetate (20b, KU-433).

In a further aspect, the compound of Formula II is selected from the group consisting of

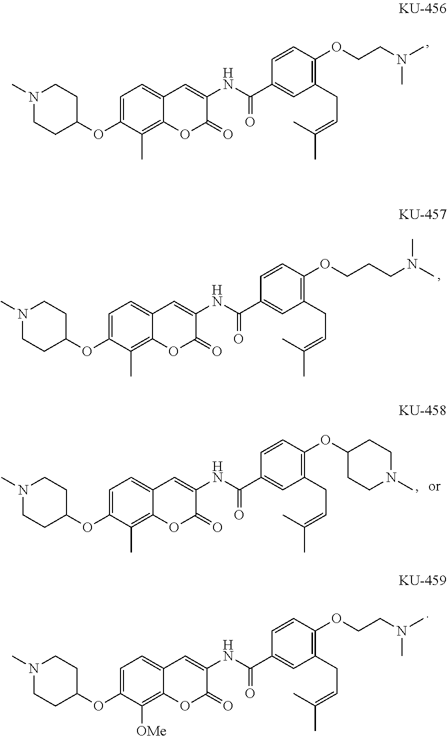

In a further aspect, the compound of Formula II is selected wherein R″ is a heterocyclic group according to:

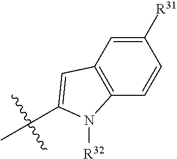

wherein R³¹ is H, halo, or alkoxy; and R³² is H or alkyl.

In another embodiment, the disclosure provides methods of treating, inhibiting, and/or preventing a symptom of PKD comprising administration of a compound of Formula III; and also provides methods for the manufacture of a medicament useful for treating, inhibiting, and/or preventing one or more symptoms of polycystic kidney disease (PKD) in a subject in need thereof, the method comprising preparation of a pharmaceutical composition useful for administering to the subject a therapeutically effective amount of a compound according to Formula III:

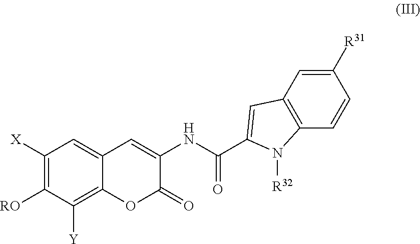

wherein X is H or —OCH₃; Y is —CH₃ or —OCH₃; R³¹ is H, Cl, —CH₂CH₂N(CH₃)₂, —CH₂CH₂CH₂N(CH₃)₂, —OCH₃, or

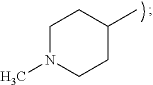

### R³² is H or —CH₃; and

R is selected from the group consisting of:

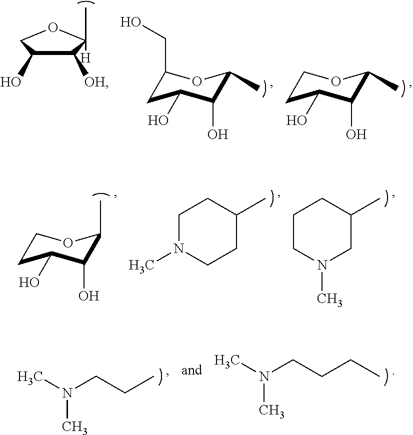

In a further aspect, the compound of Formula III is selected from the group consisting of

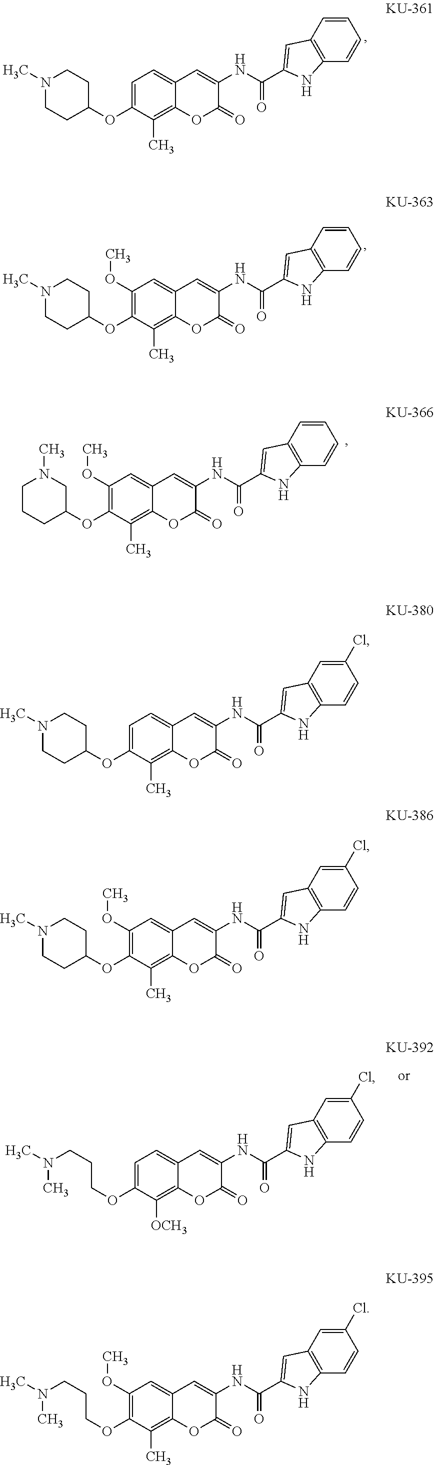

In another embodiment, the disclosure provides methods of treating, inhibiting, and/or preventing a symptom of PKD comprising administration of a compound of Formula III; and also provides methods for the manufacture of a medicament useful for treating, inhibiting, and/or preventing one or more symptoms of polycystic kidney disease (PKD) in a subject in need thereof, the method comprising preparation of a pharmaceutical composition useful for administering to the subject a therapeutically effective amount of a compound according to Formula III:

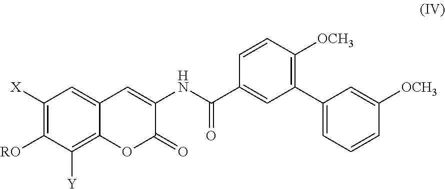

wherein X is H or —OCH₃; Y is —CH₃ or —OCH₃; and R is selected from the group consisting of: H, —COCH₃, mesylate, tosylate, —CONH₂, —CONHCH₃, —CON(CH₃)₂, —PO(OCH₃)₂, —COCH₃,

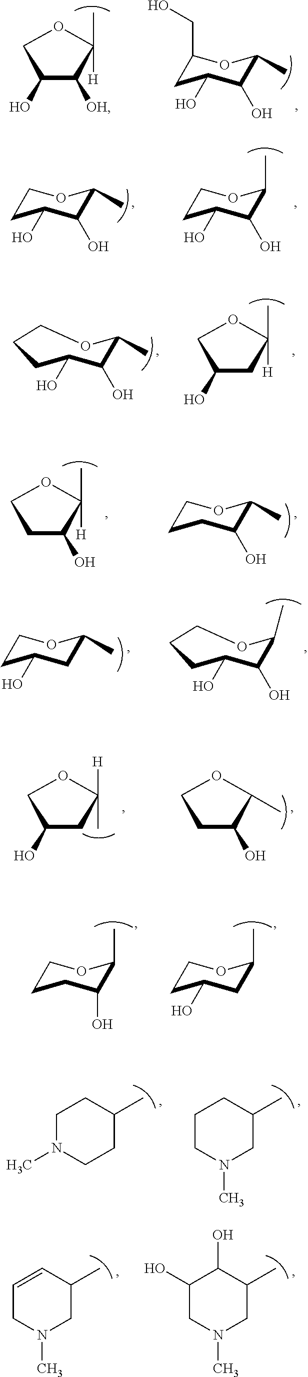

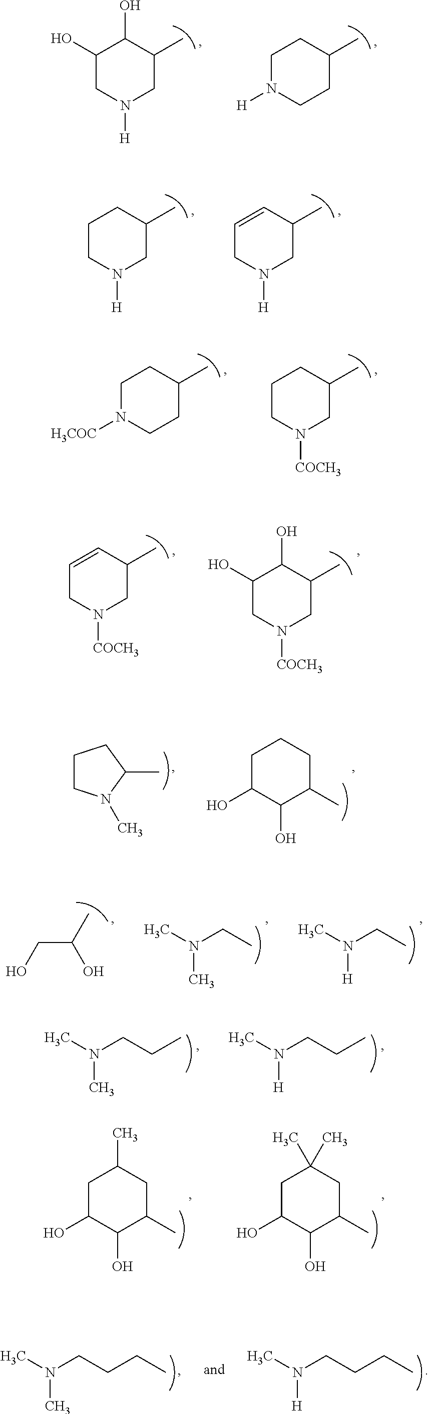

In a further aspect, the compound of Formula IV is selected from the group consisting of

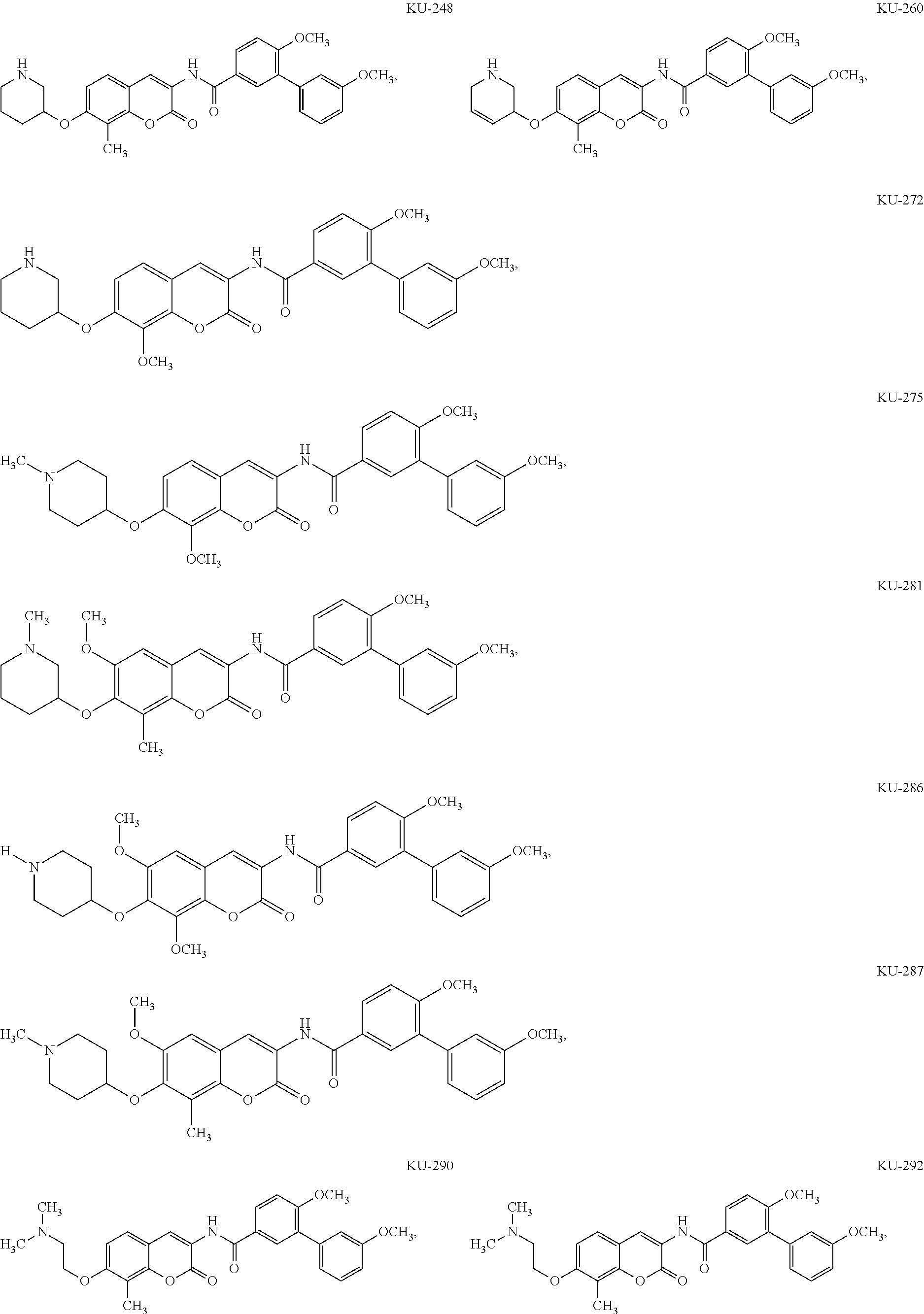

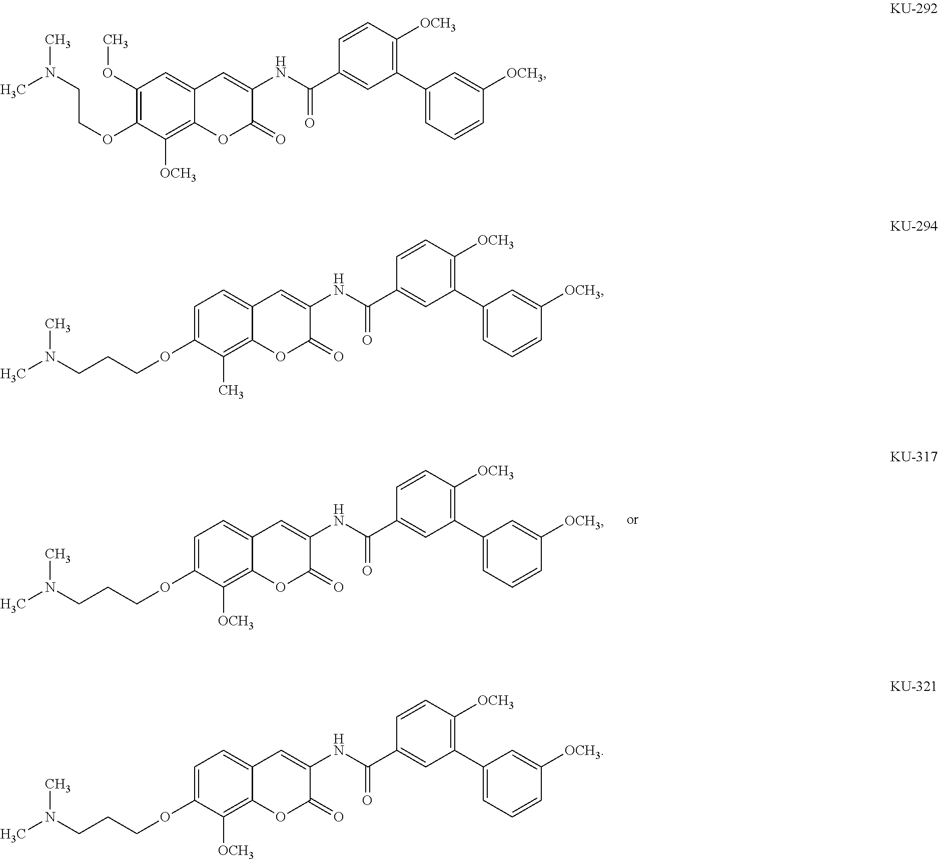

### BRIEF DESCRIPTION OF THE DRAWINGS

FIG. 1 shows dose-dependent decrease in the proliferation of cAMP or EGF stimulated ADPKD cells treated with KU-174 by MIT assay.

FIG. 2 shows dose-dependent inhibition of p-ERK in EGF stimulated ADPKD cells treated with KU-174 by Western blot.

FIG. 3A shows Western blot of mTOR pathway mediators p-Akt, Akt, p-TSC2, and TSC2 from EGF-stimulated ADPKD cells at 24 and 48 hours post-stimulation.

FIG. 3B shows Western blot of mTOR pathway mediators p-mTOR, mTOR, p-S6K and S6K EGF-stimulated cells at 24 and 48 hours.

FIG. 4 shows Western blot of Hsp 90 client proteins CFTR, C-Raf, cdk4, ErbB2 and Akt, as well as Hsp90 and GAPDH, in EGF stimulated ADPKD cells treated with KU-174 at various concentrations at 24 and 48 hours post-stimulation.

FIG. 5 shows dose-dependent inhibition of microcyst formation by ADPKD cells stimulated with EGF and forskolin in a collagen gel matrix containing cysts treated with KU-174 and fixed in 1% formaldehyde.

FIG. 6 shows total cyst surface area per well (n=6 wells per treatment) of microcysts formed by ADPKD cells stimulated with EGF and forskolin in a collagen gel matrix containing cysts treated with KU-174 and fixed in 1% formaldehyde measured using a morphometric analysis system; 1 μM KU-174 caused a significant inhibition in cyst growth, and 5 μM KU-174 completely eliminated cyst formation (*p<0.01).

FIG. 7A shows KU-174 treatment reduces the cyst burden in cAMP-treated Pkd1+/− mouse metanephric kidneys. Embryonic day 15.5 mouse kidneys were plated on Transwell membranes and treated with 100 μM cAMP along with 10 μM KU-174 or captisol (vehicle) for four days.

FIG. 7B also shows a reduction in cyst burden in different cAMP-treated Pkd1+/− mouse metanephric kidneys treated with KU-174 under the same conditions as in FIG. 7A.

### DEFINITION OF TERMS

Molecular terms, when used in this application, have their common meaning unless otherwise specified. It should be noted that the alphabetical letters used in the formulas of the present disclosure should be interpreted as the functional groups, moieties, or substituents as defined herein. Unless otherwise defined, the symbols have their ordinary and customary meaning to those skilled in the art.

As used herein, an “azasugar” refers to a sugar in which the ring-oxygen is replaced with an amino-group. The “azasugars” are preferably 1, 3 or 1,4 azasugars, and the amino group may be either a secondary or tertiary amino group. Preferred tertiary amino groups are substituted with an alkyl or acyl group. In addition, the azasugar ring may be saturated or unsaturated.

As used herein, the term “sugar” refers to a sugar group in its cyclic form, for example, those derived from furanose (5-membered ring), pyranose (6-membered ring), or oxepanose (7-membered ring). Exemplary sugars are set forth in Yu et al, Synthesis of mono- and dihydroxylated furanoses, pyranoses, and an oxepanose for the preparation of natural product analogue libraries, J. Org. Chem. 70(14):5599-605 (2005), Harris et al., Syntheses of D- and L-Mannose, Gulose, and Talose via Diastereoselective and Enantioselective Dihydroxylation Reactions, J. Org. Chem. 1999 64(9), 2982-2983 (1999); Ahmed et al., De novo enantioselective syntheses of galacto-sugars and deoxy sugars via the iterative dihydroxylation of dienoate, Org. Lett. 2005 7(4), 745-748 (2005); Haukaas et al., Enantioselective synthesis of 2-deoxy- and 2,3-dideoxyhexoses, Org. Lett. 2002 4(10), 1771-1774 (2002), all of which are incorporated by reference. Exemplary of sugar groups include threofuranosyl (from threose, a four-carbon sugar); ribofuranosyl (from ribose, a five-carbon sugar); arafuranosyl (also often referred to as arabinofuranosyl; from arabinose, a five-carbon sugar); xylofuranosyl (from xylose, a five-carbon sugar), and lyxofuranosyl (from lyxose, a five-carbon sugar). The sugar may be mono-hydroxylated or poly-hydroxylated (e.g., di-hydroxylated, tri-hydroxylated).

The term “carbamate” refers to —COONHR, wherein R as used in this definition hydrogen, alkyl, aryl, or heteroaromatic.

The term “phosphate ester” refers to —PO³R′R″, wherein R′ and R″ are independently hydrogen, alkyl, aryl, heteroaromatic.

The term “alcohol” indicates an optionally substituted hydrocarbon group having one or more hydroxy substituents. Exemplary alcohols include alkanols containing from about one up to twelve carbon atoms, with alkanols having one to up to six carbon atoms being most preferred. Exemplary of preferred aliphatic alcohols are: methanol, ethanol, 1-propanol, 2-propanol, 1-propen-2-ol, 1-butanol, 2-butanol, 2-methyl-2-propanol, 1-pentanol, 2-pentanol, 3-pentanol, 3-methyl-1-butanol, 1,2-ethandiol (ethylene glycol), 1,2,3-propantriol (glycerol), i-1,2,3,4-butantetrol (1-erythritol), and 2,2-dihydroxymethyl-1,3-propandiol (pentaerythritol). When used as a sugar mimic at the 9-position, the alcohol is preferably poly-hydroxylated.

The terms “acyl” or “Ac” refers to —COR wherein R used in this definition is hydrogen, alkyl, alkenyl, alkynyl, carbocyclic, heterocylic, aryl, or aralkyl. Most preferably, R is hydrogen, alkyl, aryl, or aralkyl.

The term “amido” indicates either a C-amido group such as —CONR′R″ or an N-amido group such as —NR′COR″ wherein R′ and R″ as used in this definition are independently hydrogen, alkyl, alkenyl, alkynyl, alkoxy, carbocyclic, heterocylic, aryl, or aralkyl. A “sulfoamido” group includes the —NR′—SO₂—R″. Most preferably, R′ is hydrogen and R″ is alkyl, aryl, heterocyclic or aralkyl.

The term “amino” signifies a primary, secondary or tertiary amino group of the formula —NR′R″ wherein R′ and R″ as used in this definition are independently hydrogen, alkyl, alkyenyl, alkynyl, aralkyl, carbocyclic, heterocyclic, aralkyl, or other amino (in the case of hydrazide) or R′ and R″ together with the nitrogen atom to which they are attached, form a ring having 4 to 8 atoms. Thus, the term “amino,” as used herein, includes unsubstituted, monosubstituted (e.g., monoalkylamino or monoarylamino), and disubstituted (e.g., dialkylamino or aralkylamino) amino groups. Amino groups include —NH₂, methylamino, ethylamino, dimethylamino, diethylamino, methyl-ethylamino, pyrrolidin-1-yl, or piperidino, morpholino, etc. Other exemplary “amino” groups forming a ring include pyrrolyl, imidazolyl, pyrazolyl, isothiazolyl, isoxazolyl, pyridyl, pyrazinyl, pyrimidinyl, pyridazinyl, indolizinyl, isoindolyl, indolyl, indazolyl, purinyl, quinolizinyl. The ring containing the amino group may be optionally substituted with another amino, alkyl, alkenyl, alkynyl, halo, or hydroxyl group.

The term “alkyl” refers to a branched or unbranched saturated hydrocarbon group of 1 to 24 carbon atoms, such as methyl, ethyl, n-propyl, isopropyl, n-butyl, isobutyl, t-butyl, octyl, decyl, tetradecyl, hexadecyl, eicosyl, tetracosyl, and the like. Preferred “alkyl” groups herein contain 1 to 12 carbon atoms. Most preferred are “lower alkyl” which refer to an alkyl group of one to six, more preferably one to four, carbon atoms. The alkyl group may be optionally substituted with an amino, alkyl, halo, or hydroxyl group.

The term “alkoxy” denotes oxy-containing groups substituted with an alkyl, or cycloalkyl group. Examples include, without limitation, methoxy, ethoxy, tert-butoxy, and cyclohexyloxy. Most preferred are “lower alkoxy” groups having one to six carbon atoms. Examples of such groups include methoxy, ethoxy, propoxy, butoxy, isopropoxy, and tert-butoxy groups.

The terms “alkenyl” and “alkynyl” refer to unsaturated aliphatic groups analogous in length and possible substitution to the alkyls described above, but that contain at least one double bond or triple bond respectively.

The term “aryl” means a carbocyclic aromatic system containing one, two, or three rings wherein such rings may be attached together in a pendant manner or may be fused. The term “fused” means that a second ring is present (i.e., attached or formed) by having two adjacent atoms in common (i.e., shared) with the first ring. The term “fused” is equivalent to the term “condensed.” The term “aryl” embraces aromatic groups such as phenyl, naphthyl, tetrahydronaphthyl, indane, and biphenyl. The aryl group may optionally be substituted with an amino, alkyl, halo, alkenyl, alkoxy, hydroxyl, carbocyclic, heterocyclic, or another aryl group. A preferred aryl is a pendant aryl according to:

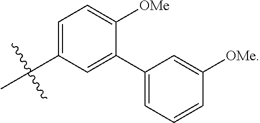

The term “aralkyl” embraces aryl-substituted alkyl moieties. Preferable aralkyl groups are “lower aralkyl” groups having aryl groups attached to alkyl groups having one to six carbon atoms. Examples of such groups include benzyl, diphenylmethyl, triphenylmethyl, phenylethyl, and diphenylethyl. The terms benzyl and phenylmethyl are interchangeable.

The term “aryloxy” embraces aryl groups, as defined above, attached to an oxygen atom. The aryloxy groups may optionally be substituted with a halo, hydroxyl, or alkyl group. Examples of such groups include phenoxy, 4-chloro-3-ethylphenoxy, 4-chloro-3-methylphenoxy, 3-chloro-4-ethylphenoxy, 3,4-dichlorophenoxy, 4-methylphenoxy, 3-trifluoromethoxyphenoxy, 3-trifluoromethylphenoxy, 4-fluorophenoxy, 3,4-dimethylphenoxy, 5-bromo-2-fluorophenoxy, 4-bromo-3-fluorophenoxy, 4-fluoro-3-methylphenoxy, 5,6,7,8-tetrahydronaphthyloxy, 3-isopropylphenoxy, 3-cyclopropylphenoxy, 3-ethylphenoxy, 4-tert-butylphenoxy, 3-pentafluoroethylphenoxy, and 3-(1,1,2,2-tetrafluoroethoxy)phenoxy.

The term “aralkoxy” embraces oxy-containing aralkyl groups attached through an oxygen atom to other groups. “Lower aralkoxy” groups are those phenyl groups attached to lower alkoxy group as described above. Examples of such groups include benzyloxy, 1-phenylethoxy, 3-trifluoromethoxybenzyloxy, 3-trifluoromethylbenzyloxy, 3,5-difluorobenzyloxy, 3-bromobenzyloxy, 4-propylbenzyloxy, 2-fluoro-3-trifluoromethylbenzyloxy, and 2-phenylethoxy.

The term “carboxyl” refers to —R′C(═O)OR″, wherein R′ and R″ as used in this definition are independently hydrogen, alkyl, alkenyl, alkynyl, carbocyclic, heterocylic, aryl, or aralkyl or R′ can additionally be a covalent bond. “Carboxyl” includes both carboxylic acids, and carboxylic acid esters. The term “carboxylic acid” refers to a carboxyl group in which R″ is hydrogen. Such acids include formic, acetic, propionic, butyric, valeric acid, 2-methyl propionic acid, oxirane-carboxylic acid, and cyclopropane carboxylic acid. The term “carboxylic acid ester” or “ester” refers to a carboxyl group in which R″ is alkyl, alkenyl, alkynyl, carbocyclic, heterocylic, aryl, or aralkyl.

The term “carbocyclic” refers to a group that contains one or more covalently closed ring structures, and that the atoms forming the backbone of the ring are all carbon atoms. The ring structure may be saturated or unsaturated. The term thus distinguishes carbocyclic from heterocyclic rings in which the ring backbone contains at least one non-carbon atom. The term carbocylic encompasses cycloalkyl ring systems.

The terms “cycloalkane” or “cyclic alkane” or “cycloalkyl” refer to a carbocyclic group in which the ring is a cyclic aliphatic hydrocarbon, for example, a cyclic alkyl group preferably with 3 to 12 ring carbons. “Cycloalkyl” includes, by way of example, cyclopropyl, cyclobutyl, cyclopentyl, cyclohexyl, cycloheptyl, or cyclooctyl, and the like. The cycloalkyl group may be optionally substituted with an amino, alkyl, halo, or hydroxyl group.

The term “ether” refers to the group —R′—O—R″ wherein R′ and R″ as used in this definition are independently hydrogen, alkyl, alkenyl, alkynyl, carbocyclic, heterocylic, aryl, or aralkyl, and R′ can additionally be a covalent bond attached to a carbon.

The terms “halo” or “halogen” refer to fluoro, chloro, bromo, or iodo, usually regarding halo substitution for a hydrogen atom in an organic compound.

The terms “heterocyclic” or “heterocycle” means an optionally substituted, saturated or unsaturated, aromatic or non-aromatic cyclic hydrocarbon group with 4 to about 12 carbon atoms, preferably about 5 to about 6, wherein 1 to about 4 carbon atoms are replaced by nitrogen, oxygen or sulfur. Exemplary heterocyclic which are aromatic include groups pyridinyl, furanyl, benzofuranyl, isobenzofuranyl, pyrrolyl, thienyl, 1,2,3-triazolyl, 1,2,4-triazolyl, indolyl, imidazolyl, thiazolyl, thiadiazolyl, pyrimidinyl, oxazolyl, triazinyl, and tetrazolyl. Exemplary heterocycles include benzimidazole, dihydrothiophene, dioxin, dioxane, dioxolane, dithiane, dithiazine, dithiazole, dithiolane, furan, indole, 3-H indazole, 3-H-indole, imidazole, indolizine, isoindole, isothiazole, isoxazole, morpholine, oxazole, oxadiazole, oxathiazole, oxathiazolidine, oxazine, oxadiazine, piperazine, piperidine, purine, pyran, pyrazine, pyrazole, pyridine, pyrimidine, pyrimidine, pyridazine, pyrrole, pyrrolidine, tetrahydrofuran, tetrazine, thiadiazine, thiadiazole, thiatriazole, thiazine, thiazole, thiomorpholine, thiophene, thiopyran, triazine, and triazole. The heterocycle may be optionally substituted with an amino, alkyl, alkenyl, alkynyl, halo, hydroxyl, carbocyclic, thio, other heterocyclic, or aryl group. Exemplary heterocyclic groups include 1-pyrrolyl, 2-pyrrolyl, 3-pyrrolyl, 1-indolyl, 2-indolyl, 3-indolyl, 1-pyridyl, 2-pyridyl, 3-pyridyl, 4-pyridyl, 1-imidazolyl, 2-imidazolyl, 3-imidazolyl, 4-imidazolyl, 1-pyrazolyl, 2 pyrazolyl, 3-pyrazolyl, 4-pyrazolyl, 5-pyrazolyl, 1-pyrazinyl, 2-pyrazinyl, 1-pyrimidinyl, 2-pyrimidinyl, 4-pyrimidinyl, 5-pyrimidinyl, 1-pyridazinyl, 2-pyridazinyl, 3-pyridazinyl, 4-pyridizinyl, 1-indolizinyl, 2-indolizinyl, 3-indolizinyl, 4-indolizinyl, 5-indolizinyl, 6-indolizinyl, 7-indolizinyl, 8-indolizinyl, 1-isoindolyl, 2-isoindolyl, 3-isoindolyl, 4-isoindolyl and 5-isoindolyl.

The term “hydroxy” or “hydroxyl” refers to the substituent —OH.

The term “oxo” shall refer to the substituent ═O

The term “nitro” means —NO₂

The term “sulfanyl” refers to —SR′ where R′ as used in this definition is hydrogen, alkyl, alkenyl, alkynyl, carbocyclic, heterocylic, aryl, or aralkyl.

The term “sulfenyl” refers to —SOR′ where R′ as used in this definition is hydrogen, alkyl, alkenyl, alkynyl, carbocyclic, heterocylic, aryl, or aralkyl.

The term “sulfonyl” refers to —SOR′ where R′ as used in this definition is hydrogen, alkyl, alkenyl, alkynyl, carbocyclic, heterocylic, aryl, or aralkyl.

“Optional” or “optionally” means that the subsequently described event or circumstance may or may not occur, and that the description includes instances where said event or circumstance occurs and instances in which it does not. “Optionally” is inclusive of embodiments in which the described conditions is present and embodiments in which the described condition is not present. For example, “optionally substituted phenyl” means that the phenyl may or may not be substituted, and that the description includes both unsubstituted phenyl and phenyl wherein there is substitution. “Optionally” is inclusive of embodiments in which the described condition is present and embodiments in which the described condition is not present.

The compounds of the present disclosure can exist in tautomeric, geometric, or stereoisomeric forms. The present disclosure contemplates all such compounds, including cis- and trans-geometric isomers, E- and Z-geometric isomers, R- and S-enantiomers, diastereomers, d-isomers, l-isomers, the racemic mixtures thereof and other mixtures thereof, as falling within the scope of the disclosure.

Also included in the family of compounds of the present disclosure are the pharmaceutically acceptable salts, esters, and prodrugs thereof. The term “pharmaceutically-acceptable salts” embraces salts commonly used to form alkali metal salts and to form addition salts of free acids or free bases. The nature of the salt is not critical, provided that it is pharmaceutically acceptable. Suitable pharmaceutically acceptable acid addition salts of compounds of the present disclosure may be prepared from inorganic acid or from an organic acid. Examples of such inorganic acids are hydrochloric, hydrobromic, hydroiodic, nitric, carbonic, sulfuric, and phosphoric acid. Appropriate organic acids may be selected from aliphatic, cycloaliphatic, aromatic, araliphatic, heterocyclic, carboxylic and sulfonic classes of organic acids, examples of which are formic, acetic, propionic, succinic, glycolic, gluconic, lactic, malic, tartaric, citric, ascorbic, glucoronic, maleic, fumaric, pyruvic, aspartic, glutamic, benzoic, anthranilic, mesylic, salicylic, p-hydroxybenzoic, phenylacetic, mandelic, embonic (pamoic), methanesulfonic, ethylsulfonic, benzenesulfonic, sulfanilic, stearic, cyclohexylaminosulfonic, algenic, and galacturonic acid. Suitable pharmaceutically-acceptable base addition salts of compounds of the present disclosure include metallic salts made from aluminum, calcium, lithium, magnesium, potassium, sodium and zinc or organic salts made from N,N′-dibenzylethyleneldiamine, choline, chloroprocaine, diethanolamine, ethylenediamine, meglumine (N-methylglucamine) and procain. All of these salts may be prepared by conventional means from the corresponding compounds of by reacting, for example, the appropriate acid or base with the compounds of the present disclosure.

As used herein, the term “pharmaceutically acceptable ester” refers to esters which hydrolyze in vivo and include, but are not limited to, those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include formates, acetates, propionates, butyrates, acrylates, and ethylsuccinates.

The term “pharmaceutically acceptable prodrugs” as used herein refers to those prodrugs of the compounds of the present disclosure which are, within the scope of sound medical judgment, suitable for use in contact with the tissues of humans and lower animals without undue toxicity, irritation, allergic response and the like, commensurate with a reasonable risk/benefit ratio, and effective for their intended use, where possible, of the compounds of the disclosure. The term “prodrug” refers to compounds that are rapidly transformed in vivo to yield the parent compound of the above formulae, for example, by hydrolysis in blood. A thorough discussion is provided in T. Higuchi and V. Stella, Prodrugs as Novel delivery Systems, Vol. 14 of the A.C.S. Symposium Series and in Edward B. Roche, ed., Bioreversible Carriers in Drug Design, American Pharmaceutical Association and Pergamon Press, (1987), both of which are incorporated by reference herein.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject novobiocin analogue or derivative from one organ, or portion of the body, to another organ, or portion of the body. Each carrier must be “acceptable” in the sense of being compatible with the other ingredients of the formulation and not injurious to the patient. Some examples of materials which may serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; (21) modified cyclodextrins such as sulfobutylether-beta-cyclodextrin, e.g., Captisol, and hydroxypropyl-beta-cyclodextrin; and (22) other non-toxic compatible substances employed in pharmaceutical formulations.

The “patient” or “subject” to be treated with the compounds of the present disclosure can be any animal, e.g., dogs, cats, mice, monkeys, rats, rabbits, horses, cows, guinea pigs, sheep, and is preferably a mammal, such as a domesticated animal or a livestock animal. In another aspect, the patient is a human.

The term “inhibit” or “inhibiting” refers to a statistically significant and measurable reduction in neurotoxicity, preferably as measured by one or more of the assays discussed herein, preferably a reduction of at least about 10% versus control, more preferably a reduction of about 50% or more, still more preferably a reduction of about 60%, 70%, 80%, 90%, or more.

The term “preventing” as used herein means that the compounds of the present disclosure are useful when administered to a patient who has not been diagnosed as possibly having the disorder or disease at the time of administration, but who would normally be expected to develop the disorder or disease or be at increased risk for the disorder or disease. The compounds of the disclosure can slow the development of the disorder or disease symptoms, delay the onset of the disorder or disease, or prevent the individual from developing the disorder or disease at all. Preventing also includes administration of the compounds of the disclosure to those individuals thought to be predisposed to the disorder or disease due to age, familial history, genetic or chromosomal abnormalities, and/or due to the presence of one or more biological markers for the disorder or disease.

The term “treating” as used herein generally means that the compounds of the disclosure can be used in humans or animals with at least a tentative diagnosis of the disorder or disease. The compounds of the disclosure can delay or slow the progression of the disorder or disease thereby giving the individual a more useful life span. The term “treatment” embraces at least an amelioration of the symptoms associated with PKD in the patient, where amelioration is used in a broad sense to refer to at least a reduction in the magnitude of a parameter, e.g., symptom, associated with the condition being treated. As such, “treatment” also includes situations where the diseased condition or disorder, or at least symptoms associated therewith, are completely inhibited, e.g., prevented from happening, or stopped, e.g., terminated, such that the patient no longer suffers from the condition or disorder, or at least the symptoms that characterize the condition or disorder.

A “therapeutically effective amount” is an amount of a compound of the present disclosure or a combination of two or more such compounds, which inhibits, totally or partially, the progression of the condition or alleviates, at least partially, one or more symptoms of the condition. A therapeutically effective amount can also be an amount that is prophylactically effective. The amount that is therapeutically effective will depend upon the patient's size and gender, the condition to be treated, the severity of the condition and the result sought. For a given patient and condition, a therapeutically effective amount can be determined by methods known to those of skill in the art. For example, in reference to the treatment of cancer using the compounds of the present disclosure, a therapeutically effective amount refers to that amount which has the effect of (1) reducing the size of the tumor, (2) inhibiting (that is, slowing to some extent, preferably stopping) tumor metastasis, (3) inhibiting to some extent (that is, slowing to some extent, preferably stopping) tumor growth, and/or, (4) relieving to some extent (or, preferably, eliminating) one or more symptoms associated with the cancer.

The present disclosure is directed to methods of treating PKD, particularly ADPKD, for example by treating, preventing or alleviating cyst formation in the kidneys by administration of a therapeutically effective amount of one or more coumarin-3-carboxamide novobiocin analogues as described herein. In one aspect, the compounds are formulated in a pharmaceutical composition for administration to the subject in need thereof.

### DETAILED DESCRIPTION

Polycystic kidney disease (PKD or PCKD, also known as polycystic kidney syndrome) is a genetic disorder largely manifested in the kidneys. PKD is characterized by cyst formation and progressive enlargement of both kidneys, leading to end-stage renal disease (ESRD). In PKD, clusters of cysts develop primarily within the kidneys. Cysts are non-cancerous round sacs containing water-like fluid. Cysts vary in size as they accumulate fluid and can grow extremely large. Autosomal dominant polycystic kidney disease (ADPKD) is the most common life-threatening hereditary disorder affecting approximately one in every 400-1000 live births.

Mutations in either of two genes, PKD1 and PKD2, are associated with ADPKD. Polycystins are the protein products of PKD1 and PKD2, which encode polycystin-1 (PC1, 460 kDa) and polycystin-2 (PC2, 110 kDa), respectively. PC1 is a transmembrane receptor-like protein, and PC2 is a calcium channel. PC1 and PC2 can interact to form functional polycystin complexes that are widely expressed in various tissue types. Polycystins have a heterogenous distribution with localization to the primary cilia expressed in epithelial cells of the kidney, liver, pancreas, and breast, the smooth muscle and endothelial cells in the vasculature and astrocytes of the brain. Polycystins also have non-ciliary distribution. Both PC1 and PC2 appear to play roles in kidney development. The PC1-PC2 complex translates mechanical or chemical stimulations into calcium influx through PC2 channels, allowing for release of calcium from intracellular stores. Belibi and Edelstein 2010.

PC1 is a large integral membrane protein, with a smaller C-terminal cytoplasmic tail. The PC1 tail was found to interact with tuberin, a TSC2 gene product. The main function of tuberin was found to be inactivation of the Ser/Thr kinase mTOR. mTOR (mammalian target of rapamycin) promotes translation via phosphorylation of two proteins, S6-kinase and 4E-BP1. mTOR activity has been linked to increased cell growth, proliferation, apoptosis, and changes in differentiation. Shillingford et al., 2006, performed colocalization experiments in vivo and results suggested mTOR may be a part of the PC1-tuberin complex. Shillingford et al. also found that cyst-lining epithelial cells exhibited high mTOR activity. These results suggest that PC1 normally suppresses mTOR activity and that defects in PC1 consequently lead to aberrant mTOR activation. Shillingford et al., 2006, The mTOR pathway is regulated by polycystin-1, and its inhibition reverses renal cystogenesis in polycystic disease. Proc Natl Acad Sci USA 103 (14), 5466-71. Rapamycin specifically and effectively inhibits mTOR. Rapamycin is clinically approved as an immunosuppressant and is mostly used in kidney transplant patients. Shillingford at al. treated two different polycystic mouse models with rapamycin for one month; in the mild, late-onset mouse model, rapamycin treatment not only stopped kidney growth, but resulted in reduction of kidney size. It was shown to be likely because of the induction of programmed cell death (apoptosis) specifically in cyst lining epithelial cells. Rapamycin had no effect in the kidneys of normal mice. Treatment of an aggressive, early onset mouse model for two weeks with rapamycin resulted in reduction of kidney size and prevented loss of kidney function. Shillingford made use of the fact that ADPKD patients frequently undergo kidney transplantation and rapamycin is clinically approved to immunosuppress kidney transplant patients.

Typically ADPKD patients who undergo kidney transplantation have three kidneys; two of which are polycystic. Shillingford retrospectively studied a group of patients treated with rapamycin who had also computed tomography (CT) scans performed at the beginning of treatment and about 2 years later. In the rapamycin group, the kidney volumes decreased by 25%, whereas there was no effect in the non-rapamycin control group. Shillingford et al., 2006.

PC2 regulates the cell cycle through direct interaction with Id2, a member of the helix-loop-helix protein family that regulates cell proliferation. The PC2-Id interaction is mediated by PC1-dependent phosphorylation of PC2. Inhibition of 1d2 expression using RNA interference corrects the hyperproliferative response of PC1 mutant cells. Therefore the effect of Id2 inhibitors such as rosiglitazone on cyst formation was suggested by Belibi and Edelstein 2010.

Therapeutic approaches to interfere with the molecular pathways of cystogenesis are the focus of a recent review by Belibi and Edelstein 2010. For example, PC1/PC2-mediated calcium influx has become one drug development target. Triptolide, and active diterpene from traditional Chinese medicine, was shown to activate PC2-mediated calcium release in the cilia, to cause cell growth arrest in murine pkd1-null cells, and to inhibit cyst formation in Pkd1−/− embryonic mice with ADPKD. Leuenroth et al., 2007, Triptolide is a traditional Chinese medicine-derived inhibitor of polycyctic kidney disease. Proc Natl Acad Sci USA 104:4389-94.

Another molecular pathway of cystogenesis involves cAMP. There is evidence demonstrating a major role for cAMP in cyst fluid accumulation. A number of agonists such as arginine vasopressin (AVP), prostaglandin E2 (PGE2), epinephrine, norepinephrine, adenosine and caffeine can result in cAMP accumulation. See for example, Yamaguchi et al., 2000; cAMP stimulates the in vitro proliferation of renal cyst epithelial cells by activating the extracellular signal-regulated kinase pathway. Kidney Int. 57:1460-1471. It has previously been shown using embryonic mouse kidneys in organ culture (metanephric organ culture) that the intracellular second messenger, 3′,5′-cyclic adenosine monophosphate (cAMP or cyclic AMP) causes cystic dilation (cyst formation) over several days in culture. This effect of cAMP was particularly significant in kidneys from a genetic model of polycystic kidney disease (PKD), the Pkd1m1bei mouse, which has a mutation in the Pkd1gene. It is believed that cAMP stimulates fluid secretion by promoting chloride transport into the tubule lumen. Chloride transport is dependent on the chloride channels, NKCC1 (which brings chloride into cells from the basolateral or blood side) and CFTR (which allows chloride to exit cells into the tubule or cyst lumen). This chloride is then followed by sodium and water causing fluid secretion into the cyst lumen. These findings were proven by using a CFTR inhibitor CFTR172inh, an NKCC1 inhibitor bumetanide, and by using kidneys from mice with inactivating mutations in the genes for CFTR and NKCC1 (Cftr and Nkcc1, respectively). It is also believed that cyst formation is dependent on increased or abnormal cell proliferation. Thus, inhibition of cyst formation should occur with inhibition of fluid secretion and cell proliferation.

Epidermal growth factor (EGF) also plays an important role in cyst epithelial cell proliferation and cyst expansion. EGFR inhibition reduces cyst formation in different animal models of PKD. Sweeney et al., 2003, Combination treatment of PKD utilizing dual inhibition of EGF-receptor activity and ligand bioavailability. Kidney Int. 64:1310-1319.

There is evidence for activation of the mTOR pathway in ADPKD. As shown schematically in Belibi and Edelstein, 2010, the PI3K-AKT pathway plays a major role in mTOR signaling. PI3K converts the lipid PIP2 into PIP3 (Phosphotidylinositol (3,4,5)-triphosphate), which localizes AKT to the membrane. The tuberous sclerosis complex 1 (TSC1; hamartin) and TSC2 (tuberin) complex is inactivated by AKT-dependent phosphorylation. Inactivation of TSC2 results in activation of mTOR via the Ras-related small GTPase (Rheb). mTOR phosphorylates p70S6K1, resulting in cell proliferation. mTOR inhibitors bind to FKBP (FK506 binding protein), which subsequently inhibits mTOR.

Kidney cyst growth can be dependent on both cyst-filling fluid secretion and abnormal cell proliferation. Models have shown that PKD can initiate by some event at a location in a tubule, and the tubule then dilates by abnormal cell growth that increases the area and volume for cyst formation. Continued cell proliferation and fluid secretion enlarge the cyst, which inhibits proper kidney function. As such, inhibiting cyst formation and growth can be used in a therapy for PKD. The phonotypic switch to PKD may be attributed to: decreased intracellular calcium; de-repression of B-Raf; and/or stimulation of the Ras/MAPK pathway by cAMP.

Genetic models of CFTR and NKCC1 have shown that cyst formation and cyst-filling fluid accumulation is dependent in chloride secretion, which provides an another potential approach to treatment of PKD. Also, abnormal cell proliferation can be stimulated in PKD kidneys by cAMP-dependent activation of the Ras/B-Raf/MEK/ERK pathway, and B-Raf is critical to the activation of this pathway. This information provides targets for therapies for PKD. Since CFTR and B-Raf are clients of Hsp90, inhibition of Hsp90 can be used to modulate CFTR and B-Raf as a therapy for PKD. Known inhibitors of Hsp90 include the anti-tumor antibiotics geldanamycin (“GDA”), radicicol (“RDC”), herbimycin A (“HB”), a 17-allylamino derivative of GDA (“17-AAG”), and the synthetic ATP analog called PU3. These molecules exert their activity by binding to the N-terminal ATP binding pocket and inhibit the ATPase activity of Hsp90. In addition, Georg et al. disclose lonidamine derivatives which may be useful for inhibiting Hsp90 or biological pathway thereof, and are potentially useful for treating PKD, U.S. Patent Application publication no. US 2009/0197911, which is incorporated herein by reference. Lonidamine, (1-(2,4,-dichlorobenzyl)-1H-indazole-3-carboxylic acid) belongs to a group of indazole-carboxylic acid compounds. These Hsp90 inhibitors may operate by binding to the N-terminal region, the C-terminal region, or another region of the homodimer that causes a conformational change.

Novobiocin (a DNA gyrase ATP binding site inhibitor) has been found to selectively bind to the C-terminal domain of Hsp90. Novobiocin, a member of the coumermycin family of antibiotics, was isolated from streptomyces and shown to manifest potent activity against Gram-positive bacteria. Novobiocin elicits antimicrobial activity through binding the ATP-binding pocket of DNA gyrase and prohibiting ATP-hydrolysis.

Certain novobiocin analogues were previously disclosed as anticancer agents and/or as neuroprotective agents and/or in the treatment of autoimmune disorders; for example, by Blagg et al. in U.S. Pat. No. 7,608,594; U.S. Pat. No. 7,622,451; U.S. Patent Application publication nos. US 2009/0187014 and US 2009/0163709; and WO/2010/096650, each of which is incorporated herein by reference. Now, it has been found that certain coumarin-3-carboxamide novobiocin analogues, such as those described herein, can be used to treat, inhibit, and/or prevent development of certain symptoms, such as cyst formation, in PKD.

Novobiocin and its derivatives and analogues inhibit the cellular chaperone Hsp90 by binding the C-terminal ATP-binding domain in contrast to classical Hsp90 inhibitors such as geldanamycin which bind the N-terminal ATP-binding pocket. Hsp90 inhibitors are known to affect the levels of multiple Hsp90 client proteins critical to cell proliferation and fluid secretion. It has been found that certain novobiocin analogues are useful in inhibiting cyst formation in ADPKD cells.

In one embodiment, the coumarin-3-carboxamide novobiocin analogues described herein are useful for treating ADPKD. In one aspect, the coumarin-3-carboxamide novobiocin analogues by inhibiting mTOR and/or Hsp90, or a biological pathway thereof. Accordingly, the method can include administering a novobiocin analogue in a therapeutically effective amount for reducing levels of mTOR pathway phosphoproteins P-mTOR, P-Akt and P-S6K, or combinations thereof. Further, the method can include administering a novobiocin analogue in a therapeutically effective amount for reducing levels of Hsp-90 client proteins CFTR, ErbB2, c-Raf and Cdk4, or combinations thereof.

One embodiment of the present disclosure is directed to a method of treating polycystic kidney disease (PKD) with a novobiocin analogue. In one aspect, the novobiocin analog comprises a substituted coumarin-3-carboxamide comprising a sugar substituent, for example, a noviose substituent. Certain compounds are disclosed as having utility as anticancer compounds, or in treatment of neurodegenerative or autoimmune disorders, by Blagg et al., in U.S. Pat. No. 7,608,594; U.S. Pat. No. 7,622,451 and US 2009/0163709.

In another aspect, the present disclosure is also directed to methods of treating PKD comprising administration of the novobiocin analog comprising a coumarin-3-carboxamide ring lacking a noviose sugar substituent. For example, coumarin-3-carboxamide compounds having small alkyl, and particularly biaryl and heterocyclic 3-carboxamide substituents that exploit hydrogen-bonding interactions with the binding pocket that typically binds the prenylated benzamide of novobiocin. See Burlison et al., Novobiocin Analogues That Manifest Anti-proliferative Activity against Several Cancer Cell Lines, J. Org. Chem., 73(6) 2130-2137 (2008) (Feb. 23, 2008 e-published); and Donnelly et al., The Design, Synthesis, and Evaluation of Coumarin Ring Derivatives of the Novobiocin Scaffold that Exhibit Antiproliferative Activity, J. Org. Chem. 73, 8901-8920 (2008), (e-published Oct. 22, 2008), both of which are incorporated by reference. Such compounds are also described as having utility as anticancer compounds, or in treatment of neurodegenerative or autoimmune disorders, in co-pending US 2009/0187014 and WO/2010/096650 by the present inventor, each of which is incorporated by reference.

For example, in one aspect, the Hsp90 inhibitor KU-174 targets the mTOR Pathway in ADPKD Cells and reduces in vitro cyst formation.

The disclosure provides methods of administering a therapeutically effective amount of a novobiocin analogue for inhibiting mTOR and/or Hsp90, or a biological pathway thereof. Accordingly, the method can include administering a novobiocin analogue in a therapeutically effective amount for reducing levels of mTOR pathway phosphoproteins P-mTOR, P-Akt and P-S6K, or combinations thereof. Further, the method can include administering a novobiocin analogue in a therapeutically effective amount for reducing levels of Hsp-90 client proteins CFTR, ErbB2, c-Raf and Cdk4, or combinations thereof.

In one embodiment, the disclosure provides methods of inhibiting the proliferation of cAMP- and EGF-stimulated ADPKD cyst-lining epithelial cells by exposing the cells to novobiocin derivatives. In one aspect, the novobiocin derivative KU-174 significantly inhibits the proliferation of cAMP- and EGF-stimulated ADPKD cyst-lining epithelial cells in a dose-dependent manner (0.1 μM to 1 μM KU-174 for 72 h) using the MTT assay. Western blotting showed decreases in P-Erk with 0.5 μM and 1 μM KU-174 at 48 h (70% and 80% decreases respectively; n=2) whereas total Erk levels remained unchanged.

In another embodiment, the disclosure provides methods of decreasing key phosphoprotein mediators of the mTOR pathway P-mTOR, P-Akt and P-S6K are decreased in ADPKD cells by exposure to novobiocin analogues, compared to control EGF treated cells. Since the mTOR pathway is inappropriately activated in PKD, it was tested whether KU-174 affected any of the key mediators of this pathway. ADPKD cells were stimulated with EGF and treated with 50 nM, 100 nM, 500 nM or 1 μM KU-174 for 24 or 48 h and the status of P-mTOR (S2448), P-Akt (S473), P-TSC2 (S939) and P-S6K (T289) were examined. After 24 h of treatment, there was no change in any of the phosphoproteins. However, by 48 h in 1 μM KU-174, P-mTOR was decreased by 56%, P-Akt by 64%, and P-S6K by 75% compared to control EGF treated cells (n=2). There was no change in P-TSC2 levels. Even though Akt is a well-known Hsp90 client protein, KU-174 did not appear to decrease total Akt levels at the concentrations examined.

In a further embodiment, the disclosure provides methods of decreasing the levels of Hsp90 client proteins CFTR, ErbB2, c-Raf and Cdk4 in a dose and time-dependent manner in ADPKD cells by administration of the novobiocin analogues described herein. In one aspect, treatment with the novobiocin analogue KU-174 caused decreases in the levels of the Hsp90 client proteins CFTR, ErbB2, c-Raf and Cdk4 in a dose and time-dependent manner.

In another embodiment, the disclosure provides a method to decrease cyst formation in ADPKD cells by treatment with novobiocin analogs. For example, the effects of KU-174 were tested on microcyst formation by ADPKD cells in a collagen gel matrix And 1 μM KU-174 inhibited cyst-formation induced by EGF and forskolin by more than 3-fold, and 5 μM KU-174 completely eliminated cyst formation (p<0.01, n=6). Based on these studies, it was concluded that novobiocin analogues, for example, KU-174, are useful as clinical candidates for the treatment of PKD.

In a further embodiment, the disclosure provides a method for treating, inhibiting, and/or preventing one or more symptoms of polycystic kidney disease (PKD) in a subject in need thereof, the method comprising administering to the subject a therapeutically effective amount of a novobiocin analogue. In one embodiment, the novobiocin analogue is a compound according to Formula I.

In a another embodiment, the disclosure provides a method for treating, inhibiting, and/or preventing one or more symptoms of polycystic kidney disease (PKD) in a subject in need thereof, the method comprising administering a pharmaceutical composition comprising a therapeutically effective amount of a novobiocin analogue. In one embodiment, the novobiocin analogue is a compound according to Formula I to the subject in need thereof.

In a further embodiment, the disclosure provides a method for the manufacture of a medicament useful for treating, inhibiting, and/or preventing one or more symptoms of polycystic kidney disease (PKD) in a subject in need thereof, the method comprising preparation of a pharmaceutical composition useful for administering to the subject a therapeutically effective amount of a novobiocin analogue, for example, a compound according to Formula I. In a specific aspect, the novobiocin analogue is KU-174; N-(7-((2S,3R,4S,5R)-3,4-dihydroxy-5-methoxy-6,6-dimethyltetrahydro-2H-pyran-2-yloxy)-8-methoxy-2-oxo-2H-chromen-3-yl)-3′,6-dimethoxybiphenyl-3-carboxamide.

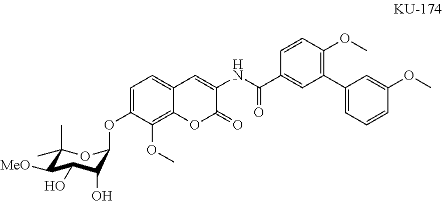

Compositions of the Present Disclosure

According to another aspect, the present disclosure provides a pharmaceutical composition, which comprises a therapeutically-effective amount of one or more compounds of the present disclosure or a pharmaceutically-acceptable salt, ester or prodrug thereof, together with a pharmaceutically-acceptable diluent or carrier.

The compositions may be formulated for any route of administration, in particular for oral, rectal, transdermal, subcutaneous, intravenous, intramuscular, intranasal, or intraperitoneal, administration. The compositions may be formulated in any conventional form, for example, as tablets, capsules, caplets, solutions, suspensions, dispersions, syrups, sprays, gels, suppositories, patches, and emulsions. Accordingly, the compounds of the present disclosure are useful in the treatment or alleviation of ADPKD, the symptoms of which can be reduced by the administration of a therapeutically effective amount of the compounds of the present disclosure.

The following examples are provided to illustrate the present disclosure and are not intended to limit the scope thereof. Those skilled in the art will readily understand that known variations of the conditions and processes of the following preparative procedures can be used to prepare these compounds.

### EXAMPLES

### Example 1

### Synthesis of Novobiocin Analogues

A library of novobiocin analogue compounds that contained both modified coumarin and sugar derivatives was prepared. The compounds were prepared as set forth in Scheme 1 below along with a procedure recently developed for the synthesis of noviose. See Yu et al., Synthesis of (−)-Noviose from 2,3-O-Isopropylidene-D-erythronolactol, J. Org. Chem. 69, 7375-7378 (2004), which is incorporated by reference.

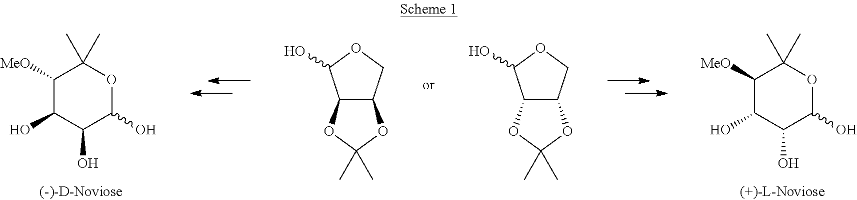

The novobiocin analogues prepared according to the scheme included modification of the coumarin ring by shortening of the amide side chain and removal of the 4-hydroxy substituent (A) (see Madhavan et al., Novel Coumarin Derivatives of Heterocyclic Compounds as Lipid Lowering Agents, Bioorg. Med. Chem. Lett. 13, 2547 (2003), which is incorporated by reference), removal of both the 4-hydroxy and amide linker (B), steric replacements of both the 4-hydroxy and benzamide ring (C), and 1,2-positional isomers of the noviosyl linkage (D and E).

These selected coumarin rings were coupled with trichloroacetimidate of noviose carbonate in the presence of boron trifluoride etherate as shown in Scheme 2 below. See Shen et al., Syntheses of Photolabile Novobiocin Analogues, Bioorg. Med. Chem. Lett. 14, 5903 (2004). The resulting cyclic carbonates (A1-E1) were treated with methanolic ammonia to provide 2′-carbamoyl (A2-E2), 3′-carbamoyl (A3-E3), and descarbamoyl products (A4-E4) in good yields. See also Yu et al., Hsp90 Inhibitors Identified from a Library of Novobiocin Analogues, J. Am. Chem. Soc. 127, 12778-12779 (2005), which is incorporated by reference.

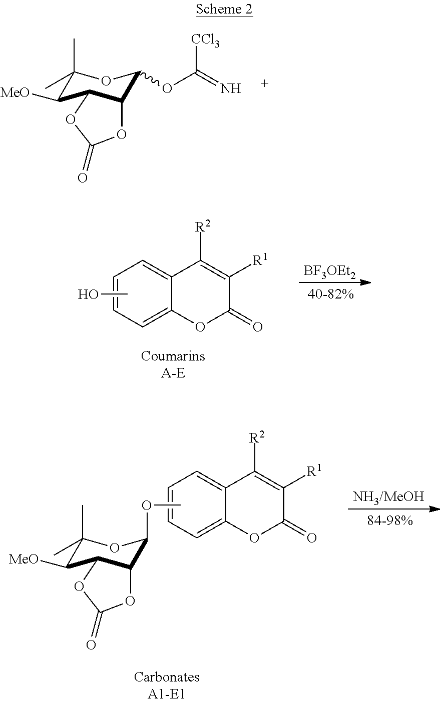

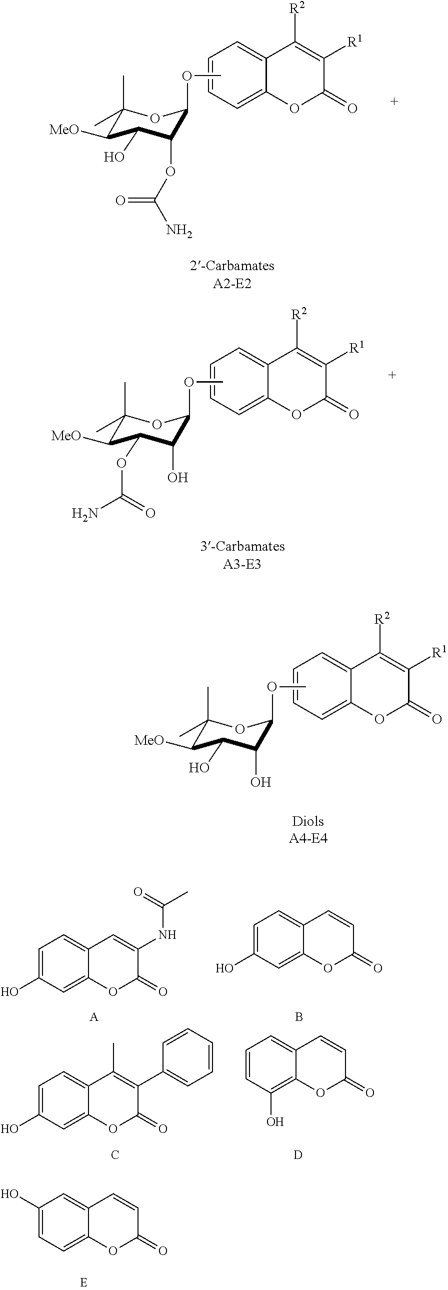

wherein R¹ in the above scheme is hydrogen, amido, amino, or aryl; and
wherein R² in the above scheme is hydrogen, alkyl, or hydroxy.

Overall, the following analogues of novobiocin were prepared, which are set forth below:

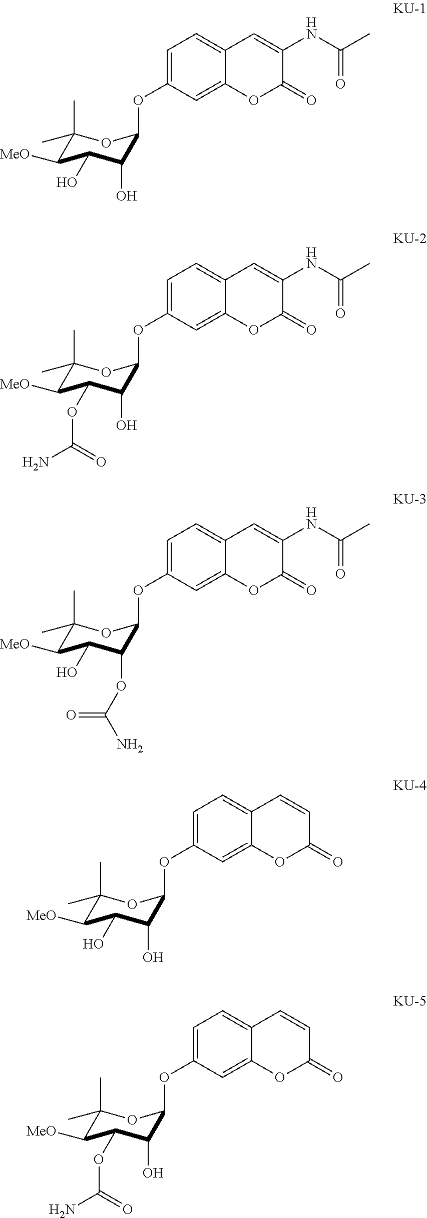

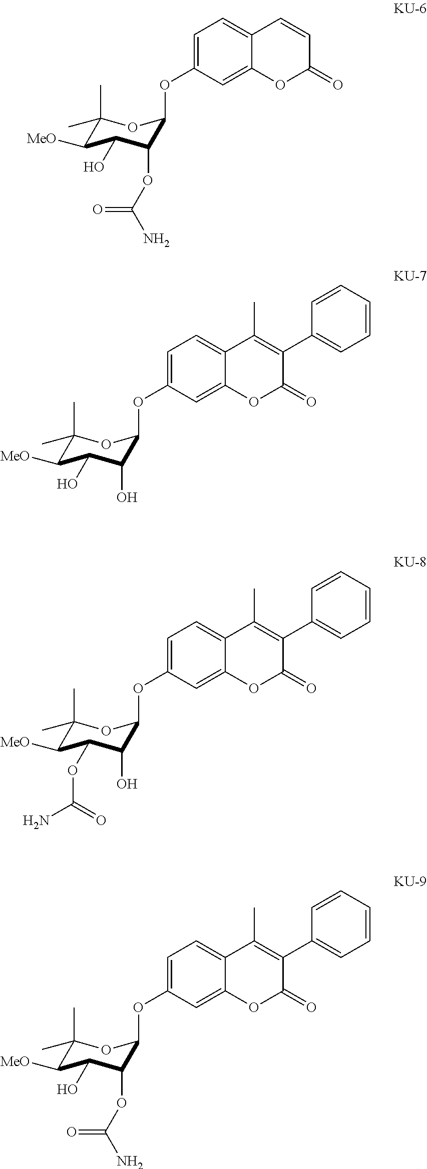

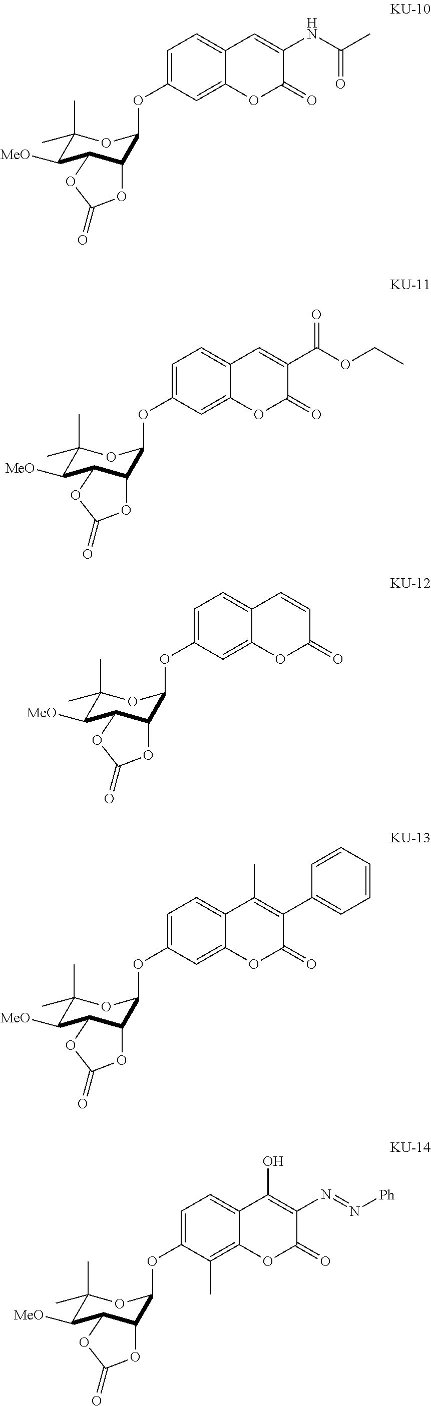

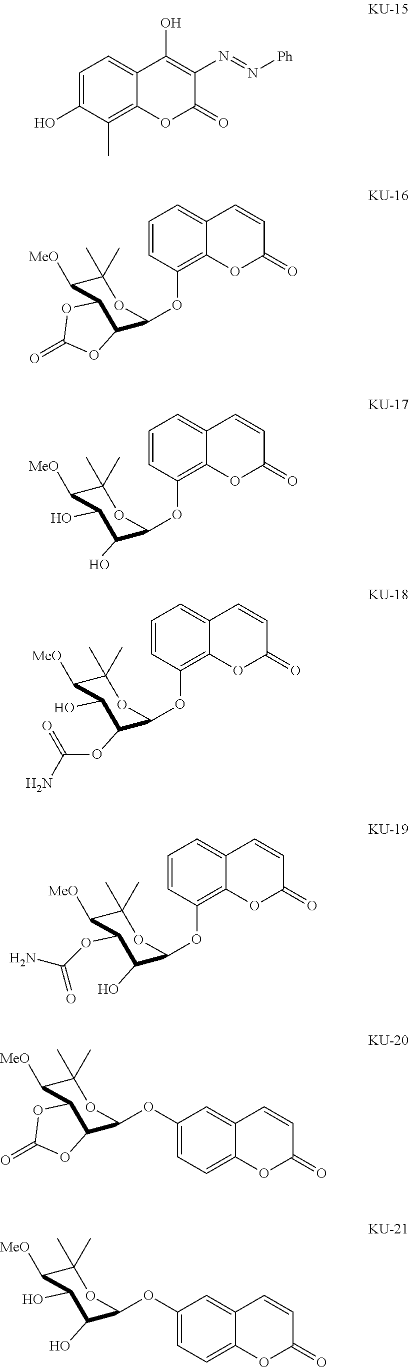

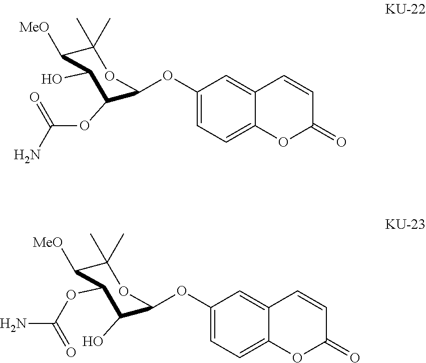

N-(7-((3aR,4R,7R,7aR)-7-methoxy-6,6-dimethyl-2-oxo-tetrahydro-3aH-[1,3]dioxolo[4,5-c]pyran-4-yloxy)-2-oxo-2H-chromen-3-yl)acetamide (A1). Noviose carbonate trichloroacetimidate (180 mg, 0.50 mmol) and 7-hydroxy-3-acetamino-coumarin A (133 mg, 0.60 mmol) were dissolved in CH₂Cl₂ (7 mL) before boron trifluoride etherate (30 μl, 0.03 mmol) was added to the suspension at 25° C. The mixture was stirred at 25° C. for eight hours and quenched with Et₃N (0.4 mL, 2.8 mmol). The solvent was removed and the residue purified by chromatography (SiO₂, 5% acetone in CH₂Cl₂) to afford A1 (134 mg, 64%) as a colorless solid: [α]²⁵_{D}=−71.0° (c, 0.1, CH₂Cl₂); ¹H NMR (CD₃Cl 400 MHz) δ 8.67 (s, 1H), 8.00 (br s, 1H), 7.46 (d, J=8.6 Hz, 1H), 7.05 (d, J=2.3 Hz, 1H), 7.00 (dd, J=2.3, 8.6 Hz, 1H), 5.82 (d, J=1.5 Hz, 1H), 5.02 (dd, J=1.5, 7.8 Hz, 1H), 4.94 (t, J=7.8 Hz, 1H), 3.62 (s, 3H), 3.30 (d, J=7.8 Hz, 1H), 2.26 (s, 3H), 1.37 (s, 3H), 1.21 (s, 3H); ¹³C NMR (CD₃Cl 100 MHz) δ 169.7, 159.2, 157.4, 153.5, 151.4, 129.2, 123.9, 122.8, 115.1, 114.6, 104.1, 94.7, 83.4, 78.3, 77.6, 77.5, 61.1, 27.9, 25.2, 22.4; IR (film) νₘₐₓ 1819, 1764, 1615, 1560, 1507, 1375, 1300, 1212, 1168, 1107, 1072, 1034, 1002, 969 cm⁻¹, HRMS (FAB⁺) m/z 420.1285 (M+H⁺, C₂₀H₂₂NO₉ requires 420.1294).

(2R,3R,4R,5R)-2-(3-acetamido-2-oxo-2H-chromen-7-yloxy)-4-hydroxy-5-methoxy-6,6-dimethyl-tetrahydro-2H-pyran-3-ylcarbamate (A2), (3R,4S,5R,6R)-6-(3-acetamido-2-oxo-2H-chromen-7-yloxy)-5-hydroxy-3-methoxy-2,2-dimethyl tetrahydro-2H-pyran-4-yl carbamate (A3) and N-(7-((2R,3R,4S,5R)-3,4-dihydroxy-5-methoxy-6,6-dimethyl-tetrahydro-2H-pyran-2-yloxy)-2-oxo-2H-chromen-3-yl)acetamide (A4). Noviosylated coumarin A1 (20 mg, 0.047 mmol) was dissolved in methanolic ammonia (7.0 M, 2 mL) at 25° C. and stirred for 24 hours. The solvent was evaporated and the residue purified by preparative HPLC (SiO₂, 20% 2-propanol in hexanes) to afford A2 (4.2 mg, 22%), A3 (8.6 mg, 42%) and A4 (3.5 mg, 20%) as colorless solids.

A2: [a]²⁵_{D}=−143.2° (c, 0.11, 50% MeOH in CH₂Cl₂); ¹HNMR (50% CD₃OD in CD₂Cl₂ 400 MHz) δ 8.58 (s, 1H), 7.44 (d, J=8.4 Hz, 1H), 7.01 (s, 1H), 6.97 (d, J=8.4 Hz, 1H), 5.59 (d, J=2.0 Hz, 1H), 5.03 (dd, J=2.0, 3.6 Hz, 1H), 4.25 (dd, J=3.6, 9.7 Hz, 1H), 3.57 (s, 3H), 3.30 (d, J=9.7 Hz, 1H), 2.19 (s, 3H), 1.31 (s, 3H), 1.13 (s, 3H); ¹³CNMR (50% CD₃OD in CD₂Cl₂ 100 MHZ) δ 168.8, 157.2, 156.4, 155.5, 149.5, 126.9, 122.9, 120.4, 112.6, 112.3, 101.6, 94.8, 82.5, 77.0, 71.9, 64.7, 59.9, 27.0, 22.1, 20.6; IR (film) νₘₐₓ 3473, 1716, 1689, 1610, 1540, 1528, 1505, 1375, 1240, cm⁻¹; HRMS (FAB⁺) m/z 437.1565 (M+H⁺, C₂₀H₂₅N₂O₉ requires 437.1560).

A3: [a]²⁵_{D}=−116.2° (c, 0.24, 50% MeOH in CH₂Cl₂); ¹HNMR (CD₃OD 400 MHz) δ 8.59 (s, 1H), 7.52 (d, J=10.8 Hz, 1H), 7.04 (s, 1H), 7.03 (d, J=10.8 Hz, 1H), 5.56 (d, J=2.4 Hz, 1H), 5.25 (dd, J=3.2, 9.8 Hz, 1H), 4.20 (dd, J=2.4, 3.2 Hz, 1H), 3.58 (s, 3H), 3.35 (d, J=9.8 Hz, 1H), 2.22 (s, 3H), 1.27 (s, 3H), 1.18 (s, 3H); ¹³CNMR (CD₃OD 100 MHZ) S171.6, 158.8, 158.7, 158.1, 151.8, 128.9, 125.6, 122.5, 114.4, 114.2, 103.1, 99.1, 81.6, 79.0, 71.8, 69.7, 60.1, 27.9, 22.9, 22.4; IR (film) νₘₐₓ 3470, 1716, 1686, 1615, 1538, 1523, 1505, 1372, 1242, 1120 cm⁻¹; HRMS (FAB⁺) m/z 437.1576 (M+H⁺, C₂₀H₂₅N₂O₉ requires 437.1560).

A4: As shown in the Scheme 3 below, the coumarin ring (2) was constructed by the condensation of commercially available benzaldehyde 1 with glycine in the presence of acetic anhydride. See Madhavan et al., Novel coumarin derivatives of heterocyclic compounds as lipid-Lowering agents, Bioorg. Med. Chem. Lett. 13, 2547 (2003). After selective deprotection, the free phenol was coupled with the trichloroacetimidate of noviose carbonate (4) (Yu et al., Synthesis of (−)-Noviose from 2,3-O-Isopropylidene-D-erythronolactol, Org. Chem. 69, 7375-7380 (2004)) in the presence of catalytic boron trifluoride etherate (Shen et al., Synthesis of Photolabile Novobiocin Analogues, Bioorg. Med. Chem. Lett. 14, 5903-5907 (2004)). KU-1/A4 was furnished in excellent yield by treatment of the cyclic carbonate 5 with triethylamine in methanol, resulting in solvolysis of the carbonate to afford the desired product.

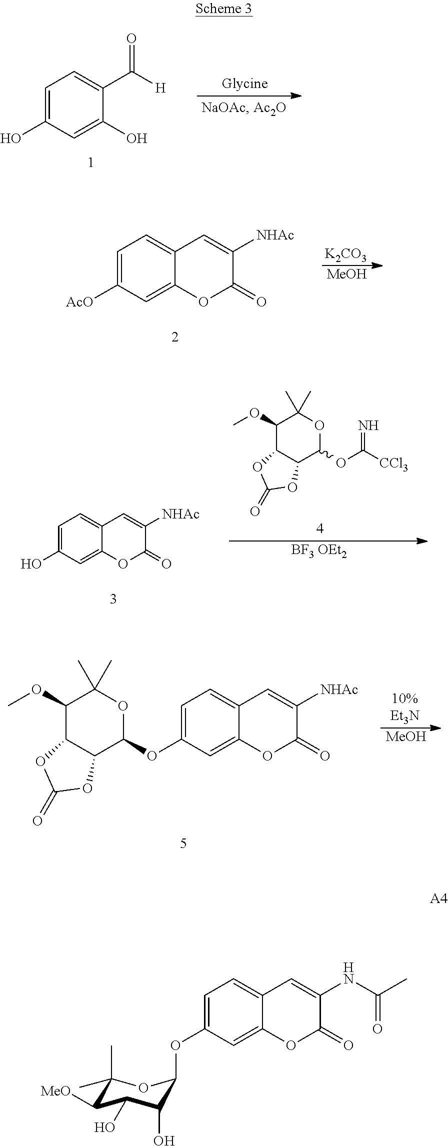

More specifically, triethylamine (0.2 mL) was added to a solution of noviosylated coumarin (45 mg, 0.10 mmol) in methanol (2 mL) at 25° C. After stirring for 48 hours, the solvent was evaporated and the residue purified by preparative TLC (SiO₂, DCM-acetone; 4:1) to afford KU-1/A4 (35 mg, 0.086 mmol, 83%) as a white solid. N-(7-((2R,3R,4S,5R)-3,4-dihydroxy-5-methoxy-6,6-dimethyl-tetrahydro-2H-pyran-2-yloxy)-2-oxo-2H-chromen-3-yl)acetamide (KU-1/A4). [α]²⁵_{D}=−351.6° (c, 0.06, 50% MeOH in CH₂Cl₂); ¹HNMR (CD₃OD 400 MHz) δ 8.58 (s, 1H), 7.51 (d, J=8.3 Hz, 1H), 7.03 (s, 1H), 7.02 (d, J=8.3 Hz, 1H), 5.55 (d, J=2.3 Hz, 1H), 4.10 (dd, J=3.3, 9.6 Hz, 1H), 4.03 (dd, J=2.4, 3.3 Hz, 1H), 3.60 (s, 3H), 3.38 (d, J=9.6 Hz, 1H), 2.21 (s, 3H), 1.30 (s, 3H), 1.13 (s, 3H); ¹³CNMR (CD₃OD 100 MHZ) δ 171.6, 158.9, 158.8, 151.8, 128.9, 125.7, 122.5, 114.3, 114.1, 103.1, 99.2, 84.2, 78.8, 71.5, 68.4, 61.1, 28.2, 22.9, 22.4; IR (film) νₘₐₓ 3326, 1714, 1674, 1613, 1558, 1553, 1108 cm⁻¹; HRMS (FAB⁺) m/z 394.1492 (M+H⁺, C₁₉H₂₄O₈ requires 394.1502).

7-((3aR,4R,7R,7aR)-7-methoxy-6,6-dimethyl-2-oxo-tetrahydro-3aH-[1,3]dioxolo[4,5-c]pyran-4-yloxy)-2H-chromen-2-one (B1). Noviose carbonate trichloroacetimidate (90 mg, 0.25 mmol) and 7-hydroxy-coumarin B (48 mg, 0.30 mmol) were dissolved in CH₂Cl₂ (2 mL) before boron trifluoride etherate (10 μl, 0.01 mmol) was added to the suspension at 25° C. The mixture was stirred at 25° C. for eight hours and quenched with Et₃N (0.1 mL, 0.7 mmol). The solvent was removed and the residue purified by chromatography (SiO₂, 2% acetone in CH₂Cl₂) to afford BI (66 mg, 73%) as a colorless solid: [α]²⁵_{D}=−85.6° (c, 1.15, CH₂Cl₂); ¹HNMR (CDCl₃ 400 MHz) δ 7.69 (d, J=9.5 Hz, 1H), 7.43 (d, J=8.6 Hz, 1H), 7.05 (d, J=2.3 Hz, 1H), 6.95 (dd, J=2.3, 8.6 Hz, 1H), 6.34 (d, J=9.5 Hz, 1H), 5.84 (d, J=1.3 Hz, 1H), 5.03 (dd, J=1.3, 7.7 Hz, 1H), 4.94 (t, J=7.7 Hz, 1H), 3.62 (s, 3H), 3.30 (d, J=7.7 Hz, 1H), 1.37 (s, 3H), 1.20 (s, 3H); ¹³CNMR (CDCl₃ 100 MHZ) δ 161.2, 158.9, 155.9, 153.5, 143.5, 129.4, 114.7, 114.4, 113.7, 104.4, 94.6, 83.4, 78.3, 77.8, 77.5, 61.0, 27.9, 22.4; IR (film) νₘₐₓ 1809, 1730, 1612, 1171, 1157, 1109 cm⁻¹; HRMS (FAB⁺) m/z 363.1083 (M+H⁺, C₁₈H₁₉O₈ requires 363.1080).

(3R,4S,5R,6R)-5-hydroxy-3-methoxy-2,2-dimethyl-6-(2-oxo-2H-chromen-7-yloxy)-tetrahydro-2H-pyran-4-yl carbamate (B2), (2R,3R,4R,5R)-4-hydroxy-5-methoxy-6,6-dimethyl-2-(2-oxo-2H-chromen-7-yloxy)-tetrahydro-2H-pyran-3-ylcarbamate (B3) and 7-((2R,3R,4S,5R)-3,4-dihydroxy-5-methoxy-6,6-dimethyl-tetrahydro-2H-pyran-2-yloxy)-2H-chromen-2-one (B4). Noviosylated coumarin B1 (25 mg, 0.07 mmol) was dissolved in methanolic ammonia (7.0 M, 2 mL) at 25° C. and stirred for 24 hours. The solvent was evaporated and the residue purified by preparative TLC (SiO₂, 25% acetone in methylene chloride) to afford B2 (4.3 mg, 16%), B3 (14.5 mg, 52%) and B4 (4.0 mg, 17%) as colorless solids.

B2: [a]²⁵_{D}=−85.1° (c, 0.71, 50% MeOH in CH₂Cl₂); ¹HNMR (CD₃OD 400 MHz) δ 7.91 (d, J=9.5 Hz, 1H), 7.58 (dd, J=1.3, 9.0 Hz, 1H), 7.04 (s, 1H), 7.03 (d, J=9.0 Hz, 1H), 6.30 (d, J=9.5 Hz, 1H), 5.65 (d, J=2.1 Hz, 1H), 5.04 (dd, J=2.6, 3.4 Hz, 1H), 4.28 (dd, J=3.4, 9.9 Hz, 1H), 3.62 (s, 3H), 3.39 (d, J=9.5 Hz, 1H), 1.35 (s, 3H), 1.15 (s, 3H); ¹³CNMR (CD₃OD 100 MHZ) δ 161.7, 159.7, 157.5, 155.3, 144.1, 129.1, 113.6, 113.4, 112.8, 103.0, 96.4, 83.9, 78.5, 73.4, 66.2, 60.8, 28.0, 21.8; IR (film) νₘₐₓ 3438, 2982, 2932, 1731, 1616, 1403, 1338, 1280, 1117, 1002, 963 cm⁻¹; HRMS (FAB⁺) m/z 380.1333 (M+H⁺, C₁₇H₂₁O₇ requires 380.1345).

B3: [a]²⁵_{D}=−111.8° (c, 0.18, 50% MeOH in CH₂Cl₂); ¹HNMR (CD₃OD 400 MHz) δ 7.91 (d, J=9.5 Hz, 1H), 7.58 (d, J=8.3 Hz, 1H), 7.05 (s, 1H), 7.04 (d, J=8.3 Hz, 1H), 6.30 (d, J=9.9 Hz, 1H), 5.59 (d, J=2.4 Hz, 1H), 5.25 (dd, J=3.2, 9.8 Hz, 1H), 4.20 (dd, J=2.4, 3.2 Hz, 1H), 3.59 (d, J=9.5 Hz, 1H), 3.57 (s, 3H), 1.36 (s, 3H), 1.17 (s, 3H); ¹³CNMR (CD₃OD 100 MHZ) δ 161.7, 159.9, 157.7, 155.3, 144.2, 129.1, 113.6, 113.5, 112.7, 102.9, 98.6, 81.1, 78.6, 71.4, 69.3, 60.6, 27.5, 22.0; IR (film) νₘₐₓ 3359, 2979, 2937, 1710, 1615, 1317, 1120, 1092, 995 cm⁻¹; HRMS (FAB⁺) m/z 380.1327 (M+H⁺, C₁₇H₂₁O₇ requires 380.1345).

B4: [a]²⁵_{D}=−129.4° (c, 0.18, 50% MeOH in CH₂Cl₂); ¹HNMR (CD₃OD 400 MHz) δ 7.91 (d, J=9.5 Hz, 1H), 7.57 (dd, J=2.4, 10.4 Hz, 1H), 7.02 (m, 2H), 6.27 (dd, J=4.5, 9.5 Hz, 1H), 5.57 (d, J=2.4 Hz, 1H), 4.11 (dd, J=3.3, 9.5 Hz, 1H), 4.03 (dd, J=2.4, 3.3 Hz, 1H), 3.60 (s, 3H), 3.39 (d, J=9.5 Hz, 1H), 1.35 (s, 3H), 1.12 (s, 3H); ¹³CNMR (CD₃OD 100 MHZ) δ 161.7, 160.9, 155.4, 144.2, 129.0, 113.5, 113.4, 112.6, 102.9, 98.8, 83.7, 78.4, 71.1, 67.9, 60.7, 27.7, 22.0; IR (film) νₘₐₓ 3415, 2984, 2934, 1730, 1718, 1707, 1615, 1118, 999, 957 cm⁻¹; HRMS (FAB⁺) m/z 337.11279 (M+H⁺, C₁₇H₂₁O₇ requires 337.1287).

7-((3aR,4R,7R,7aR)-7-methoxy-6,6-dimethyl-2-oxo-tetrahydro-3aH-[1,3]dioxolo[4,5-c]pyran-4-yloxy)-4-methyl-3-phenyl-2H-chromen-2-one (C1). Noviose carbonate trichloroacetimidate (90 mg, 0.25 mmol) and 7-hydroxy-4-methyl-3-phenyl-coumarin C (76 mg, 0.30 mmol) were dissolved in CH₂Cl₂ (2 mL) before boron trifluoride etherate (10 μL, 0.01 mmol) was added to the suspension at 25° C. The mixture was stirred at 25° C. for eight hours and quenched with Et₃N (0.1 mL, 0.7 mmol). The solvent was removed and the residue purified by chromatography (SiO₂, 1% acetone in CH₂Cl₂) to afford C1 (92 mg, 73%) as a colorless solid: [α]²⁵_{D}=−75.8° (c, 1.41, CH₂Cl₂); ¹HNMR (CDCl₃ 400 MHz) δ 7.80 (d, J=9.6 Hz, 1H), 7.44 (m, 3H), 7.33 (m, 2H), 7.09 (d, J=2.4 Hz, 1H), 7.01 (dd, J=2.4, 5.2 Hz, 1H), 5.84 (d, J=1.3 Hz, 1H), 5.03 (dd, J=1.3, 7.7 Hz, 1H), 4.94 (t, J=7.7 Hz, 1H), 3.62 (s, 3H), 3.30 (d, J=7.7 Hz, 1H), 2.31 (s, 3H), 1.37 (s, 3H), 1.20 (s, 3H); ¹³CNMR (CDCl₃ 100 MHZ) δ 161.0, 158.0, 153.9, 153.0, 147.4, 134.3, 130.0 (2C), 128.3 (2C), 128.0, 126.2, 125.2, 115.6, 113.0, 103.7, 94.1, 82.9, 77.8, 76.7, 76.5, 60.5, 27.4, 22.0, 16.5; IR (film) νₘₐₓ 1874, 1715, 1612, 1564, 1507, 1383, 1262, 1167, 1130, 1113, 1070, 1033, 1006, 968, 936 cm⁻¹; HRMS (FAB⁺) m/z 453.1554 (M+H⁺, C₂₅H₂₅O₈ requires 453.1549).

(3R,4S,5R,6R)-5-hydroxy-3-methoxy-2,2-dimethyl-6-(4-methyl-2-oxo-3-phenyl-2H-chromen-7-yloxy)-tetrahydro-2H-pyran-4-ylcarbamate (C2), (2R,3R,4R,5R)-4-hydroxy-5-methoxy-6,6-dimethyl-2-(4-methyl-2-oxo-3-phenyl-2H-chromen-7-yloxy)-tetrahydro-2H-pyran-3-yl carbamate (C3) and 7-((2R,3R,4S,5R)-3,4-dihydroxy-5-methoxy-6,6-dimethyl-tetrahydro-2H-pyran-2-yloxy)-4-methyl-3-phenyl-2H-chromen-2-one (C4). Noviosylated coumarin Cl (25 mg, 0.055 mmol) was dissolved in methanolic ammonia (7.0 M, 2 mL) at 25° C. and stirred for 24 hours. The solvent was evaporated and the residue purified by preparative TLC (SiO₂, 25% acetone in methylene chloride) to afford C2 (6.3 mg, 25%), C3 (13.7 mg, 53%) and C4 (3.0 mg, 13%) as colorless solids.

C2: [a]²⁵_{D}=−72.9° (c, 0.19, 50% MeOH in CH₂Cl₂); ¹HNMR (CD₃OD 400 MHz) δ 7.80 (d, J=9.0 Hz, 1H), 7.43 (m, 3H), 7.32 (m, 2H), 7.10 (m, 2H), 5.69 (d, J=1.8 Hz, 1H), 5.06 (dd, J=2.1, 3.2 Hz, 1H), 4.30 (dd, J=3.2, 9.7 Hz, 1H), 3.63 (s, 3H), 3.40 (d, J=9.7 Hz, 1H), 2.31 (s, 3H), 1.36 (s, 3H), 1.18 (s, 3H); ¹³CNMR (CD₃OD 100 MHZ) S162.2, 159.7, 158.0, 154.2, 149.2, 135.1, 130.3 (2C), 128.4 (2C), 128.1, 127.0, 124.7, 115.3, 113.7, 103.2, 96.8, 84.4, 78.9, 73.8, 66.7, 61.3, 28.4, 22.3, 15.8; IR (film) νₘₐₓ 3474, 2986, 2924, 1713, 1605, 1382, 1355, 1263, 1124, 1001, 967 cm⁻¹; HRMS (FAB⁺) m/z 470.1821 (M+H⁺, C₂₅H₂₈NO₈ requires 470.1815).

C3: [a]25_{D}=−92.3° (c, 0.28, 50% MeOH in CH₂Cl₂); ¹HNMR (CD₃OD 400 MHz) δ 7.75 (d, J=9.5 Hz, 1H), 7.45 (m, 3H), 7.34 (m, 2H), 7.06 (m, 2H), 5.63 (d, J=2.4 Hz, 1H), 5.18 (dd, J=3.2, 9.6 Hz, 1H), 4.18 (dd, J=2.4, 3.2 Hz, 1H), 3.54 (s, 3H), 3.40 (d, J=9.5 Hz, 1H), 2.27 (s, 3H), 1.35 (s, 3H), 1.16 (s, 3H); ¹³CNMR (CD₃CN 125 MHZ) δ 160.7, 159.0, 156.0, 153.8, 148.0, 135.2, 130.1 (2C), 128.1 (2C), 127.7, 126.7, 124.4, 114.9, 113.1, 103.1, 98.2, 81.0, 78.4, 71.3, 69.0, 60.7, 27.7, 22.4, 15.8; IR (film) νₘₐₓ 3459, 3331, 2981, 2925, 1714, 1606, 1379, 1335, 1263, 1124, 1072 cm⁻¹; HRMS (FAB⁺) m/z 470.1811 (M+C₂₅H₂₈NO₈ requires 470.1815).

C4: [a]²⁵_{D}=−86.0° (c, 0.12, 50% MeOH in CH₂Cl₂); ¹HNMR (CD₃OD 400 MHz) δ 7.80 (d, J=9.6 Hz, 1H), 7.44 (m, 3H), 7.33 (m, 2H), 7.09 (m, 2H), 5.60 (d, J=1.9 Hz, 1H), 4.12 (dd, J=3.3, 9.5 Hz, 1H), 4.05 (dd, J=2.4, 3.1 Hz, 1H), 3.61 (s, 3H), 3.40 (d, J=9.5 Hz, 1H), 2.32 (s, 3H), 1.37 (s, 3H), 1.15 (s, 3H); ¹³CNMR (CD₃OD 100 MHZ) δ 161.9, 159.6, 153.8, 149.1, 134.7, 129.9 (2C), 127.9 (2C), 127.7, 126.5, 124.1, 114.7, 113.4, 102.7, 98.8, 83.8, 78.4, 71.1, 68.0, 60.7, 27.8, 22.0, 15.4; IR (film) νₘₐₓ 3403, 2977, 2924, 1717, 1607, 1558, 1505, 1381, 1260, 1124, 992 cm⁻¹; HRMS (FAB⁺) m/z 427.1750 (M+H⁺, C₂₄H₂₇O₇ requires 427.1757).

8-(7-Methoxy-6,6-dimethyl-2-oxo-tetrahydro-[1,3]dioxolo[4,5-c]pyran-4-yloxy)-chromen-2-one (D1) Noviose carbonate trichloroacetimidate (176 mg, 0.49 mmol) and 8-hydroxy-coumarin D (95 mg, 0.59 mmol) were dissolved in CH₂Cl₂ (5 mL). Boron trifluoride etherate (20 μL, 0.08 mmol) was added to the suspension at 25° C. The resulting slurry was stirred at 25° C. for 10 hours before the solvent was removed and the residue purified by chromatography (SiO₂, 1% MeOH in CHCl₃) to afford D1 (85 mg, 40%) as a colorless solid: [α]_{D}³¹=57° (c=0.1, 50% MeOH in CH₂Cl₂); ¹H NMR (CDCl₃, 500 MHz) δ 7.69 (d, J=9.6 Hz, 1H), 7.31 (t, J=9.1 Hz, 1H), 7.23 (dd, J=2.8 Hz, 9.0 Hz, 1H), 7.16 (d, J=2.8 Hz, 1H), 6.47 (d, J=9.6 Hz, 1H), 5.77 (d, J=1.0 Hz, 1H), 5.03 (dd, J=1.2 Hz, 7.8 Hz, 1H), 4.95 (t, J=7.7 Hz, 1H), 3.62 (s, 3H), 3.30 (d, J=7.7 Hz, 1H), 1.37 (s, 3H), 1.20 (s, 3H); ¹³C NMR (CDCl₃, 125 MHz) δ 160.6, 153.1, 152.1, 149.4, 142.9, 120.8, 119.3, 118.0, 117.4, 113.3, 94.5, 82.9, 77.9, 77.2, 76.5, 60.5, 27.5, 22.0; IR (film) νₘₐₓ 3054, 2987, 1817, 1730, 1572, 1422, 1166, 1112, 1040, 896, 739 cm⁻¹; HRMS (FAB⁺) m/z 363.1088 (M+H⁺, C₁₈H₁₉O₈ requires m/z 363.1080).

Carbamic acid 4-hydroxy-5-methoxy-6,6-dimethyl-2-(2-oxo-2H-chromen-8-yloxy)-tetrahydro-pyran-3-yl ester (D2), carbamic acid 5-hydroxy-3-methoxy-2,2-dimethyl-6-(2-oxo-2H-chromen-8-yloxy)-tetrahydro-pyran-4-yl ester (D3), 8-(3,4-Dihydroxy-5-methoxy-6,6-dimethyl-tetrahydro-pyran-2-yloxy)-chromen-2-one (D4) D1 (17 mg, 0.047 mmol) was dissolved in methanolic ammonia (2.0 M, 5 mL, 10 mmol) at 25° C. and stirred for five hours before the solvent was removed. The residue was purified by preparative TLC (SiO₂, 25% acetone in CH₂Cl₂) to afford D2 (3.8 mg, 21%), D3 (5.5 mg, 31%), and D4 (7.2 mg, 46%) as colorless solids.

D2: [a]_{D}³¹=−19° (c=0.1, 50% MeOH in CH₂Cl₂); ¹HNMR (CD₃OD in CD₂Cl₂, 500 MHz) δ 7.79 (d, J=9.6 Hz, 1H), 7.26 (m, 3H), 6.43 (d, J=9.6 Hz, 1H), 5.59 (d, J=2.0 Hz, 1H), 5.05 (dd, J=2.1 Hz, 3.4 Hz, 1H), 4.28 (m, 2H), 3.61 (s, 3H), 3.32 (m, 1H), 1.34 (s, 3H), 1.18 (s, 3H); ¹³C NMR (CD₃OD in CDCl₃, 100 MHz) δ 162.1, 158.0, 153.6, 149.3, 144.6, 121.1, 119.9, 117.7, 116.6, 113.6, 97.1, 84.5, 78.8, 74.1, 66.7, 61.4, 28.6, 22.4; IR (film) νₘₐₓ 3054, 2987, 1729, 1422, 896, 739, 705 cm⁻¹; HRMS (ESI⁺) m/z 380.1356 (M+H⁺, C₁₈H₂₂NO₈ requires m/z 380.1345).

D3: [α]_{D}³¹=−69° (c=0.1, 50% MeOH in CH₂Cl₂); NMR (CD₃OD in CD₂Cl₂, 500 MHz) δ 7.84 (d, J=9.6 Hz, 1H), 7.30 (m, 3H), 6.44 (d, J=9.5 Hz, 1H), 5.51 (d, J=2.3 Hz, 1H), 5.28 (dd, J=3.2 Hz, 9.8 Hz, 1H), 4.21 (m, 1H), 3.56 (s, 1H), 3.55 (s, 3H), 1.35 (s, 3H), 1.20 (s, 3H); ¹³C NMR (CD₃OD in CDCl₃, 125 MHz) δ 161.8, 157.4, 153.3, 148.7, 144.2, 120.8, 119.3, 117.4, 116.2, 113.2, 98.9, 81.3, 78.6, 71.5, 69.5, 60.8, 27.9, 22.3; IR (film) νₘₐₓ 3054, 2987, 1732, 1422, 896, 742 cm⁻¹; HRMS (ESI⁺) m/z 380.1348 (M+H⁺, C₁₈H₂₂NO₈ requires m/z 380.1345).

D4: [a]_{D}³¹=−91° (c=0.1, 50% MeOH in CH₂Cl₂); ¹HNMR (CD₃OD in CD₂Cl₂, 500 MHz) δ 7.82 (d, J=9.5 Hz, 1H), 7.26 (m, 3H), 6.43 (d, J=9.5 Hz, 1H), 5.50 (d, J=2.3 Hz, 1H), 4.12 (dd, J=3.4 Hz, 9.3 Hz, 1H), 4.05 (d, J=2.4 Hz, 1H), 3.59 (s, 3H), 3.33 (m, 1H), 1.35 (s, 3H), 1.15 (s, 3H); ¹³C NMR (CD₃OD in CDCl₃, 125 MHz) δ 161.7, 153.4, 148.6, 144.2, 120.7, 119.3, 117.3, 116.1, 113.1, 98.9, 83.8, 78.3, 71.1, 68.0, 60.9, 28.0, 22.2; IR (film) νₘₐₓ 3455, 3053, 2988, 1704, 1568, 1112, 738 cm⁻¹; HRMS (FAB⁺) m/z 337.1267 (M+H⁺, C₁₇H₂₁O₇ requires m/z 337.1287).

6-(7-Methoxy-6,6-dimethyl-2-oxo-tetrahydro-[1,3]dioxolo[4,5-c]pyran-4-yloxy)-chromen-2-one (E1) Noviose carbonate trichloroacetimidate (150 mg, 0.42 mmol) and 6-hydroxycoumarin E (67 mg, 0.42 mmol) were dissolved in CH₂Cl₂ (4 mL). Boron trifluoride etherate (20 μL, 0.06 mmol) was added to the suspension at 25° C. The resulting slurry was stirred at 25° C. for 10 hours before the solvent was removed and the residue purified by chromatography (SiO₂, 1% MeOH in CHCl₃) to afford E1 (63 mg, 42%) as a colorless solid: [α]_{D}³¹=−59° (c=0.1, 50% MeOH in CH₂Cl₂); ¹H NMR (CDCl₃, 500 MHz) δ 7.69 (d, J=9.6 Hz, 1H), 7.30 (d, J=9.0 Hz, 1H), 7.23 (dd, J=2.7 Hz, 9.0 Hz, 1H), 7.16 (d, J=2.7 Hz, 1H), 6.47 (d, J=9.6 Hz, 1H), 5.77 (m, 1H), 5.02 (dd, J=1.0 Hz, 7.8 Hz, 1H), 4.95 (d, J=7.7 Hz, 1H), 3.61 (s, 3H), 3.30 (d, J=7.7 Hz, 1H), 1.37 (s, 3H), 1.23 (s, 3H); ¹³C NMR (CDCl₃, 125 MHz) δ 160.6, 153.1, 152.1, 149.4, 142.9, 120.8, 119.3, 118.0, 117.4, 113.3, 94.5, 82.9, 77.9, 77.2, 76.5, 60.5, 27.5, 22.0; IR (film) νₘₐₓ 3054, 2987, 1818, 1730, 1422, 896, 739, 705 cm⁻¹; HRMS (FAB⁺) m/z 363.1109 (M+H⁺, C₁₈H₁₉O₈ requires m/z 363.1080).

Carbamic acid 5-hydroxy-3-methoxy-2,2-dimethyl-6-(2-oxo-2H-chromen-6-yloxy)-tetrahydro-pyran-4-yl ester (E2), carbamic acid 4-hydroxy-5-methoxy-6,6-dimethyl-2-(2-oxo-2H-chromen-6-yloxy)-tetrahydro-pyran-3-yl ester (E3), 6-(3,4-Dihydroxy-5-methoxy-6,6-dimethyl-tetrahydro-pyran-2-yloxy)-chromen-2-one (E4) E1 (17 mg, 0.047 mmol) was dissolved in methanolic ammonia (7.0 M, 5 mL, 35 mmol) at 25° C. and stirred for five hours before the solvent was removed. The residue was purified by preparative TLC (SiO₂, 25% acetone in CH₂Cl₂) to afford compound E2 (7.8 mg, 34%), E3 (9.9 mg, 43%), and E4 (4.7 mg, 23%) as colorless solids.

E2: [a]_{D}³¹=−45° (c=0.1, 50% MeOH in CH₂Cl₂). ¹H NMR (CD₃OD in CD₂Cl₂, 500 MHz) δ 7.82 (d, J=9.6 Hz, 1H), 7.27 (m, 3H), 6.44 (d, J=9.5 Hz, 1H), 5.60 (d, J=2.0 Hz, 1H), 5.05 (dd, J=2.0 Hz, 3.4 Hz, 1H), 4.28 (m, 1H), 3.61 (s, 3H), 3.32 (m, 1H), 1.34 (s, 3H), 1.18 (s, 3H); ¹³C NMR (CD₃OD in CD₂Cl₂, 125 MHz) δ 161.6, 157.2, 153.1, 148.8, 143.9, 120.8, 119.3, 117.4, 116.4, 113.2, 96.7, 84.1, 78.4, 73.7, 66.3, 61.3, 28.4, 22.2; IR (film) νₘₐₓ 3054, 2987, 1729, 1422, 896, 738, 705 cm⁻¹; HRMS (ESI⁺) m/z 380.1327 (M+H⁺, C₁₈H₂₂NO₈ requires m/z 380.1345).

E3: [a]_{D}⁻=−80° (c=0.1, 50% MeOH in CH₂Cl₂); ¹H NMR (CD₃OD in CD₂Cl₂, 500 MHz) δ 7.79 (d, J=9.5 Hz, 1H), 7.28 (d, J=2.3 Hz, 2H), 7.25 (s, 1H), 6.43 (d, J=9.5 Hz, 1H), 5.50 (d, J=2.3 Hz, 1H), 5.26 (dd, J=3.2 Hz, 9.8 Hz, 1H), 4.21 (t, J=2.7 Hz, 1H), 3.56 (m, 1H), 3.55 (s, 3H), 1.35 (s, 3H), 1.19 (s, 3H); ¹³C NMR (CD₃OD in CD₂Cl₂, 125 MHz) δ 159.5, 155.0, 151.1, 146.8, 141.8, 118.7, 117.3, 115.4, 114.4, 111.2, 96.7, 79.3, 76.6, 69.6, 67.4, 59.0, 26.0, 20.4; IR (film) νₘₐₓ 3054, 2987, 1731, 1422, 1265, 896, 742 cm⁻¹; HRMS (ESI⁺) m/z 380.1324 (M+H⁺, C₁₈H₂₂NO₈ requires m/z 380.1345).

E4: [α]_{D}⁻=−89° (c=0.05, 50% MeOH in CH₂Cl₂); ¹H NMR (CD₃OD in CD₂O₂, 400 MHz) δ 7.83 (d, J=9.6 Hz, 1H), 7.26 (m, 3H), 6.44 (d, J=9.5 Hz, 1H), 5.50 (d, J=2.3 Hz, 1H), 4.12 (dd, J=3.4 Hz, 9.3 Hz, 1H), 4.05 (d, J=2.4 Hz, 1H), 3.59 (s, 3H), 3.33 (m, 1H), 1.34 (s, 3H), 1.14 (s, 3H); ¹³C NMR (CD₃OD in CD₂Cl₂, 125 MHz) δ 162.1, 153.8, 149.2, 144.5, 121.3, 119.8, 117.8, 116.8, 113.5, 99.3, 84.4, 78.8, 71.6, 68.5, 61.6, 28.6, 22.8; IR (film) νₘₐₓ 3454, 3054, 2987, 1705, 1568, 1422, 1111, 896, 738 cm⁻¹; HRMS (FAB⁺) m/z 337.1275 (M+Fᵣ, C₁₇H₂₁O₇ requires m/z 337.1287).

### Example 2

### Amide Bond Side Chain Modifications

Modifications of the amide side chain allow for an in depth study of the hydrophobic cavity that binds to this portion of KU-1/A4 and the analogous benzamide of novobiocin. As such, analogues of KU-1/A4 with increasingly larger hydrophobic groups by the use of different commercially available or readily synthesized anhydrides, such as those anhydrides shown in the Scheme 4 below. See Khoo, L. E., Synthesis of Substituted 3-Aminocoumarins from Ethyl N-2-Hydroxyarylideneglycinates, Syn. Comm. 29, 2533-2538 (1999), which is incorporated by reference.

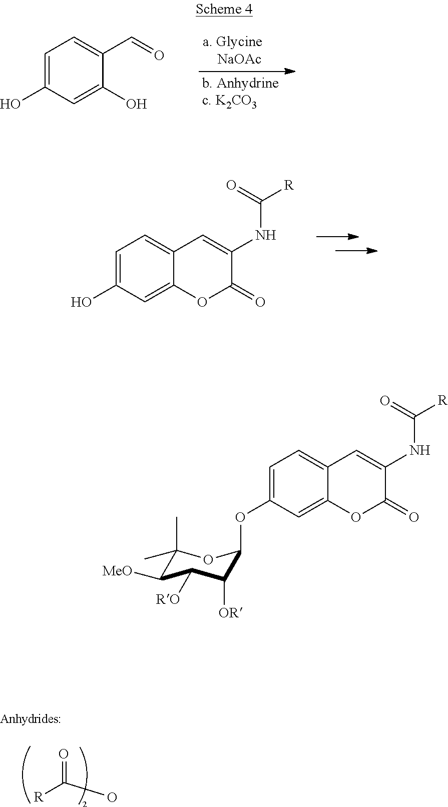

wherein in the Scheme 4, R is hydrogen, alkyl, alkenyl, alkynyl, aryl, carbocylic, heterocyclic, aryl, or aralkyl; and preferably R is hydrogen, alkyl, aryl, and aralkyl;
and wherein R′ is hydrogen or CONH₂.
As part of this example, the amide linkage can also be reversed to determine the optimal profile of this functionality. As set forth in the Scheme 5 below, the 7-hydroxy-3-ethyl ester coumarin can be hydrolyzed to afford the corresponding acid, which can be coupled with amines that mimic the same side chains used in the KU-1/A4 amide studies for direct comparison of biological activity. Once coupled, the free phenols can be noviosylated as described earlier to afford the cyclic carbonate products. Treatment of the carbonate with methanolic ammonia can give the diol, 2- and 3-carbamoyl products as shown in the scheme below wherein in the scheme R is hydrogen, alkyl, alkenyl, alkynyl, aryl, carbocylic, heterocyclic, aryl, or aralkyl; and R′ is hydrogen or CONH₂. See Shen et al., Synthesis of Photolabile Novobiocin Analogues, Bioorg. Med. Chem. Lett. 14, 5903-5906 (2004), which is incorporated by reference.

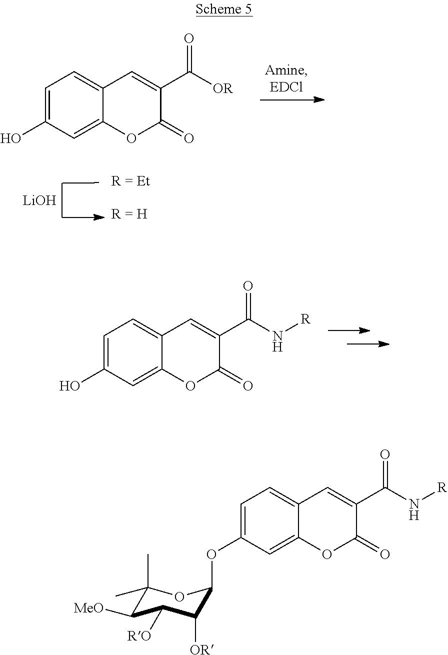

### Example 3

### Des(Dimethyl) and Desmethoxy Sugar Analogues

Modifications to the gem-dimethyl groups and the methyl ether on the noviose moiety can be prepared. In this example, the des(dimethyl) and desmethoxy sugar analogues can be prepared. Using KU-/1/A4 as an example in Scheme 6 below, 2,3-O-isopropylidene-L-erythronolactol can be converted to the corresponding alkene by Wittig olefination. Dihydroxylation can afford the syn diol as noted in the earlier synthesis of noviose. See Yu et al., Synthesis of (−)-Noviose from 2,3-O-Isopropylidene-D-erythronolactol, J. Org. Chem. 69 7375-7378 (2004). Protection of the primary alcohol, followed by alkylation of the secondary alcohol can afford the orthogonally protected molecule. Selective removal of the benzyl group and oxidation of the resultant alcohol can give the aldehyde. Treatment of this aldehyde with aqueous sulfuric acid can remove the acid-labile protecting groups while simultaneously promoting cyclization. Id.

Similarly, the desmethoxy compound can be prepared from the appropriately functionalized lactone (Stewart et al., 2-Deoxy-L-Ribose from an L-Arabino-1,5-lactone, Tetrahedron Assym. 13, 2667-2672 (2002)) by the addition of excess methyl Grignard to provide the primary and tertiary alcohol product. Oxidation of the primary alcohol can give the lactone, which can be reduced to the lactol before deprotection with aqueous sulfuric acid to yield the desmethoxy product. Once obtained, these sugars can be treated with carbonyl diimidazole to furnish the cyclic carbonates before coupling with the coumarin phenol. This set of conditions is based on previous work towards the preparation of novobiocin photoaffinity probes. See Shen et al., Synthesis of Photolabile Novobiocin Analogues, Bioorg. Med. Chem. Lett. 14 5903-5906 (2004).

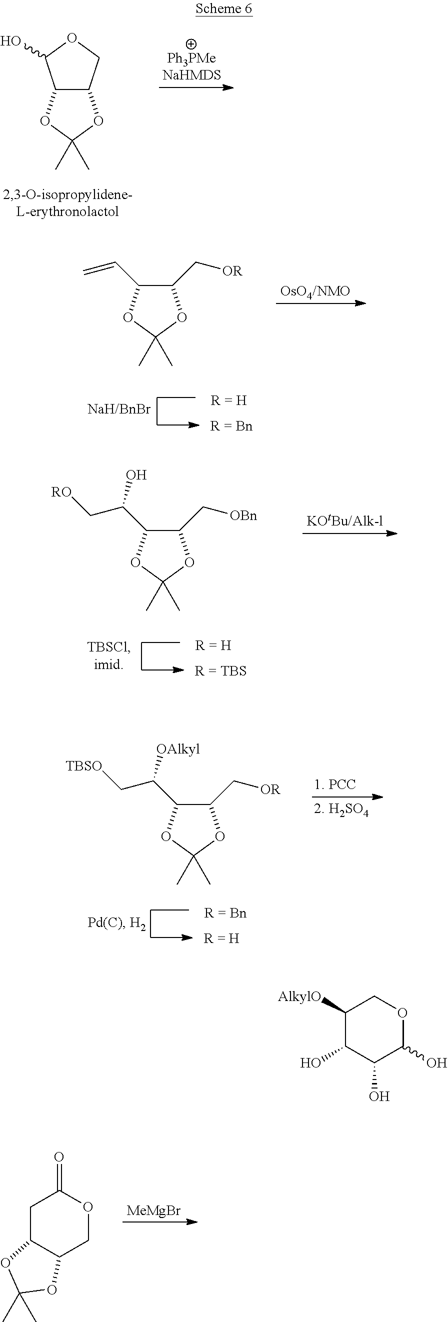

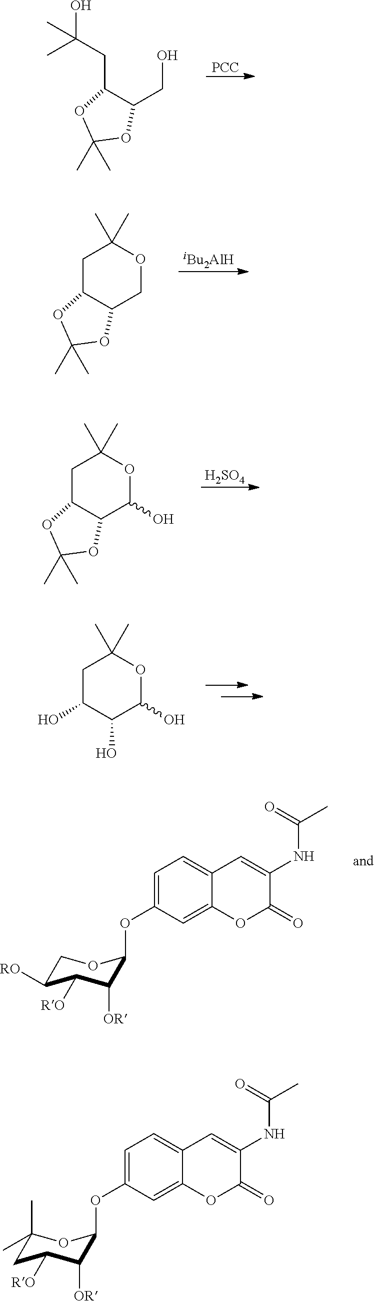

wherein preferably R is lower alkyl; and
wherein R′ is preferably hydrogen or —CONH₂.

It will be appreciated that other demethylated and/or dealkoxylated derivatives can be prepared in accordance with the above scheme, in addition to the modified KU-1/A4 derivatives shown above. The amide side chain, and sugar may be modified in coumarin ring analogues of novobiocin in accordance with the other examples shown herein.

### Example 4

### Coumarin Side Chains

The I analogues are directed to other side-chains extending from the coumarin ring. As an example, the KU-1/A4 coumarin ring can be prepared from 2,4-dihydroxy-5-nitrobenzaldehyde (see Chandrashekhar et al., g-substitution in the resorcinol nucleus, VI Formylation of 4-nitro and 2-nitro resorcinols, Proc. Ind. Acad. Sci. 29A 227-230 (1949)) and 2,4-dihydroxy-5-methoxybenzaldehyde (see Demyttenaere et al., Synthesis of 6-methoxy-4H-1-benzopyran-7-ol, a character donating component of the fragrance of Wisteria sinensis, Tetrahedron 58 2163-2166 (2002)) according to the procedure of Khoo et al., Synthesis of substituted 3-aminocoumarins from ethyl N-2-Hydroxyarylideneglycinates, Syn. Commun. 29 2533-2538 (1999), as generally set forth in Scheme 7 below. The o-hydroxybenzaldehyde can be treated with ethyl glycine under acidic conditions to afford the corresponding free amine upon basic workup. Both the amino and hydroxyl functionalities can be acylated with the same anhydrides as shown above. Subsequent hydrolysis of the phenolic ester can provide the coumarin amide, which can be coupled directly with noviose carbonate as described previously.

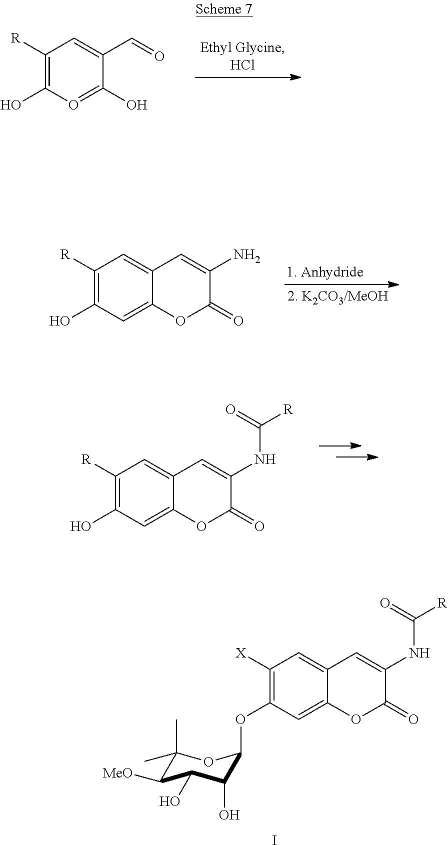

wherein in the scheme R is hydrogen, alkyl, alkenyl, alkynyl, aryl, carbocylic, heterocyclic, aryl, or aralkyl; wherein X is alkyl, alkenyl, alkynyl, aryl, aralkyl, alkoxy, halogen, or nitro.

### Example 5

### Chlorobiocin Analogues

This example involves the modification of the carbohydrate reside. More specifically, analogues similar to that of novobiocin's chlorinated pyrollic ester, chlorobiocin, can be prepared.

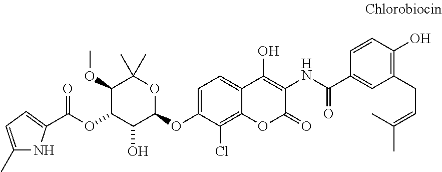

As an example, compound KU-1/A4 can be prepared, and then coupled with a variety of acids to selectively afford the equatorial acylated alcohols. Selective acylation is based upon previous studies aimed at the preparation of photolabile derivatives of novobiocin. See Shen et al., Synthesis of Photolabile Novobiocin Analogues, Bioorg. Med. Chem. Lett. 14 5903-5906 (2004), which is incorporated by reference. These acids can include the pyrrolic acid found in chlorobiocin as well as several other that are shown in Scheme 8A below. Exemplary acids include pyrrolic acids, indolic acids, pyridinic acids, benzoic acids, salicylic acid, para-hydrobenzoic acid, thiobenzoic acid, and pyrazolic acid. In one aspect, the sugar can be modified to include a functional group according to the formula —R′—OR″, wherein R′ is a covalent bond or alkyl, and R″ is an acyl group. Most preferably, the acyl derivative comprises the group —COR wherein R is alkyl, aryl, aralkyl, or an aromatic heterocyclic group. Alkylated, aralkylated, thiolated, halogenated, and hydroxylated pyroles, indoles, pyridines, and pyrazoles are attached to the sugar ring as shown in Scheme 8A below.

In another aspect, various substituents can be added to the amine of the carbamate side chain. As an example, carbonate KU-9/A1 can be prepared and amines added to provide the 3′-carbamoyl products as generally set forth in the Scheme 8B below. Thus, in one aspect the sugar can be modified to include a functional group according to the formula —R′OR″, wherein R′ is a covalent bond or alkyl, and R″ is C-amido. Most preferably, the C-amido group is —CONR′R″ wherein R′ is H, and R″ is alkyl, aryl, aralkyl, or an aromatic heterocyclic group. Pyroles, halogenated benzyls and pyridines, and alkyl groups are shown as the modified side chain of the sugar in Scheme 8B below.

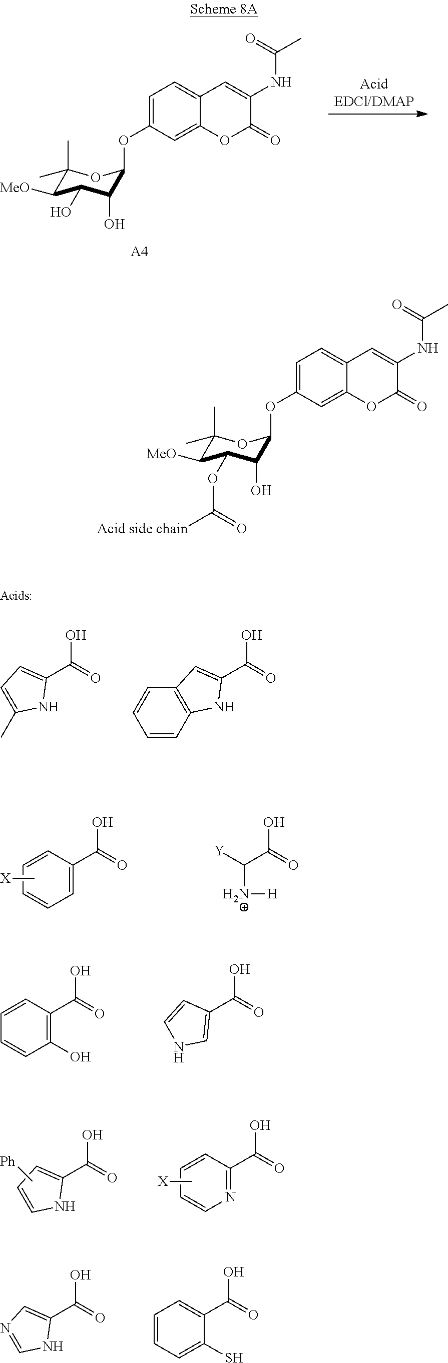

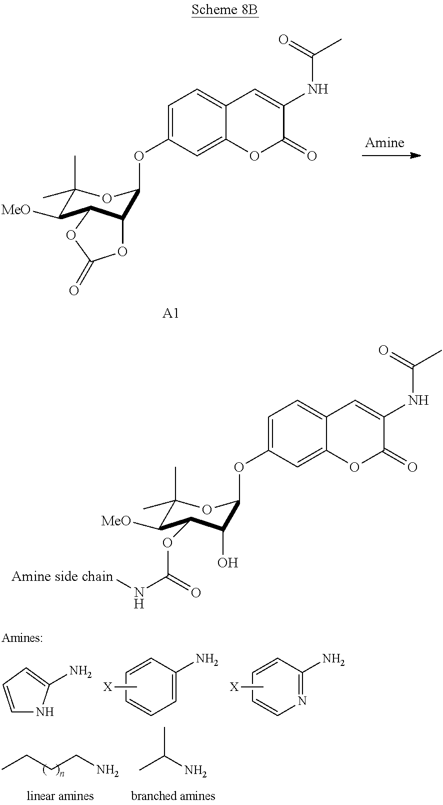

In Schemes 8A and 8B, X is alkyl, alkenyl, alkynyl, hydroxyl, halo, and n is an integer, preferably 0, 1, 2, 3, or 4.

### Example 6

### Furanose and Pyranose Novobiocin Derivatives

In this example, various pyranose and furanose coumarin derivatives can be prepared. These selected compounds are shown in below and include ester, amide, sulfonic ester, phosphonic ester, carbamoyl, sulfonamide, and hydroxyl derivatives.

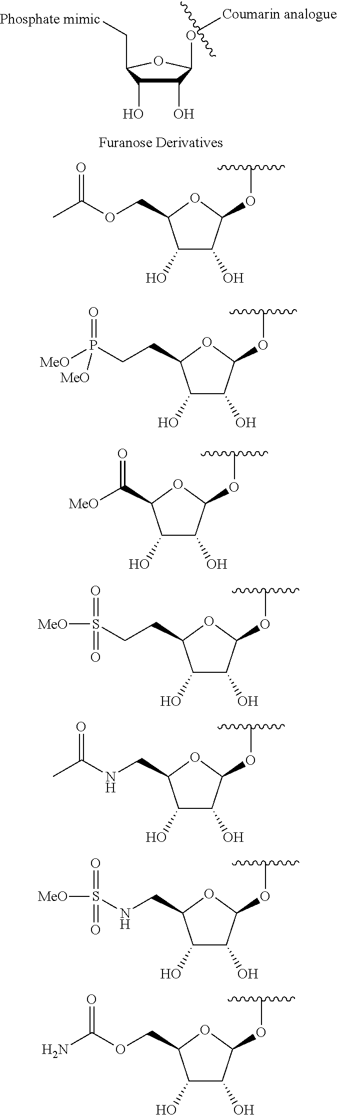

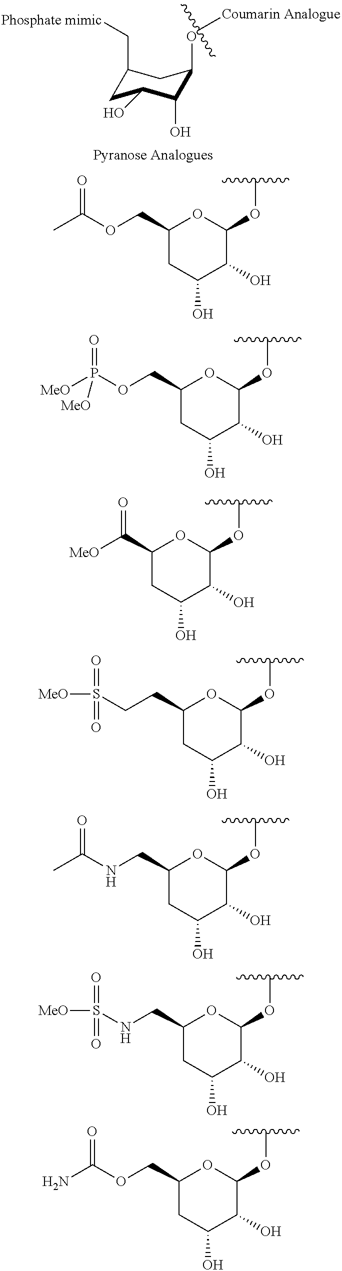

Furanose Derivatives

The o-acetyl derivative can be prepared from ribose (9.1, Scheme 9). Treatment of the ribose hemiacetal with benzyl alcohol and hydrochloric gas can provide the benzyloxyacetal, 9.2. See Pigro et al., Readily available carbohydrate-derived imines and amides as chiral ligands for asymmetric catalysis, Tetrahedron 58 5459-5466 (2002).

Subsequent reaction with carbonyl diimidazole can furnish the 2,3-cyclic carbonate (9.3), (See Peixoto et al., Synthesis of Isothiochroman 2,2-dioxide and 1,2-benzoxathiin 2,2-dioxide Gyrase B Inhibitors, Tetrahedron Lett. 41 1741-1745 (2000)) allowing the primary alcohol to react with acetyl chloride in the following step. Debenzylation, followed by conversion to the trichloroacetimidate 9.5 (See Peixoto et al., Synthesis of Isothiochroman 2,2-dioxide and 1,2-benzoxathiin 2,2-dioxide Gyrase B Inhibitors, Tetrahedron Lett. 41 1741-1745 (2000)) can furnish a suitable substrate for coupling with the KU-1/A4 coumarin ring system. As noted in previous work, coupling of trichloroacetimidates with phenols in the presence of catalytic boron trifluoride affords one stereoisomer (9.6), which results from attack of the intermediate oxonium species away from the sterically crowded cyclic carbonate. See Shen et al., Synthesis of Photolabile Novobiocin Analogues, Bioorg. Med. Chem. Lett. 14 5903-5906 (2004). It has been previously observed that treatment of similar cyclic carbonates with methanolic triethylamine readily provides the corresponding diol products (9.7) in high yields (greater than 80%).

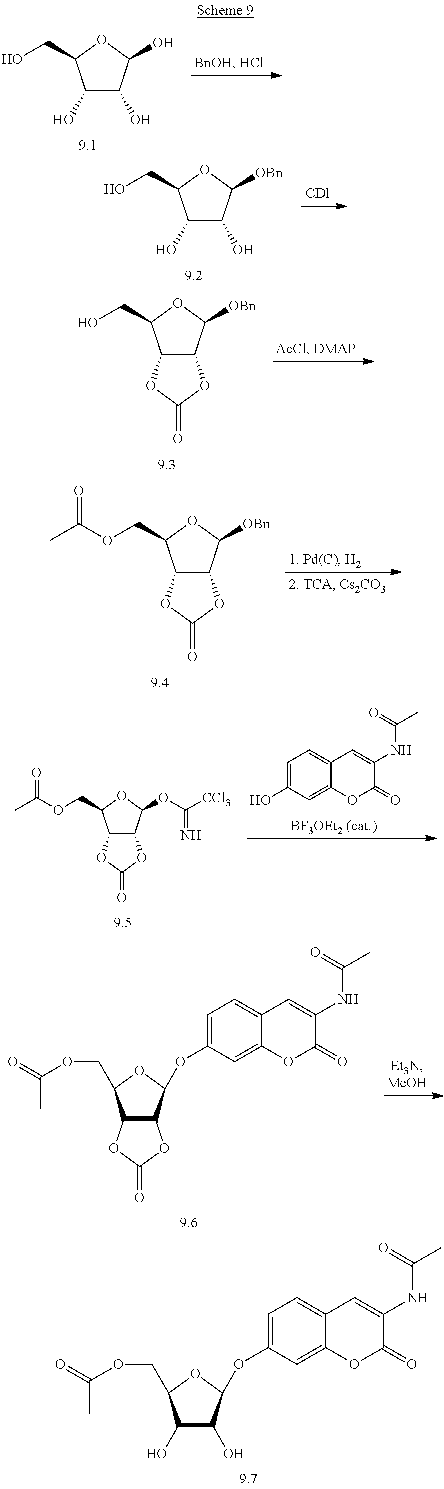

The remaining furanose derivatives can be prepared from benzyl-protected ribose carbonate (9.3, Scheme 10). Both the sulfonamide and N-acetyl analogues can be furnished by conversion of primary alcohol (9.3) to the corresponding azide by a Mitsunobu reaction with bis(azido)zinc pyridine complex. See Viaud et al., Zinc azide mediated Mitsunobu substitution, An expedient method for the one pot azidation of alcohols, Synthesis 130-132 (1990). The resulting azide (10.1) can be reduced, and the primary amine converted to the sulfonamide and N-acetyl functionalities, 10.2 and 10.3, respectively. See Hansson et al., Synthesis of Beta-benzyl N-(tert-butoxycarbonyl)-L-erythro-Beta-(benzyloxy)aspartate from (R,R)-(+)-tartaric acid, J. Org. Chem. 51 4490-4492 (1986). To prepare methyl ester 10.4, the free alcohol can be oxidized directly to the acid, followed by methylation. Carbamate 10.5 can also be prepared from the same alcohol, simply by treatment with trichloroacetyl isocyanate according to the procedure of Kocovsky, Carbamates: a method of synthesis and some synthetic applications, Tetrahedron Lett. 27 5521-5524 (1986). Both the sulfonic ester and the phosphonic ester can be prepared by conversion of 9.3 to iodide 10.6, followed by generation of the requisite enolate to displace the halide. See Callant et al., An efficient preparation and the intramolecular cyclopropanation of Beta-diazo-Beta-ketophosphonates and Beta-diazophosphonoacetates, Syn. Commun. 14 155-161 (1984). Subsequent treatment with palladium (0) and an amine can lead to allyl removal followed by decarboxylation to form 10.10 and 10.8. See Guibe, Allyl esters and their use in complex natural product syntheses, Tetrahedron 54 2967-3041 (1998).

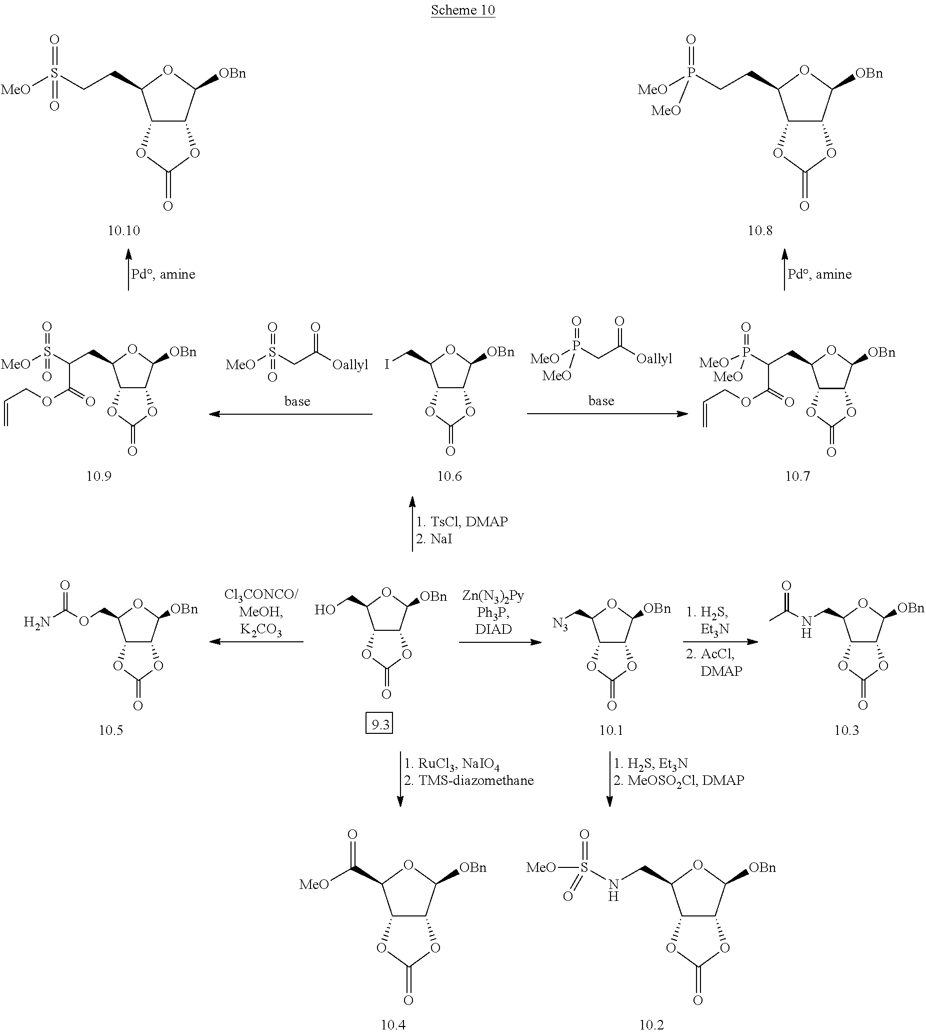

Pyranose Derivatives

The pyranose derivatives, which resemble noviose and a ring-expanded ribose ring, can be prepared by our recently reported synthesis of 11.1. See Yu et al., Synthesis of Mono- and dihydroxylated furanoses, pyranoses, and an oxepanose for the Preparation of Natural Product Analogue Libraries, J. Org. Chem. 70 5599-5605 (2005), which is incorporated by reference in its entirety. The pyranose derivatives can be prepared in a similar manner from the known dihydropyrone (See Ahmed et al., Total synthesis of the microtubule stabilizing antitumor agent laulimalide and some nonnatural analogues: The power of Sharpless' Asymmetric Epoxidation, J. Org. Chem. 68 3026-3042 (2003)), which is available in four steps from commercially available triacetyl D-glucal (Roth et al., Synthesis of a chiral synhton for the lactone portion of compactin and mevinolin, Tetrahedron Lett. 29 1255-12158 (1988)). The pyranose can be furnished by Sharpless asymmetric DI hydroxylation (SAD) of the olefin to give the product in high diastereomer excess (Kolb et al., Catalytic Asymmetric Dihydroxylation, Chem. Rev. 94 2483-2547 (1994)), which can be converted to the cyclic carbonate at a later time.

Reduction of the lactone with diisobutyl aluminum hydride can give lactol 11.2, which upon treatment with benzyl alcohol and hydrochloric gas can give the benzyloxyacetal 11.3, as shown in Scheme 11. Similar studies have been used to prepare noviose from arabinose using an identical sequence of steps. See Peixoto et al., Synthesis of Isothiochroman 2,2-dioxide and 1,2-benzoxathiin 2,2-dioxide Gyrase B Inhibitors, Tetrahedron Lett. 41 1741-1745 (2000). The corresponding diol can be treated with carbonyl diimidazole to yield cyclic carbonate 11.4. The primary alcohol can be converted to the same functionalities as shown in the scheme above, using the chemistry depicted for the furanose derivatives.

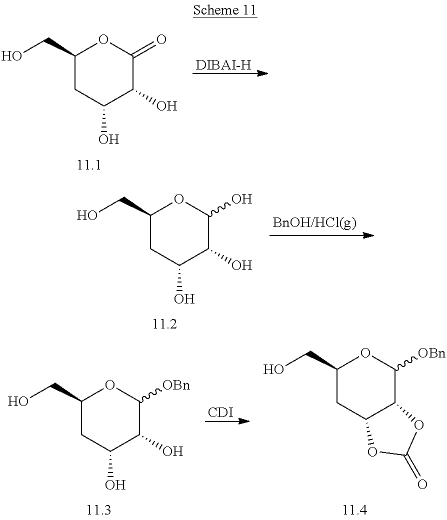

Once the benzyl protected pyranose derivatives are prepared, they can undergo hydrogenolysis to afford the hemiacetal. Treatment of the lactol with trichloroacetonitrile can furnish the corresponding trichloroacetimidate for subsequent coupling with the requisite coumarin/coumarin analogue. The procedure outlined herein illustrates the success of coupling such compounds with the coumarin phenol and this procedure can be used to prepare the corresponding analogues as described herein.

Using the foregoing schemes, the syntheses of eight protected pyranose analogues that include mono- and dihydroxylated variants of both ring-expanded and ring contracted analogues was performed. All eight of these compounds were orthogonally protected, such that the hemi-acetal could be coupled directly to the coumarin phenol as used similarly for the construction of A4. Subsequent removal of the protecting group(s) or treatment of the cyclic carbonate with ammonia, can afford the corresponding diol or carbamate products as demonstrated earlier.

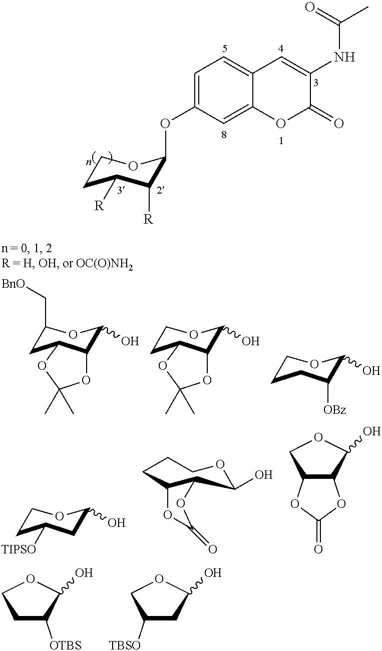

### Example 7.4

### Deshydroxy and 8-Desmethyl Analogues

In this example, the 4-deshydroxy and 8-desmethyl variants of novobiocin can be prepared along with the 8-methyl and 4-hydroxy analogues of KU-2/A3 (3′ carbamate) as shown below. Not only can the 3′-carbamoyl derivatives of these compounds be prepared, but also the corresponding diols for direct comparison to KU-1/A4 (diol).

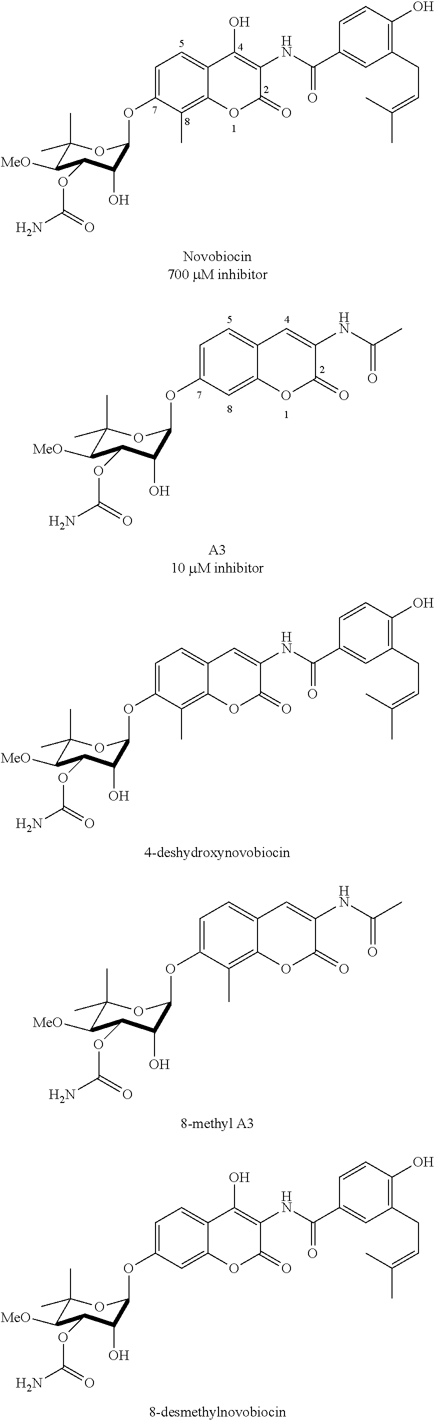

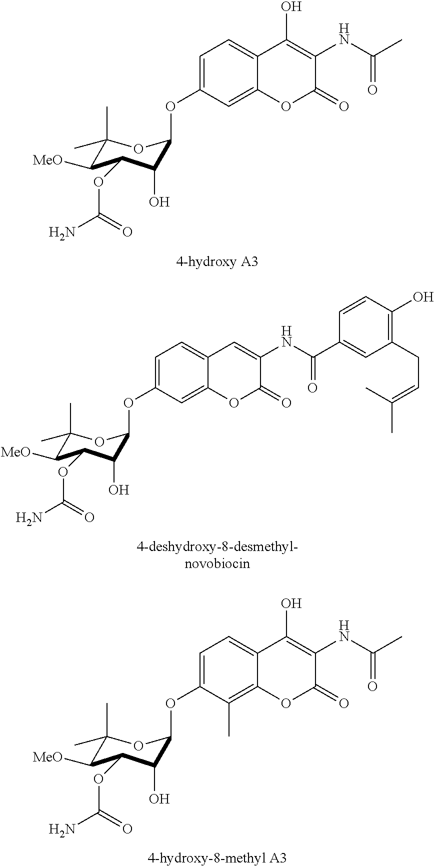

More specifically, 4-deshydroxynovobiocin can be prepared from 3-N-acetyl-7-hydroxy-8-methyl coumarin and the known carboxylic acid as set forth in Scheme 12. Spencer et al., Novobiocin. IV. Synthesis of Dihydronovobiocic Acid and Cyclonovobiocic Acid, J. Am. Chem. Soc. 78 2655-2656 (1956). Coupling of these two substrates can provide the amide, which can be treated with noviose carbonate in analogous fashion to other reported syntheses of novobiocin. See Vaterlaus et al., Die Synthese des Novobiocins, Experientia 19 383-391 (1963); and Vaterlaus et al., Novobiocin III Die Glykosidsynthese des Novobiocins, Helv. Chim. Acta 47 390-398 (1964). Likewise, 8-desmethyl-novobiocin can be prepared from 4,7-dihydroxycoumarin and the diazonium salt to afford the masked amino group similar to our syntheses of photolabile derivatives. See Shen et al., Synthesis of Photolabile Novobiocin Analogues, Bioorg. Med. Chem. Lett. 14 5903-5906 (2004).

The 7-hydroxyl can undergo selective noviosylation and the diazine can be reduced. The corresponding amine can be coupled with the known carboxylic acid and the carbonate opened with methanolic ammonia to give both 3-carbamoyl and diol derivatives. 4-Deshydroxy-8-desmethylnovobiocin can be constructed from 3-amino-7-hydroxycoumarin in analogous fashion as depicted in the scheme below. The KU-1/A4 and KU-2/A3 analogues incorporating the same coumarin functionalities can be prepared by an identical method (see Khoo, Synthesis of Substituted 3-Aminocoumarins from Ethyl N-2-Hydroxyarylideneglycinates, Syn. Comm. 29 2533-2538 (1999)) using acetic anhydride in lieu of the prenylated 4-hydroxybenzoic acid. Des(carbamoyl) derivatives of these compounds can also be prepared by removal of the cyclic carbonate with triethylamine in methanol, which affords similar products in stoichiometric yields.

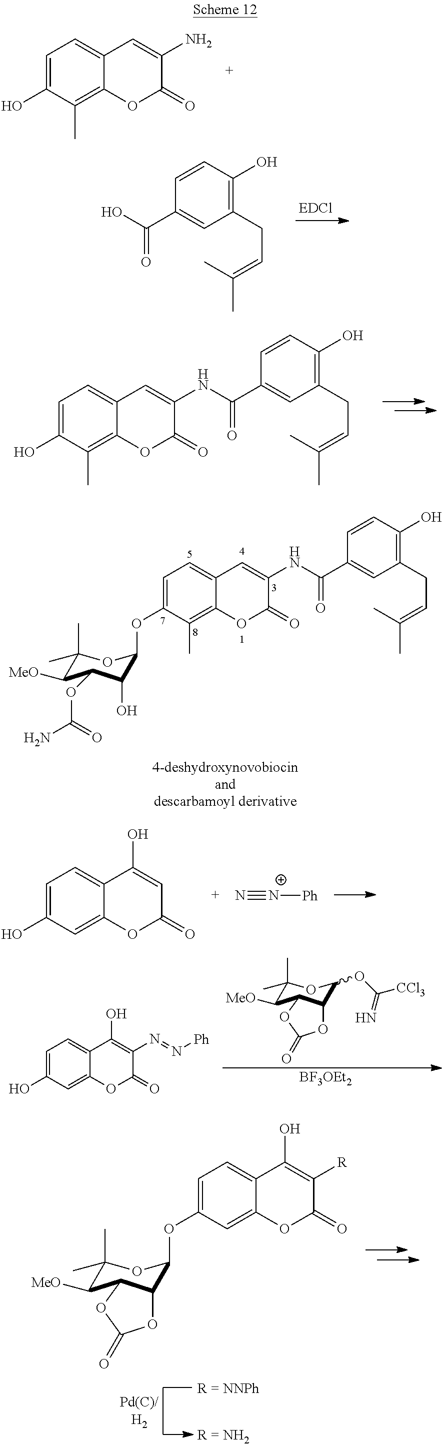

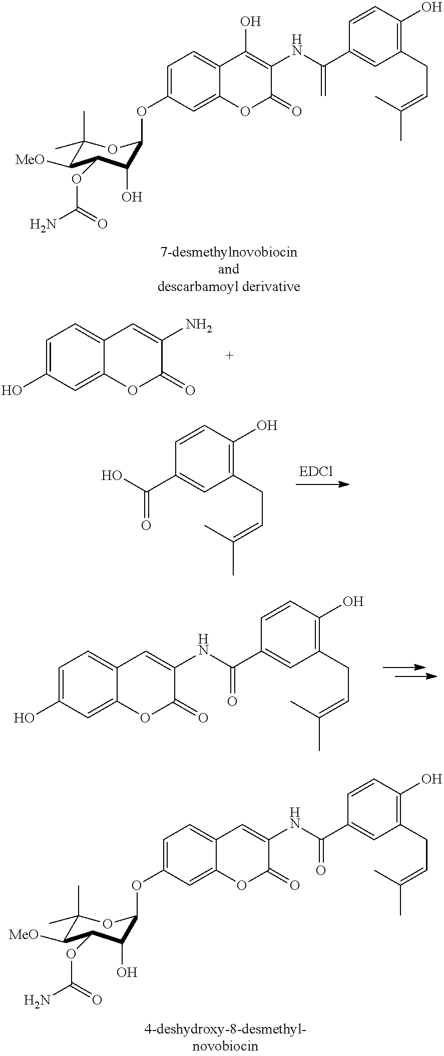

The substituted benzamide side chain of novobiocin was prepared from methyl 3-allyl-4-hydroxybenzoate, 5 in Scheme 13 below. Attempts to perform cross-metathesis on this substrate failed as complexation with the Grubbs' catalyst appeared to have occurred with the orthophenol substrate. Therefore, the phenol was temporarily masked as the acetate, which allowed for a productive cross-metathesis reaction between 2-methyl-2-butene and the allyl appendage in excellent yield to provide the prenylated benzoic ester, 7. The ester product (7) was then hydrolyzed and the phenol reprotected as the acetate to prevent subsequent ester formation. Attempts to couple the unprotected phenol as well as the benzoic acid directly with the coumarin amine resulted in the formation of a complex mixture of products that produced only trace amounts of the desired amide. Therefore, acid 9 was converted to the corresponding acid chloride (10) in high yield following standard conditions.

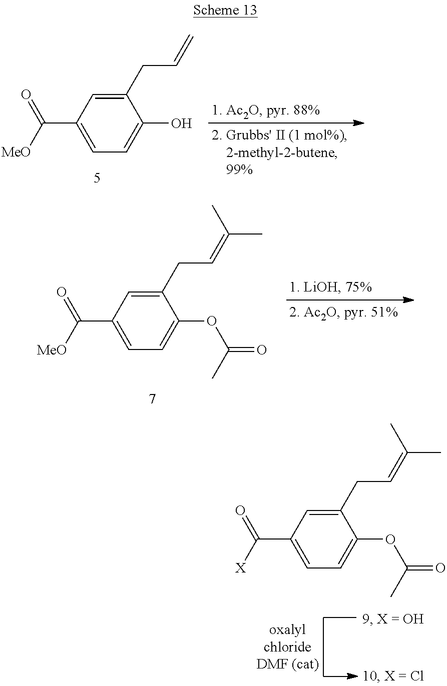

Preparation of the 4-deshydroxy coumarin ring was achieved by the condensation of 2-methylresorcinol (11) with the CBz-protected vinylagous carbamate 12, which produced the desired coumarin 13, in modest yield in Scheme 14 below. The phenol was then noviosylated with the trichloroacetimidate of noviose (14) in the presence of catalytic amounts of boron trifluoride etherate to generate 15 in good yield. Hydrogenolysis of the benzyl carbonate afforded the amine 16, which was readily coupled with the acid chloride 10 to give 17 in good yield. Both the acetate and the cyclic carbonate were removed and modified, respectively, to give the desired 3′-carbamoyl product, 4-deshydroxynovobiocin (DHN1) in good yield. Alternatively, the acetate and cyclic carbonate could be readily hydrolyzed to yield the desired 3′-descarbamoyl-4-deshydroxynovobiocin product (DHN2) in a single step upon treatment with methanolic triethylamine.

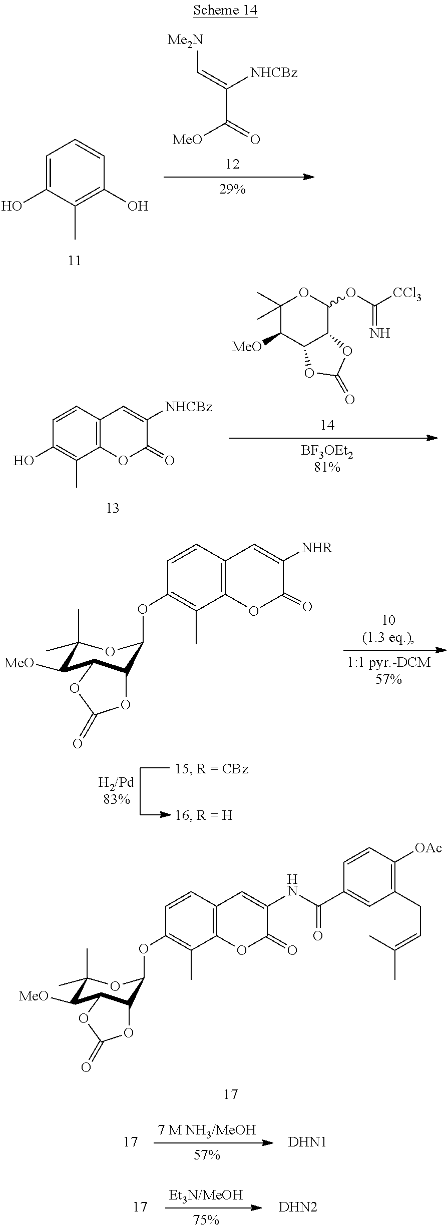

Methyl 3-allyl-4-hydroxybenzoate (5). A mixture of methyl-4-allyloxy-benzoate (4.74 g, 24.7 mmol) in N,N-diethylaniline (10 mL) was heated at reflux for 48 hours and cooled to room temperature. The mixture was diluted with diethyl ether (50 mL), washed with aqueous HCl (10% v/v, 3×20 mL), dried (Na₂SO₄), filtered and concentrated. The residue was purified by chromatography (10:1→5:1, hexanes:EtOAc) to afford 5 (3.55 g, 75%) as an off-white solid: ¹H NMR (CDCl₃, 400 MHz) δ 7.86-7.84 (m, 2H), 6.85 (dd, J=2.8, 7.7 Hz, 1H), 6.07-5.95 (m, 1H), 5.68 (s, 1H), 5.21-5.15 (m, 2H), 3.88 (s, 3H), 3.45 (d, J=6.3 Hz, 2H).

Methyl 4-acetoxy-3-allylbenzoate (6). Acetic anhydride (200 μL, 218 mg, 2.13 mmol) was added dropwise to a solution of phenol 5 (315 mg, 1.64 mmol) in pyridine (1.5 mL) at room temperature. The mixture was stirred for 14 hours before the solvent was removed. The residue was purified by chromatography (10:1, hexanes:EtOAc) to afford 6 (337 mg, 88%) as a colorless oil: ¹H NMR (CDCl₃, 400 MHz) δ 7.95-7.90 (m, 2H), 7.11 (d, J=8.2 Hz, 1H), 5.92-5.83 (m, 1H), 5.12-5.03 (m, 2H), 3.88 (s, 3H), 3.33 (d, J=6.5 Hz, 2H), 2.29 (s, 3H); ¹³C NMR (CDCl₃, 100 MHz) δ 169.2, 166.8, 153.0, 135.6, 132.7, 132.4, 129.4, 128.4, 123.0, 117.2, 52.6, 35.0, 21.3; IR (neat) νₘₐₓ 3080, 3005, 2980, 2953, 2916, 2845, 1765, 1722, 1639, 1609, 1589, 1493, 1437, 1418, 1369, 1285, 1263, 1190, 1163, 1121 cm⁻¹; HRMS (ESI⁺) m/z 235.1076 (M+H⁺, C₁₃H₁₅O₄ requires m/z 235.0970).

Methyl 4-acetoxy-3-(3-methylbut-2-enyl)benzoate (7). Grubbs' second generation catalyst (11 mg, 0.0130 mmol, 1 mol %) was added to a solution of acetate 6 (305 mg, 1.30 mmol) in a 1/10 solution of DCM/2-methyl-2-butene (5.5 mL). The mixture was stirred 14 hours and was concentrated. The residue was purified by chromatography (10:1, hexanes:EtOAc) to afford 7 (339 mg, 99%) as a colorless oil: ¹H NMR (CDCl₃, 400 MHz) δ 7.92 (d, J=1.9 Hz, 1H), 7.88 (dd, J=1.9, 8.4 Hz, 1H), 7.07 (d, J=8.4 Hz, 1H), 5.22 (td, J=1.3, 7.1 Hz, 1H), 3.87 (s, 3H), 3.25 (d, J=7.1 Hz, 2H), 2.24 (s, 3H), 1.72 (s, 3H), 1.68 (s, 3H); ¹³C NMR (CDCl₃, 100 MHz) δ 169.2, 166.9, 153.0, 134.3, 134.1, 132.3, 129.0, 128.3, 122.9, 121.4, 52.5, 29.2, 26.1, 21.2, 18.2; IR (neat) νₘₐₓ 2970, 2953, 2916, 2856, 1765, 1724, 1609, 1589, 1493, 1437, 1369, 1285, 1263, 1204, 1192, 1165, 1111 cm⁻¹; HRMS (ESI+) m/z 263.1296 (M+H⁺, C₁₅H₁₉O₄ requires m/z 263.1283).

4-Hydroxy-3-(3-methylbut-2-enyl)benzoic acid (8). Lithium hydroxide (85 mg, 2.02 mmol) was added to a mixture of methyl ester 7 (106 mg, 0.405 mmol) in 0.5 mL of a 3/1/1 THF/MeOH/H₂O solution. The reaction mixture was stirred at reflux for 14 hours, cooled to room temperature, and diluted with THF (1 mL). The solution was acidified the solution to pH=3 by the dropwise addition of 6 M HCl. The layers were separated and the organic phase was dried (Na₂SO₄), filtered, and concentrated to afford acid 8 (63 mg, 75%) as a red oil that was suitable for use without further purification.

4-Acetoxy-3-(3-methylbut-2-enyl)benzoic acid (9). Acetic anhydride (1 mL) was added dropwise to a solution of acid 8 (178 mg, 2.00 mmol) in pyridine (3 mL) at room temperature. After stirring for 48 hours, the mixture was poured into water (6 mL) and acidified to pH=2 by the dropwise addition of 6 M HCl. The suspension was extracted with EtOAc (2×10 mL), and the combined organic fractions were dried (Na₂SO₄), filtered and concentrated. The residue was purified by chromatography (5:1, hexanes:EtOAc) to afford acetate 9 (223 mg, 51%) as a white solid: ¹H NMR (CDCl₃, 400 MHz) δ 8.01-7.96 (m, 2H), 7.15 (dd, J=3.5, 8.2 Hz, 1H), 5.24 (tt, J=1.3, 7.2 Hz, 1H), 3.30 (d, J=7.2 Hz, 2H), 2.34 (s, 3H), 1.97 (s, 3H), 1.76 (s, 3H).

Benzyl 7-hydroxy-8-methyl-2-oxo-2H-chromen-3-ylcarbamate (13). 2-Methyl rescorcinol (1.20 g, 9.71 mmol) was added to a solution of vinyl carbamate 12 (2.7 g, 9.71 mmol) in acetic acid (50 mL). The mixture was stirred at reflux for 48 hours, cooled to room temperature, and filtered. The solid was recrystallized from methanol and H₂O to afford 13 (1.30 g, 41%) as a yellow solid: ¹H NMR (DMSO, 400 MHz) δ 10.30 (s, 1H), 9.10 (s, 1H), 8.12 (s, 1H), 7.46-7.30 (m, 6H), 6.85 (d, J=8.4 Hz, 1H), 5.17 (s, 2H), 2.16 (s, 3H).

Benzyl-7-((3aR,4R,7R,7aR)-7-methoxy-6,6-dimethyl-2-oxotetrahydrdo-3aH-[1.3]dioxolo[4,5-c]pyran-4-yloxy)-8-methyl-2-oxo-2H-chromen-3-ylcarbamate (15). Boron trifluoride etherate (61 μL, 69 mg, 0.49 mmol, 30 mol %) was added dropwise to a solution of (3aR,4S,7R,7aR)-7-methoxy-6,6-dimethyl-2-oxo-tetrahydro-3aH-[1,3]dioxolo[4,5-c]pyran-4-yl 2,2,2-trichloroacetimidate (14, 588 mg, 1.62 mmol) and benzyl 7-hydroxy-8-methyl-2-oxo-2H-chromen-3-yl carbamate (13, 527 mg, 1.62 mmol) in DCM (16 mL). After the mixture stirred for 14 hours, three drops of Et₃N were added and the mixture concentrated. The residue was purified by chromatography (DCM-100:1, CH₂Cl₂:acetone) to afford 15 (670 mg, 81%) as a yellow foam: [α]²²_{D}=−19.7° (c=1.54, 20% MeOH in DCM); ¹H NMR (CDCl₃, 400 MHz) δ 8.27 (s, 1H), 7.85 (s, 1H), 7.55-7.35 (m, 5H), 7.29 (d, J=2.9 Hz, 1H), 7.11 (d, J=8.7 Hz, 1H), 5.77 (d, J=1.9 Hz, 1H), 5.23 (s, 2H), 5.05 (d, J=1.9 Hz, 1H), 4.95 (t, J=7.7 Hz, 1H), 3.59 (s, 3H), 3.30 (d, J=7.6 Hz, 1H), 2.27 (s, 3H), 1.34 (s, 3H), 1.19 (s, 3H); ¹³C NMR (CDCl₃, 100 MHz) δ 159.0, 155.2, 153.6, 153.6, 149.2, 136.0, 129.1 (2C), 129.0, 128.7 (2C), 125.8, 122.6, 122.1, 115.2, 115.1, 111.6, 94.8, 83.3, 78.4, 77.6, 77.0, 67.9, 61.0, 27.9, 22.6, 8.8; IR (film) νₘₐₓ 3402, 3319, 3063, 3034, 2984, 2939, 2839, 1817, 1709, 1634, 1609, 1587, 1522, 1456, 1383, 1366, 1331, 1296, 1263, 1229, 1205, 1175 cm⁻¹; HRMS (ESI+) m/z 526.1688 (M+H⁺, C₂₇H₂₈NO₁₀ requires m/z 526.1713).

3-Amino-7-((3aR,4R,7R,7aR)-7-methoxy-6,6-dimethyl-2-oxo oxotetrahydrdo-3aH-[1.3]dioxolo[4,5-c]pyran-4-yloxy)-8-methyl-2H-chromen-2-one (16). Palladium on carbon (10%, 67 mg) was added to a solution of carbamate 15 (670 mg, 1.31 mmol) in THF (13 mL). The suspension stirred for 6 hours under a hydrogen atmosphere and was filtered through a plug of silica gel. The solvent was removed and the residue purified by chromatography (100:1→50:1, CH₂Cl₂:acetone) to afford 16 (425 mg, 83%) as a pale yellow foam: [α]²³_{D}=−26.4° (c=0.780, 20% MeOH in DCM); ¹HNMR (CDCl₃, 400 MHz) δ 7.10 (d, J=8.6 Hz, 1H), 7.05 (d, J=8.6 Hz, 1H), 6.68 (s, 1H), 5.73 (d, J=2.0 Hz, 1H), 5.04 (dd, J=2.0, 7.9 Hz, 1H), 4.95 (t, J=7.7 Hz, 1H), 4.11 (s, 2H), 3.54 (s, 3H), 3.29 (d, J=7.6 Hz, 1H), 2.28 (s, 3H), 1.34 (s, 3H), 1.21 (s, 3H); ¹³C NMR (CDCl₃, 200 MHz) δ 159.6, 153.3, 153.0, 148.1, 130.2, 122.7, 116.1, 114.8, 111.9, 111.0, 94.5, 83.0, 78.0, 77.3, 76.4, 60.6, 27.5, 22.2, 8.6; IR (film) νₘₐₓ 3462, 3362, 2984, 2937, 2839, 1807, 1707, 1636, 1595, 1497, 1387, 1371, 1331, 1263, 1169, 1109, 1078, 1036 cm⁻¹; HRMS (ESI+) m/z 392.1357 (M+H⁺, C₁₉H₂₂NO₈ requires m/z 392.1346).

4-((7-((3aR,4R,7R,7aR)-7-Methoxy-6,6-dimethyl-2-oxotetrahydrdo-3aH-[1.3]dioxolo[4,5-c]pyran-4-yloxy)-8-methyl-2-oxo-2H-chromen-3-ylcarbamoyl)-2-(3-methylbut-2-enyl)phenyl acetate (17). Oxalyl chloride (15 mg, 119 μmol) was added to a solution of benzoic acid 9 (28 mg, 113 μmol) in CH₂Cl₂ (0.5 mL), followed by the addition of catalytic DMF. After stirring for 2.5 hours, the acid chloride (10) was concentrated. The yellow solid was redissolved in CH₂Cl₂ (0.5 mL) and added dropwise over three minutes to a stirred solution of aniline 16 (34 mg, 87 μmol) in pyridine (0.5 mL) at 0° C. The resulting solution was stirred at room temperature for 3.5 hours and concentrated. The residue was purified by preparative TLC (SiO₂, 40:1, CH₂Cl₂:acetone) to afford 17 (31 mg, 57%) as a colorless solid: [α]22_{D}=−21.7° (c=0.840, 20% MeOH in CH₂Cl₂); ¹H NMR (CDCl₃, 500 MHz) δ 8.72 (s, 1H), 8.64 (s, 1H), 7.73 (d, J=2.5 Hz, 1H), 7.69 (dd, J=2.5, 8.0 Hz, 1H), 7.29 (d, J=6.8 Hz, 1H) 7.11 (d, J=8.0 Hz, 1H), 7.07 (d, J=9.0 Hz, 1H), 5.72 (d, J=2.0 Hz, 1H), 5.18-5.14 (m, 1H), 4.99 (dd, J=1.5, 7.5 Hz, 1H), 4.89 (t, J=8.0 Hz, 1H), 3.53 (s, 3H), 3.26-3.22 (m, 3H), 2.27 (s, 3H), 2.23 (s, 3H), 1.70 (s, 3H), 1.66 (s, 3H), 1.29 (s, 3H), 1.13 (s, 3H); ¹³C NMR (CDCl₃, 125 MHz) δ 167.9, 164.4, 158.1, 154.1, 152.2, 151.1, 148.1, 133.6, 133.3, 131.0, 128.6, 124.9, 123.1, 122.0 (2C), 121.2, 119.6, 113.8, 113.7, 110.2, 93.3, 81.9, 76.9, 76.3, 75.6, 59.5, 27.8, 26.5, 24.7, 21.1, 19.9, 16.9, 7.4; IR (film) νₘₐₓ 3400, 2982, 2935, 2856, 1811, 1763, 1715, 1674, 1634, 1607, 1526, 1489, 1437, 1369, 1250, 1202, 1175, 1111, 1090 cm⁻¹; HRMS (ESI+) m/z 622.2277 (M+H⁺, C₃₃H₃₆NO₁₁ requires m/z 622.2289).

(3R,4S,5R,6R)-5-Hydroxy-6-(3-(4-hydroxy-3-(3-methylbut-2-enyl)benzylamino)-8-methyl-2-oxo-2H-chromen-7-yloxy)-3-methoxy-2,2-dimethyltetrahydro-2H-pyran-4-yl carbamate (DHN1). A solution of carbonate 17 (32 mg, 52 μmol) in 7 M methanolic ammonia (2 mL) was stirred for 14 hours. The solvent was removed and the residue purified by preparative TLC (SiO₂, 25:1, CH₂Cl₂:methanol, developed 7 times) to afford DHN2 (2.5 mg, 9%) and 4-deshydroxynovobiocin (DHN1, 17.5 mg, 57%) as colorless solids; DHN1: [α]³¹_{D}=−20.3° (c=0.300, 10% MeOH in CH₂Cl₂); ¹H NMR (CDCl₃, 400 MHz) δ 8.70 (s, 1H), 7.62 (d, J=2.3 Hz, 1H), 7.56 (dd, J=2.3, 8.4 Hz, 1H), 7.29 (d, J=8.8 Hz, 1H), 7.13 (d, J=8.8 Hz, 1H), 6.81 (d, J=8.4 Hz, 1H), 5.50 (d, J=2.3 Hz, 1H), 5.34-5.25 (m, 2H), 4.25 (t, J=2.6 Hz, 1H), 3.53-3.51 (m, 1H), 3.50 (s, 3H), 3.34 (dd, J=3.2, 8.9 Hz, 2H), 2.99 (s, 1H), 2.94 (s, 1H), 2.27 (s, 3H), 1.74 (s, 3H), 1.71 (s, 3H), 1.33 (s, 3H), 1.13 (s, 3H); ¹³C NMR (CDCl₃, 125 MHz) δ 166.3, 159.6, 158.9, 156.8, 155.9, 149.0, 133.9, 129.0, 128.6, 126.4, 125.6, 124.6, 124.1, 121.8, 121.4, 114.9, 114.4, 114.1, 111.2, 98.3, 81.4, 78.9, 72.2, 69.6, 61.5, 29.7, 29.3, 27.2, 22.6, 17.9, 8.2; IR (film) νₘₐₓ 3400, 3379, 3360, 2978, 2928, 2853, 1709, 1659, 1632, 1605, 1528, 1504, 1367, 1254, 1136, 1117, 1086 cm⁻¹; HRMS (ESI+) m/z 597.2434 (M+H⁺, C₃₁H₃₇N₂O₁₀ requires m/z 297.2448). The structure of DNH1 is:

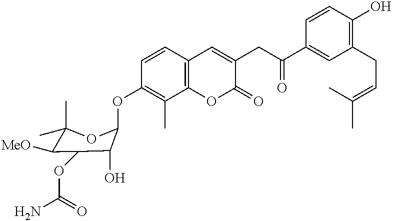

N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)-4-hydroxy-3-(3-methylbut-2-enyl)benzylamide (DHN2). A solution of carbonate 17 (12 mg, 19.3 μmol) in 10/1 methanol/Et₃N (220 μL) was stirred for 14 hours. The solvent was removed and the residue purified by preparative TLC (SiO₂, 10:1, CH₂Cl₂:methanol) to afford DHN2 (8 mg, 75%) as a colorless solid: [α]³¹_{D}=−12.9° (c=0.310, 10% MeOH in DCM); ¹H NMR (CDCl₃, 400 MHz) δ 8.78 (s, 1H), 8.66 (s, 1H), 7.71 (d, J=2.2 Hz, 1H), 7.68 (dd, J=2.2, 8.3 Hz, 1H), 7.33 (d, J=8.8 Hz, 1H), 7.19 (d, J=8.8 Hz, 1H), 6.90 (d, J=8.3 Hz, 1H), 6.05 (s, 1H), 5.61 (d, J=1.6 Hz, 1H), 5.33 (t, J=7.1 Hz, 1H), 4.27-4.23 (m, 2H), 3.61 (s, 3H), 3.45-3.35 (m, 3H), 2.77 (s, 1H), 2.67 (s, 1H), 2.05 (s, 3H), 1.80 (s, 3H), 1.79 (s, 3H), 1.38 (s, 3H), 1.14 (s, 3H); ¹³C NMR (CDCl₃, 125 MHz) δ 165.9, 159.5, 158.3, 155.9, 149.0, 135.8, 129.5, 127.5, 126.9, 125.9, 125.8, 124.2, 122.0, 120.9, 115.9, 114.2, 114.1, 111.2, 97.7, 84.3, 78.6, 71.2, 68.6, 62.0, 29.6, 29.3, 25.8, 22.5, 18.0, 8.2; IR (film) νₘₐₓ 3402, 2974, 2928, 2854, 1717, 1701, 1645, 1605, 1526, 1506, 1367, 1254, 1088 cm⁻¹; HRMS (ESI+) m/z 554.2363 (M+H⁺, C₃₀H₃₆NO₉ requires m/z 554.2390). The structure of DNH2 is:

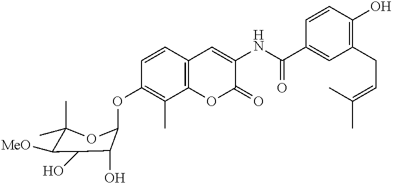

### Example 8

### Benzamide Novobiocin Derivatives

This example involved the preparation of novobiocin analogues with highly substituted benzamides. As shown in the scheme below, the derivatives were assembled from three components: noviose carbonate (see Shen et al., Syntheses of Photolabile Novobiocin Analogues, Bioorg. Med. Chem. Lett. 14 5903 (2004)), 8-methylcoumarin (Toplak et al., The synthesis of methyl 2-(benzyloxycarbonyl)amino-3-dimethylaminopropenoate. The synthesis of trisubstituted pyrroles, 3-amino-2H-pyran-2-ones, fused 2H-pyran-2-ones and 4H-pyridin-4-ones, J. Hetero. Chem. 36 225-235 (1999), and a series of substituted benzoic acids. Previously, it had been demonstrated that the trichloroacetimidate of noviose carbonate couples directly to the coumarin phenol to afford the α-anomer in excellent yield (see Shen et al., Syntheses of Photolabile Novobiocin Analogues, Bioorg. Med. Chem. Lett. 14 5903 (2004)). Commercially available benzoic acids were chosen that contained various functionalities in an effort to probe for steric and electronic interactions with the putative hydrophobic pocket that is believed to bind to this region of novobiocin. The following shows the retrosynthesis of the novobiocin analogues:

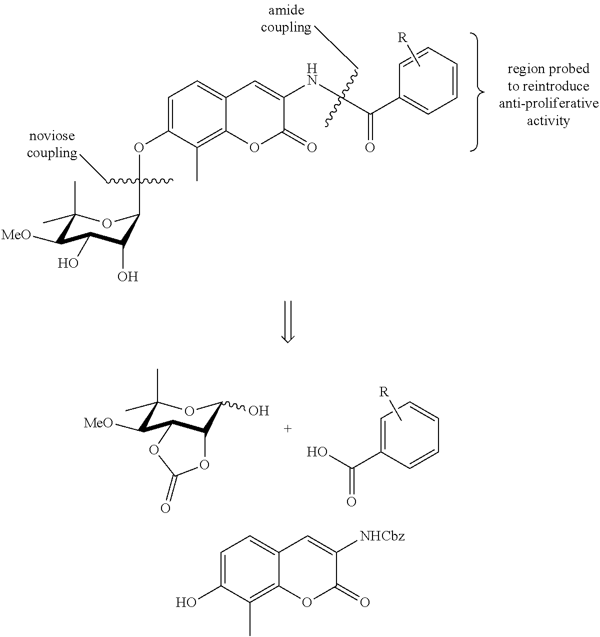

More specifically, novobiocin analogues were prepared by the condensation of N,N-dimethylformamide dimethyl acetal (2) with Cbz-protected glycine (1) to produce the vinylagous carbmate, 3 as set forth in Scheme 15 below. See Robinson et al., Highly enantioselective synthesis of alpha,beta-diaminopropanoic acid derivatives using a catalytic asymmetric hydrogenation approach, J. Org. Chem. 66 4141-4147 (2001). The 8-methylcoumarin 5 was prepared by a modified Pechmann condensation of 2-methylresorcinol (4) with 3. Toplak et al., J. Hetero. Chem. 36 225-235 (1999). The resulting phenol was noviosylated with the trichloroaceimidate of noviose carbonate (6) (Yu et al., Synthesis of (−)-Noviose from 2,3-O-Isopropylidene-D-erythronolactol, J. Org. Chem., 69 7375-7378 (2004)) in the presence of catalytic boron trifluoride etherate to give 7 in good yield. See Shen et al., Synthesis of Photolabile Novobiocin Analogues, Bioorg. Med. Chem. Lett. 145903-5906 (2004). The benzyl carbonate was removed via hydrogenolysis to produce aminocoumarin 7, which proved to be a versatile intermediate throughout this project. The amine was readily coupled to a preselected library of benzoic acids in the presence of N-(3-dimethylamino-propyl)-N′-ethylcarbodiimide hydrochloride (EDCI) and 4-DMAP. During the course of the investigation, it was determined that utilization of 4-DMAP led to bisacylation, which proved difficult to separate from the monoacylated product. Therefore pyridine was employed as the base and provided exclusively monoacylated products. With the desired benzamides in hand, the cyclic carbonates underwent solvolysis with triethylamine in methanol to give the diol in excellent yield. To complete the small library of inhibitors, aryl nitro compounds (15-17) were subjected to hydrogenation to afford the corresponding anilines.

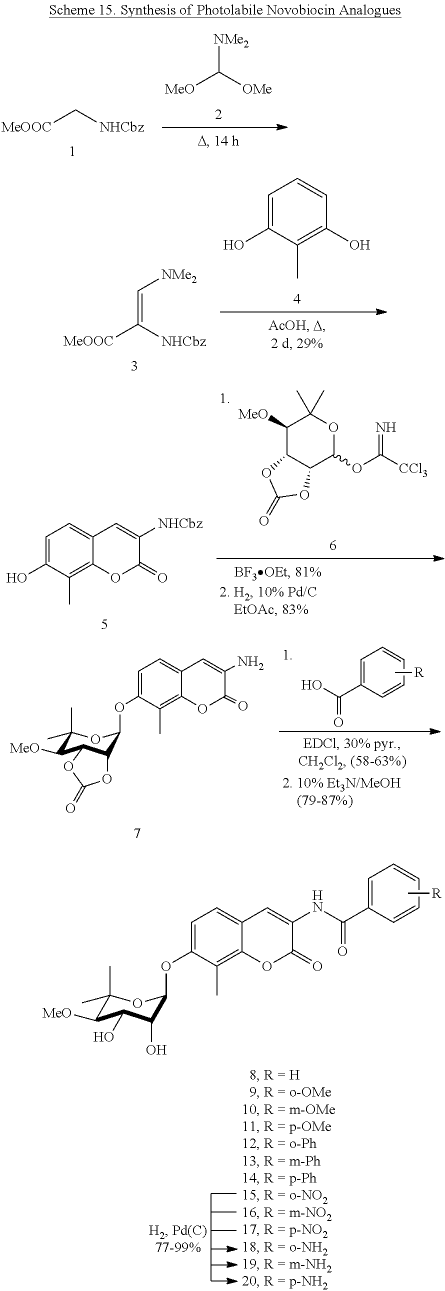

General EDCI coupling procedure A: N-(3-Dimethylamino-propyl)-N-ethylcarbodiimide hydrochloride (3 eq) was added to a solution of aminocoumarin 7 (1 eq), benzoic acid (3 eq) and 4-DMAP (2.0 eq) in CH₂Cl₂ at room temperature. The solution was stirred for 14 hours, concentrated and the residue purified via preparative TLC or column chrotography (SiO₂, 40:1; CH₂Cl₂:acetone) to afford the benzamide.

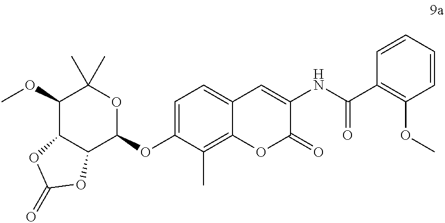

2-Methoxy-N-(7-((3aR,4R,7R,7aR)-7-methoxy-6,6-dimethyl-2-oxotetrahydro-3aH-[1,3]dioxolo[4,5-c]pyran-4-yloxy)-8-methyl-2-oxo-2H-chromen-3-yl)benzamide (9a). Colorless solid (66%): [α]²⁴_{D}=−29.6° (c=0.61, 20% MeOH in CH₂Cl₂); ¹HNMR (400 MHz, CDCl₃) δ 8.77 (s, 1H), 8.18 (d, J=7.8 Hz, 1H), 7.46 (t, J=7.8 Hz, 1H), 7.27 (d, J=8.3 Hz, 1H), 7.09-7.02 (m, 2H), 6.99 (d, J=8.3 Hz, 1H), 5.71 (s, 1H), 4.99 (d, J=7.9 Hz, 1H), 4.91 (t, J=7.9 Hz, 1H), 4.05 (s, 3H), 3.53 (s, 3H), 3.24 (d, J=7.9 Hz, 1H), 2.23 (s, 3H), 1.29 (s, 3H), 1.14 (s, 3H); ¹³C NMR (100 MHz, CDCl₃) δ 164.5, 159.6, 158.1, 155.3, 153.6, 149.5, 134.3, 132.6, 126.4, 124.4, 123.5, 121.9, 121.3, 115.5, 115.1, 112.0, 111.5, 94.8, 83.3, 78.4, 77.4, 77.1, 61.0, 56.6, 27.9, 22.6, 8.8; IR (film) νₘₐₓ 3308, 3055, 2986, 2939, 2930, 1817, 1807, 1707, 1655, 1603, 1533, 1481, 1466, 1367, 1263, 1232 cm⁻¹; HRMS (ESI+) m/z 526.1691 (M+H⁺, C₂₇H₂₈NO₁₀ requires m/z 526.1713).

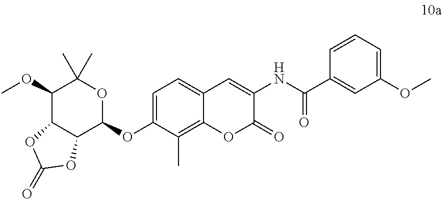

3-Methoxy-N-(7-((3aR,4R,7R,7aR)-7-methoxy-6,6-dimethyl-2-oxotetrahydro-3aH-[1,3]dioxolo[4,5-c]pyran-4-yloxy)-8-methyl-2-oxo-2H-chromen-3-yl)benzamide (10a). Colorless solid (43%): [α]²⁵_{D}=−27.1° (c=1.22, 20% MeOH in CH₂Cl₂); ¹H NMR (400 MHz, CDCl₃) δ 8.82 (s, 1H), 8.76 (s, 1H), 7.51-7.44 (m, 3H), 7.39 (d, J=8.7 Hz, 1H), 7.19-7.11 (m, 2H), 5.81 (d, J=1.8 Hz, 1H), 5.08 (dd, J=1.8, 7.8 Hz, 1H), 4.98 (t, J=7.8 Hz, 1H), 3.91 (s, 3H), 3.62 (s, 3H), 3.33 (d, J=7.8 Hz, 1H), 2.32 (s, 3H), 1.38 (s, 3H), 1.22 (s, 3H); ¹³C NMR (100 MHz, CDCl₃) δ 166.3, 160.5, 159.6, 155.6, 153.6, 149.5, 135.5, 130.4, 126.3, 124.5, 122.6, 119.3, 119.0, 115.3, 115.1, 112.9, 111.7, 94.8, 83.3, 78.3, 77.4, 77.1, 61.0, 55.9, 27.9, 22.6, 8.9; IR (film) νₘₐₓ 3402, 3057, 2988, 2939, 2839, 1819, 1809, 1709, 1676, 1609, 1526, 1487, 1369, 1263, 1175, 1153, 1097, 1076 cm⁻¹; HRMS (ESI+) m/z 526.1695 (M+H⁺, C₂₇H₂₈NO₁₀ requires m/z 526.1713).

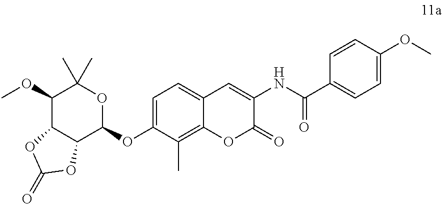

4-Methoxy-N-(7-((3aR,4R,7R,7aR)-7-methoxy-6,6-dimethyl-2-oxotetrahydro-3 aH-[1,3]dioxolo[4,5-c]pyran-4-yloxy)-8-methyl-2-oxo-2H-chromen-3-yl)benzamide (11a). Colorless solid (35%): [α]²⁴_{D}=−25.8° (c=0.69, 20% MeOH in CH₂Cl₂); ¹H NMR (400 MHz, CDCl₃) δ 8.81 (s, 1H), 8.71 (s, 1H), 7.91 (d, J=8.8 Hz, 2H), 7.38 (d, J=8.7 Hz, 1H), 7.16 (d, J=8.7 Hz, 1H), 7.02 (d, J=8.8 Hz, 2H), 5.81 (d, J=1.8 Hz, 1H), 5.08 (dd, J=1.9, 7.9 Hz, 1H), 4.98 (t, J=7.8 Hz, 1H), 3.91 (s, 3H), 3.62 (s, 3H), 3.33 (d, J=7.8 Hz, 1H), 2.35 (s, 3H), 1.38 (s, 3H), 1.23 (s, 3H); ¹³C NMR (100 MHz, CDCl₃) δ 165.9, 163.4, 159.7, 155.5, 153.6, 149.4, 129.5 (3C), 126.3, 126.2, 124.1, 122.8, 115.3, 114.5 (2C), 111.7, 94.8, 83.3, 78.3, 77.4, 77.1, 61.0, 55.9, 27.9, 22.6, 8.9; IR (film) νₘₐₓ 3406, 2984, 2937, 2839, 1811, 1709, 1670, 1607, 1529, 1506, 1367, 1246, 1175 cm⁻¹; HRMS (ESI+) m/z 526.1690 (M+C₂₇H₂₈NO₁₃ requires m/z 526.1713).

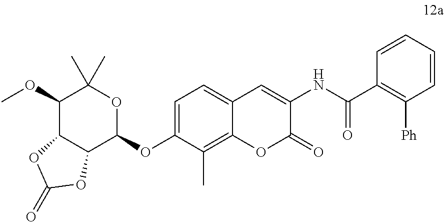

N-(7-((3aR,4R,7R,7aR)-7-Methoxy-6,6-dimethyl-2-oxotetrahydro-3aH-[1,3]dioxolo[4,5-c]pyran-4-yloxy)-8-methyl-2-oxo-2H-chromen-3-yl)biphenyl-2-carboxamide (12a). Colorless solid (35%): [α]²⁶_{D}=−22.8° (c=0.15, 20% MeOH in CH₂Cl₂); ¹H NMR (400 MHz, CDCl₃) δ 8.70 (s, 1H), 7.93 (s, 1H), 7.80 (d, J=7.8 Hz, 1H), 7.61-7.36 (m, 8H), 7.33 (d, J=8.7 Hz, 1H), 7.13 (d, J=8.7 Hz, 1H), 5.79 (d, J=1.8 Hz, 1H), 5.6 (dd, J=1.8, 7.9 Hz, 1H), 4.97 (t, J=7.9 Hz, 1H), 3.61 (s, 3H), 3.32 (d, J=7.9 Hz, 1H), 2.28 (s, 3H), 1.37 (s, 3H), 1.21 (s, 3H); ¹³C NMR (100 MHz, CDCl₃) δ 168.8, 158.8, 155.4 (2C), 140.6, 140.0, 135.1, 131.5, 131.1, 129.4 (2C), 129.2 (2C), 129.1 (2C), 128.6, 128.2, 126.2, 124.0, 122.4, 115.2, 115.1, 111.5, 94.7, 83.3, 78.3, 77.4, 77.1, 61.0, 27.9, 22.6, 8.8; IR (film) νₘₐₓ 3375, 2984, 2935, 1815, 1715, 1672, 1609, 1516, 1367, 1256, 1171, 1111, 1094, 1076 cm⁻¹; HRMS (ESI+) m/z 572.1893 (M+H⁺, C₃₂H₃₀NO₉ requires m/z 572.1921).

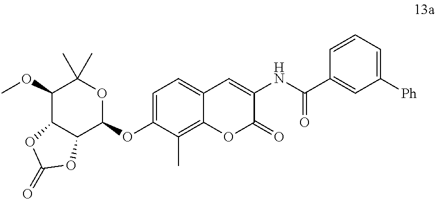

N-(7-((3aR,4R,7R,7aR)-7-Methoxy-6,6-dimethyl-2-oxotetrahydro-3 aH-[1,3]dioxolo[4,5-c]pyran-4-yloxy)-8-methyl-2-oxo-2H-chromen-3-yl)biphenyl-3-carboxamide (13a). Colorless solid (58%): [α]²⁵_{D}=−19.2° (c=0.12, 20% MeOH in CH₂Cl₂); NMR (400 MHz, CD₂Cl₂) δ 8.86 (s, 1H), 8.83 (s, 1H), 8.18 (t, J=1.7 Hz, 1H), 7.99-7.85 (m, 2H), 7.71 (dd, J=1.4, 8.5 Hz, 2H), 7.64 (t, J=7.8 Hz, 1H), 7.54 (t, J=7.4 Hz, 2H), 7.46 (dd, J=1.7, 7.4 Hz, 2H), 7.21 (d, J=8.7 Hz, 1H), 5.86 (d, J=2.2 Hz, 1H), 5.11 (dd, J=2.2, 7.8 Hz, 1H), 5.02 (t, J=7.8 Hz, 1H), 3.62 (s, 3H), 3.39 (d, J=7.8 Hz, 1H), 2.35 (s, 3H), 1.40 (s, 3H), 1.25 (s, 3H); ¹³C NMR (100 MHz, CD₂Cl₂) δ 166.2, 159.4, 155.6, 153.6, 149.6, 142.4, 140.3, 134.9, 131.4, 129.7, 129.3 (3C), 128.3, 127.6 (2C), 126.3, 126.2, 124.2, 122.8, 115.2, 115.1, 111.7, 94.9, 83.2, 78.3, 77.5, 77.0, 60.8, 27.7, 22.4, 8.5; IR (film) νₘₐₓ 3398, 3063, 2984, 2934, 1809, 1713, 1674, 1607, 1522, 1369, 1261, 1236, 1173, 1155, 1097, 1078 cm⁻¹; HRMS (ESI+) m/z 572.1901 (M+H⁺, C₃₂H₃₀NO₉ requires m/z 572.1921).

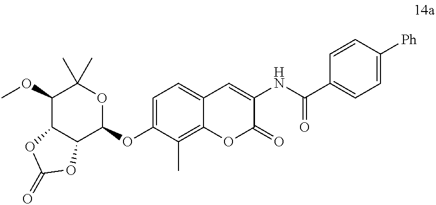

N-(7-((3aR,4R,7R,7aR)-7-Methoxy-6,6-dimethyl-2-oxotetrahydro-3 aH-[1,3]dioxolo[4,5-c]pyran-4-yloxy)-8-methyl-2-oxo-2H-chromen-3-yl)biphenyl-4-carboxamide (14a). Colorless solid (32%): [α]²⁵_{D}=−17.3° (c=0.08, 20% MeOH in CH₂Cl₂); ¹H NMR (400 MHz, CDCl₃) δ 8.86 (s, 1H), 8.83 (s, 1H), 8.02 (d, J=8.4 Hz, 2H), 7.76 (d, J=8.4 Hz, 2H), 7.67 (dd, J=1.3, 7.8 Hz, 2H), 7.51 (t, J=7.8 Hz, 2H), 7.44 (d, J=7.3 Hz, 1H), 7.41 (d, J=8.4 Hz, 1H), 7.18 (d, J=8.4 Hz, 1H), 5.82 (d, J=1.8 Hz, 1H), 5.09 (dd, J=1.8, 7.9 Hz, 1H), 4.99 (t, J=7.9 Hz, 1H), 3.62 (s, 3H), 3.34 (d, J=7.6 Hz, 1H), 2.39 (s, 3H), 1.39 (s, 3H), 1.23 (s, 3H); ¹³C NMR (100 MHz, CDCl₃) δ 165.8, 159.5, 155.6, 153.6, 149.5, 145.4, 140.0, 132.8, 129.4 (2C), 128.8, 128.1 (2C), 127.8 (2C), 127.2 (2C), 126.2, 124.1, 122.8, 115.2, 115.1, 111.7, 94.8, 83.2, 78.3, 77.5, 77.0, 60.8, 27.7, 22.4, 8.6; IR (film) νₘₐₓ 3400, 3032, 2986, 2935, 2851, 1811, 1713, 1672, 1609, 1529, 1512, 1367, 1248, 1173, 1095 cm⁻¹; HRMS (ESI+) m/z 572.1924 (M+H⁺, C₃₂H₃₀NO₉ requires m/z 572.1921).

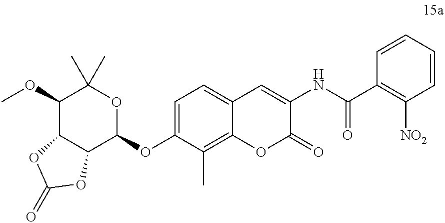

N-(7-((3aR,4R,7R,7aR)-7-Methoxy-6,6-dimethyl-2-oxotetrahydro-3aH-[1,3]dioxolo[4,5-c]pyran-4-yloxy)-8-methyl-2-oxo-2H-chromen-3-yl)-2-nitrobenzamide (15a). Yellow solid (74%): [α]²⁶_{D}=−19.5° (c=0.55, 20% MeOH in CH₂Cl₂); NMR (400 MHz, CDCl₃) δ 8.79 (s, 1H), 8.41 (s, 1H), 8.17 (dd, J=0.9, 8.1 Hz, 1H), 7.80-7.64 (m, 3H), 7.41 (d, J=8.7 Hz, 1H), 7.17 (d, J=8.7 Hz, 1H), 5.82 (d, J=1.8 Hz, 1H), 5.09 (dd, J=1.8, 7.9 Hz, 1H), 4.98 (t, J=7.9 Hz, 1H), 3.61 (s, 3H), 3.34 (d, J=7.9 Hz, 1H), 2.32 (s, 3H), 1.38 (s, 3H), 1.13 (s, 3H); ¹³C NMR (100 MHz, CDCl₃) δ 165.4, 159.2, 155.9, 153.6, 149.7, 146.8, 134.4, 132.2, 131.7, 128.8, 126.5, 125.3, 122.2, 115.3; 114.8, 111.8, 94.7, 83.3, 78.3, 77.7, 77.5, 77.1, 61.0, 27.9, 22.6, 8.8; IR (film) νₘₐₓ 3379, 3310, 3088, 2986, 2937, 2885, 2841, 1809, 1713, 1676, 1607, 1529, 1371, 1348, 1252, 1171, 1105, 1086, 1072, 1036, 1003 cm⁻¹; HRMS (ESI+) m/z 541.1441 (M+H⁺, C₂₆H₂₅N₂O₁₁ requires m/z 541.1458).

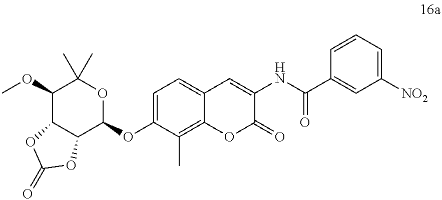

N-(7-((3aR,4R,7R,7 aR)-7-Methoxy-6,6-dimethyl-2-oxotetrahydro-3aH-[1,3]dioxolo[4,5-c]pyran-4-yloxy)-8-methyl-2-oxo-2H-chromen-3-yl)-3-nitrobenzamide (16a). Yellow solid (71%): [α]²⁶_{D}=−28.4° (c=0.29, 20% MeOH in CH₂O₂); NMR (400 MHz, CD₂Cl₂) δ 8.83 (s, 1H), 8.82 (s, 1H) 8.77 (t, J=1.9 Hz, 1H), 8.46 (td, J=1.9, 8.2 Hz, 1H), 8.27 (d, J=8.2 Hz, 1H), 7.78 (t, J=8.2 Hz, 1H), 7.46 (d, J=8.7 Hz, 1H), 7.21 (d, J=8.7 Hz, 1H), 5.86 (d, J=2.1 Hz, 1H), 5.12 (dd, J=2.1, 8.0 Hz, 1H), 5.03 (t, J=8.0 Hz, 1H), 3.62 (s, 3H), 3.39 (d, J=8.0 Hz, 1H), 2.33 (s, 3H), 1.40 (s, 3H), 1.18 (s, 3H); ¹³C NMR (100 MHz, CD₂Cl₂) δ 163.9, 159.3, 155.9, 153.6, 149.7, 148.9, 135.8, 133.1, 130.6, 127.1, 126.4, 125.1, 122.8, 122.2, 115.3, 114.8, 111.7, 94.8, 83.2, 78.3, 77.4, 77.0, 60.8, 27.7, 22.4, 8.6; IR (film) νₘₐₓ 3516, 3389, 3088, 3065, 2986, 2939, 2837, 1809, 1713, 1674, 1607, 1529, 1371, 1350, 1249, 1173, 1109, 1090, 1036 cm⁻¹; HRMS (ESI+) m/z 563.1249 (M+Na⁺, C₂₆H₂₄N₂O₁₁Na requires m/z 563.1278).

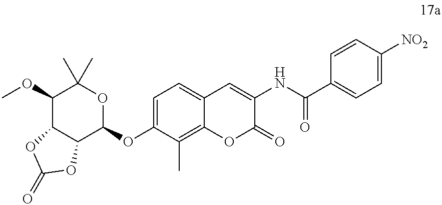

N-(7-((3aR,4R,7R,7aR)-7-Methoxy-6,6-dimethyl-2-oxotetrahydro-3aH-[1,3]dioxolo[4,5-c]pyran-4-yloxy)-8-methyl-2-oxo-2H-chromen-3-yl)-4-nitrobenzamide (17a). Yellow solid (95%): [α]²⁴_{D}=−29.5° (c=0.20, 20% MeOH in CH₂Cl₂); ¹H NMR (400 MHz, CD₂Cl₂) δ 8.83 (d, J=2.2 Hz, 2H), 8.39 (dd, J=1.7, 8.3 Hz, 2H) 8.13 (dt, J=2.2, 8.3 Hz, 2H), 7.47 (d, J=8.8 Hz, 1H), 7.22 (d, J=8.8 Hz, 1H), 5.86 (d, J=2.1 Hz, 1H), 5.12 (dd, J=2.1, 7.8 Hz, 1H), 5.03 (t, J=7.8 Hz, 1H), 3.62 (s, 3H), 3.39 (d, J=7.8 Hz, 1H), 2.34 (s, 3H), 1.40 (s, 3H), 1.24 (s, 3H); ¹³C NMR (100 MHz, CD₂Cl₂) δ 164.3, 159.3, 155.9, 153.6, 150.4, 149.7, 139.6, 128.8 (2C), 126.4, 125.1, 122.4 (2C), 122.2, 115.3, 114.8, 111.7, 94.8, 83.2, 78.3, 77.5, 77.0, 60.8, 27.7, 22.4, 8.5; IR (film) νₘₐₓ 3383, 3364, 3105, 2982, 2945, 2833, 1811, 1709, 1672, 1605, 1529, 1371, 1346, 1177, 1109, 1092, 1028 cm⁻¹; HRMS (ESI+) m/z 563.1273 (M+Na⁺, C₂₆H₂₄N₂O₁₁Na requires m/z 563.1278).

General procedure for solvolysis of the cyclic carbonate: Et3N (10% total volume) was added dropwise to a solution of cyclic carbonate in methanol. The resulting mixture was stirred for 14 hours, and then concentrated. The residue was purified via preparative TLC or column chromatography (SiO₂, 4:1; CH₂Cl₂:acetone).

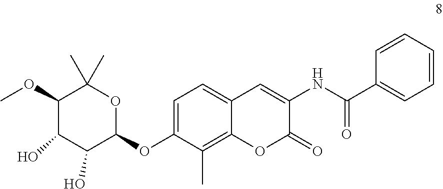

N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)benzamide (8). Colorless solid (83%): [α]²⁴_{D}=−25.2° (c=0.16, 20% MeOH in CH₂Cl₂); ¹HNMR (400 MHz, 20% CD₃OD in CD₂Cl₂) δ 8.77 (s, 1H), 7.89 (d, J=7.4 Hz, 2H) 7.59 (t, J=7.4 Hz, 1H), 7.53 (d, J=6.3 Hz, 1H), 7.51 (d, J=7.4 Hz, 1H), 7.39 (d, J=8.7 Hz, 1H), 7.21 (d, J=8.7 Hz, 1H), 5.55 (d, J=2.2 Hz, 1H), 4.18-4.08 (m, 2H), 3.57 (s, 3H), 3.33-3.31 (m, 1H), 2.27 (s, 3H), 1.35 (s, 3H), 1.10 (s, 3H); ¹³C NMR (100 MHz, 20% CD₃OD in CD₂Cl₂) δ 164.5, 157.7, 154.6, 147.6, 132.1, 130.7, 127.2 (2C), 125.5 (2C), 124.2, 123.1, 120.0, 112.5, 112.1, 110.0, 96.7, 82.5, 76.9, 69.5, 66.8, 60.0, 27.0, 20.6, 6.3; IR (film) νₘₐₓ 3400, 3088, 3065, 2978, 2926, 2853, 1713, 1668, 1607, 1526, 1493, 1369, 1252, 1092, 1080 cm'; HRMS (ESI+) m/z 470.1826 (M+H⁺, C₂₅H₂₈NO₈ requires m/z 470.1815).

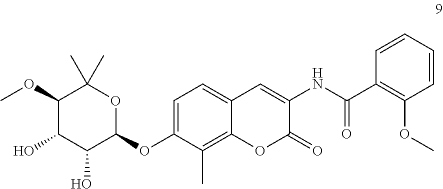

N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)-2-methoxybenzamide (9). Colorless solid (74%): [α]²⁵_{D}=−16.1° (c=0.16, 20% MeOH in CH₂Cl₂); ¹HNMR (400 MHz, 20% CD₃OD in CD₂Cl₂) δ 8.79 (s, 1H), 8.15 (d, J=7.9 Hz, 1H), 7.57 (t, J=7.8 Hz, 1H), 7.38 (d, J=8.7 Hz, 1H), 7.20 (d, J=8.7 Hz, 1H), 7.16-7.10 (m, 2H), 5.55 (s, 1H) 4.20-4.14 (m, 2H), 4.11 (s, 3H), 3.58 (s, 3H), 3.35 (d, J=8.0 Hz, 1H), 2.28 (s, 3H), 1.34 (s, 3H), 1.11 (s, 3H); ¹³C NMR (100 MHz, 20% CD₃OD in CD₂Cl₂) δ 164.9, 160.2, 158.4, 156.7, 149.8, 134.6, 132.4, 126.3, 125.3, 123.0, 122.9, 121.9, 121.2, 114.7, 112.5, 111.7, 99.1, 84.8, 79.2, 71.9, 69.0, 62.1, 56.8, 29.1, 22.9, 8.5; IR (film) νₘₐₓ 3373, 2947, 2835, 2525, 1641, 1630, 1610, 1448, 1412, 1398 cm^{-l}; HRMS (ESI+) m/z 500.1893 (M+H⁺, C₂₆H₃₀NO₉ requires m/z 500.1921).

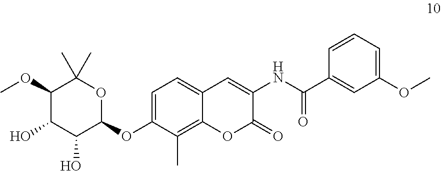

N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)-3-methoxybenzamide (10). Colorless solid (59%): [α]²⁴D=−16.9° (c=0.81, 20% MeOH in CH₂Cl₂); NMR (400 MHz, 20% CD₃OD in CD₂Cl₂) δ 8.70 (s, 1H), 7.42-7.33 (m, 4H), 7.19 (d, J=8.7 Hz, 1H), 7.13 (d, J=8.7 Hz, 1H), 5.52 (s, 1H), 4.19-4.05 (m, 2H), 3.84 (s, 3H), 3.55 (s, 3H), 3.32 (d, J=7.8 Hz, 1H), 2.24 (s, 3H), 1.30 (s, 3H), 1.07 (s, 3H); ¹³C NMR (100 MHz, 20% CD₃OD in CD₂Cl₂) δ 167.1, 160.8, 160.1, 157.1, 150.0, 135.9, 130.6, 126.5, 126.1, 122.2, 119.6, 118.9, 114.8, 114.4, 113.3, 111.9, 99.2, 84.8, 79.3, 71.9, 69.1, 62.1, 56.0, 29.1, 22.9, 8.4; IR (film) νₘₐₓ 3400, 3082, 2980, 2937, 2835, 1709, 1670, 1607, 1526, 1369, 1259, 1138, 1090 cm⁻¹; HRMS (ESI+) m/z 500.1899 (M+H⁺, C₂₆H₃₀NO₉ requires m/z 500.1921).

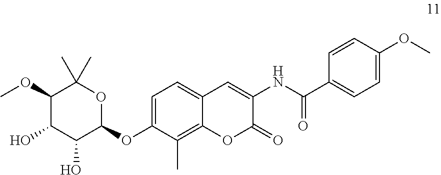

N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)-4-methoxybenzamide (11). Colorless solid (75%): [α]²⁵_{D}=−13.0° (c=0.10, 20% MeOH in CH₂Cl₂); ¹H NMR (400 MHz, CD₂Cl₂) δ 8.78 (s, 1H), 8.68 (s, 1H), 7.91 (dt, J=2.6, 6.9 Hz, 2H), 7.40 (d, J=8.7 Hz, 1H), 7.22 (d, J=8.7 Hz, 1H), 7.03 (dt, J=2.6, 8.9 Hz, 2H), 5.63 (d, J=2.0 Hz, 1H), 4.30-4.23 (m, 2H), 3.91 (s, 3H), 3.62 (s, 3H), 3.38 (d, J=8.8 Hz, 1H), 2.98-2.70 (m, 2H), 2.31 (s, 3H), 1.39 (s, 3H), 1.16 (s, 3H); ¹³C NMR (100 MHz, CD₂Cl₂) δ 165.7, 163.3, 159.7, 156.3, 149.4 (2C), 129.4, 126.3, 126.0, 124.2, 122.4, 114.5 (2C), 114.4 (2C), 111.5, 98.3, 84.6, 78.9, 71.5, 68.9, 62.1, 55.9, 29.2, 22.7, 8.5; IR (film) νₘₐₓ 3404, 2976, 2934, 2841, 1607, 1506, 1369, 1248, 1176, 1091 cm⁻¹; HRMS (ESI+) m/z 500.1896 (M+C₂₆H₃₀NO₉ requires m/z 500.1921).

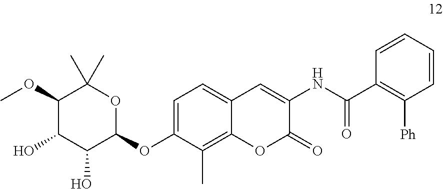

N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)biphenyl-2-carboxamide (12). Colorless solid (55%): [α]²⁴_{D}=−7.2° (c=0.13, 20% MeOH in CH₂Cl₂); ¹H NMR (400 MHz, CD₂Cl₂) δ 8.77 (s, 1H), 7.99 (s, 1H), 7.78 (d, J=8.0 Hz, 1H), 7.65-7.33 (m, 9H), 7.20 (d, J=8.8 Hz, 1H), 5.61 (d, J=1.8 Hz, 1H), 4.28-4.21 (m, 2H), 3.61 (s, 3H), 3.37 (d, J=8.7 Hz, 1H), 2.25 (s, 3H), 1.38 (s, 3H), 1.14 (s, 3H); ¹³C NMR (125 MHz, CD₂Cl₂) δ 168.5, 158.8, 156.3, 149.5, 140.6, 140.2, 135.4, 131.3, 131.1, 129.1 (4C), 129.0, 128.3, 128.0, 126.0, 124.0, 122.1, 114.5, 114.2, 111.4, 98.2, 84.6, 78.8, 71.6, 68.9, 62.1, 29.3, 22.6, 8.4; IR (film) νₘₐₓ 3379, 3059, 2982, 2932, 2831, 1713, 1668, 1607, 1520, 1367, 1258, 1113, 1092 cm⁻¹; HRMS (ESI+) m/z 546.2100 (M+H⁺, C₃₁H₃₂NO₈ requires m/z 546.2128).

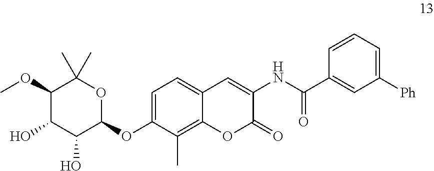

N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)biphenyl-3-carboxamide (13). Colorless solid (75%): [α]²⁴_{D}=−24.3° (c=0.09, 20% MeOH in CH₂Cl₂); ¹H NMR (400 MHz, 20% CD₃OD in CD₂Cl₂) δ 8.84 (s, 1H), 8.82 (s, 1H), 8.17 (s, 1H), 7.91 (d, J=7.7 Hz, 1H), 7.86 (t, J=6.7 Hz, 1H), 7.71 (dd, J=1.3, 7.7 Hz, 2H), 7.63 (t, J=7.7 Hz, 1H), 7.53 (t, J=7.7 Hz, 2H), 7.44 (t, J=7.5 Hz, 2H), 7.25 (d, J=8.7 Hz, 1H), 5.64 (s, 1H), 4.31-4.25 (m, 2H), 3.62 (s, 3H), 3.38 (d, J=8.7 Hz, 1H), 2.32 (s, 3H), 1.39 (s, 3H), 1.16 (s, 3H); ¹³C NMR (125 MHz, 20% CD₃OD in CD₂Cl₂) δ 166.9, 159.8, 156.8, 149.7, 145.0, 142.4, 140.3, 134.8, 131.3, 129.7, 129.3 (2C), 128.2, 127.6 (2C), 126.2 (2C), 125.7, 121.9, 114.5, 114.1, 111.6, 98.9, 84.5, 78.9, 71.6, 68.7, 61.8, 28.8, 22.6, 8.6; IR (film) νₘₐₓ 3458, 3400, 3060, 2982, 2930, 2854, 1713, 1668, 1628, 1607, 1526, 1367, 1265, 1238, 1095, 1082 cm⁻¹; HRMS (ESI+) m/z 546.2112 (M+H⁺, C₃₁H₃₂NO₈ requires m/z 546.2128).

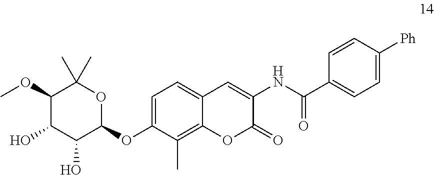

N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)biphenyl-4-carboxamide (14). Colorless solid (80%): [α]²⁶_{D}=−7.3° (c=0.06, 20% MeOH in CH₂Cl₂); ¹H NMR (400 MHz, 20% CD₃OD in CD₂Cl₂) 8.87 (s, 1H), 8.78 (s, 1H), 7.98 (d, J=8.4 Hz, 2H), 7.75 (d, J=8.4 Hz, 2H), 7.65 (dd, J=1.3, 7.8 Hz, 2H), 7.47 (t, J=7.4 Hz, 2H), 7.38 (d, J=7.8 Hz, 2H), 7.21 (d, J=8.4 Hz, 1H), 5.55 (d, J=2.1 Hz, 1H), 4.18-4.08 (m, 2H), 3.80 (s, 3H), 3.35-3.30 (m, 1H), 2.27 (s, 3H), 1.32 (s, 3H), 1.07 (s, 3H); ¹³C NMR (100 MHz, 20% CD₃OD in CD₂Cl₂) δ 159.9, 156.7, 148.9, 145.8, 138.7, 129.3 (2C), 128.6, 128.1 (3C), 127.8 (2C), 127.5 (3C), 126.2, 125.3, 122.8, 114.5, 114.2, 111.6, 98.8, 84.5, 78.9, 71.6, 68.8, 61.9, 28.9, 22.6, 8.3; IR (film) νₘₐₓ 3404, 3059, 3032, 2978, 2932, 2835, 1709, 1666, 1609, 1531, 1416, 1367, 1265, 1252, 1095, 1078 cm⁻¹; HRMS (ESI+) m/z 546.2140 (M+H⁺, C₃₁H₃₂NO₈ requires m/z 546.2128).

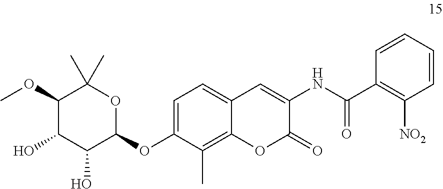

N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)-2-nitrobenzamide (15). Prepared as described above with the exception that the product was purified by preparative TLC (SiO₂, 1:1 hexanes:ethyl acetate, developed 5 times) to afford 15 (44%) as a yellow solid: [α]²³_{D}=−15.9° (c=0.15, 20% MeOH in CH₂Cl₂); ¹H NMR (400 MHz, 20% CD₃OD in CD₂Cl₂) δ 8.75 (s, 1H), 8.15 (dd, J=0.9, 8.1 Hz, 1H), 7.79-7.61 (m, 3H), 7.37 (d, J=8.8 Hz, 1H), 7.20 (d, J=8.8 Hz, 1H), 5.56 (d, J=2.0 Hz, 1H), 4.19-4.08 (m, 2H), 3.59 (s, 3H), 3.35 (d, J=9.1 Hz, 1H), 2.25 (s, 3H), 1.34 (s, 3H), 1.09 (s, 3H); ¹³C NMR (125 MHz, CD₂Cl₂) δ 165.1, 159.2, 156.8, 149.8, 146.9, 134.4, 132.2, 131.8, 128.9, 126.4, 125.6, 125.2, 122.0, 114.6, 114.0, 111.6, 98.2, 84.6, 78.9, 71.6, 69.0, 62.1, 29.3, 22.6, 8.5; IR (film) νₘₐₓ 3441, 3387, 2984, 2934, 1713, 1674, 1607, 1529, 1371, 1348, 1256, 1105, 1084 cm⁻¹; HRMS (ESI+) m/z 515.1669 (M+H⁺, C₂₅H₂₇N₂O₁₀ requires m/z 515.1666).

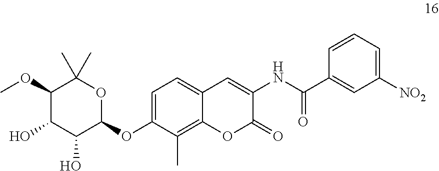

N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)-3-nitrobenzamide (16). Yellow solid (73%): [α]²⁵_{D}=−15.7° (c=0.26, 20% MeOH in CH₂Cl₂); ¹H NMR (400 MHz, CDCl₃) δ 8.71 (s, 2H), 8.36 (d, J=1.0, 8.2 Hz, 1H) 8.20 (d, J=1.0, 8.2 Hz, 1H), 7.68 (t, J=8.0 Hz, 1H), 7.31 (d, J=8.7 Hz, 1H), 7.15 (d, J=8.7 Hz, 1H), 5.51 (d, J=2.0 Hz, 1H), 4.14-4.05 (m, 2H), 3.59 (s, 3H), 3.31 (d, J=9.1 Hz, 1H), 2.33 (s, 3H), 1.29 (s, 3H), 1.10 (s, 3H); ¹³C NMR (100 MHz, CDCl₃) δ 164.3, 159.7, 157.0, 149.7, 148.8, 135.7, 133.2, 130.5, 127.1, 126.7, 126.4, 122.9, 121.4, 114.6, 113.8, 111.7, 98.7, 84.5, 79.0, 71.5, 68.8, 62.1, 29.2, 22.7, 8.5; IR (film) νₘₐₓ 3362, 2986, 2949, 2837, 1705, 1645, 1635, 1605, 1554, 1531, 1371, 1346, 1253, 1136, 1117, 1003, 1080, 1018 cm⁻¹; HRMS (ESI+) m/z 537.1477 (M+Na⁺, C₂₅H₂₆N₂O₁₀Na requires m/z 537.1485).

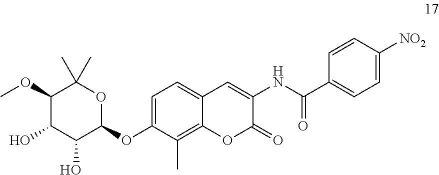

N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)-4-nitrobenzamide (17). Yellow solid (79%): [α]²⁶_{D}=−13.1° (c=0.16, 20% MeOH in CH₂Cl₂); ¹HNMR (400 MHz, 20% CD₃OD in CD₂Cl₂) δ 9.18 (s, 1H), 8.79 (dd, J=1.9, 6.9 Hz, 2H) 8.56 (dd, J=1.9, 6.9 Hz, 2H), 7.85 (d, J=8.7 Hz, 1H), 7.67 (d, J=8.7 Hz, 1H), 6.00 (d, J=2.2 Hz, 1H), 4.60 (dd, J=3.4, 9.4 Hz, 1H), 4.56 (t, J=3.4 Hz, 1H), 4.02 (s, 3H), 3.80 (d, J=9.4 Hz, 1H), 2.71 (s, 3H), 1.75 (s, 3H), 1.61 (s, 3H); ¹³C NMR (100 MHz, 20% CD₃OD in CD₂Cl₂) δ 165.0, 159.6, 157.1, 150.3, 149.9, 139.7, 129.0 (2C), 127.0, 126.4, 124.2 (2C), 121.5, 114.5, 113.8, 111.7, 98.9, 84.4, 79.0, 71.6, 68.7, 61.8, 28.8, 22.5, 8.1; IR (film) νₘₐₓ 3381, 3053, 2947, 2835, 1699, 1666, 1603, 1524, 1373, 1346, 1252, 1113, 1086, 1018 cm⁻¹; HRMS (ESI+) m/z 537.1486 (M+Na⁺, C₂₅H₂₆N₂O₁₀Na requires m/z 537.1485).

General procedure for reduction of nitro group: Palladium on carbon (10%, 0.1 eq) was added to a solution of 15, 16 or 17 (1 eq) in THF at room temperature. The suspension was stirred for six hours under a hydrogen atmosphere, filtered through a plug of SiO₂, and eluted with THF. The eluent was concentrated and the residue purified by preparative TLC (SiO₂, 100:1→50:1; CH₂Cl₂:acetone) to afford the aniline.

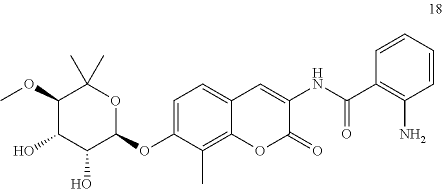

2-Amino-N-(7-((2R,3R,4S,5R)-3,4-dihydroxy-5-methoxy-6,6-dimethyltetrahydro-2H-pyran-2-yloxy)-8-methyl-2-oxo-2H-chromen-3-yl)benzamide (18). Colorless solid (90%): [α]²³_{D}=−17.6° (c=0.09, 20° Z MeOH in CH₂Cl₂); NMR (500 MHz, 20% CD₃OD in CD₂Cl₂) δ 8.64 (s, 1H), 7.48 (dd, J=1.0, 7.5 Hz, 1H), 7.31 (d, J=9.0 Hz, 1H), 7.24-7.17 (m, 1H), 7.14 (d, J=8.5 Hz, 1H), 6.70 (d, J=8.5 Hz, 1H), 6.66 (t, J=7.5 Hz, 1H), 5.48 (d, J=2.0 Hz, 1H), 4.09 (dd, J=3.3, 9.5 Hz, 1H), 4.04 (t, J=3.3 Hz, 1H), 3.50 (s, 3H), 3.28-3.25 (m, 1H), 2.21 (s, 3H), 1.26 (s, 3H), 1.03 (s, 3H); ¹³C NMR (125 MHz, 20% CD₃OD in CD₂Cl₂) δ 168.4, 159.9, 156.5, 149.6, 149.5, 133.6, 127.9, 126.0, 124.6, 122.1, 118.0, 117.2, 115.3, 114.5, 114.3, 111.6, 98.8, 84.5, 78.9, 71.6, 68.8, 61.9, 28.9, 22.6, 8.3; IR (film) νₘₐₓ 3470, 3408, 3362, 2978, 2926, 2853, 1707, 1657, 1609, 1520, 1450, 1408, 1367, 1263, 1242 1088 cm⁻¹; HRMS (ESI+) m/z 485.1919 (M+H⁺, C₂₅H₂₉N₂O₈ requires m/z 485.1924).

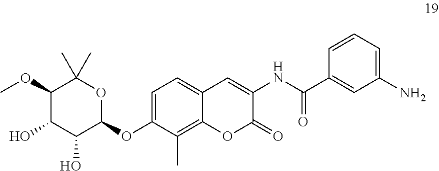

3-Amino-N-(7-((2R,3R,4S,5R)-3,4-dihydroxy-5-methoxy-6,6-dimethyltetrahydro-2H-pyran-2-yloxy)-8-methyl-2-oxo-2H-chromen-3-yl)benzamide (19). Colorless solid (77%): [α]²⁶_{D}=−24.3° (c=0.07, 20% MeOH in CH₂Cl₂); ¹H NMR (400 MHz, 20% CD₃OD in CD₂Cl₂) δ 8.79 (s, 1H), 8.76 (s, 1H), 7.41 (d, J=8.7 Hz, 1H), 7.32-7.20 (m, 4H), 6.92 (d, J=6.3 Hz, 1H), 5.58 (d, J=1.6 Hz, 1H), 4.20-4.11 (m, 2H), 3.60 (s, 3H), 3.35 (d, J=9.5 Hz, 1H), 2.30 (s, 3H), 1.35 (s, 3H), 1.17 (s, 3H); ¹³C NMR (200 MHz, 20% CD₃OD in CD₂Cl₂) δ 166.8, 159.4, 156.3, 149.2, 147.7, 134.7, 129.6, 125.7, 124.8, 121.6, 118.9, 116.3, 114.1, 113.8, 113.2, 111.2, 98.4, 84.1, 78.5, 71.2, 68.4, 61.4, 28.5, 22.2, 7.8; IR (film) νₘₐₓ 3404, 2986, 2949, 2843, 1634, 1607, 1520, 1367, 1261, 1111, 1016 cm⁻¹; HRMS (ESI+) m/z 507.1740 (M+Na⁺, C₂₅H₂₈N₂O₈Na requires m/z 507.1743).

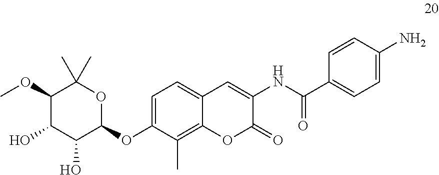

4-Amino-N-(7-((2R,3R,4S,5R)-3,4-dihydroxy-5-methoxy-6,6-dimethyltetrahydro-2H-pyran-2-yloxy)-8-methyl-2-oxo-2H-chromen-3-yl)benzamide (20). Yellow solid (77%): [α]²⁶_{D}=−15.9° (c=0.30, 20% MeOH in CH₂Cl₂); ¹H NMR (400 MHz, CDCl₃) δ 8.76 (s, 1H), 8.61 (s, 1H), 7.75 (dd, J=1.8, 6.8 Hz, 2H), 7.31 (d, J=8.7 Hz, 1H), 7.17 (d, J=8.7 Hz, 1H), 6.73 (td, J=1.8, 6.8 Hz, 2H), 5.60 (d, J=1.5 Hz, 1H), 4.28-4.24 (m, 2H), 4.16 (s, 2H), 3.62 (s, 3H), 3.39 (d, J=8.8 Hz, 1H), 3.17 (s, 1H), 2.90 (s, 1H), 2.27 (s, 3H), 1.39 (s, 3H), 1.14 (s, 3H); ¹³C NMR (100 MHz, CDCl₃) δ 166.2, 159.9, 156.7, 150.9, 149.3, 129.5 (2C), 126.1, 124.2, 123.3, 122.4, 114.7 (2C), 114.6 (2C), 111.5, 98.3, 84.7, 79.0, 71.6, 69.0, 62.3, 29.6, 22.9, 8.8; IR (film) νₘₐₓ 3381, 2980, 2941, 2839, 1697, 1634, 1607, 1531, 1510, 1367, 1252, 1184, 1092, 1078, 1020, 993, 966 cm⁻¹; HRMS (ESI+) m/z 485.1940 (M+H⁺, C₂₅H₂₉N₂O₈ requires m/z 485.1924).

### Example 9

### Preparation of Novobiocin Analogues with Various Linkers

Simultaneous with the investigation of aryl substitutes, modification of the amide and tether functionalities is performed as shown in Scheme 16 below. Sulfonamide 21 was assembled by sulfonylation of amine 7 from the prior example with benzenesulfonyl chloride, the carbonate of which was subjected to solvolysis to provide the resulting diol. The CBz-containing product 24, was obtained by direct solvolysis of 5. Amides 22, 23, and 25 were prepared by coupling the appropriate acid with amine 7 in the presence of EDCI and pyridine, followed by solvolysis of the cyclic carbonate.

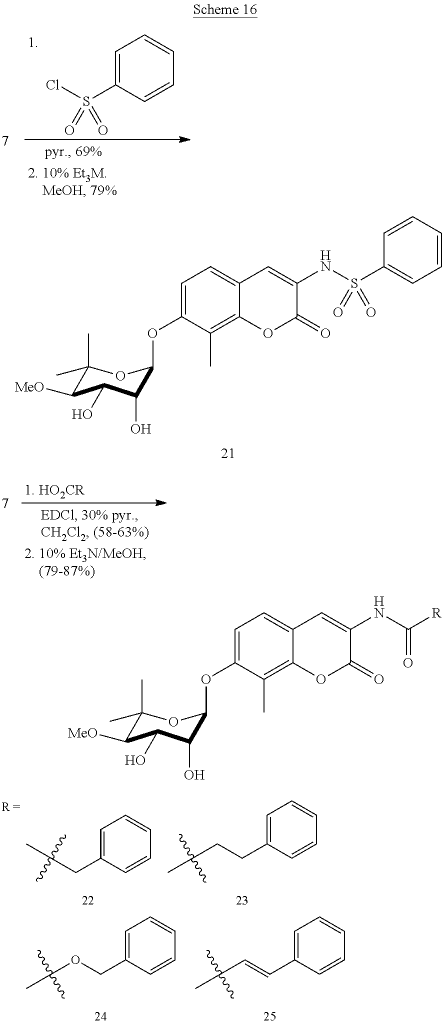

More specifically, compound 21 was prepared as follows:

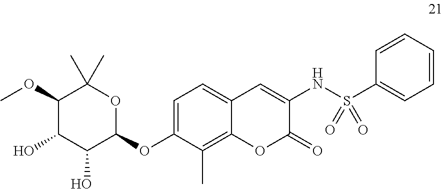

N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)benzenesulfonamide (21). Benzenesulfonyl chloride (8 μL, 10.7 mg, 60.2 μmol) was added to a solution of 21.4 mg (54.7 μmol) aniline 7 in 0.5 mL of pyridine at room temperature. The reaction mixture was stirred for 14 hours and then concentrated. The residue was purified via preparative TLC (40:1 CH₂Cl₂:acetone) to afford 20 mg (69%) of the sulfonamide as a glassy solid. The cyclic carbonate (20 mg) was dissolved in 0.5 mL of methanol and 0.1 mL of Et₃N was added dropwise. The reaction mixture was stirred for 14 hours at room temperature before concentration. The residue was purified by preparative TLC (10:1; CH₂Cl₂:methanol) to afford 15 mg (79%) of 21 as a white solid. [α]²⁴_{D}=−5.1° (c=0.42, 20% MeOH in CH₂Cl₂); ¹H NMR (400 MHz, CD₂Cl₂) δ 7.88 (dd, J=0.8, 8.0 Hz, 2H), 7.75 (s, 1H), 7.58 (t, J=7.2 Hz, 1H), 7.50 (d, J=7.2 Hz, 1H), 7.48 (d, J=7.2 Hz, 1H), 7.41 (s, 1H), 7.31 (d, J=8.8 Hz, 1H), 7.17 (d, J=8.8 Hz, 1H), 5.57 (d, J=2.0 Hz, 1H), 4.24-4.17 (m, 2H), 3.57 (s, 3H), 3.34 (d, J=8.8 Hz, 1H), 2.53 (s, 2H), 2.18 (s, 3H), 1.34 (s, 3H), 1.08 (s, 3H); ¹³C NMR (100 MHz, CD₂Cl₂) δ 159.1, 156.9, 150.1, 139.2, 134.0, 129.7 (2C), 127.6 (2C), 126.0, 125.7, 120.9, 114.7, 113.5, 111.6, 98.3, 84.5, 78.9, 71.6, 69.0, 62.1, 29.2, 22.7, 8.4; IR (film) νₘₐₓ 3439, 3429, 2982, 2930, 2853, 1713, 1630, 1609, 1499, 1464, 1448, 1369, 1327, 1285, 1261, 1167, 1113, 1088 cm⁻¹; HRMS (ESI+) m/z 506.1490 (M+H⁺, C₂₄H₂₈NO₉S requires m/z 506.1485).

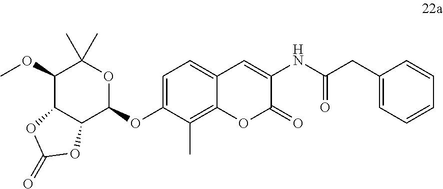

N-(7-((3aR,4R,7R,7aR)-7-Methoxy-6,6-dimethyl-2-oxotetrahydro-3 aH-[1,3]dioxolo[4,5-c]pyran-4-yloxy)-8-methyl-2-oxo-2H-chromen-3-yl)-2-phenylacetamide (22a). N-(3-Dimethylamino-propyl)-N-ethylcarbodiimide hydrochloride (34 mg, 72 μmol) was added to a solution of aminocoumarin 7 (28 mg, 72 μmol) and phenyl acetic acid (24 mg, 179 μmol) in CH₂Cl₂ at room temperature. The solution was stirred for 14 hours, concentrated and the residue purified via preparative TLC (SiO₂, 40:1; CH₂Cl₂:acetone) to afford the 24 mg (66%) of 22a as a colorless solid: [α]²⁴_{D}=−28.5° (c=0.39, CH₂Cl₂); ¹H NMR (800 MHz, CD₂Cl₂) δ 8.62 (s, 1H), 8.00 (s, 1H), 7.36 (t, J=8.0 Hz, 2H), 7.33-7.26 (m, 4H), 7.08 (d, J=8.8 Hz, 1H), 5.74 (d, J=1.6 Hz, 1H), 5.01 (dd, J=1.6, 8.0 Hz, 1H), 4.92 (t, J=8.0 Hz, 1H), 3.72 (s, 2H), 3.53 (s, 3H), 3.30 (d, J=8.0 Hz, 1H), 2.22 (s, 3H), 1.31 (s, 3H), 1.15 (s, 3H); ¹³C NMR (200 MHz, CD₂Cl₂) δ 171.8, 160.6, 157.0, 155.1, 151.0, 136.2, 131.3 (2C), 130.9 (2C), 129.4, 127.6, 125.4, 124.0, 116.6, 116.5, 113.1, 96.3, 84.7, 79.9, 79.0, 78.5, 62.3, 46.5, 29.2, 23.9, 10.0; IR (film) νₘₐₓ 3333, 3088, 3063, 3030, 2984, 2935, 2851, 1809, 1717, 1684, 1609, 1522, 1369, 1261, 1173, 1111, 1088, 1036 cm⁻¹; HRMS (ESI+) m/z 510.1755 (M+H⁺, C₂₇H₂₈NO₉ requires m/z 510.1764).

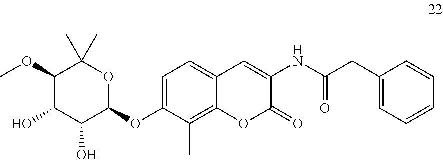

N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)-2-phenylacetamide (22). Prepared by the general cyclic carbonate solvolysis procedure described above to afford 22 (87%) as a colorless solid: [α]²⁴_{D}=−15.1° (c=0.37, CH₂Cl₂); ¹H NMR (800 MHz, CD₂Cl₂) δ 8.58 (s, 1H), 8.04 (s, 1H), 7.40-7.31 (m, 5H), 7.29 (d, J=8.8 Hz, 1H), 7.15 (d, J=8.8 Hz, 1H), 5.56 (d, J=0.8 Hz, 1H), 4.24-4.20 (m, 2H), 3.76 (s, 2H), 3.57 (s, 3H), 3.34 (d, J=8.8 Hz, 1H), 2.95 (s, 1H), 2.83 (s, 1H), 2.22 (s, 3H), 1.31 (s, 3H), 1.10 (s, 3H); ¹³C NMR (200 MHz, CD₂Cl₂) δ 171.9, 160.7, 157.9, 151.0, 136.2, 131.3 (2C), 130.9 (2C), 129.4, 127.5, 125.9, 123.5, 116.0, 115.7, 113.0, 99.8, 86.1, 80.4, 73.1, 70.5, 63.6, 46.5, 30.8, 24.2, 10.0; IR (film) νₘₐₓ 3367, 3339, 3086, 3063, 3030, 2980, 2932, 2853, 2831, 1715, 1684, 1607, 1526, 1369, 1263, 1113, 1084 cm⁻¹; HRMS (ESI+) m/z 484.1982 (M+H⁺, C₂₆H₃₀NO₈ requires m/z 484.1971).

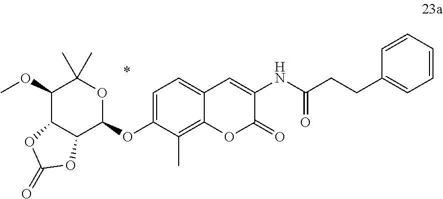

N-(7-((3aR,4R,7R,7aR)-7-Methoxy-6,6-dimethyl-2-oxotetrahydro-3aH-[1,3]dioxolo[4,5-c]pyran-4-yloxy)-8-methyl-2-oxo-2H-chromen-3-yl)-3-phenylpropanamide (23a). Prepared by the general EDCI coupling procedure described above to afford 23a (21 mg, 58%) as a colorless solid: [α]²³_{D}=−23.4° (c=0.32, 20% MeOH in CH₂Cl₂); ¹H NMR (400 MHz, CD₂Cl₂) δ 8.65 (s, 1H), 7.97 (s, 1H), 7.36 (d, J=8.7 Hz, 1H), 7.32-7.13 (m, 5H), 7.15 (d, J=8.7 Hz, 1H), 5.81 (d, J=2.1 Hz, 1H), 5.08 (dd, J=2.1, 8.0 Hz, 1H), 4.98 (t, J=8.0 Hz, 1H), 3.60 (s, 3H), 3.35 (d, J=8.0 Hz, 1H), 3.04 (t, J=7.7 Hz, 2H), 2.75 (t, J=7.7 Hz, 2H), 2.28 (s, 3H), 1.36 (s, 3H), 1.20 (s, 3H); ¹³C NMR (125 MHz, CD₂Cl₂) δ 170.4, 158.0, 154.3, 152.4, 148.3, 139.9, 128.1 (2C), 127.7 (2C), 125.4, 124.8, 122.7, 121.4, 113.9 (2C), 110.4, 93.6, 82.0, 77.2, 76.4, 75.8, 59.6, 38.7, 31.2, 28.6, 21.9, 7.3; IR (film) νₘₐₓ 3327, 3086, 3063, 3026, 2982, 2930, 2851, 1811, 1717, 1684, 1607, 1522, 1371, 1259, 1173, 1111, 1086, 1036, 1005 cm⁻¹; HRMS (ESI+) m/z 524.1912 (M+H⁺, C₂₈H₃₀NO₉ requires m/z 524.1921).

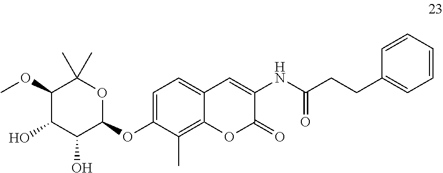

N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)-3-phenylpropanamide (23). Prepared by the general cyclic carbonate solvolysis procedure described above to afford 23 (16 mg, 79%) as a colorless solid: [α]²⁴_{D}=−14.6° (c=0.49, CH₂Cl₂); ¹HNMR (800 MHz, CD₂Cl₂) δ 8.60 (s, 1H), 7.95 (s, 1H), 7.31-7.24 (m, 3H), 7.23 (d, J=7.2 Hz, 2H), 7.18 (t, J=8.7 Hz, 1H), 7A5 (d, J=8.8 Hz, 1H), 5.56 (d, J=2.4 Hz, 1H), 4.23-4.17 (m, 2H), 3.56 (s, 3H), 3.33 (d, J=8.8 Hz, 1H), 3.01 (t, J=8.0 Hz, 2H), 2.85 (s, 1H), 2.76 (s, 1H), 2.72 (t, J=8.0 Hz, 2H), 2.22 (s, 3H), 1.33 (s, 3H), 1.10 (s, 3H); ¹³C NMR (200 MHz, CD₂Cl₂) δ 173.2, 160.8, 157.8, 150.9, 142.5, 130.4 (2C), 130.2 (2C), 128.2, 127.5, 125.8, 123.6, 116.0, 115.8, 113.0, 99.8, 86.1, 80.4, 73.1, 70.5, 63.6, 40.9, 33.0, 30.8, 24.2, 10.0; IR (film) νₘₐₓ 3427, 3391, 3325, 3080, 3086, 3061, 2932, 2833, 1709, 1684, 1607, 1529, 1377, 1223, 1113, 1084 cm⁻¹; HRMS (ESI+) m/z 498.2140 (M+H⁺, C₂₇H₃₂NO₈ requires m/z 498.2128).

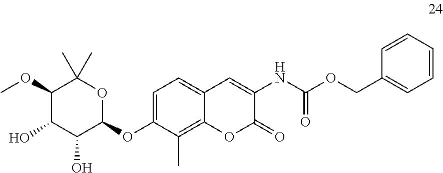

Benzyl 7-((2R,3R,4S,5R)-3,4-dihydroxy-5-methoxy-6,6-dimethyltetrahydro-2H-pyran-2-yloxy)-8-methyl-2-oxo-2H-chromen-3-ylcarbamate (24). Et₃N (10% total volume) was added dropwise to a solution of cyclic carbonate 7a (25 mg, 48 μmol) in methanol (0.6 mL) at room temperature. The resulting mixture was stirred for 14 hours and then concentrated. The residue was purified via preparative TLC (SiO₂, 4:1; CH₂Cl₂:acetone) to afford 24 (19 mg, 82%) as a white solid: [α]²⁵_{D}=−11.3° (c=0.84, 20% MeOH in CH₂Cl₂); ¹H NMR (500 MHz, 20% CD₃OD in CD₂Cl₂) δ 8.17 (s, 1H), 7.35-7.21 (m, 6H), 7.10 (d, J=9.0 Hz, 1H), 5.46 (d, J=2.5 Hz, 1H), 5.13 (s, 2H), 4.07-4.00 (m, 2H), 3.56 (s, 3H), 3.25 (d, J=9.0 Hz, 1H), 2.17 (s, 3H), 1.24 (s, 3H), 1.01 (s, 3H); ¹³C NMR (125 MHz, 20% CD₃OD in CD₂Cl₂) δ 157.1, 154.1, 151.7, 147.1, 134.2, 126.7 (2C), 126.5, 126.3 (2C), 123.5, 120.9, 120.0, 112.2, 112.0, 109.3, 96.7, 82.3, 76.7, 69.5, 66.6, 65.5, 59.7, 26.7, 20.4, 6.0; IR (film) νₘₐₓ 3443, 3421, 2982, 2936, 2836, 2525, 1701, 1632, 1609, 1456, 1416, 1360, 1288, 1115, 1086 cm⁻¹; HRMS (ESI+) m/z 522.1721 (M+Na⁺, C₂₆H₂₉NO₉Na requires m/z 522.1740).

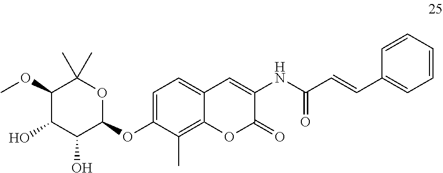

N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)cinnamamide (25). N-(3-Dimethylamino-propyl)-N-ethylcarbodiimide hydrochloride (44 mg, 227 μmol) was added to a solution of aminocoumarin 7 (36 mg, 91 μmol) and trans-cinnamic acid (27 mg, 182 μmol) in CH₂Cl₂ containing 30% pyridine at room temperature. The solution was stirred for 14 hours, concentrated and the residue purified via preparative TLC (SiO₂, 40:1; CH₂Cl₂:acetone) to afford the 30 mg (63%) of the amide as an off-white solid. The cyclic carbonate (30 mg) was dissolved in 0.8 mL of methanol and 0.1 mL of Et₃N was added dropwise. The reaction mixture was stirred for 14 hours and then was concentrated. The residue was purified by preparative TLC (10:1; CH₂Cl₂:methanol) to afford 25 (29 mg, 86%) as a colorless solid: [α]²⁶_{D}=−41.7° (c=0.18, DMSO); ¹H NMR (500 MHz, 20% CD₃OD in CD₂Cl₂) δ 8.69 (s, 1H), 7.66 (d, J=16.0 Hz, 1H), 7.56 (d, J=6.5 Hz, 2H), 7.38-7.30 (m, 4H), 7.17 (d, J=8.5 Hz, 1H), 6.80 (d, J=16.0 Hz, 1H), 5.51 (s, 1H), 4.19-4.02 (m, 2H), 3.53 (s, 3H), 3.30 (d, J=9.0 Hz, 1H), 2.23 (s, 3H), 1.28 (s, 3H), 1.06 (s, 3H); ¹³C NMR (125 MHz, 20% CD₃OD in CD₂Cl₂) δ 164.8, 157.9, 155.7, 148.8, 140.8, 134.7, 131.9, 129.0 (2C), 127.8 (2C), 125.9, 124.0, 122.1, 122.0, 113.3, 112.8, 110.8, 98.5, 83.4, 77.8, 70.8, 67.8, 61.0, 28.5, 22.9, 8.1; IR (film) νₘₐₓ 3447, 3412, 3385, 3071, 3059, 2924, 2853, 1701, 1609, 1412, 1373, 1262, 1180, 1113, 1082, 1059, 1022 cm⁻¹; HRMS (ESI+) m/z 496.1967 (M+H⁺, C₂₇H₃₀NO₈ requires m/z 496.1971).

### Example 10

### Preparation of Biaryl Compounds

In an effort to incorporate the structure-activity relationships into more efficacious inhibitors, a small library of novobiocin derivatives was prepared. The library explored optimization of the benzamide that contained a p-methoxy and a m-phenyl substituent. It was believed that this set of compounds could be expeditiously prepared by the coupling of a 3-iodo-4-methoxy benzoic acid with 7, to produce an intermediate (27) upon which the incorporation of additional phenyl substituents could be pursued for elucidation of structure-activity relationships as shown in the scheme below:

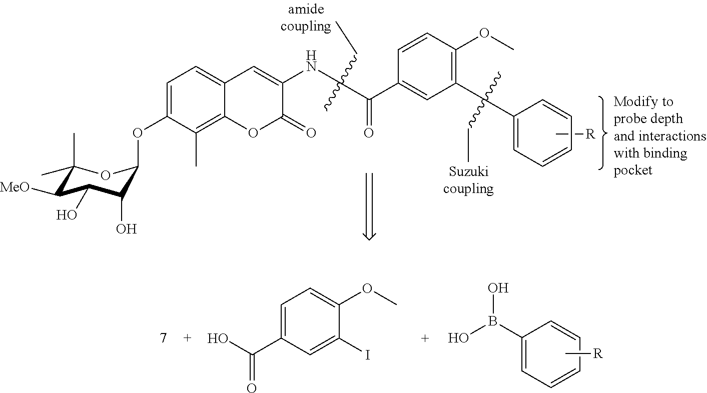

To prepare the library of compounds, the Suzuki precursor 27 was prepared by coupling aminocoumarin 7 with benzoic acid 26 in the presence of EDCI and pyridine as shown in Scheme 17 below. The biaryl substituents also included various hydrogen bond acceptors and donors to further probe key binding interactions with Hsp90. After an extensive survey of experimental conditions, it was found that dichloro[1,1′-bis(diphenylphosphino)ferrocene]palladium(II) dichloromethane [Pd(dppf)Cl₂] in the presence of substituted phenyl boronic acids and 2 M potassium carbonate in dioxane at 50° C. provided the most reproducible cross-coupling. See Greenfield et al., Convenient synthesis of functionalized terphenyls, Tetrahedron Lett. 44 2729-2732 (2003). To complete the synthesis, the carbonates were removed upon solvolysis with methanolic triethylamine.

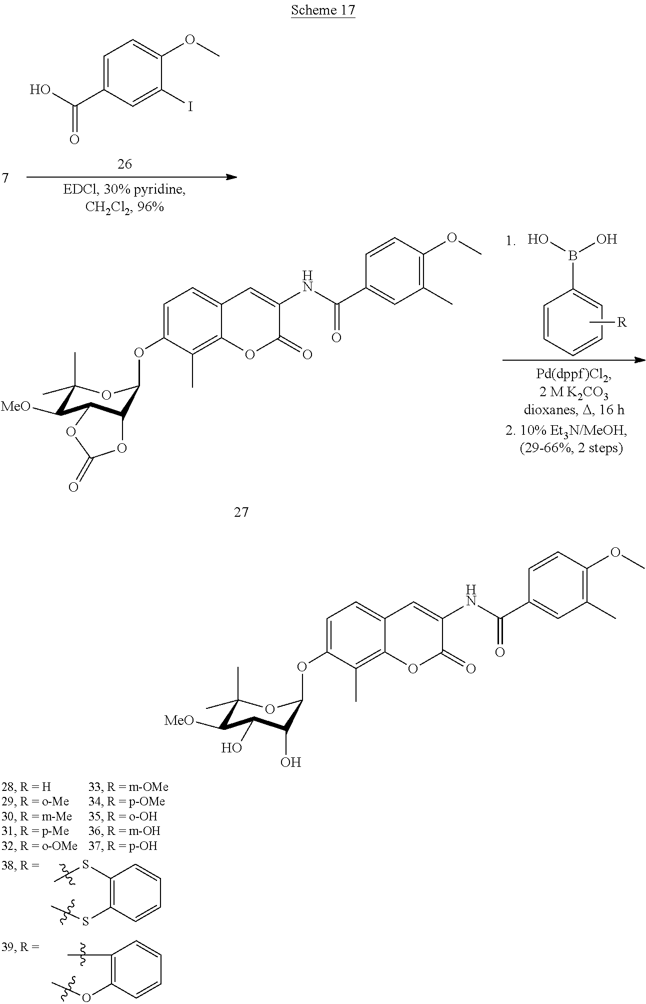

More specifically, the following compounds were prepared:

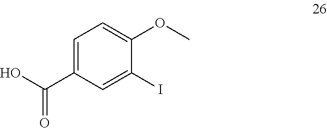

3-Iodo-4-methoxybenzoic acid (26). Lithium hydroxide (72 mg, 1.71 mmol) was added to a mixture of methyl 3-iodo-4-methoxybenzoate (100 mg, 0.342 mmol) in 3.0 mL of a 3:1:1 THF-MeOH-water solution at room temperature. The mixture was stirred for eight hours in the dark and than diluted with H₂O (2 mL). The solution was acidified to pH=2 by the dropwise addition of concentrated HCl. The solution was extracted twice with EtOAc (10 mL portions) and the combined organic layers dried (Na₂SO₄), filtered, and concentrated to afford acid 26 (95 mg, 100%) as a yellow solid that was suitable for use without further purification: ¹H NMR (400 MHz, DMSO) δ 8.24 (d, J=2.0 Hz, 1H), 7.92 (dd, J=2.0, 8.7 Hz, 1H), 7.07 (d, J=8.7 Hz, 1H), 3.88 (s, 3H).

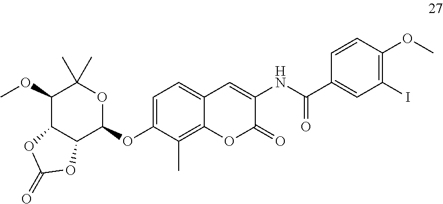

3-Iodo-4-methoxy-N-(7-((3aR,4R,7R,7aR)-7-methoxy-6,6-dimethyl-2-oxotetrahydro-3aH-[1,3]dioxolo[4,5-c]pyran-4-yloxy)-8-methyl-2-oxo-2H-chromen-3-yl)benzamide (27). Prepared by the procedure used for compound 24 to afford 27 (96%) as a yellow solid: [α]²⁵_{D}=−13.9° (c=0.17, CH₂Cl₂); ¹H NMR (400 MHz, DMSO) δ 9.69 (s, 1H), 8.47 (d, J=2.0 Hz, 1H), 8.37 (d, J=2.0 Hz, 1H), 8.00 (dd, J=2.0, 8.6 Hz, 1H), 7.60 (d, J=8.6 Hz, 1H), 7.17 (d, J=8.8 Hz, 1H), 7.12 (d, J=8.8 Hz, 1H), 6.08 (d, J=3.3 Hz, 1H), 5.20-5.11 (m, 2H), 3.91 (s, 3H), 3.83-3.75 (m, 1H), 3.49 (s, 3H), 2.23 (s, 3H), 1.29 (s, 3H), 1.12 (s, 3H); ¹³C NMR (100 MHz, DMSO) δ 164.9, 161.5, 158.8, 156.0, 154.4, 150.3, 139.3, 130.8, 129.6, 128.2, 126.9, 122.7, 114.8, 114.6, 112.1, 111.8, 94.2, 86.7, 82.3, 78.5, 77.2, 76.9, 60.7, 57.6, 27.7, 23.6, 9.0; IR (film) νₘₐₓ 3406, 3096, 3067, 2982, 2937, 2843, 1811, 1701, 1670, 1607, 1593, 1526, 1487, 1367, 1256, 1171, 1095, 1078, 1038, 1007 cm⁻¹; HRMS (ESI+) m/z 652.0691 (M+H⁺, C₂₇H₂₇NO₁₀I requires m/z 652.0680).

General procedure for Suzuki coupling and solvolysis of the cyclic carbonate: Aryl iodide 27 (1.0 eq), 2 M K₂CO_{3(aq)} (3.0 eq) and the aryl boronic acid were dissolved in dioxane before PdCl₂(dppf).CHCl₃ (3 mol %) was added to the solution at room temperature. The resulting solution was stirred at room temperature for 30 minutes and then warmed to 55° C. for 3 to 16 hours. After which, the mixture was concentrated, filtered through a pad of silica gel (eluted with 40:1; CH₂Cl₂:acetone) and purified via preparative TLC (SiO₂, 40:1; CH₂Cl₂:acetone). The resulting product was dissolved in methanol containing 10% Et₃N and stirred for 14 hours before concentrating. The residue was purified by preparative TLC (4:1; CH₂Cl₂:acetone) to afford the corresponding diol.

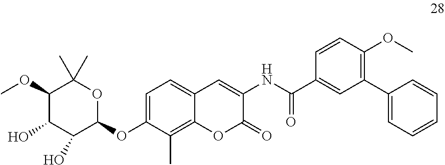

N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)-6-methoxybiphenyl-3-carboxamide (28). Colorless solid (46%, 2 steps): [0]²⁵_{D}=−8.7° (c=0.23, CH₂Cl₂); NMR (800 MHz, CD₂Cl₂) δ 8.78 (s, 1H), 8.69 (s, 1H), 7.93 (dd, J=4.4, 8.0 Hz, 1H), 7.87 (d, J=2.4 Hz, 1H), 7.55 (d, J=8.0 Hz, 2H), 7.45 (t, J=8.0 Hz, 2H), 7.93 (dd, J=4.4, 8.0 Hz, 2H), 7.21 (d, J=8.8 Hz, 1H), 7.10 (d, J=8.8 Hz, 2H), 5.60 (s, 1H), 4.27-4.20 (m, 2H), 3.90 (s, 3H), 3.59 (s, 3H), 3.36 (d, J=9.6 Hz, 1H), 2.73 (s, 2H), 2.28 (s, 3H), 1.36 (s, 3H), 1.13 (s, 3H); ¹³C NMR (200 MHz, CD₂Cl₂) δ 167.2, 161.7, 161.2, 157.9, 151.0, 139.4, 133.0, 131.8, 131.5 (3C), 130.0 (2C), 129.4, 128.1, 127.6, 125.8, 124.0, 116.1, 116.0, 113.0 (2C), 99.7, 86.1, 80.4, 73.1, 70.5, 63.7, 57.8, 30.8, 24.2, 10.1; IR (film) νₘₐₓ 3402, 3086, 3055, 3028, 2974, 2934, 2849, 2837, 1709, 1670, 1607, 1526, 1504, 1489, 1367, 1265, 1231, 1095 cm⁻¹; HRMS (ESI+) m/z 576.2231 (M+H⁺, C₃₂H₃₄NO₉ requires m/z 576.2234).

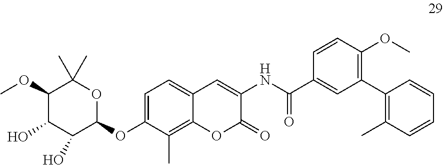

N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)-6-methoxy-2′-methylbiphenyl-3-carboxamide (29). Colorless solid (45%, 2 steps): [α]²⁴_{D}=−17.0° (c=0.23, CH₂Cl₂); ¹H NMR (400 MHz, CD₂Cl₂) δ 8.78 (s, 1H), 8.68 (s, 1H), 7.98 (dd, J=2.4, 8.6 Hz, 1H), 7.72 (d, J=2.4 Hz, 1H), 7.39 (d, J=8.7 Hz, 1H), 7.33-7.16 (m, 5H), 7.10 (d, J=8.7 Hz, 1H), 5.61 (d, J=1.8 Hz, 1H), 4.28-4.22 (m, 2H), 3.96 (s, 3H), 3.59 (s, 3H), 3.36 (d, J=8.7 Hz, 1H), 2.85 (s, 1H), 2.78 (s, 1H), 2.28 (s, 3H), 2.14 (s, 3H), 1.36 (s, 3H), 1.13 (s, 3H); ¹³C NMR (125 MHz, CD₂Cl₂) δ 165.6, 160.4, 159.6, 156.3, 149.4, 138.0, 137.3, 131.6, 130.3 (2C), 130.0, 128.8, 128.1, 126.3, 126.0 (2C), 124.1, 122.4, 114.5, 114.4, 111.5, 111.0, 98.2, 84.6, 78.8, 71.6, 69.0, 62.1, 56.1, 29.3, 22.7, 20.0, 8.5; IR (film) νₘₐₓ 3404, 3057, 2974, 2930, 2837, 1713, 1672, 1607, 1526, 1501, 1487, 1367, 1265, 1231, 1094 cm⁻¹; HRMS (ESI+) m/z 590.2390 (M+H⁺, C₃₃H₃₆NO₉ requires m/z 590.2390).

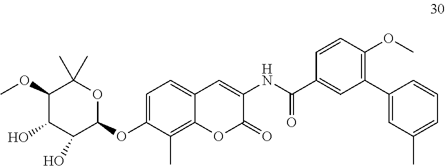

N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)-6-methoxy-3′-methylbiphenyl-3-carboxamide (30). Colorless solid (46%, 2 steps): [α]²⁴_{D}=−14.1° (c=0.17, CH₂Cl₂); ¹H NMR (400 MHz, CD₂Cl₂) δ 8.79 (s, 1H), 8.70 (s, 1H), 7.91 (dd, J=2.4, 8.6 Hz, 1H), 7.86 (d, J=2.4 Hz, 1H), 7.41 (d, J=8.6 Hz, 1H), 7.36-7.30 (m, 3H), 7.21 (d, J=6.0 Hz, 2H), 7.10 (d, J=8.7 Hz, 1H), 5.61 (d, J=1.9 Hz, 1H), 4.27-4.20 (m, 2H), 3.90 (s, 3H), 3.59 (s, 3H), 3.37 (d, J=8.8 Hz, 1H), 2.80 (s, 2H), 2.43 (s, 3H), 2.29 (s, 3H), 1.35 (s, 3H), 1.14 (s, 3H); ¹³C NMR (125 MHz, CD₂Cl₂) δ 164.5, 159.1, 158.5, 155.2, 148.3, 137.1, 136.6, 130.4, 129.4, 129.1, 127.4, 127.2 (2C), 125.9, 125.3, 124.9, 123.0, 121.3, 113.4, 113.3, 110.3, 110.2, 97.1, 83.4, 77.7, 70.5, 67.8, 61.0, 55.0, 28.1, 21.5, 20.4, 7.3; IR (film) νₘₐₓ 3402, 3084, 3051, 2974, 2930, 2839, 1713, 1668, 1607, 1526, 1502, 1367, 1267, 1238, 1092 cm⁻¹; HRMS (ESI+) m/z 590.2390 (M+H⁺, C₃₃H₃₆NO₉ requires m/z 590.2390).

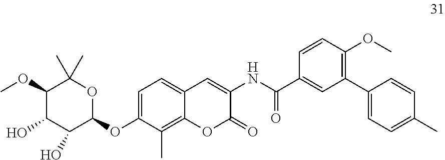

N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)-6-methoxy-4′-methylbiphenyl-3-carboxamide (31). Colorless solid (66%, 2 steps): [α]²⁴_{D}=−16.8° (c=0.10, CH₂Cl₂); ¹H NMR (500 MHz, CD₂Cl₂) δ 8.76 (s, 1H), 8.67 (s, 1H), 7.88 (dd, J=2.0, 8.5 Hz, 1H), 7.83 (d, J=2.0 Hz, 1H), 7.41 (d, J=8.0 Hz, 2H), 7.36 (d, J=8.5 Hz, 1H), 7.24 (d, J=8.0 Hz, 2H), 7.18 (d, J=8.5 Hz, 1H), 7.07 (d, J=8.5 Hz, 1H), 5.58 (s, 1H), 4.25-4.18 (m, 2H), 3.87 (s, 3H), 3.56 (s, 3H), 3.33 (d, J=9.0 Hz, 1H), 2.70 (s, 1H), 2.65 (s, 1H), 2.39 (s, 3H), 2.26 (s, 3H), 1.34 (s, 3H), 1.11 (s, 3H); ¹³C NMR (125 MHz, CD₂Cl₂) δ 165.7, 160.2, 159.6, 156.3, 149.5, 137.8, 134.9, 131.4, 130.1, 129.8 (2C), 129.2 (2C), 128.3, 126.5, 126.1, 124.1, 122.5, 114.5, 114.4, 111.5, 111.3, 98.2, 84.6, 78.8, 71.6, 69.0, 62.1, 56.2, 29.3, 22.7, 21.3, 8.5; IR (film) νₘₐₓ 3404, 3084, 2972, 2926, 2853, 2841, 1713, 1668, 1605, 1520, 1367, 1265, 1232, 1094 cm⁻¹; HRMS (ESI+) m/z 590.2388 (M+H⁺, C₃₃H₃₆NO₉ requires m/z 590.2390).

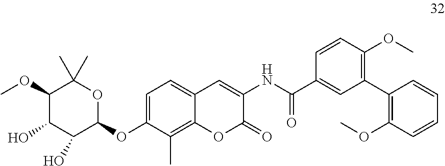

N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)-2′,6-dimethoxybiphenyl-3-carboxamide (32). Colorless solid (62%, 2 steps): [α]²⁵_{D}=−12.2° (c=0.30, CH₂Cl₂); ¹H NMR (500 MHz, CD₂Cl₂) δ 8.75 (s, 1H), 8.66 (s, 1H), 7.92 (dd, J=2.5, 9.0 Hz, 1H), 7.76 (d, J=2.5 Hz, 1H), 7.39-7.33 (m, 2H), 7.23 (dd, J=2.0, 7.5 Hz, 1H), 7.18 (d, J=8.5 Hz, 1H), 7.07 (d, J=9.0 Hz, 1H), 7.04-6.98 (m, 2H), 5.58 (d, J=2.5 Hz, 1H), 4.27-4.20 (m, 2H), 3.84 (s, 3H), 3.77 (s, 3H), 3.57 (s, 3H), 3.33 (d, J=9.0 Hz, 1H), 2.75 (d, J=2.5 Hz, 1H), 2.67 (d, J=2.5 Hz, 1H), 2.26 (s, 3H), 1.34 (s, 3H), 1.11 (s, 3H); ¹³C NMR (125 MHz, CD₂Cl₂) δ 164.5, 159.7, 158.4, 156.3, 155.1, 148.3, 130.5, 129.6, 128.4, 127.5 (2C), 125.8, 124.9 (2C), 122.8, 121.3, 119.5, 113.3 (2C), 110.3, 110.2, 110.0, 97.0, 83.4, 77.7, 70.5, 67.8, 61.0, 55.1, 54.8, 28.1, 21.5, 7.3; IR (film) νₘₐₓ 3404, 3080, 3057, 2930, 2835, 1709, 1670, 1607, 1526, 1502, 1487, 1367, 1265, 1244, 1094 cm¹; HRMS (ESI+) m/z 606.2346 (M+H⁺, C₃₃H₃₆NO₁₀ requires m/z 606.2339).

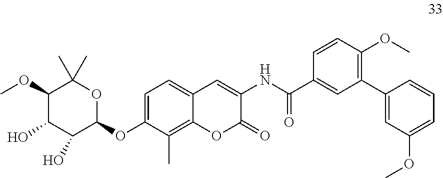

N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)-3′,6-dimethoxybiphenyl-3-carboxamide (33). Colorless solid (52%, 2 steps): [α]²⁵_{D}=−15.8° (c=0.43, CH₂Cl₂); NMR (500 MHz, CD₂Cl₂) δ 8.76 (s, 1H), 8.68 (s, 1H), 7.91 (dd, J=2.5, 8.5 Hz, 1H), 7.87 (d, J=2.5 Hz, 1H), 7.39-7.31 (m, 2H), 7.19 (d, J=8.5 Hz, 1H), 7.13-7.05 (m, 3H), 6.92 (dd, J=2.5, 8.5 Hz, 1H), 5.59 (d, J=2.0 Hz, 1H), 4.28-4.20 (m, 2H), 3.89 (s, 3H), 3.84 (s, 3H), 3.58 (s, 3H), 3.35 (d, J=9.0 Hz, 1H), 2.84 (d, J=1.5 Hz, 1H), 2.74 (d, J=3.0 Hz, 1H), 2.27 (s, 3H), 1.35 (s, 3H), 1.12 (s, 3H); ¹³C NMR (125 MHz, CD₂Cl₂) δ 164.5, 159.0, 158.6, 158.4, 155.2, 148.3, 138.1, 130.1, 129.1, 128.3, 127.4, 125.3, 124.9, 123.0, 121.3, 121.2, 114.5, 113.3, 113.2, 112.1, 110.3, 110.2, 97.1, 83.4, 77.7, 70.4, 67.8, 61.0, 55.1, 54.5, 28.1, 21.5, 7.3; IR (film) νₘₐₓ 3404, 3078, 3057, 2974, 2934, 2835, 1709, 1670, 1607, 1526, 1502, 1367, 1256, 1244, 1113, 1094, 1051, 1022 cm⁻¹; HRMS (ESI+) m/z 606.2346 (M+H⁺, C₃₃H₃₆NO₁₀ requires m/z 606.2339).

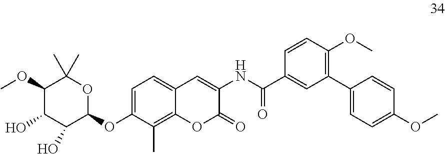

N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)-4′,6-dimethoxybiphenyl-3-carboxamide (34). Colorless solid (46%, 2 steps): [α]²⁶_{D} 0.49, CH₂Cl₂); ¹H NMR (500 MHz, CD₂Cl₂) δ 8.74 (s, 1H), 8.66 (s, 1H), 7.86 (dd, J=2.5, 8.5 Hz, 1H), 7.82 (d, J=2.0 Hz, 1H), 7.46 (dd, J=2.0, 6.5 Hz, 2H), 7.35 (d, J=8.5 Hz, 1H), 7.17 (d, J=8.5 Hz, 1H), 7.05 (d, J=8.5 Hz, 1H), 6.96 (dd, J=2.5, 6.5 Hz, 1H), 5.57 (d, J=2.0 Hz, 1H), 4.24-4.17 (m, 2H), 3.86 (s, 3H), 3.83 (s, 3H), 3.56 (s, 3H), 3.33 (d, J=9.0 Hz, 1H), 2.25 (s, 3H), 1.34 (s, 3H), 1.11 (s, 3H); ¹³C NMR (125 MHz, CD₂Cl₂) δ 164.6, 159.0, 158.5, 158.4, 155.1, 148.3, 129.9 (3C), 128.9 (2C), 126.9, 125.3, 124.9, 122.9, 121.3, 113.3 (2C), 112.7 (2C), 110.3, 110.1, 97.0, 83.4, 77.7, 70.4, 67.8, 61.0, 55.0, 54.5, 28.1, 21.5, 7.3; IR (film) νₘₐₓ 3404, 3082, 3057, 2934, 2837, 1709, 1668, 1607, 1518, 1493, 1265, 1248, 1232, 1118, 1111, 1094, 1080 cm⁻¹; HRMS (ESI+) m/z 606.2328 (M+H⁺, C₃₃H₃₆NO₁₀ requires m/z 606.2339).

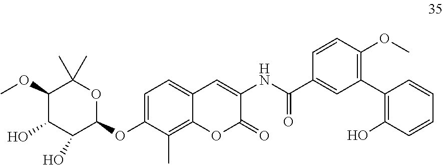

N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)-2′-hydroxy-6-methoxybiphenyl-3-carboxamide (35). Colorless solid (59%, 2 steps): [α]²⁴_{D}=−13.4° (c=0.50, 20% MeOH in CH₂Cl₂); ¹HNMR (400 MHz, CD₂Cl₂) δ 8.73 (s, 1H), 8.67 (s, 1H), 7.98 (dd, J=2.3, 8.6 Hz, 1H), 7.88 (d, J=2.3 Hz, 1H), 7.39-7.25 (m, 3H), 7.18 (d, J=8.8 Hz, 1H), 7.15 (d, J=8.8 Hz, 1H), 7.06 (dd, J=1.1, 7.4 Hz, 1H), 7.04 (d, J=8.1 Hz, 1H), 5.94 (s, 1H), 5.57 (d, J=2.1 Hz, 1H), 4.26-4.17 (m, 2H), 3.95 (s, 3H), 3.58 (s, 3H), 3.35 (d, J=9.0 Hz, 1H), 2.85 (s, 1H), 2.75 (s, 1H), 2.27 (s, 3H), 1.36 (s, 3H), 1.10 (s, 3H); ¹³C NMR (125 MHz, CD₂Cl₂) δ 164.2, 158.5, 158.4, 155.2, 153.0; 148.3, 130.6 (2C), 128.9, 128.0, 126.5, 126.1, 125.0, 124.7, 123.2, 121.0, 120.1, 116.2, 113.3, 113.1, 110.5, 110.3, 96.7, 83.4, 77.7, 70.4, 67.8, 60.9, 55.5, 28.3, 22.2, 8.5; IR (film) νₘₐₓ 3400, 3391, 3090, 2984, 2928, 2849, 1709, 1661, 1651, 1605, 1526, 1495, 1452, 1367, 1267, 1238, 1217, 1180, 1140, 1094 cm⁻¹; HRMS (ESI+) m/z 592.2183 (M+H⁺, C₃₂H₃₄NO₁₀ requires m/z 592.2183).

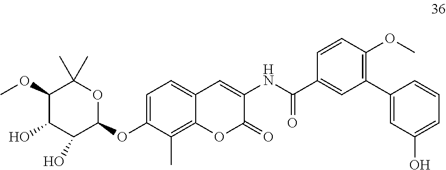

N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)-3′-hydroxy-6-methoxybiphenyl-3-carboxamide (36). Colorless solid (34%, 2 steps): [α]²⁴_{D}=−13.5° (c=0.16, 20% MeOH in CH₂Cl₂); ¹H NMR (500 MHz, 20% CD₃OD in CD₂Cl₂) δ 8.78 (s, 1H), 8.76 (s, 1H), 7.91 (dd, J=2.4, 8.6 Hz, 1H), 7.86 (d, J=2.4 Hz, 1H), 7.40 (d, J=8.7 Hz, 1H), 7.29-7.19 (m, 2H), 7.10 (d, J=8.7 Hz, 1H), 7.02 (dd, J=1.0, 7.6 Hz, 1H), 6.99 (t, J=2.0 Hz, 1H), 6.83 (td, J=1.0, 8.0 Hz, 1H), 5.56 (d, J=2.1 Hz, 1H), 4.19-4.09 (m, 2H), 3.89 (s, 3H), 3.58 (s, 3H), 3.34-3.32 (m, 1H), 2.28 (s, 3H), 1.33 (s, 3H), 1.10 (s, 3H); ¹³C NMR (125 MHz, 20% CD₃OD in CD₂Cl₂) δ 165.2, 159.2, 158.8, 156.0, 155.6, 148.5, 138.1, 130.3, 129.1, 128.4, 127.4, 125.2, 125.1, 124.0, 121.0, 120.2, 115.7, 113.7, 113.4, 113.2, 110.6, 110.4, 97.7, 83.5, 77.8, 70.5, 67.7, 68.9, 55.0, 27.9, 21.6, 7.2; IR (film) νₘₐₓ 3400, 2922, 2851, 1707, 1647, 1630, 1605, 1528, 1501, 1369, 1250, 1095 cm⁻¹; HRMS (ESI+) m/z 592.2191 (M+H⁺, C₃₂H₃₄NO₁₀ requires m/z 592.2183).

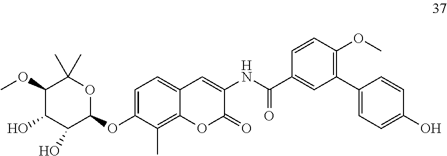

N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)-4′-hydroxy-6-methoxybiphenyl-3-carboxamide (37). Colorless solid (29%, 2 steps): [α]²⁴_{D}=−21.7° (c=0.06, 20% MeOH in CH₂Cl₂); ¹H NMR (500 MHz, 20% CD₃OD in CD₂Cl₂) δ 8.80 (s, 1H), 8.74 (s, 1H), 7.87 (dd, J=2.3, 8.7 Hz, 1H), 7.82 (d, J=2.3 Hz, 1H), 7.42-7.35 (m, 3H), 7.21 (d, J=8.8 Hz, 1H), 7.08 (d, J=8.6 Hz, 1H), 6.86 (d, J=8.6 Hz, 1H), 5.55 (d, J=2.0 Hz, 1H), 4.16-4.09 (m, 2H), 3.88 (s, 3H), 3.57 (s, 3H), 3.33 (d, J=9.4 Hz, 1H), 2.27 (s, 3H), 1.32 (s, 3H), 1.09 (s, 3H); ¹³C NMR (125 MHz, 20% CD₃OD in CD₂Cl₂) δ 164.5, 158.4, 158.0, 155.0, 154.7, 147.6, 129.4, 129.1 (2C), 128.0, 127.0, 126.0, 124.3, 124.2, 123.2, 120.2, 113.3 (2C), 112.5, 112.3, 109.7, 109.4, 96.9, 82.6, 77.0, 69.7, 66.8, 59.9, 54.1, 27.0, 20.7, 6.3; IR (film) νₘₐₓ 3402, 3394, 2997, 2922, 2851, 1707, 1653, 1605, 1520, 1495, 1369, 1265, 1234, 1095 cm⁻¹; HRMS (ESI+) m/z 592.2176 (M+H⁺, C₃₂H₃₄NO₁₀ requires m/z 592.2183).

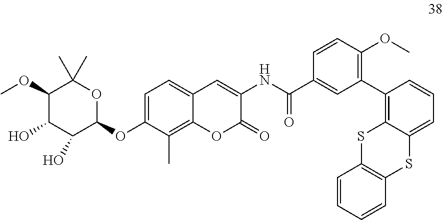

N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)-4-methoxy-3-(thianthren-1-yl)benzamide (38). Colorless solid (51%, 2 steps): [α]²⁶_{D}=−12.0° (c=0.82, CH₂Cl₂); ¹H NMR (500 MHz, 20% CD₃OD in CD₂Cl₂) δ 8.81 (s, 1H), 8.73 (s, 1H), 8.08 (d, J=7.6 Hz, 1H), 7.80 (s, 1H), 7.61 (d, J=6.4 Hz, 1H), 7.56 (d, J=6.4 Hz, 1H), 7.42-7.16 (m, 8H), 5.63 (s, 1H), 4.28-4.22 (m, 2H), 3.94 (s, 3H), 3.61 (s, 3H), 3.38 (d, J=8.0 Hz, 1H), 2.95 (s, 1H), 2.87 (s, 1H), 2.30 (s, 3H), 1.39 (s, 3H), 1.16 (s, 3H); ¹³C NMR (125 MHz, 20% CD₃OD in CD₂Cl₂) δ 165.1, 160.1, 159.2, 155.9, 153.5, 149.1, 138.0, 136.3, 136.1, 135.8, 135.6, 129.9, 129.7, 129.6, 129.2, 128.8, 128.5, 127.8, 127.6, 127.4, 126.0, 125.7, 123.9, 122.0, 114.1, 114.0, 111.1, 110.7, 97.8, 84.2, 78.5, 71.2, 68.6, 61.7, 55.9, 28.9, 22.3, 8.1; IR (film) νₘₐₓ 3400, 3055, 2976, 2932, 2837, 1709, 1670, 1605, 1526, 1497, 1439, 1367, 1261, 1234, 1111, 1094 cm⁻¹; HRMS (ESI+) m/z 714.1822 (M+H⁺, C₃₈H₃₆NO₉S₂ requires m/z 714.1832).

### Example 11

### Synthesis of Heterocyclic Novobiocin Analogues

In an effort to incorporate the structure-activity relationships into more efficacious inhibitors, a small library of novobiocin derivatives was prepared. This library focused on the incorporation of heterocycles into the benzamide region in order to investigate hydrogen bond donor/acceptor interactions and the effects of rigidity as suggested by initial findings.

The novobiocin derivatives were prepared by coupling commercially available carboxylic acids with aminocoumarin 7 from the above examples via treatment with EDCI and pyridine, the carbonates of the resulting molecules were then solvolyzed with methanolic triethylamine to afford the requisite diols, 40-47 as shown in the scheme 18 below.

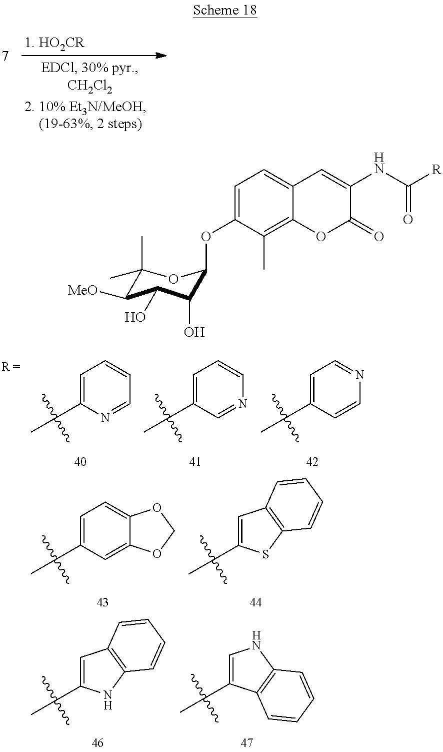

General EDCI coupling procedure B: N-(3-Dimethylamino-propyl)-N′-ethylcarbodiimide hydrochloride (2.5 eq) was added to a solution of aminocoumarin 7 (1.0 eq), carboxylic acid (2.0 eq) in CH₂Cl₂ containing 30% pyridine at room temperature. The solution was stirred for 14 hours, concentrated and the residue purified via preparative TLC (SiO₂, 40:1; CH₂Cl₂:acetone) to afford the amide. The resulting product was dissolved in methanol containing 10% Et₃N and stirred for 14 hours at room temperature. The mixture was concentrated and the residue was purified by preparative TLC (10:1; CH₂Cl₂:methanol or 4:1; CH₂Cl₂:acetone) to afford the corresponding diol.

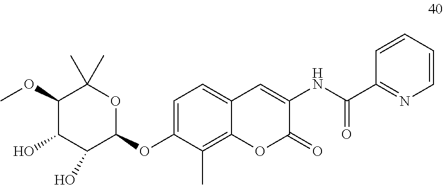

N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)picolinamide (40). Yellow solid (56%, 2 steps): [α]²⁵_{D}=−18.8° (c=0.48, 20% MeOH in CH₂Cl₂); ¹H NMR (500 MHz; 20% CD₃OD in CD₂Cl₂) δ 8.76 (s, 1H), 8.66 (d, J=4.5 Hz, 1H), 8.18 (d, J=7.5 Hz, 1H), 7.93 (dt, J=1.5, 7.5 Hz, 1H), 7.54-7.49 (m, 1H), 7.37 (d, J=8.5 Hz, 1H), 7.19 (d, J=8.5 Hz, 1H), 5.53 (d, J=2.0 Hz, 1H), 4.14 (dd, J=3.5, 9.5 Hz, 1H), 4.09 (t, J=3.5 Hz, 1H), 3.55 (s, 3H), 3.32 (d, J=9.5 Hz, 1H), 2.27 (s, 3H), 1.31 (s, 3H), 1.08 (s, 3H); ¹³C NMR (125 MHz, 20% CD₃OD in CD₂Cl₂) δ 165.0, 160.8, 158.1, 151.1, 150.6, 150.3, 139.5, 128.7, 127.4, 126.4, 123.8, 123.0, 115.9, 115.5, 112.9, 100.2, 85.8, 80.2, 72.9, 70.1, 63.1, 30.2, 23.9, 9.5; IR (film) νₘₐₓ 3421, 3065, 2982, 2932, 2837, 2476, 1717, 1682, 1626, 1607, 1522, 1458, 1414, 1375, 1265, 1173, 1134, 1097, 1080 cm⁻¹; HRMS (ESI+) m/z 471.1751 (M+H⁺, C₂₄H₂₇N₂O₈ requires m/z 471.1767).

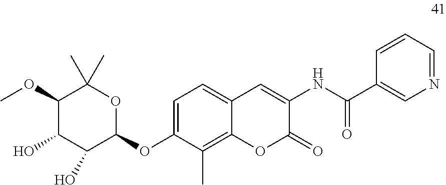

N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)nicotinamide (41). Colorless solid (52%, 2 steps): [α]25ₚ=−16.1° (c=0.31, 20% MeOH in CH₂Cl₂); ¹H NMR (400 MHz, CD₂Cl₂) δ 9.13 (s, 1H), 8.80-8.75 (m, 1H), 8.77 (s, 1H), 8.73 (s, 1H), 8.20 (dd, J=1.6, 7.2 Hz, 1H), 7.47 (dd, J=4.8, 7.6 Hz, 1H), 7.38 (d, J=8.4 Hz, 1H), 7.21 (d, J=8.4 Hz, 1H), 5.60 (d, J=2.0 Hz, 1H), 4.27-4.17 (m, 2H), 3.58 (s, 3H), 3.35 (d, J=8.4 Hz, 1H), 2.75 (s, 2H), 2.27 (s, 3H), 1.36 (s, 3H), 1.12 (s, 3H); ¹³C NMR (125 MHz, 20% CD₃OD in CD₂Cl₂) δ 166.0, 160.9, 158.3, 154.0, 151.1, 149.8, 137.2, 131.7, 127.6 (2C), 125.6, 123.0, 115.9, 115.3, 113.0, 100.1, 85.8, 80.2, 72.9, 70.1, 63.2, 30.2, 23.9, 9.5; IR (film) νₘₐₓ 3394, 3092, 3065, 2980, 2928, 2854, 2833, 1711, 1672, 1605, 1531, 1371, 1258, 1132, 1113, 1095, 1082 cm'; HRMS (ESI+) m/z 471.1763 (M+H⁺, C₂₄H₂₇N₂O₈ requires m/z 471.1767).

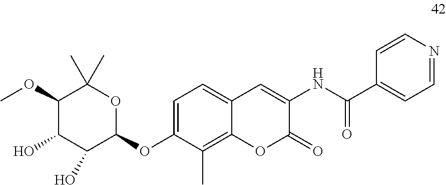

N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)isonicotinamide (42). Yellow solid (52%, 2 steps): [α]²⁵_{D}=−14.3° (c=0.67, 20% MeOH in CH₂Cl₂); ¹H NMR (800 MHz, 20% CD₃OD in CD₂Cl₂) δ 8.83-8.74 (m, 3H), 7.87-7.80 (m, 2H), 7.42 (d, J=8.0 Hz, 1H), 7.24 (d, J=8.0 Hz, 1H), 5.58 (d, J=2.4 Hz, 1H), 4.20-4.11 (m, 2H), 3.60 (s, 3H), 3.36 (d, J=9.6 Hz, 1H), 2.28 (s, 3H), 1.35 (s, 3H), 1.12 (s, 3H); ¹³C NMR (200 MHz, 20% CD₃OD in CD₂Cl₂) δ 164.2, 159.1, 156.7, 150.2 (3C), 149.5, 141.5, 126.4, 126.0, 121.3, 121.0, 114.1, 113.4, 111.3, 98.5, 84.1, 78.6, 71.2, 68.4, 61.4, 28.5, 22.2, 7.8; IR (film) νₘₐₓ 3393, 3055, 2982, 2932, 2835, 1709, 1674, 1628, 1607, 1529, 1373, 1258, 1134, 1111, 1095, 1080 cm⁻¹; HRMS (ESI+) m/z 471.1748 (M+H⁺, C₂₄H₂₇N₂O₈ requires m/z 471.1767).

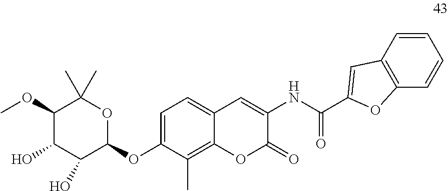

N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)benzofuran-2-carboxamide (43). Colorless solid (63%, 2 steps): [α]²⁴_{D}=−16.8° (c=0.19, 20% MeOH in CH₂Cl₂); ¹HNMR (400 MHz, 20% CD₃OD in CD₂Cl₂) δ 8.70 (s, 1H), 7.68 (dd, J=0.8, 8.8 Hz, 1H), 7.59 (dd, J=0.8, 8.8 Hz, 1H), 7.58 (s, 1H), 7.45 (dt, J=0.8, 7.6 Hz, 1H), 7.37 (d, J=8.8 Hz, 1H), 7.29 (dt, J=0.8, 7.6 Hz, 1H), 7.19 (d, J=8.8 Hz, 1H), 5.52 (d, J=2.4 Hz, 1H), 4.12 (dd, J=3.4, 9.2 Hz, 1H), 4.07 (dd, J=3.4, 9.2 Hz, 1H), 3.54 (s, 3H), 3.30 (d, J=9.2 Hz, 1H), 2.25 (s, 3H), 1.29 (s, 3H), 1.06 (s, 3H); ¹³C NMR (125 MHz, 20% CD₃OD in CD₂Cl₂) δ 161.5, 159.8, 159.0, 157.5, 151.8, 150.0, 130.1, 129.9, 128.3, 127.8, 126.4, 125.2, 123.3, 116.6, 116.0, 114.4, 114.2, 113.7, 100.9, 86.5, 81.0, 73.6, 70.8, 63.8, 30.8, 24.6, 10.2; IR (film) νₘₐₓ 3404, 3385, 2986, 2935, 2511, 1717, 1670, 1626, 1607, 1576, 1548, 1418, 1377, 1265, 1115, 1092 cm⁻¹; HRMS (ESI+) m/z 532.1566 (M+Na⁺, C₂₇H₂₇NO₉Na requires m/z 532.1583).

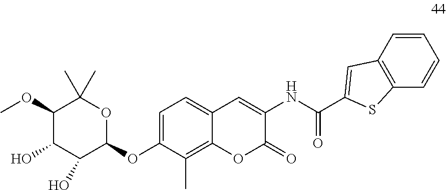

N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)benzo[b]thiophene-2-carboxamide (44). Colorless solid (42%, 2 steps): [α]²⁵_{D}=−24.3° (c=0.23, 20% MeOH in CH₂Cl₂); NMR (400 MHz, 20% CD₃OD in CD₂Cl₂) δ 8.66 (s, 1H), 8.00 (s, 1H), 7.88 (t, J=7.4 Hz, 2H), 7.45-7.33 (m, 3H), 7.19 (d, J=8.8 Hz, 1H), 5.53 (d, J=2.0 Hz, 1H), 4.12 (d, J=9.2 Hz, 1H), 4.09-4.05 (m, 1H), 3.54 (s, 3H), 3.31 (d, J=9.2 Hz, 1H), 2.25 (s, 3H), 1.30 (s, 3H), 1.07 (s, 3H); ¹³C NMR (125 MHz, 20% CD₃OD in CD₂Cl₂) δ 161.6, 156.9, 149.0, 145.3, 141.8, 139.5, 138.0, 127.2, 126.6, 126.2, 126.0, 125.8, 125.5, 123.0, 121.3, 114.7, 114.2, 111.7, 98.9, 84.5, 78.9, 71.6, 68.8, 61.7, 28.8, 22.6, 8.1; IR (film) νₘₐₓ 3414, 3381, 2986, 2932, 2526, 1690, 1649, 1632, 1601, 1529, 1375, 1254, 1240, 1194, 1084 cm⁻¹; HRMS (ESI+) m/z 526.1532 (M+H⁺, C₂₇H₂₈NO₈S requires m/z 526.1536).

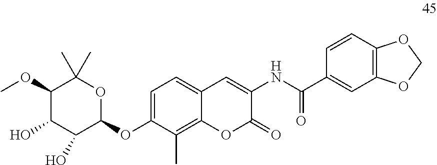

N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)benzo[d][1,3]dioxole-5-carboxamide (45). Colorless solid (39%, 2 steps): [α]²⁶_{D}=−15.9° (c=0.27, 20% MeOH in CH₂Cl₂); ¹H NMR (500 MHz, 20% CD₃OD in CD₂Cl₂) δ 8.64 (s, 1H), 7.39 (d, J=8.0 Hz, 1H), 7.31 (s, 1H), 7.30 (d, J=8.0 Hz, 1H), 7.14 (d, J=8.5 Hz, 1H), 6.84 (d, J=8.5 Hz, 1H), 5.99 (s, 2H), 5.48 (s, 1H), 4.08 (d, J=9.5 Hz, 1H), 4.04 (d, J=2.0 Hz, 1H), 3.48 (s, 3H), 3.25 (d, J=9.5 Hz, 1H), 2.20 (s, 3H), 1.22 (s, 3H), 1.12 (s, 3H); ¹³C NMR (125 MHz, 20% CD₃OD in CD₂Cl₂) δ 164.7, 158.7, 155.6, 150.6, 148.4, 147.7, 127.2, 125.1, 123.9, 121.5, 121.0, 113.5, 113.2, 110.6, 107.5, 106.8, 101.5, 97.7, 83.5, 77.9, 70.5, 67.8, 60.8, 27.9, 21.6, 7.2; IR (film) νₘₐₓ 3404, 3111, 3035, 2980, 2926, 2853, 1701, 1607, 1528, 1485, 1444, 1406, 1369, 1254, 1132, 1113, 1086, 1038 cm⁻¹; HRMS (ESI+) m/z 514.1708 (M+H⁺, C₂₆H₂₈N₂O₁₀ requires m/z 514.1713).

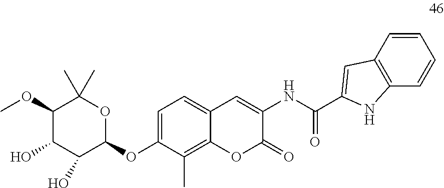

N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)-1H-indole-2-carboxamide (46). Yellow solid (31%, 2 steps): [α]²⁵_{D}=−18.2° (c=0.22, 20% MeOH in CH₂Cl₂); ¹HNMR (800 MHz, 20% CD₃OD in CD₂Cl₂) δ 8.69 (s, 1H), 7.66 (d, J=8.0 Hz, 1H), 7.46 (d, J=8.0 Hz, 1H), 7.36 (d, J=9.0 Hz, 1H), 7.27 (t, J=7.5 Hz, 1H), 7.20 (d, J=9.0 Hz, 1H), 7.17 (s, 1H), 7.10 (t, J=7.5 Hz, 1H), 5.53 (d, J=2.0 Hz, 1H), 4.17-4.08 (m, 2H), 3.55 (s, 3H), 3.31 (d, J=9.5 Hz, 1H), 2.26 (s, 3H), 1.30 (s, 3H), 1.08 (s, 3H); ¹³C NMR (200 MHz, 20% CD₃OD in CD₂Cl₂) δ 162.1, 161.0, 158.0, 150.9, 139.0, 131.8, 129.1, 127.3, 126.6 (2C), 123.7, 123.1, 122.3, 115.8, 115.5, 113.7, 112.9, 106.9, 100.2, 85.8, 80.2, 72.9, 70.1, 63.1, 30.1, 23.9, 9.4; IR (film) νₘₐₓ 3443, 3421, 3404, 3003, 2986, 2935, 1609, 1541, 1364, 1263, 1105, 1082 cm⁻¹; HRMS (ESI+) m/z 509.1916 (M+H⁺, C₂₇H₂₉N₂O₈ requires m/z 509.1924).

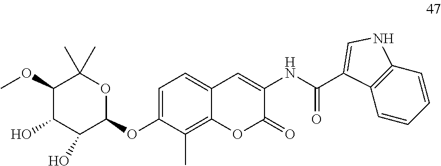

N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)-1H-indole-3-carboxamide (47). Colorless solid (19%, 2 steps): [α]²⁶_{D}=−11.4° (c=0.18, 20% MeOH in CH₂Cl₂); ¹H NMR (500 MHz, 20% CD₃OD in CD₂Cl₂) δ 8.75 (s, 1H), 8.12 (dd, J=2.0, 6.5 Hz, 1H), 7.97 (s, 1H), 7.49 (dd, J=2.0, 6.5 Hz, 1H), 7.38 (d, J=8.5 Hz, 1H), 7.31-7.24 (m, 2H), 7.20 (d, J=8.5 Hz, 1H), 5.55 (d, J=2.5 Hz, 1H), 4.16 (dd, J=4.5, 9.5 Hz, 1H), 4.12 (t, J=4.5 Hz, 1H), 3.57 (s, 3H), 3.33-3.31 (m, 1H), 2.28 (s, 3H), 1.33 (s, 3H), 1.10 (s, 3H); ¹³C NMR (125 MHz, 20% CD₃OD in CD₂Cl₂) δ 165.7, 161.3, 157.6, 150.6, 138.4, 131.0, 127.1, 126.4, 125.1, 124.5, 123.8, 123.5, 121.4, 115.8 (2C), 113.9, 112.8, 112.7, 100.0, 85.9, 80.1, 72.9, 70.1, 63.2, 30.2, 23.9, 9.6; IR (film) νₘₐₓ 3439, 3418, 3394, 2957, 2924, 2853, 1636, 1529, 1437, 1379, 1261, 1180, 1128, 1082, 1020 cm⁻¹; HRMS (ESI+) m/z 509.1924 (M+H⁺, C₂₇H₂₉N₂O₈ requires m/z 509.1924).

### Example 12

### Synthesis of 5-, 6-, and 8-Alkyl(Oxy) Resorconol Precursors for Novobiocin Analogues

In the following example, modifications at the 5-, 6-, and 8-position of the coumarin ring were made in order to mimic those at the 6-, 7-, and 3-position of guanine. Various resorcinol precursors were therefore made.

To generate the resorcinol precursors with substitutions at the 4-position, which result in coumarin ring systems with appendages at the 6-position, the phenols of benzaldehyde 1 were protected as the corresponding ethers as shown in Scheme 19 below. The resulting benzaldehydes (2a-b) (Nabaei-Bidhendi et al., Convenient synthesis of polyhydroxy flavonoids, J. Indian Chem. Soc. 67 43-45 (1990)) were converted to their formate esters via Baeyer-Villiger oxidation, and then hydrolyzed to afford phenols 3a-b. See Horvath, R. F.; Chan, T. H. J. Org. Chem. 52 4489-4494 (1986); Miyake et al., Synthesis and Biological Activity of Arthrographol and Related Compounds, Heterocycles 43 665-674 (1996). O-Alkylation with the requisite alkyl iodide proceeded in good yield and generated a series of protected 4-substituted resorcinolic ethers (4a-c). Ortho-lithiation of 4a-c, followed by alkylation with methyl iodide provided the 2-methyl protected resorcinols, 5a-c. See Carreno, M. C.; Garcia Ruano, J. L.; Toledo, M. A.; Urbano, A. Tetrahedron: Asymmetry 8 913-921 (1997). Deprotection of the alkoxy ethers by exposure to acidic conditions gave resorcinols 6a-c. See Wang, Y.; Tan, W.; Li, W. Z.; Li, Y. J. Nat. Prod. 64 196-199 (2001).

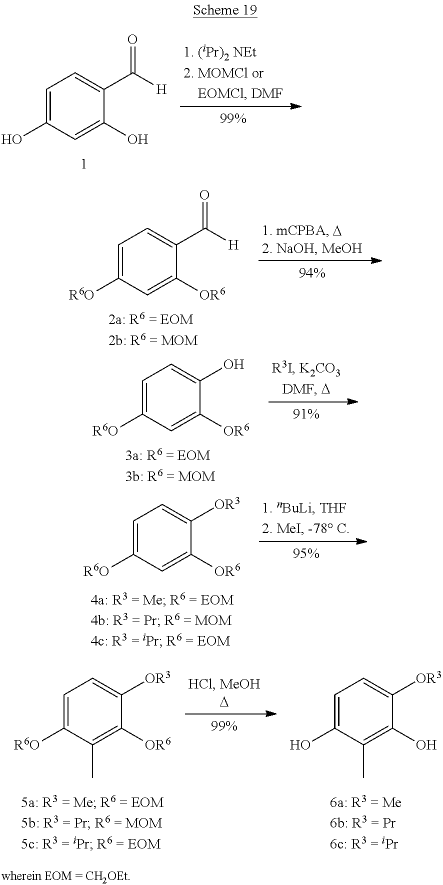

2,4-Bis(ethoxymethoxy)benzaldehyde (2a): N,N-Diisopropylethylamine (25.3 mL, 145 mmol) was slowly added to 2,4-dihydroxybenzaldehyde (5.00 g, 36.2 mmol) in anhydrous N,N-dimethylformamide (100 mL) over five minutes at room temperature. After 30 minutes, the solution was cooled to 0° C. and chloromethyl ethyl ether (14.2 mL, 145 mmol) was added and the mixture warmed to room temperature over 12 hours. The reaction was quenched by the addition of saturated aqueous NH₄Cl solution and extracted with EtOAc (3×50 mL). The combined organic fractions were washed with saturated aqueous NaCl, dried (Na₂SO₄), filtered, and concentrated. The residue was purified via column chromatography (SiO₂, 5:1→1:1 Hexane:EtOAc) to give 2a as a brown amorphous solid (9.10 g, 99%): ¹H NMR (CDCl₃, 400 MHz) δ 10.34 (d, J=2.4 Hz, 1H), 7.81 (dd, J=8.7, 2.8 Hz, 1H), 6.89 (t, J=2.5 Hz, 1H), 6.74 (m, 1H), 5.34 (d, J=2.8, 2H), 5.28 (d, J=2.8, 2H), 3.81-3.71 (m, 4H), 1.28-1.22 (m, 6H).

2,4-Bis(ethoxymethoxy)phenol (3a): A solution of 2a (3.78 g, 12.0 mmol) in anhydrous CH₂Cl₂ (4.0 mL) was slowly added to mCPBA (70%) (3.26 g, 13.2 mmol) in anhydrous CH₂Cl₂ (16.3 mL) at 0° C. The resulting solution was warmed to room temperature, then refluxed for 12 hours. After cooling to room temperature, the resulting solution was washed with saturated aqueous NaHCO₃ solution (3×20 mL) and 10% aqueous Na₂S₂O₃ (30 mL). Combined organic fractions were dried (Na₂SO₄), filtered, and concentrated. The residue was re-dissolved in MeOH (5 mL) and stirred with excess 10% aqueous NaOH for three hours at room temperature. The pH was adjusted to 2 with 6 M HCl and the solution was extracted with CH₂Cl₂ (3×10 mL). Combined organic fractions were dried (Na₂SO₄), filtered, and concentrated to give 3a as an orange oil (8.21 g, 94%): ¹H NMR (CDCl₃, 500 MHz) [symbol] 6.89-6.85 (m, 2H), 6.67 (dd, J=8.8, 2.7 Hz, 1H), 5.81 (d, J=6.6 Hz, 1H), 5.23 (s, 2H), 5.15 (s, 2H), 3.80-3.73 (m, 4H), 1.29-1.24 (m, 6H); ¹³C NMR (CDCl₃, 125 MHz) S151.0, 145.0, 141.5, 115.2, 110.6, 106.0, 94.9, 94.2, 64.8, 64.1, 15.1, 15.1; IR (film) νₘₐₓ 3362, 2887, 1460, 1286, 1162, 735 cm⁻¹; HRMS (ESI+) m/z: [M+Na]⁺ calcd for C₁₂H₁₈O₅, 265.1052. Found, 265.1045.

2,4-Bis(methoxymethoxy)phenol (3b): Benzaldehyde 2b (700 mg; 3.11 mmol) in CHCl₃ (1.80 mL) at 0° C. was treated with mCPBA (70% w/w, 1.61 g, 9.33 mmol). After 10 minutes, the solution was warmed to room temperature, then refluxed for 12 hours. Upon cooling to room temperature, the solution was washed with saturated aqueous NaHCO₃ (3×10 mL), saturated aqueous Na₂SO₃ (20 mL), saturated aqueous NaCl, was dried (Na₂SO₄), filtered, and concentrated. The residue was dissolved in MeOH (5 mL) and stirred with excess triethylamine for three hours at room temperature. The solvent was concentrated and the residue purified by column chromatography (SiO₂, 4:1→3:1 Hexane:EtOAc) to afford 3b as a yellow oil (320 mg, 50%): ¹H NMR (CDCl₃, 400 MHz) δ 6.87 (d, J=8.9 Hz, 1H), 6.86 (s, 1H), 6.67 (dd, J=11.5, 2.8 Hz, 1H), 5.21 (s, 2H), 5.11 (s, 2H), 3.54 (s, 3H), 3.50 (s, 3H).

2,4-Bis(ethoxymethoxy)-1-methoxybenzene (4a): Potassium carbonate (14.3 g, 103 mmol) was added to 3a (2.50 g, 10.3 mmol) in N,N-dimethylformamide (103 mL). After 10 minutes, methyl iodide (6.43 mL, 103 mmol) was added and the solution was heated to reflux for 12 hours. Upon cooling to room temperature, the solution was extracted with EtOAc (3×50 mL); combined organic fractions were washed with saturated aqueous NaCl, dried (Na₂SO₄), and concentrated. The residue was purified by column chromatography (SiO₂, 4:1 Hexane:EtOAc) to afford 4a as a yellow oil (2.40 g, 91%): ¹H NMR (CDCl₃, 500 MHz) δ 6.87 (d, J=2.8 Hz, 1H), 6.72 (d, J=8.9 Hz, 1H), 6.60 (dd, J=13.3, 1.7 Hz, 1H), 5.18 (s, 2H), 5.07 (s, 2H), 3.76 (s, 3H), 3.72-3.69 (m, 2H), 3.68-3.63 (m, 2H), 1.17-1.13 (m, 6H); ¹³C NMR (CDCl₃, 125 MHz) δ 150.7, 146.2, 143.9, 111.2, 107.9, 105.8, 93.2, 93.0, 63.3, 63.0, 55.4, 14.1, 14.0; IR (film) νₘₐₓ 2976, 2932, 2899, 2835, 1595, 1508, 1393, 1227, 1153, 1103, 1080, 1009, 847 cm⁻¹; HRMS (ESI+) m/z: [M+Na]⁺ calcd for C₁₃H₂₀O₅, 279.1208. Found, 279.1181.

2,4-Bis(methoxymethoxy)-1-propoxybenzene (4b): Potassium carbonate (322 mg, 2.33 mmol) was added to 3b (50 mg, 0.233 mmol) in N,N-dimethylformamide (2.33 mL) at room temperature. After 10 minutes, iodopropane (226 μL, 2.33 mmol) was added and the solution was heated to reflux for 12 hours. Upon cooling to room temperature, the solution was extracted with EtOAc (3×10 mL); combined organic fractions were washed with saturated aqueous NaCl, dried (Na₂SO₄), filtered, and concentrated. The residue was purified by column chromatography (SiO₂, 5:1 Hexane:EtOAc) to afford 4b as a yellow oil (36.4 mg, 61%): ¹H NMR (CD₂Cl₂, 400 MHz) δ 6.87 (s, 1H), 6.84 (d, J=2.9 Hz, 1H), 6.68 (dd, J=11.7, 2.8 Hz, 1H), 5.19 (s, 2H), 5.12 (s, 2H), 3.93 (t, J=6.6 Hz, 2H), 3.53 (s, 3H), 3.49 (s, 3H), 1.86-1.78 (m, 2H), 1.06 (t, J=7.5 Hz, 3H); ¹³C NMR (CDCl₃, 125 MHz) δ 150.6, 146.5, 143.8, 113.7, 108.5, 106.6, 94.7, 94.2, 70.3, 55.2, 54.9, 21.6, 9.5; IR (film) νₘₐₓ 2961, 2826, 1595, 1506, 1400, 1261, 1154, 1013, 1076, 924, 800 cm⁻¹; HRMS (ESI+) m/z: [M+H]⁺ calcd for C₁₃H₂₀O₅, 257.1389. Found, 257.1410; [M+Na]⁺ calcd for C₁₃H₂₀O₅, 279.1208. Found, 279.1165.

2,4-Bis(ethoxymethoxy)-1-isopropoxybenzene (4c): Potassium carbonate (2.85 g, 20.7 mmol) was added to 3a (500 mg, 2.07 mmol) in N,N-dimethylformamide (4.10 mL) at room temperature. After 10 minutes, 2-iodopropane (2.06 mL, 20.7 mmol) was added and the solution was heated to reflux for 12 hours. Upon cooling to room temperature, the solution was extracted with EtOAc (3×20 mL); combined organic fractions were washed with saturated aqueous NaCl, dried (Na₂SO₄), filtered, and concentrated. The residue was purified via column chromatography (SiO₂, 5:1→1:1 Hexane:EtOAc) to afford 4c as a yellow oil (0.32 g, 55%): ¹H NMR (CD₂Cl₂, 400 MHz) δ 6.87 (s, 1H), 6.86 (d, J=4.9 Hz, 1H), 6.66 (dd, J=11.6, 3.4 Hz, 1H), 5.23 (s, 2H), 5.17 (s, 2H), 4.44-4.38 (m, 1H), 3.83-3.72 (m, 4H), 1.33 (s, 3H), 1.31 (s, 3H), 1.27-1.23 (m, 6H); ¹³C NMR (CDCl₃, 125 MHz) δ 152.4, 149.1, 143.2, 118.6, 109.5, 107.5, 94.4, 93.9, 72.8, 64.3, 64.1, 22.2 (2C), 15.1, 15.1; IR (film) νₘₐₓ 2976, 1591, 1504, 1528, 1391, 1258, 1217, 1107, 1011, 847 cm⁻¹; HRMS (ESI⁺) m/z: [M+H]⁺ calcd for C₁₅H₂₄O₅, 285.1702. Found, 285.1746; [M+Na]⁺ calcd for C₁₅H₂₄O₅, 307.1522. Found, 307.1310.

1,3-Bis(ethoxymethoxy)-4-methoxy-2-methylbenzene (5a): A solution of 4a (632 mg, 2.27 mmol) in anhydrous THF (1.94 mL) was added dropwise to a solution of ⁿBuLi (2.5 M in hexanes, 1.48 mL, 3.70 mmol) in anhydrous THF (1.62 mL) at room temperature. After one hour, the solution was cooled to −78° C. and methyl iodide (620 μL, 9.87 mmol) was added. The resulting solution was warmed to room temperature over 12 hours, and the reaction was quenched by the addition of saturated aqueous NH₄Cl. Water (5 mL) was added and the solution was extracted with CH₂Cl₂ (3×10 mL). Combined organic fractions were dried (Na₂SO₄), filtered, and concentrated. The residue was purified via column chromatography (SiO₂, 8:1→5:1 Hexane:EtOAc) to afford 5a as a yellow oil (353 mg, 53%): ¹HNMR (CDCl₃, 500 MHz) δ 6.74 (d, J=9.0 Hz, 1H), 6.60 (d, J=9.0 Hz, 1H) 5.10 (s, 2H), 5.05 (s, 2H), 3.78 (q, J=7.1 Hz, 2H), 3.72 (s, 3H), 3.67 (q, J=7.1 Hz, 2H), 2.14 (s, 3H), 1.18-1.15 (m, 6H); ¹³C NMR (CDCl₃, 125 MHz) δ 149.1, 146.5, 121.7, 109.0, 108.4, 96.2, 93.0, 64.3, 63.1, 55.1, 28.7, 14.2, 14.1, 8.8; IR (film) νₘₐₓ 2918, 2359, 1487, 1260, 1248, 1082, 1055, 945, 798 cm⁻¹; HRMS (ESI⁺) m/z: [M+Na]⁺ calcd for C₁₄H₂₂O₅, 293.1365. Found, 293.1357.

1,3-Bis(methoxymethoxy)-2-methyl-4-propoxybenzene (5b): A solution of 4b (165 mg, 0.64 mmol) in anhydrous THF (520 μL) was added dropwise to a solution of ⁿBuLi (2.5 M in hexanes, 390 μL, 0.97 mmol) in anhydrous THF (420 μL) at room temperature. After 1 hour, the solution was cooled to −78° C. and methyl iodide (160 μL, 2.58 mmol) was added. The resulting solution was warmed to room temperature over 12 hours, and the reaction was quenched by the addition of saturated aqueous NH₄Cl. Water (5 mL) was added and the solution was extracted with CH₂Cl₂ (3×10 mL). Combined organic fractions were dried (Na₂SO₄), filtered, and concentrated. The residue was purified via column chromatography (SiO₂, 6:1 Hexane:EtOAc) to afford 5b as a yellow oil (166 mg, 95%): ¹H NMR (CDCl₃, 500 MHz) δ 6.66 (d, J=9.0 Hz, 1H), 6.60 (d, J=9.0 Hz, 1H), 5.02 (s, 2H), 5.00 (s, 2H), 3.80-3.77 (m, 2H), 3.49 (s, 3H), 3.47 (s, 3H), 2.14 (d, J=7.1 Hz, 3H), 1.73-1.69 (m, 2H), 0.94 (t, J=7.5 Hz, 3H); ¹³C NMR (CDCl₃, 125 MHz) δ 148.5, 148.5, 147.3, 145.6, 126.7, 123.0, 112.8, 110.8, 110.4, 99.2, 57.7, 57.6, 21.2, 10.9, 10.0; IR (film) νₘₐₓ 2957, 2924, 2853, 1738, 1597, 1487, 1468, 1391, 1335, 1231, 1157, 974, 798 cm'; HRMS (ESI⁺) m/z: [M+H]⁺ calcd for C₁₄H₂₂O₅, 271.1545. Found, 271.1558.

1,3-Bis(ethoxymethoxy)-4-isopropoxy-2-methylbenzene (5c): A solution of 4c (190 mg, 0.67 mmol) in anhydrous THF (530 μL) was added dropwise to a solution of ⁿBuLi (2.5 M in hexanes, 410 μL, 1.00 mmol) in anhydrous THF (440 μL) at room temperature. After one hour, the solution was cooled to −78° C. and methyl iodide (170 μL, 2.67 mmol) was added. The resulting solution was warmed to room temperature over 12 hours, and the reaction was quenched by the addition of saturated aqueous NH₄Cl. Water (5 mL) was added and the solution was extracted with CH₂Cl₂ (3×10 mL). Combined organic fractions were dried (Na₂SO₄), filtered, and concentrated. The residue was purified via column chromatography (SiO₂, 6:1 Hexane:EtOAc) to afford 5c as a yellow oil (157 mg, 79%): ¹HNMR (CDCl₃, 500 MHz) δ 6.70 (d, J=9.0 Hz, 1H), 6.61 (d, J=9.0 Hz, 1H), 5.10 (s, 2H), 5.08 (s, 2H), 4.34 (quintet, J=6.1 Hz, 1H), 3.78 (q, J=7.1 Hz, 2H), 3.67 (q, J=7.1 Hz, 2H), 2.13 (s, 3H), 1.23 (d, J=6.1 Hz, 6H), 1.24-1.15 (m, 6H); ¹³C NMR (CDCl₃, 125 MHz) δ 150.3, 146.6, 145.3, 122.7, 113.7, 110.1, 97.3, 94.0, 71.5, 65.4, 64.2, 29.4, 22.2, 15.2, 15.2, 9.9; IR (film) νₘₐₓ 2924, 2853, 2359, 2339, 1591, 1483, 1113, 1057, 974 cm⁻¹; HRMS (ESI⁺) m/z: [M+H]⁺ calcd for C₁₆H₂₆O₅, 299.1858. Found, 299.1909.

4-Methoxy-2-methylbenzene-1,3-diol (6a): A solution of 5a (910 mg, 3.37 mmol) in MeOH (28.0 mL) at room temperature was treated dropwise with 3 M HCl (9.00 mL, 26.9 mmol), then heated to reflux for one hour. Water (30 mL) was added and the solution was extracted with EtOAc (3×30 mL). Combined organic fractions were washed with saturated aqueous NaCl, dried (Na₂SO₄), filtered, and concentrated. The residue was purified via column chromatography (SiO₂, 6:1 Hexane:EtOAc) to afford 6a as a red amorphous solid (509 mg, 98%): ¹HNMR (Acetone-d₆, 500 MHz) [symbol] 7.68 (s, 1H), 7.24 (s, 1H), 6.60 (d, J=11 Hz, 1H), 6.29 (d, J=11 Hz, 1H), 3.74 (s, 3H), 2.09 (s, 3H); ¹³C NMR (CDCl₃, 125 MHz) δ 144.7, 142.1, 139.7, 115.6, 110.2, 108.6, 55.6, 7.6; IR (film) νₘₐₓ 3583, 2920, 2359, 1616, 1259, 1090, 1020, 798 cm⁻¹.

2-Methyl-4-propoxybenzene-1,3-diol (6b): A solution of 5b (580 mg, 2.15 mmol) in MeOH (17.9 mL) was treated dropwise with 3 M HCl (630 μL, 17.2 mmol), then heated to reflux for one hour. Water (20 mL) was added and the solution was extracted with EtOAc (3×20 mL). Combined organic fractions were washed with saturated aqueous NaCl, dried (Na₂SO₄), and concentrated to afford 6b as a red amorphous solid (387 mg, 99%). ¹H NMR (CDCl₃, 500 MHz) [symbol] 6.51 (d, J=8.7 Hz, 1H), 6.21 (d, J=8.6 Hz, 1H), 5.74 (s, 1H), 4.36 (s, 1H), 3.87-3.85 (m, 2H), 2.09 (s, 3H), 1.75-1.71 (m, 2H), 0.96 (t, J=7.5 Hz, 3H); ¹³C NMR (CDCl₃, 125 MHz) δ 147.5, 143.8, 139.0, 109.8, 108.4, 103.8, 70.2, 21.6, 9.5, 7.3; IR (film) νₘₐₓ 3520, 3360, 2966, 2880, 2359, 2341, 1636, 1236, 1068, 785, 750 cm⁻¹.

4-Methoxybenzene-1,3-diol (6c): A solution of 5c (157 mg, 0.53 mmol) in MeOH (4.40 mL) at room temperature was treated dropwise with 3 M HCl (1.40 mL, 4.21 mmol), then heated to reflux for one hour. Water (5 mL) was added and the solution was extracted with EtOAc (3×10 mL). Combined organic fractions were washed with saturated aqueous NaCl, dried (Na₂SO₄), filtered, and concentrated to afford 6c as a red amorphous solid (95 mg, 99%): ¹H NMR (CDCl₃, 500 MHz) [symbol] 6.54 (d, J=8.7 Hz, 1H), 6.21 (d, J=8.7 Hz, 1H), 5.78 (s, 1H), 4.37-4.32 (m, 1H), 2.09 (s, 3H), 1.25 (d, J=6.1 Hz, 6H); ¹³C NMR (CDCl₃, 125 MHz) δ 147.7, 144.9, 137.5, 110.8, 109.8, 104.0, 71.7, 21.3 (2C); IR (film) νₘₐₓ 3526, 2974, 2924, 2853, 1717, 1607, 1475, 1238, 1113, 1067, 928, 887, 791 cm⁻¹.

To generate resorcinol precursors with substitutions at the 5-position, the phenols of 5-methoxy resorcinol 7 were once again protected as the corresponding alkoxy ethers, 8, as shown in Scheme 20 below. Ortho-lithiation of 8, followed by treatment with methyl iodide, led to installation of a methyl group at the 2-position of 9. See Carreno, M. C.; Garcia Ruano, J. L.; Toledo, M. A.; Urbano, A. Tetrahedron: Asymmetry 8 913-921 (1997). Acidic deprotection was employed to afford resorcinol 10. See Wang, Y.; Tan, W.; Li, W. Z.; Li, Y. J. Nat. Prod. 64 196-199 (2001).

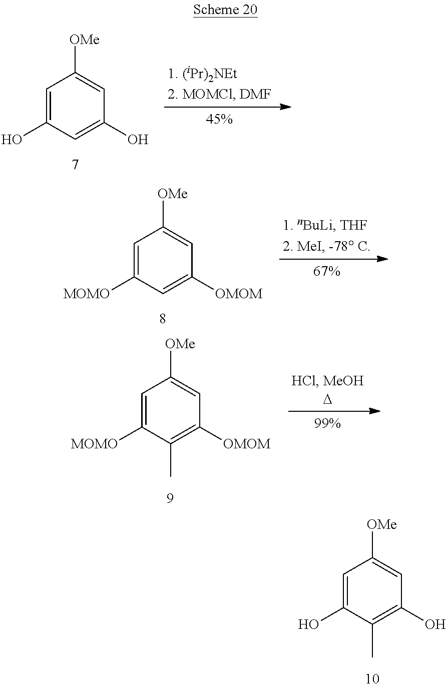

1-Methoxy-3,5-bis(methoxymethoxy)benzene (8): N,N-diisopropylethylamine (3.15 mL, 18.1 mmol) was added to 5-methoxybenzene-1,3-diol (634 mg, 4.52 mmol) in anhydrous N,N-dimethylformamide (12.6 mL) over five minutes at room temperature. After 30 minutes, the solution was cooled to 0° C., methoxy methylchloride (3.02 mL, 18.1 mmol) was added, and the solution was warmed to room temperature over 12 hours. The reaction was quenched by the addition of saturated aqueous NaHCO₃ at 0° C. and extracted with EtOAc (3×10 mL). Combined organic fractions were washed with saturated aqueous NaCl, dried (Na₂SO₄), filtered, and concentrated. The residue was purified via column chromatography (SiO₂, 6:1→4:1 Hexane:EtOAc) to afford 8 as a yellow amorphous solid (441 mg, 43%): ¹H NMR (CDCl₃, 500 MHz) [symbol] 6.29 (t, J=2.2 Hz, 1H), 6.21 (d, J=2.2 Hz, 2H), 5.07 (s, 4H), 3.69 (s, 3H), 3.40 (s, 6H); ¹³C NMR (CDCl₃, 125 MHz) [symbol] 161.394, 159.0 (2C), 97.2, 96.2 (2C), 94.5 (2C), 56.1, 55.4 (2C); IR (film) νₘₐₓ 2997, 2955, 2903, 2827, 1601, 1475, 1400, 1215, 1194, 1146, 1032, 991, 924, 829, 685 cm⁻¹; HRMS (ESI⁺) m/z: [M+Na]⁺ calcd for C₁₁H₁₆O₅, 251.0895. Found, 251.0910.

5-methoxy-1,3-bis(methoxymethoxy)-2-methylbenzene (9): A solution of 8 (441 mg, 1.93 mmol) in anhydrous THF (1.55 mL) was added dropwise to a solution of ⁿBuLi (2.5 M in hexanes, 1.16 mL, 2.90 mmol) in anhydrous THF (1.26 mL) at room temperature. After one hour, the solution was cooled to −78° C. and methyl iodide (480 μL, 7.73 mmol) was added. The resulting solution was warmed to room temperature over 12 hours, and the reaction was quenched by the addition of saturated aqueous NH₄Cl. Water (5 mL) was added and the solution was extracted with CH₂Cl₂ (3×10 mL). Combined organic fractions were dried (Na₂SO₄), filtered, and concentrated. The residue was purified via column chromatography (SiO₂, 6:1→4:1; Hexane:EtOAc) to afford 9 as a yellow oil (314 mg, 67%): ¹H NMR (CDCl₃, 500 MHz) [symbol] 6.38 (d, J=2.2 Hz, 1H), 6.24 (d, J=2.1 Hz, 1H), 5.08 (d, J=3.6 Hz, 2H), 5.06 (d, J=2.6 Hz, 2H), 3.72 (s, 3H), 3.40 (s, 6H), 1.97 (s, 3H); ¹³C NMR (CDCl₃, 125 MHz) [symbol] 160.3, 157.8, 155.4, 108.2, 93.9, 93.8, 93.7, 92.9, 55.0, 55.0, 54.6, 7.0; IR (film) νₘₐₓ 2953, 2934, 2905, 1597, 1497, 1396, 1215, 1144, 1126, 1074, 1059, 1028, 922, 822 cm⁻¹; HRMS (ESI⁺) m/z: [M+H]⁺ calcd for C₁₂H₁₈O₅, 243.1233. Found, 243.1223.

5-Methoxy-2-methylbenzene-1,3-diol (10): A solution of 9 (314 mg, 1.30 mmol) in MeOH (10.8 mL) at room temperature was treated dropwise with 3 M HCl (3.46 mL, 10.3 mmol), then heated to reflux for one hour. Water (11 mL) was added and the solution was extracted with EtOAc (3×15 mL). Combined organic fractions were washed with saturated aqueous NaCl, dried (Na₂SO₄), filtered, and concentrated to afford 10 as a red amorphous solid (177 mg, 99%): ¹H NMR (CDCl₃, 500 MHz) [symbol] 8.17 (s, 1H), 6.09 (d, J=1.6 Hz, 1H), 6.04 (s, 1H), 3.67 (d, J=9.9 Hz, 3H), 2.08 (d, J=4.1 Hz, 3H); ¹³C NMR (CDCl₃, 125 MHz) [symbol] 160.3, 157.8, 155.4, 108.4, 93.9, 93.8, 55.0, 7.0; IR (film) νₘₐₓ 3445, 2924, 2853, 2359, 2332, 1653, 1636, 1456, 1080, 1022, 798, 669 cm⁻¹; HRMS (ESI⁺) m/z: [2M+H]⁺ calcd for C₈H₁₀O₃, 309.1338. Found, 309.1332.

To generate the resorcinol precursors with aryl substituents at the 2-position, the phenols of resorcinol 11 were protected as the corresponding alkoxy ethers, 12, as shown in the scheme below. Subsequent ortho-lithiation of 12, followed by the addition of benzyl bromide provided the benzyl derivative, 13. See Carreno, M. C.; Garcia Ruano, J. L.; Toledo, M. A.; Urbano, A. Tetrahedron: Asymmetry 8 913-921 (1997). Removal of the ether protecting groups gave diphenol 14. See Wang, Y.; Tan, W.; Li, W. Z.; Li, Y. J. Nat. Prod. 64 196-199 (2001). The anion of resorcinol 12 was also employed to construct the corresponding 2-iodide via reaction with iodine to yield 15. See Ruenitz, P. C.; Bagley, J. R.; Nanavati, N. T. J. Med. Chem. 31 1471-1475 (1988). A Suzuki coupling in the presence of biaryl ligand S-Phos, was used to generate biaryl 16, which underwent deprotection⁴⁶ to provide 17. See Milne, J. E.; Buchwald, S. L. J. Am. Chem. Soc. 126 13028-13032 (2004).

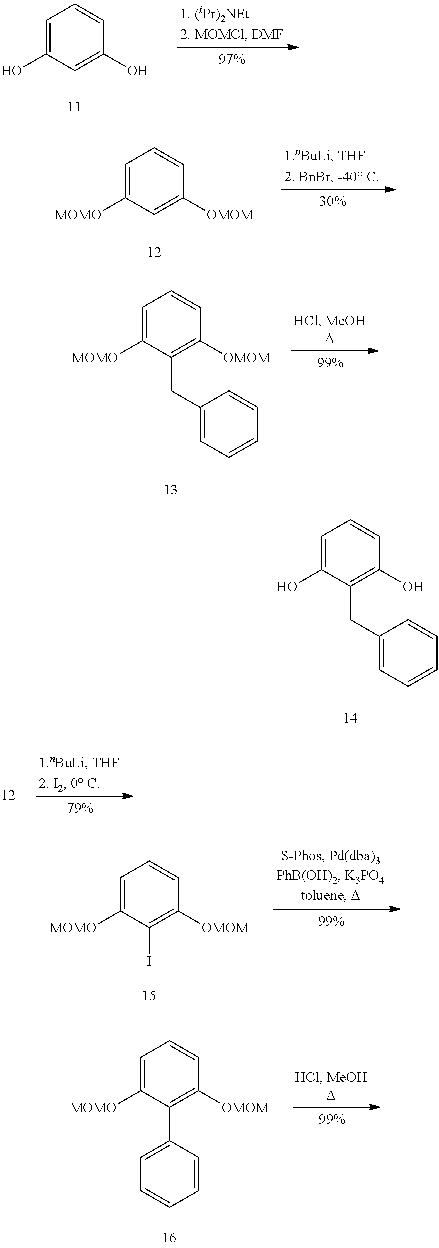

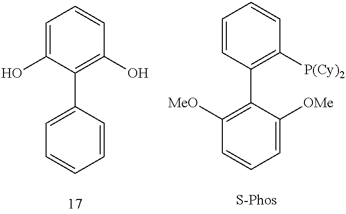

1,3-Bis(methoxymethoxy)benzene (12): Sodium hydride (872 mg, 36.3 mmol) was added to resorcinol (1.00 g, 9.08 mmol) in anhydrous N,N-dimethylformamide (25.4 mL) at 0° C. After 30 minutes, methoxy methylchloride (2.76 mL, 36.3 mmol) was added and the resulting solution was warmed to room temperature over 12 hours. The reaction was cooled to 0° C., quenched by the addition of saturated aqueous NaHCO₃, and extracted with EtOAc (3×30 mL). Combined organic fractions were washed with saturated aqueous NaCl, dried (Na₂SO₄), filtered, and concentrated. The residue was purified via column chromatography (SiO₂, 4:1 Hexane:EtOAc) to afford 12 as a yellow oil (1.75 g, 97%): NMR (CDCl₃, 400 MHz) [symbol] 7.25-7.20 (m, 1H), 6.80 (d, J=2.3 Hz, 1H), 6.75 (dd, J=8.2, 2.4 Hz, 2H), 5.20 (s, 4H), 3.51 (s, 6H).

2-Benzyl-1,3-bis(methoxymethoxy)benzene (13): A solution of 12 (500 mg, 2.52 mmol) in anhydrous THF (2.02 mL) was added dropwise to a solution of ⁿBuLi (2.5 M in hexanes, 1.51 mL, 3.78 mmol) in anhydrous THF (1.65 mL) at room temperature. After one hour, the solution was cooled to −40° C. and benzyl bromide (1.22 mL, 10.10 mmol) was added. The resulting solution was warmed to room temperature over 12 hours, and the reaction was quenched by the addition of saturated aqueous NH₄Cl. Water (5 mL) was added and the solution was extracted with CH₂Cl₂ (3×10 mL). Combined organic fractions were dried (Na₂SO₄), filtered, and concentrated. The residue was purified via column chromatography (SiO₂, 4:1; Hexane:EtOAc) to afford 13 as a yellow oil (214 mg, 30%): ¹H NMR (CDCl₃, 500 MHz) [symbol] 7.17 (d, J=7.9 Hz, 2H), 7.17-7.12 (m, 2H), 7.06-7.02 (m, 2H), 6.71 (d, J=8.3 Hz, 2H), 5.09 (s, 4H), 4.00 (s, 2H), 3.29 (s, 6H); ¹³C NMR (CDCl₃, 125 MHz) [symbol] 155.9 (2C), 141.6, 128.5 (2C), 128.0 (2C), 127.5, 125.4, 119.4, 197.7 (2C), 94.3 (2C), 56.0 (2C), 29.1; IR (film) νₘₐₓ 2953, 2930, 1595, 1470, 1452, 1254, 1153, 1097, 1043, 941, 922, 727, 698 cm⁻¹; HRMS (ESI⁺) m/z: [M+Na]⁺ calcd for C₁₇H₂₀O₄, 311.1259. Found, 311.1201.

2-Benzylbenzene-1,3-diol (14): A solution of 13 (214 mg, 0.74 mmol) in MeOH (6.20 mL) was treated dropwise with 3 M HCl (0.22 mL, 5.92 mmol), then heated to reflux for one hour. Water (10 mL) was added and the solution was extracted with EtOAc (3×15 mL). Combined organic fractions were washed with saturated aqueous NaCl, dried (Na₂SO₄), and concentrated to afford 14 as a red amorphous solid (149 mg, 99%). ¹H NMR (CDCl₃, 400 MHz) [symbol] 7.31 (d, J=6.6 Hz, 4H), 7.25-7.19 (m, 1H), 7.01 (t, J=8.1 Hz, 1H), 6.44 (d, J=8.1 Hz, 2H), 4.82 (s, 2H), 4.09 (s, 2H).

2-Iodo-1,3-bis(methoxymethoxy)benzene (15): n-Butyllithium (2.5 M in hexanes, 0.22 mL, 0.56 mmol) was added to a solution of 12 (100 mg, 0.50 mmol) in anhydrous THF (790 μL) at 0° C. After 5 minutes, iodine (141 mg, 0.56 mmol) in anhydrous THF (320 μL) was added. After two hours at room temperature, the reaction was quenched via dropwise addition of MeOH and the solvent was concentrated. Water (5 mL) was added and the solution was extracted with EtOAc (3×10 mL). Combined organics were washed with saturated aqueous Na₂S₂O₃, saturated aqueous NaCl, dried (Na₂SO₄), filtered, and concentrated to afford 15 as a brown oil (129 mg, 79%): NMR (CDCl₃, 100 MHz) [symbol] 7.25-7.18 (m, 1H), 6.79-6.71 (m, 2H), 5.27 (s, 2H), 5.18 (s, 2H), 3.54 (s, 3H), 3.50 (s, 3H); IR (film) νₘₐₓ 2953, 2924, 2853, 1458, 1377 cm⁻¹.

2,6-Bis(methoxymethoxy)biphenyl (16): Anhydrous toluene (2.0 mL) was added to a flask charged with Pd₂(dba)₃ (56.3 mg, 0.062 mmol), dicyclohexyl(2′,6′-dimethoxybiphenyl-2-yl)phosphine (50.5 mg, 0.12 mmol), phenylboronic acid (281 mg, 2.31 mmol), and potassium phosphate (979 mg, 4.61 mmol) at room temperature. After 15 minutes, a solution of 15 (500 mg, 1.54 mmol) in anhydrous toluene (1.0 mL) was added and the resulting solution was heated to reflux for 12 hours. Upon cooling to room temperature, ether was added, the solution was filtered through SiO₂ and concentrated to give 16 as a colorless amorphous solid (418 mg, 99%): ¹HNMR (CDCl₃, 500 MHz) [symbol] 7.35-7.28 (m, 2H), 7.28-7.25 (m, 2H), 7.18-7.15 (m, 2H), 6.83 (d, J=8.3 Hz, 2H), 4.96 (s, 4H), 3.24 (s, 6H); ¹³C NMR (CDCl₃, 125 MHz) [symbol] 155.3, 155.0, 134.3, 130.8, 129.5, 128.7, 128.0, 127.6, 126.8, 122.6, 109.4 (2C), 94.9 (2C), 56.0 (2C); IR (film) νₘₐₓ 2955, 2928, 2901, 2359, 2341, 1587, 1466, 1439, 1400, 1244, 1153, 1099, 1080, 1041, 922, 764, 733, 700 cm⁻¹; HRMS (ESI⁺) m/z: [M+Na]⁺ calcd for C₁₆H₁₈O₄, 297.1103. Found, 297.1052.

Biphenyl-2,6-diol (17): A solution of 16 (400 mg, 1.46 mmol) in MeOH (12.0 mL) at room temperature was treated dropwise with 3 M HCl (430 μL, 11.7 mmol), then heated to reflux for one hour. Water (15 mL) was added and the solution was extracted with EtOAc (3×20 mL). Combined organic fractions were washed with saturated aqueous NaCl, dried (Na₂SO₄), filtered, and concentrated to afford 17 as an orange amorphous solid (269 mg, 99%): ¹H NMR (CDCl₃, 400 MHz) [symbol] 7.60 (d, J=7.6 Hz, 2H), 7.53-7.49 (m, 1H), 7.46-7.44 (m, 2H), 7.18 (t, J=8.2 Hz, 1H), 6.62 (d, J=8.2 Hz, 2H), 4.84 (s, 1H), 4.83 (s, 1H).

To generate resorcinol precursors with alkyl substitutions at the 2-position, pyragallol (18) was O-alkylated with methyl iodide to generate 2-methoxy resorcinol amongst an inseparable mixture of regioisomers as shown in the scheme below. The mixture was subsequently subjected to coumarin formation and the corresponding products isolated. Preparation of 2-ethyl resorcinol (21) from 2,6-dihydroxyacetophenone (20) was accomplished according to published procedures. See Elliger, C. A. Synth. Commun. 15 1315-1324 (1985). The following scheme shows the synthesis of 2-methoxy resorcinol and 2-ethyl resorcinol.

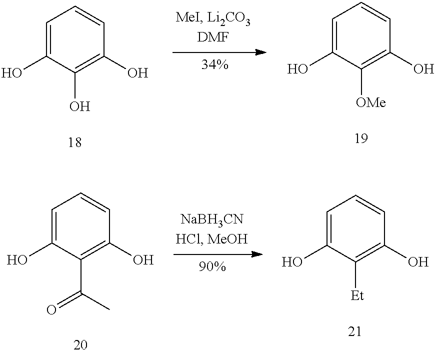

2-Methoxybenzene-1,3-diol (19): Lithium carbonate (281 mg, 1.98 mmol) was added to pyrogallol (100 mg, 0.79 mmol) in N,N-dimethylformamide (3.0 mL) at room temperature. After five minutes, methyl iodide (130 μL, 1.98 mmol) was added and the resulting solution was heated to 50° C. for 48 hours. Upon cooling to room temperature, water (20 mL) was added and the solution was extracted with EtOAc (3×20 mL). Combined organic fractions were dried (Na₂SO₄), filtered, and concentrated. The residue was purified via column chromatography (SiO₂, 5:1→1:1 Hexane:EtOAc) to afford 19 as a colorless amorphous solid (44.2 mg, 34%): ¹H NMR (CDCl₃, 400 MHz) [symbol] 6.89 (td, J=8.0, 0.9 Hz, ′H), 6.53 (dd, J=8.2, 0.8 Hz, 2H), 5.83 (bs, 2H), 3.90 (s, 3H).

### Example 13

### Preparation of 5-, 6-, and 8-Modified Novobiocin Analogues

In this example, 5-, 6-, and 8-modified novobiocin analogues were prepared. More specifically, once resorcinols 6a-c, 10, 14, 17, 19, and 21 were obtained from the Example above, the corresponding coumarins 23a-h were synthesized through a modified Pechmann condensation with eneamine 22 as previously described. See Robinson, A. J.; Lim, C. Y.; He, L.; Ma, P.; Li, H.-Y. J. Org. Chem. 66 4141-4147 (2001); Toplak, R.; Svete, J.; Stanovnik, B.; Grdadolnik, S. G. J. Hetero. Chem. 36 225-235 (1999). The resulting coumarin phenols were noviosylated with the trichloroacetimidate of noviose cyclic carbonate (24) in the presence of catalytic boron trifluoride etherate to generate scaffolds 25a-h in good yield. See Shen, G.; Yu, X. M.; Blagg, B. S. J. Bioorg. Med. Chem. Lett. 14 5903-5906 (2004). The benzyl carbonate was removed via hydrogenolysis to produce the aminocoumarin, which was readily coupled with preselected benzoic acids in the presence of N-(3-dimethylamino-propyl)-N′-ethylcarbodiimide hydrochloride (EDCI) and pyridine. Benzoic acids were chosen based on previously determined SAR trends reported by Burlison and co-workers. See Burlison, J. A.; Avila, C.; Vielhauer, G.; Lubbers, D. J.; Holzbeierlein, J.; Blagg, B. S. J. J. Org. Chem. 73 2130-2137 (2008). The cyclic carbonates were treated with triethylamine in methanol to give the solvolyzed products, 26a-p in moderate to good yield over three steps as shown in Scheme 21.

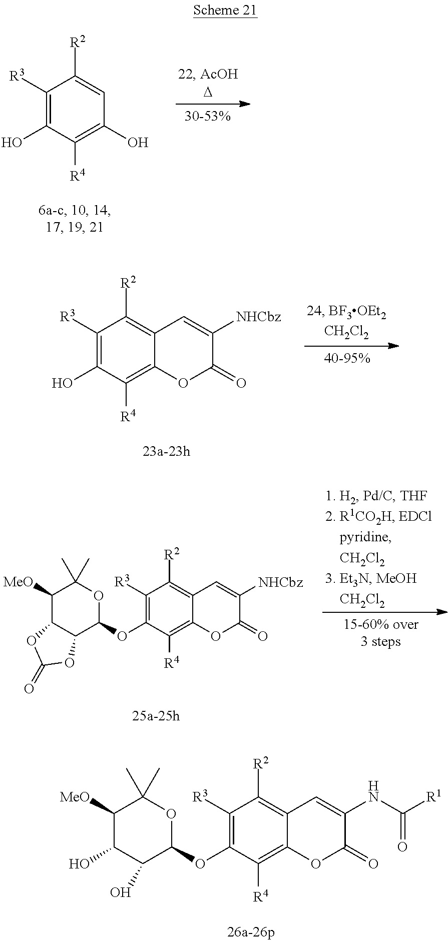

Table of R Subsitutents

| Compound | R¹ | R² | R³ | R⁴ |
|---|---|---|---|---|
| 23a | n/a | H | OMe | Me |
| 23b | n/a | H | OPr | Me |
| 23c | n/a | H | OⁱPr | Me |
| 23d | n/a | OMe | H | Me |
| 23e | n/a | H | H | Bn |
| 23f | n/a | H | H | Ph |
| 23g | n/a | H | H | OMe |
| 23h | n/a | H | H | Et |
| 25a | n/a | H | OMe | Me |
| 25b | n/a | H | OPr | Me |
| 25c | n/a | H | OⁱPr | Me |
| 25d | n/a | OMe | H | Me |
| 25e | n/a | H | H | Bn |
| 25f | n/a | H | H | Ph |
| 25g | n/a | H | H | OMe |
| 25h | n/a | H | H | Et |
| 26a | biaryl | H | OMe | Me |
| 26b | biaryl | H | OPr | Me |
| 26c | biaryl | H | OⁱPr | Me |
| 26d | biaryl | OMe | H | Me |
| 26e | biaryl | H | H | Bn |
| 26f | biaryl | H | H | Ph |
| 26g | biaryl | H | H | OMe |
| 26h | biaryl | H | H | Et |
| 26i | 2-indole | H | OMe | Me |
| 26j | 2-indole | H | OPr | Me |
| 26k | 2-indole | H | OⁱPr | Me |
| 26l | 2-indole | OMe | H | Me |
| 26m | 2-indole | H | H | Bn |
| 26n | 2-indole | H | H | Ph |
| 26o | 2-indole | H | H | OMe |
| 26p | 2-indole | H | H | Et |

wherein the 22, 24, biaryl, and 2-indole are:

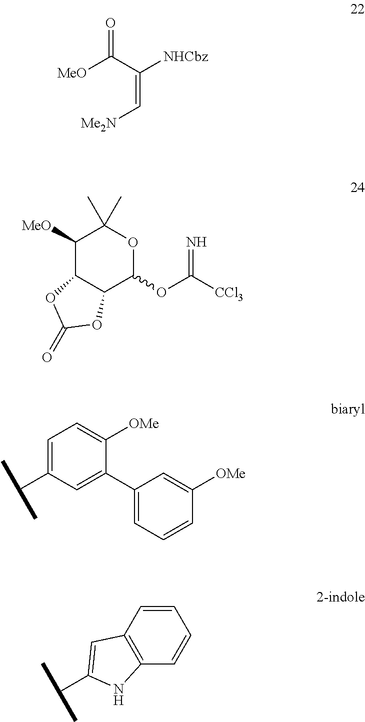

Benzyl 7-hydroxy-6-methoxy-8-methyl-2-oxo-2H-chromen-3-ylcarbamate (23a): A solution of 6a (183 mg, 1.19 mmol) and enamine 22 (331 mg, 1.19 mmol) in glacial acetic acid (7.40 mL) was heated to reflux for 40 hours. Upon cooling to room temperature, the solution was extracted with EtOAc (3×20 mL); combined organic fractions were dried (Na₂SO₄), filtered, and concentrated. The residue was purified via column chromatography (SiO₂, 100:1 CH₂Cl₂:Acetone) to afford 23a as a yellow amorphous solid (195 mg, 46%): ¹H NMR (CDCl₃, 400 MHz) [symbol] 8.27 (s, 1H), 7.54 (s, 1H), 7.43-7.37 (m, 4H), 6.77 (s, 1H), 6.07 (s, 1H), 5.25 (s, 2H), 3.96 (s, 3H), 2.37 (s, 3H); ¹³C NMR (CDCl₃, 125 MHz) δ 159.0, 153.3 (2C), 145.7, 144.1, 144.0, 135.7, 128.7, 128.5, 128.2 (2C), 122.5, 121.6, 112.1, 111.6, 104.5, 67.4, 56.3, 8.2; IR (film) νₘₐₓ 2910, 2359, 2339, 1693, 1537, 1354, 1209, 1078, 1024 cm⁻¹; HRMS (ESI⁺) m/z: [M+Na]⁺ calcd for C₁₉H₁₇NO₆, 378.0954. Found, 378.0936.

Benzyl 7-hydroxy-8-methyl-2-oxo-6-propoxy-2H-chromen-3-ylcarbamate (23b): A solution of 6b (390 mg, 2.14 mmol) and enamine 22 (596 mg, 2.14 mmol) in glacial acetic acid (13.4 mL) was heated to reflux for 36 hours. Upon cooling to room temperature, the precipitated yellow solid was collected by filtration, washed with water, recrystallized from MeOH/water, and extracted with EtOAc (3×20 mL). Combined organic fractions were washed with saturated aqueous NaCl, dried (Na₂SO₄), filtered, and concentrated. The residue was purified via column chromatography (SiO₂, 100:1 CH₂Cl₂:Acetone) and recrystallized from MeOH/water to afford 23b as a yellow amorphous solid (278 mg, 34%): ¹H NMR (CD₂Cl₂, 400 MHz) [symbol] 8.26 (s, 1H), 7.56 (s, 1H), 7.47-7.38 (m, 5H), 6.84 (s, 1H), 6.28 (s, 1H), 5.25 (s, 2H), 4.09 (t, J=6.6 Hz, 2H), 2.36 (s, 3H), 1.93-1.88 (m, 2H), 1.10 (t, J=7.4 Hz, 3H); ¹³C NMR (CDCl₃, 125 MHz) δ 158.0, 152.2, 144.9, 142.9, 142.3, 134.6, 129.0, 127.6, 127.5 (2C), 127.2 (2C), 121.5, 120.5, 110.9, 110.5, 104.4, 69.8, 66.3, 21.4, 9.4, 7.1; IR (film) νₘₐₓ 2957, 2920, 2851, 2359, 2341, 1693, 1537, 1358, 1277, 1080, 1024, 910 cm⁻¹; HRMS (ESI⁺) m/z: [M+H]⁺ calcd for C₂₁H₂₁NO₆, 384.1447. Found, 384.1447.

Benzyl 7-hydroxy-6-isopropoxy-8-methyl-2-oxo-2H-chromen-3-ylcarbamate (23c): A solution of 6c (142 mg, 0.78 mmol) and enamine 22 (217 mg, 0.78 mmol) in glacial acetic acid (4.90 mL) was heated to reflux for 40 hours. Upon cooling to room temperature, the solution was extracted with EtOAc (3×10 mL); combined organic fractions were dried (Na₂SO₄), filtered, and concentrated. The residue was purified via column chromatography (SiO₂, 40:1 CH₂Cl₂:Acetone) to afford 23c as a yellow amorphous solid (159 mg, 53%): ¹H NMR (CD₂Cl₂, 400 MHz) [symbol] 8.26 (s, 1H), 7.56 (s, 1H), 7.44-7.38 (m, 5H), 6.85 (s, 1H), 6.31 (s, 1H), 5.25 (s, 2H), 4.66 (quintet, J=6.1 Hz, 1H), 2.35 (s, 3H), 1.42 (d, J=6.0 Hz, 6H); ¹³C NMR (CDCl₃, 125 MHz) δ 159.1, 154.9, 146.7, 143.9 (2C), 142.0, 135.7, 128.7 (2C), 128.5 128.2 (2C), 122.6 (2C), 111.6, 107.0, 72.3, 67.4, 22.1 (2C), 8.2; IR (film) νₘₐₓ 3400, 2924, 2853, 2359, 1817, 1699, 1524, 1412, 1354, 1300, 1221, 1204, 1113, 1076, 1022, 824 cm⁻¹; HRMS (ESI⁺) m/z: [M+H]⁺ calcd for C₂₁H₂₁NO₆, 384.1447. Found, 384.1452.

Benzyl 7-hydroxy-5-methoxy-8-methyl-2-oxo-2H-chromen-3-ylcarbamate (23d): A solution of 10 (251 mg, 1.63 mmol) and enamine 22 (680 mg, 2.44 mmol) in glacial acetic acid (10.2 mL) was heated to reflux for 40 hours. Upon cooling to room temperature, the solution was extracted with EtOAc (3×15 mL); combined organic fractions were dried (Na₂SO₄), filtered, and concentrated. The residue was purified via column chromatography (SiO₂, 40:1→20:1; CH₂Cl₂:Acetone) to afford 23d as a yellow amorphous solid (204 mg, 35%): ¹H NMR (CD₂Cl₂, 400 MHz) [symbol] 8.48 (s, 1H), 7.46-7.38 (m, 6H), 6.38 (s, 1H), 5.25 (s, 2H), 5.15 (s, 1H), 3.87 (s, 3H), 2.23 (s, 3H); ¹³C NMR (CDCl₃, 125 MHz) [symbol] 159.1, 155.8, 154.3, 153.2, 149.8, 137.0, 128.7, 128.6, 128.6, 128.2, 128.2, 109.3, 109.0, 108.5, 105.6, 96.9, 70.8, 60.2, 7.3; IR (film) νₘₐₓ 3406, 2935, 2837, 1713, 1670, 1607, 1529, 1501, 1364, 1242, 1101, 1051, 991, 966, 735 cm⁻¹; HRMS (ESI⁺) m/z: [M+Na]⁺ calcd for C₁₉H₁₇NO₆, 378.0954. Found, 378.0974.

Benzyl 8-benzyl-7-hydroxy-2-oxo-2H-chromen-3-ylcarbamate (23e): A solution of 14 (115 mg, 0.57 mmol) and enamine 22 (160 mg, 0.57 mmol) in glacial acetic acid (4.00 mL) was heated to reflux for 40 hours. Upon cooling to room temperature, the solution was extracted with EtOAc (3×10 mL); combined organic fractions were dried (Na₂SO₄), filtered, and concentrated. The residue was purified via column chromatography (SiO₂, 100:1; CH₂Cl₂:Acetone), followed by recrystallization from MeOH to afford 23e as an orange amorphous solid (296 mg, 48%): ¹H NMR (CD₂Cl₂, 400 MHz) [symbol] 8.29 (s, 1H), 7.53 (s, 1H), 7.46-7.38 (m, 4H), 7.37-7.27 (m, 4H), 7.23-7.19 (m, 2H), 7.01 (t, J=8.1 Hz, 1H), 6.86 (d, J=8.4 Hz, 1H), 6.46 (d, J=8.1 Hz, 1H), 5.25 (s, 2H), 4.25 (s, 2H), 4.06 (s, 1H); ¹³C NMR (CDCl₃, 125 MHz) [symbol] 157.7, 154.3, 153.9, 152.2, 148.0, 137.9, 134.5, 127.6, 127.6, 127.6, 127.6, 127.5, 127.3, 127.2, 127.4, 126.6, 125.4, 125.3, 121.4, 120.4, 114.0, 112.6, 66.5, 27.5; IR (film) νₘₐₓ 3381, 2957, 2928, 2359, 2341, 1693, 1607, 1526, 1466, 1454, 1383, 1366, 1219, 1204, 1076, 1045, 764, 737, 700 cm⁻¹; HRMS (ESI⁺) m/z: [M+H]⁺ calcd for C₂₄H₁₉NO₅, 402.1341. Found, 402.1341.

Benzyl 7-hydroxy-2-oxo-8-phenyl-2H-chromen-3-ylcarbamate (23f): A solution of 17 (400 mg, 2.15 mmol) and enamine 22 (598 mg, 2.15 mmol) in glacial acetic acid (14.3 mL) was heated to reflux for 40 hours. Upon cooling to room temperature, the solution was extracted with EtOAc (3×30 mL); combined organic fractions were dried (Na₂SO₄), filtered, and concentrated. The residue was purified via column chromatography (SiO₂, 100:1; CH₂Cl₂:Acetone), then recrystallized from MeOH to afford 23f as an orange amorphous solid (264 mg, 27%): ¹H NMR (CDCl₃, 500 MHz) [symbol] 8.25 (s, 1H), 7.51-7.48 (m, 2H), 7.43-7.40 (m, 2H), 7.35-7.29 (m, 8H), 6.94 (d, J=8.6 Hz, 1H), 5.16 (s, 2H); ¹³C NMR (CDCl₃,125 MHz) [symbol] 158.5 (2C), 154.3, 153.2, 147.7, 135.6, 130.9, 130.6, 130.5, 129.8, 129.4, 129.2 (2C), 128.7 (2C), 128.6, 128.3, 127.8, 122.2, 121.6, 113.3, 113.5, 67.5; IR (film) νₘₐₓ 3398, 2957, 2926, 2854, 1815, 1699, 1601, 1524, 1383, 1366, 1308, 1215, 1045, 1009, 764, 750, 698 cm⁻¹; HRMS (ESI⁺) m/z: [M+H]⁺ calcd for C₂₃H₁₇NO₅, 388.1185. Found, 388.1214.

Benzyl 7-hydroxy-8-methoxy-2-oxo-2H-chromen-3-ylcarbamate (23g): A solution of 19 (1.10 g, 7.86 mmol) and enamine 22 (2.18 g, 7.86 mmol) in glacial acetic acid (60.0 mL) was heated to reflux for 90 hours. Upon cooling to room temperature, the solution was extracted with EtOAc (3×50 mL); combined organic fractions were washed with saturated aqueous NaCl, dried (Na₂SO₄), filtered, and concentrated. The residue was purified via column chromatography (SiO₂, 11:1; Hexane:EtOAc→EtOAc) then recrystallized from MeOH/water to afford 23g as a colorless amorphous solid (207 mg, 7.7%): ¹H NMR (CDCl₃, 400 MHz) [symbol] 8.30 (s, 1H), 7.50 (s, 1H), 7.43-7.36 (m, 5H), 7.13 (d, J=8.6 Hz, 1H), 6.97 (d, J=7.9 Hz, 1H), 6.04 (s, 1H), 5.21 (s, 2H), 4.13 (s, 3H); ¹³C NMR (Acetone-d₆, 100 MHz) [symbol] 157.3, 153.3, 151.5 (2C), 144.1, 136.5, 134.4, 128.4 (2C), 128.1, 128.0 (2C), 122.7, 121.6, 113.6, 113.2, 66.7, 60.7; IR (film) νₘₐₓ 2920, 2851, 2405, 2357, 1707, 1605, 1522, 1458, 1385, 1364, 1275, 1259, 1213, 1088, 1047, 750 cm⁻¹; HRMS (ESI⁺) m/z: [M+Na]⁺ calcd for C₁₈H₁₅NO₆, 364.0797. Found, 364.0776.

Benzyl 8-ethyl-7-hydroxy-2-oxo-2H-chromen-3-ylcarbamate (23h): A solution of 21 (1.40 g, 10.1 mmol) and enamine 22 (2.80 g, 10.1 mmol) in glacial acetic acid (50.0 mL) was heated to reflux for 12 hours. Upon cooling to room temperature, the solvent was concentrated. The residue was purified via column chromatography (SiO₂, 4:1→2:1; Hexane:EtOAc), then recrystallized from acetone/hexanes to afford 23h as a colorless amorphous solid (600 mg, 17%). ¹H NMR (DMSO-d₆, 400 MHz) [symbol] 9.07 (s, 1H), 8.12 (s, 1H), 7.46-7.32 (m, 6H), 6.86 (d, J=8.4 Hz, 1H), 5.18 (s, 2H), 2.72 (q, J=7.6 Hz, 2H), 1.11 (t, J=7.6 Hz, 3H); ¹³C NMR (DMSO-d₆, 125 MsHz) [symbol] 158.0, 157.1, 153.8 (2C), 149.5, 136.4, 128.4 (2C), 127.9, 127.8, 127.2, 125.8, 120.4, 116.6, 112.8, 111.4, 66.1, 15.7, 13.5; IR (film) νₘₐₓ 3391, 3339, 2964, 2870, 2357, 1732, 1682, 1620, 1524, 1506, 1454, 1364, 1277, 1188, 1097, 1024, 752, 698 cm⁻¹; HRMS (ESI⁺) m/z: [M+H]⁺ calcd for C₁₉H₁₇NO₅, 340.1185. Found, 340.1181.

Benzyl 6-methoxy-7-((3aR,4S,7R,7aR)-7-methoxy-6,6-dimethyl-2-oxotetrahydro-3aH-[1,3]dioxolo[4,5-c]pyran-4-yloxy)-8-methyl-2-oxo-2H-chromen-3-ylcarbamate (25a): Boron trifluoride etherate (5.30 μL, 0.042 mmol) was added to 23a (50.0 mg, 0.14 mmol) and (3aR,4S,7R,7aR)-7-methoxy-6,6-dimethyl-2-oxo-tetrahydro-3aH-[1.3]dioxolo[4,5-c]pyran-4-yl 2,2,2-trichloroacetimidate (171 mg, 0.47 mmol) in anhydrous CH₂Cl₂ (3.00 mL). After stirring at room temperature for 14 hours, triethylamine (150 μL) was added and the solvent was concentrated. The residue was purified via column chromatography (SiO₂, 40:1 CH₂Cl₂:Acetone) to give 25a as a colorless foam (74.0 mg, 95%): ¹H NMR (CD₂Cl₂, 400 MHz) [symbol] 8.29 (s, 1H), 7.64 (s, 1H), 7.47-7.39 (m, 5H), 6.91 (s, 1H), 5.52 (d, J=3.4 Hz, 1H), 5.26 (s, 2H), 5.23 (dd, J=8.4, 3.5 Hz, 1H), 4.95 (t, J=8.2 Hz, 1H), 3.92 (s, 3H), 3.60 (s, 3H), 3.33 (d, J=8.0 Hz, 1H), 2.42 (s, 3H), 1.38 (s, 3H), 1.33 (s, 3H); ¹³C NMR (CDCl₃, 125 MHz) δ 157.6, 152.7, 152.1, 148.1, 144.8, 141.8, 134.5, 127.8, 127.7, 127.5, 127.3, 122.3, 120.2, 119.8, 115.1. 109.6, 105.2, 98.3, 82.0, 77.1, 66.5, 65.5, 59.4, 57.4, 55.1, 26.0, 20.9, 8.9; IR (film) νₘₐₓ 2957, 2928, 2854, 2359, 2341, 1817, 1709, 1522, 1464, 1389, 1371, 1205, 1174, 1111, 1072, 1034, 957, 800 cm⁻¹; HRMS (ESI⁺) m/z: [M+H]⁺ calcd for C₂₈H₂₉NO₁₁, 556.1819. Found, 556.1822.

Benzyl 7-((3aR,4S,7R,7aR)-7-methoxy-6,6-dimethyl-2-oxotetrahydro-3aH-[1,3]dioxolo[4,5-c]pyran-4-yloxy)-8-methyl-2-oxo-6-propoxy-2H-chromen-3-ylcarbamate (25b): Boron trifluoride etherate (16.7 μL, 0.13 mmol) was added to 23b (170 mg, 0.44 mmol) and (3aR,4S,7R,7aR)-7-methoxy-6,6-dimethyl-2-oxo-tetrahydro-3aH-[1.3]dioxolo[4,5-c]pyran-4-yl 2,2,2-trichloroacetimidate (643 mg, 1.77 mmol) in anhydrous CH₂Cl₂ (11.1 mL). After stirring at room temperature for 48 hours, triethylamine (150 μL) was added and the solvent was concentrated. The residue was purified via column chromatography (SiO₂, 100:1→40:1 CH₂Cl₂:Acetone) to give 25b as a colorless foam (246 mg, 95%): NMR (CDCl₃, 500 MHz) [symbol] 8.17 (s, 1H), 7.35-7.27 (m, 5H), 6.84 (s, 1H), 5.96 (s, 1H), 5.15 (s, 2H), 4.99 (d, J=7.5 Hz, 1H), 4.59 (d, J=9.7 Hz, 1H), 4.23 (d, J=9.6 Hz, 1H), 3.97 (t, J=6.6 Hz, 1H), 3.82-3.75 (m, 2H), 3.37 (s, 3H), 1.84-1.79 (m, 2H), 1.51 (s, 3H), 1.41 (s, 3H), 1.18 (s, 3H), 1.00 (t, J=7.5 Hz, 3H); ¹³C NMR (CDCl₃, 125 MHz) δ 157.8, 154.3, 152.2, 151.8, 146.7, 144.2, 142.9, 134.6, 127.8, 127.6, 127.5, 127.5, 127.2, 121.0, 120.9, 111.1, 105.3, 101.7, 91.6, 85.7, 82.8, 80.0, 69.8, 58.1, 54.8, 28.3, 28.2, 22.4, 21.3, 9.4; IR (film) νₘₐₓ 2961, 2939, 2906, 2359, 2341, 1811, 1757, 1726, 1522, 1445, 1371, 1267, 1175, 1113, 1086, 825, 768 cm⁻¹; HRMS (ESI⁺) m/z: [M+Na]⁺ calcd for C₃₀H₃₃NO₁₁, 606.1952. Found, 606.1950.

Benzyl 6-isopropoxy-7-((3aR,4S,7R,7aR)-7-methoxy-6,6-dimethyl-2-oxotetrahydro-3aH-[1,3]dioxolo[4,5-c]pyran-4-yloxy)-8-methyl-2-oxo-2H-chromen-3-ylcarbamate (25c): Boron trifluoride etherate (1.30 μL, 0.010 mmol) was added to 23c (13.0 mg, 0.034 mmol) and (3aR,4S,7R,7aR)-7-methoxy-6,6-dimethyl-2-oxo-tetrahydro-3aH-[1.3]dioxolo[4,5-c]pyran-4-yl-2,2,2-trichloroacetimidate (83.0 mg, 0.23 mmol) in anhydrous CH₂Cl₂ (1.30 mL). After stirring at room temperature for 1.5 hours, triethylamine (150 μL) was added and the solvent was concentrated. The residue was purified via column chromatography (SiO₂, 40:1 CH₂Cl₂:Acetone) to give 25c as a colorless foam (19.0 mg, 95%): ¹H NMR (CDCl₃, 500 MHz) [symbol] 8.17 (s, 1H), 7.51 (s, 1H), 7.35 (s, 1H), 7.34-7.33 (m, 4H), 6.74 (s, 1H), 5.54 (dd, J=9.2, 1.2 Hz, 1H), 5.16 (s, 2H), 4.87-4.84 (m, 1H), 4.73 (dd, J=7.9, 1.9 Hz, 1H), 4.51 (quintet, J=6.0 Hz, 1H), 3.52 (s, 3H), 3.28 (d, J=4.8 Hz, 1H), 2.33 (s, 3H), 1.80-1.77 (m, 6H), 1.30 (s, 3H), 1.27 (s, 3H); ¹³C NMR (CDCl₃, 125 MHz) δ 161.6, 158.6, 153.2 153.1, 147.1, 146.8, 142.5, 135.5, 128.7, 128.6, 128.3, 123.3, 121.4, 121.1, 116.2, 108.6, 99.4, 83.1, 79.9, 76.1, 74.7, 72.2, 68.0, 60.5, 27.1, 25.6, 21.9, 21.6, 21.0, 10.1; IR (film) νₘₐₓ 2955, 2922, 2853, 2359, 2339, 1819, 1711, 1520, 1464, 1375, 1171, 1111, 1034, 962, 822, 766 cm⁻¹; HRMS (ESI⁺) m/z: [M+H]⁺ calcd for C₃₀H₃₃NO₁₁, 584.2132. Found, 584.2111.

Benzyl 5-methoxy-7-((3aR,4R,7R,7aR)-7-methoxy-6,6-dimethyl-2-oxotetrahydro-3aH-[1,3]-dioxolo[4,5-c]pyran-4-yloxy)-8-methyl-2-oxo-2H-chromen-3-ylcarbamate (25d): Boron trifluoride etherate (18.5 μL, 0.15 mmol) was added to 23d (174 mg, 0.49 mmol) and (3aR,4S,7R,7aR)-7-methoxy-6,6-dimethyl-2-oxo-tetrahydro-3aH-[1.3]dioxolo[4,5-c]pyran-4-yl 2,2,2-trichloroacetimidate (621 mg, 1.71 mmol) in anhydrous CH₂Cl₂ (11.0 mL). After stirring at room temperature for 14 hours, triethylamine (150 μL) was added and the solvent was concentrated. The residue was purified via column chromatography (SiO₂, 40:1 CH₂Cl₂:Acetone) to give 25d as a colorless foam (200 mg, 74%): ¹H NMR (CDCl₃, 500 MHz) [symbol] 8.49 (s, 1H), 7.34-7.27 (m, 5H), 6.67 (s, 1H), 6.60 (s, 1H), 5.69 (s, 2H), 5.16 (d, J=5.3 Hz, 1H), 4.89 (t, J=7.8 Hz, 1H), 4.63 (dd, J=7.9, 2.4 Hz, 1H), 3.83 (s, 3H), 3.37 (s, 3H), 3.15 (d, J=8.0 Hz, 1H), 2.16 (s, 3H), 2.16 (s, 3H), 2.12 (s, 3H); ¹³C NMR (CDCl₃, 125 MHz) [symbol] 158.9, 156.0, 155.2, 154.2, 153.1 (2C), 149.4, 135.7, 128.7 (2C), 128.5, 128.2 (2C), 120.8, 117.4, 106.6, 105.4, 94.6, 94.1, 82.9, 67.4, 60.6, 60.6, 56.1, 56.0, 22.2, 22.0, 7.9; IR (film) νₘₐₓ 2955, 2924, 2853, 1817, 1713, 1526, 1209, 1105, 1072, 1034, 976, 808 cm⁻¹; HRMS (ESI⁺) m/z: [M+H]⁺ calcd for C₂₈H₂₉NO₁₁, 556.1819. Found, 556.1826.

Benzyl 8-benzyl-7-((3aR,4R,7R,7aR)-7-methoxy-6,6-dimethyl-2-oxotetrahydro-3aH-[1,3]dioxolo[4,5-c]pyran-4-yloxy)-2-oxo-2H-chromen-3-ylcarbamate (25e): Boron trifluoride etherate (7.80 μL, 0.062 mmol) was added to 23e (80.0 mg, 0.21 mmol) and (3aR,4S,7R,7aR)-7-methoxy-6,6-dimethyl-2-oxo-tetrahydro-3aH-[1.3]dioxolo[4,5-c]pyran-4-yl-2,2,2-trichloroacetimidate (299 mg, 0.83 mmol) in anhydrous CH₂Cl₂ (5.20 mL). After stirring at room temperature for 48 hours, triethylamine (150 μL) was added and the solvent was concentrated. The residue was purified via column chromatography (SiO₂, 40:1 CH₂Cl₂:Acetone) to give 25e as a colorless foam (47.0 mg, 39%): ¹H NMR (CDCl₃, 500 MHz) [symbol] 8.22 (s, 1H), 7.46 (s, 1H), 7.35-7.27 (m, 5H), 7.17-7.06 (m, 5H), 6.86 (d, J=10 Hz, 1H), 6.01 (d, J=10 Hz, 1H), 5.65 (d, J=1.6 Hz, 1H), 5.23 (s, 2H), 5.16 (s, 2H), 4.77-4.70 (m, 1H), 4.10 (s, 1H), 3.50 (s, 3H), 3.28 (s, 1H), 3.16 (d, J=7.4 Hz, 1H), 1.25 (s, 3H), 1.18 (s, 3H); ¹³C NMR (CDCl₃, 125 MHz) [symbol] 155.1, 153.2 (2C), 153.1, 148.6 (2C), 139.6 (2C), 128.7, 128.6 (2C), 128.4 (2C), 128.3 (2C), 128.3 (2C), 126.5, 126.2, 123.1, 122.4, 121.7, 117.6, 114.9, 111.5, 94.7, 82.8, 67.6, 60.6 (2C), 29.7, 27.6, 21.9; IR (film) νₘₐₓ 2926, 2854, 2359, 2341, 1811, 1709, 1607, 1522, 1456, 1381, 1366, 1259, 1209, 1171, 1078, 1049, 968, 766, 700 cm⁻¹; HRMS (ESI⁺) m/z: [M+H]⁺ calcd for C₃₃H₃₁NO₁₀, 602.2026. Found, 602.2053.

Benzyl 7-((3aR,4R,7R,7aR)-7-methoxy-6,6-dimethyl-2-oxotetrahydro-3aH-[1,3]dioxolo[4,5-c]pyran-4-yloxy)-2-oxo-8-phenyl-2H-chromen-3-ylcarbamate (250: Boron trifluoride etherate (14.6 μL, 0.12 mmol) was added to 23f (155 mg, 0.39 mmol) and (3aR,4S,7R,7aR)-7-methoxy-6,6-dimethyl-2-oxo-tetrahydro-3aH-[1.3]dioxolo[4,5-c]pyran-4-yl 2,2,2-trichloroacetimidate (560 mg, 1.55 mmol) in anhydrous CH₂Cl₂ (9.70 mL). After stirring at room temperature for 48 hours, triethylamine (150 μL) was added and the solvent was concentrated. The residue was purified via column chromatography (SiO₂, 100:1→40:1 CH₂Cl₂:Acetone) to give 25f as a colorless foam (225 mg, 99%): ¹H NMR (CD₂Cl₂, 400 MHz) [symbol] 8.37 (s, 1H), 7.75-7.73 (m, 2H), 7.60-7.36 (m, 10H), 7.32 (d, J=8.8 Hz, 1H), 5.77 (d, J=1.7 Hz, 1H), 5.26 (s, 2H), 4.76-4.68 (m, 1H), 4.36-4.28 (m, 1H), 3.56 (s, 3H), 3.28 (d, J=7.2 Hz, 1H), 1.37 (s, 3H), 1.31 (s, 3H); ¹³C NMR (CDCl₃, 125 MHz) [symbol] 157.2, 153.0 (2C), 152.1 (2C), 152.1, 134.4, 129.9, 129.8, 129.4, 127.7 (2C), 127.5, 127.2 (2C), 127.1 (2C), 127.0, 126.3, 121.5 (2C), 120.3, 111.2 (2C), 93.9, 81.9, 66.5, 59.4 (3C), 20.9 (2C); IR (film) νₘₐₓ 3400, 2959, 2926, 2853, 2359, 2341, 1819, 1715, 1601, 1522, 1381, 1366, 1261, 1215, 1173, 1111, 1059, 970, 800, 700 cm⁻¹; HRMS (ESI⁺) m/z: [M+H]⁺ calcd for C₃₂H₂₉NO₁₀, 588.1870. Found, 588.1846.

Benzyl 8-methoxy-7-((3aR,4S,7R,7aR)-7-methoxy-6,6-dimethyl-2-oxotetrahydro-3aH-[1,3]dioxolo[4,5-c]pyran-4-yloxy)-2-oxo-2H-chromen-3-ylcarbamate (25g): Boron trifluoride etherate (17.3 μL, 0.14 mmol) was added to 23g (157 mg, 0.46 mmol) and (3aR,4S,7R,7aR)-7-methoxy-6,6-dimethyl-2-oxo-tetrahydro-3aH-[1.3]dioxolo[4,5-c]pyran-4-yl 2,2,2-trichloroacetimidate (665 mg, 1.83 mmol) in anhydrous CH₂Cl₂ (11.5 mL). After stirring at room temperature for 24 hours, triethylamine (150 μL) was added and the solvent was concentrated. The residue was purified via column chromatography (SiO₂, 40:1→10:1 CH₂Cl₂:Acetone) to give 25g as a colorless foam (237 mg, 95%): ¹HNMR (CDCl₃, 500 MHz) [symbol] 8.20 (s, 1H), 7.48 (s, 1H), 7.33-7.29 (m, 5H), 7.09 (dd, J=14.2, 8.8 Hz, 2H), 5.72 (d, J=1.8 Hz, 1H), 5.16 (s, 2H), 5.02 (dd, J=7.8, 1.8 Hz, 1H), 4.89 (t, J=7.8 Hz, 1H), 3.88 (s, 3H), 3.52 (s, 3H), 3.21 (d, J=7.8 Hz, 1H), 1.27 (s, 3H) 1.17 (s, 3H); ¹³C NMR (CDCl₃, 125 MHz) [symbol] 158.0, 153.3, 153.2, 153.1, 149.8, 143.8, 137.1, 135.5, 128.7 (2C), 128.6, 128.3 (2C), 122.7, 122.2, 121.4, 116.1, 113.7, 95.3, 74.7, 72.9, 67.6, 61.9, 60.7, 60.6, 29.7, 29.4; IR (film) νₘₐₓ 3400, 3319, 2984, 2935, 2359, 1815, 1715, 1609, 1526, 1464, 1383, 1364, 1285, 1213, 1175, 1111, 1063, 968, 764, 737, 700 cm⁻¹; HRMS (ESI⁺) m/z: [M+Na]⁺ calcd for C₂₇H₂₇NO₁₁, 564.1482. Found, 564.1455.

Benzyl 8-ethyl-7-((3aR,4R,7R,7aR)-7-methoxy-6,6-dimethyl-2-oxotetrahydro-3aH-[1,3]dioxolo[4,5-c]pyran-4-yloxy)-2-oxo-2H-chromen-3-ylcarbamate (25h): Boron trifluoride etherate (19.0 μL, 0.15 mmol) was added to 23h (171 mg, 0.51 mmol) and (3aR,4S,7R,7 aR)-7-methoxy-6,6-dimethyl-2-oxo-tetrahydro-3aH-[1.3]dioxolo[4,5-c]pyran-4-yl-2,2,2-trichloroacetimidate (183 mg, 0.51 mmol) in anhydrous CH₂Cl₂ (11.0 mL). After stirring at room temperature for 24 hours, triethylamine (150 μL) was added and the solvent was concentrated. The residue was purified via column chromatography (SiO₂, 40:1 CH₂Cl₂:Acetone) to give 25h as a colorless foam (138 mg, 51%): ¹HNMR (CD₂Cl₂, 400 MHz) [symbol] 8.29 (s, 1H), 7.62 (s, 1H), 7.47-7.38 (m, 5H), 7.36 (d, J=8.8 Hz, 1H), 7.19 (d, J=8.4 Hz, 1H), 5.81 (d, J=2.4 Hz, 1H), 5.25 (s, 2H), 5.10 (dd, J=8.0, 2.0 Hz, 1H), 5.02 (t, J=7.8 Hz, 1H), 3.55 (s, 3H), 3.41 (d, J=7.2 Hz, 1H), 2.87 (q, J=7.4 Hz, 2H), 1.42 (s. 3H), 1.28 (s, 3H), 1.21 (t, J=7.4 Hz, 3H); ¹³C NMR (CD₂Cl₂, 100 MHz) [symbol] 158.5, 154.8, 153.2, 153.1, 148.4, 136.0, 128.6 (2C), 128.4, 128.1, 125.6, 122.4, 121.5, 120.7, 114.8, 111.4, 94.8, 82.7, 77.9, 77.2, 76.6, 67.3, 60.3, 27.3, 22.3, 16.4, 13.6; IR (film) νₘₐₓ 3400, 2980, 2937, 2359, 2339, 1817, 1711, 1607, 1524, 1383, 1366, 1227, 1205, 1175, 1101, 1040, 906, 768, 737, 700 cm⁻¹; [M+Na]⁺ calcd for C₂₈H₂₉NO₁₀, 562.1689. Found, 562.1689.

N-(7-((2S,3R,4S,5R)-3,4-dihydroxy-5-methoxy-6,6-dimethyltetrahydro-2H-pyran-2-yloxy)-6-methoxy-8-methyl-2-oxo-2H-chromen-3-yl)-3′,6-dimethoxybiphenyl-3-carboxamide (26a): Palladium on carbon (10%, 20.0 mg) was added to 25a (100 mg, 0.18 mmol) in anhydrous THF (5.00 mL) and the solution was placed under an atmosphere of H₂. After 6.5 hours, the solution was filtered through SiO₂ (1:1 CH₂Cl₂:Acetone) and the eluent was concentrated to afford a yellow solid, which was used without further purification (56.0 mg, 75%). EDCI (21.4 mg, 0.11 mmol) and 3′,6-dimethoxybiphenyl-3-carboxylic acid (23.1 mg, 0.089 mmol) were added to the amine (18.7 mg, 0.045 mmol) in 30% pyridine/CH₂Cl₂ (0.70 mL). After 12 hours, the solvent was concentrated and the residue purified via column chromatography (SiO₂, 40:1 CH₂Cl₂:Acetone) to afford a colorless solid, which was used without further purification (10.5 mg, 36%). Triethylamine (150 μL) was added to the carbonate (10.4 mg, 0.016 mmol) in MeOH (2.50 mL). After 12 hours, the solvent was concentrated and the residue purified via column chromatography (SiO₂, 20:1; CH₂Cl₂:MeOH) to afford 26a as a colorless amorphous solid (2.00 mg, 20%, 5% over 3 steps): ¹H NMR (CDCl₃, 500 MHz) [symbol] 8.73 (s, 1H), 8.70 (d, J=5.4 Hz, 1H), 7.84 (td, J=6.2, 2.4 Hz, 1H), 7.82 (s, 1H), 7.30 (t, J=8.0 Hz, 1H), 7.06 (d, J=7.8 Hz, 1H), 7.03-7.00 (m, 2H), 6.88-6.86 (m, 1H), 6.81 (s, 1H), 4.99 (d, J=6.6 Hz, 1H), 4.24 (t, J=4.2 Hz, 1H), 4.00 (dd, J=6.5, 3.7 Hz, 1H), 3.90 (s, 3H), 3.86 (s, 3H), 3.83 (s, 3H), 3.80 (d, J=7.4 Hz, 1H), 3.45 (s, 3H), 3.08 (d, J=4.7 Hz, 1H), 2.67 (s, 1H), 2.42 (s, 3H), 1.28 (d, J=8.1 Hz, 3H), 1.18 (s, 3H); ¹³C NMR (CDCl₃, 125 MHz) δ 164.6, 158.9, 158.3, 158.2, 148.2, 145.6, 142.5, 137.5, 130.1, 129.0, 128.2, 127.2, 124.9, 122.5, 122.2, 121.2, 121.0, 115.1, 144.2, 112.1, 110.0, 105.4, 101.3, 81.7, 76.8, 69.0, 68.0, 59.1, 55.3, 54.9, 54.3, 28.3, 28.2, 9.1; IR (film) νₘₐₓ 2961, 2928, 1713, 1670, 1601, 1464, 1383, 1261, 1094, 1022, 798, 700 cm⁻¹; HRMS (ESI⁺) m/z: [M+H]⁺ calcd for C₃₄H₃₇NO₁₁, 636.2445. Found, 636.2477.

N-(7-((2S,3R,4S,5R)-3,4-dihydroxy-5-methoxy-6,6-dimethyltetrahydro-2H-pyran-2-yloxy)-8-methyl-2-oxo-6-propoxy-2H-chromen-3-yl)-3′,6-dimethoxybiphenyl-3-carboxamide (26b): Palladium on carbon (10%, 85.0 mg) was added to 25b (425 mg, 0.7283 mmol) in anhydrous THF (4.90 mL) and the solution was placed under an atmosphere of H₂. After 6.5 hours, the solution was filtered through SiO₂ (1:1 CH₂Cl₂:Acetone) and the eluent was concentrated to afford a yellow solid, which was used without further purification (325 mg, 99%). EDCI (116 mg, 0.60 mmol) and 3′,6-dimethoxybiphenyl-3-carboxylic acid (125 mg, 0.4821 mmol) were added to the amine (108 mg, 0.2410 mmol) in 30% pyridine/CH₂Cl₂ (6.70 mL). After 12 hours, the solvent was concentrated and the residue purified via column chromatography (SiO₂, 3:1 Hexane:Ether→20:1 CH₂Cl₂:Acetone) to afford a colorless solid, which was used without further purification (51.0 mg, 31%). Triethylamine (150 μL) was added to the carbonate (51.0 mg, 0.074 mmol) in MeOH (2.50 mL). After 48 hours, the solvent was concentrated and the residue purified via column chromatography (SiO₂, 40:1 CH₂Cl₂:Acetone) to afford 26b as a colorless amorphous solid (22.8 mg, 47%, 14% over 3 steps): ¹H NMR (CD₂Cl₂, 400 MHz) [symbol] 8.79 (s, 1H), 8.78 (s, 1H), 7.96 (dd, J=8.6, 2.4 Hz, 1H), 7.91 (d, J=2.4 Hz, 1H), 7.39 (t, J=7.9 Hz, 1H), 7.16-7.11 (m, 2H), 6.97-6.94 (m, 2H), 5.97 (s, 1H), 5.14 (d, J=6.5 Hz, 1H), 4.31 (t, J=3.5 Hz, 1H), 4.12-4.06 (m, 2H), 4.03 (dd, J=6.8, 1.8 Hz, 1H), 3.93 (s, 3H), 3.88 (s, 3H), 3.65 (s, 1H), 3.53 (s, 3H), 3.17 (d, J=4.8 Hz, 1H), 2.80 (s, 1H), 2.48 (s, 3H), 1.95-1.90 (m, 2H), 1.37 (s, 3H), 1.35 (s, 3H), 1.11 (t, J=7.4 Hz, 3H); ¹³C NMR (CDCl₃, 125 MHz) [symbol] 165.0, 164.6, 158.8, 158.3, 147.7, 145.7, 142.3, 137.8, 137.5, 131.3, 129.0, 128.2, 127.2, 124.9, 122.3, 121.2, 121.0, 115.111, 114.2, 112.1, 110.0, 106.2, 101.1, 81.7, 70.0, 69.0, 68.0, 64.8, 59.1, 54.9, 54.3, 24.7, 24.0, 21.3, 9.5, 9.1; IR (film) νₘₐₓ 3398, 3196, 2964, 2935, 2359, 2330, 1705, 1580, 1526, 1504, 1381, 1242, 1124, 1094, 939, 808, 760, 735 cm⁻¹; HRMS (ESI⁺) m/z: [M+H]⁺ calcd for C₃₆H₄₁NO₁₁, 664.2758. Found, 664.2754.

N-(7-((2S,3R,4S,5R)-3,4-dihydroxy-5-methoxy-6,6-dimethyltetrahydro-2H-pyran-2-yloxy)-6-isopropoxy-8-methyl-2-oxo-2H-chromen-3-yl)-3′,6-dimethoxybiphenyl-3-carboxamide (26c): Palladium on carbon (10%, 11 mg) was added to 25c (54.5 mg, 0.093 mmol) in anhydrous THF (600 μL) and the solution was placed under an atmosphere of H₂. After 12 hours, the solution was filtered through SiO₂ (1:1 CH₂Cl₂:Acetone) and the eluent was concentrated to afford a yellow solid, which was used without further purification (42.0 mg, 99%). EDCI (14.9 mg, 0.078 mmol) and 3′,6-dimethoxybiphenyl-3-carboxylic acid (16 mg, 0.062 mmol) were added to the amine (14.0 mg, 0.031 mmol) in 30% pyridine/CH₂Cl₂ (900 μL). After 12 hours, the solvent was concentrated and the residue purified via column chromatography (SiO₂, 3:1 Hexane:Ether 40:1 CH₂Cl₂:Acetone) to afford a colorless solid, which was used without further purification (17.5 mg, 82%). Triethylamine (150 μL) was added to the carbonate (17.5 mg, 0.025 mmol) in MeOH (2.50 mL) and CH₂Cl₂ (2.50 mL). After 12 hours, the solvent was concentrated and the residue purified via column chromatography (SiO₂, 10:1 CH₂Cl₂:Acetone) to afford 26c as a colorless amorphous solid (6.0 mg, 35%, 28% over 3 steps): ¹H NMR (CD₂Cl₂, 500 MHz) [symbol] 8.69 (s, 1H), 8.67 (s, 1H), 7.84 (dd, J=8.6, 2.4 Hz, 1H), 7.80 (d, J=2.4 Hz, 1H), 7.29 (d, J=8.0 Hz, 1H), 7.25 (t, J=7.9 Hz, 1H), 7.03-7.01 (m, 2H), 6.87 (s, 1H), 6.87-6.83 (m, 1H), 4.96 (d, J=6.8 Hz, 1H), 4.61-4.56 (m, 1H), 4.19 (t, J=4.0 Hz, 1H), 3.89 (dd, J=6.8, 3.7 Hz, 1H), 3.82 (s, 3H), 3.76 (s, 3H), 3.75 (s, 1H), 3.41 (s, 3H), 3.34 (s, 1H), 3.03 (d, J=4.5 Hz, 1H), 2.36 (s, 3H), 1.33 (t, J=6.2 Hz, 6H), 1.25 (s, 3H), 1.23 (s, 3H); ¹³C NMR (CD₂Cl₂, 125 MHz) [symbol] 164.6, 159.1, 158.6, 158.3, 146.7, 146.3, 142.5, 138.1, 130.2, 129.1, 128.3, 127.4, 125.2, 122.8, 122.2, 121.2, 121.1, 115.5, 144.5, 112.1, 110.2, 108.4, 101.4, 81.9, 77.0, 71.1, 69.2, 68.3, 59.1, 55.1, 54.5, 28.7, 28.6, 20.8, 20.8, 9.1; IR (film) νₘₐₓ 2924, 2854, 2359, 2341, 1734, 1684, 1653, 1558, 1541, 1522, 1506, 1458, 1387, 1339, 1286, 1244, 1113, 912, 797 cm⁻¹; HRMS (ESI⁺) m/z: [M+Na]⁺ calcd for C₃₆H₄₁NO₁₁, 686.2578. Found, 686.2610.

N-(7-((2R,3R,4S,5R)-3,4-dihydroxy-5-methoxy-6,6-dimethyltetrahydro-2H-pyran-2-yloxy)-5-methoxy-8-methyl-2-oxo-2H-chromen-3-yl)-3′,6-dimethoxybiphenyl-3-carboxamide (26d): Palladium on carbon (10%, 40 mg) was added to 25d (200 mg, 0.36 mmol) in anhydrous THF (2.40 mL) and the solution was placed under an atmosphere of H₂. After 12 hours, the solution was filtered through SiO₂ (1:1 CH₂Cl₂:Acetone) and the eluent was concentrated to afford a yellow solid, which was used without further purification (150 mg, 99%). EDCI (57.5 mg, 0.30 mmol) and 3′,6-dimethoxybiphenyl-3-carboxylic acid (62 mg, 0.24 mmol) were added to the amine (50.6 mg, 0.12 mmol) in 30% pyridine/CH₂Cl₂ (3.30 mL). After 12 hours, the solvent was concentrated and the residue purified via column chromatography (SiO₂, 3:1 Hexane:Ether→40:1→10:1 CH₂Cl₂:Acetone) to afford a colorless solid, which was used without further purification (25.2 mg, 32%). Triethylamine (150 μL) was added to the carbonate (25.2 mg, 0.038 mmol) in MeOH (2.0 mL) and CH₂Cl₂ (2.0 mL). After 48 hours, the solvent was concentrated and the residue purified via column chromatography (SiO₂, 40:1 CH₂Cl₂:Acetone) to afford 26d as a colorless amorphous solid (17.0 mg, 70%, 22% over 3 steps): ¹HNMR (CD₂Cl₂, 400 MHz) [symbol] 9.02 (s, 1H), 8.97 (s, 1H), 8.66 (s, 1H), 7.96 (dd, J=8.6, 2.4 Hz, 1H), 7.91-7.90 (m, 1H), 7.39 (t, J=7.9 Hz, 1H), 7.16-7.11 (m, 2H), 6.96 (dd, J=8.3, 2.6 Hz), 6.85 (d, J=5.5 Hz, 1H), 5.70 (d, J=2.1 Hz, 1H), 4.36-4.33 (m, 1H), 4.27 (m, 1H), 3.99 (s, 3H), 3.93 (s, 3H), 3.88 (s, 3H), 3.62 (s, 3H), 3.41-3.38 (m, 1H), 2.24 (s, 3H), 1.41 (s, 3H), 1.19 (s, 3H); ¹³C NMR (CDCl₃, 125 MHz) [symbol] 164.2, 158.7, 158.6, 158.3, 155.3, 153.5, 148.6, 137.6, 129.9, 128.9, 128.1, 127.1, 125.1, 121.0, 119.4, 118.9, 114.2, 114.2, 112.1, 110.0, 104.9, 103.7, 96.7, 92.9, 83.2, 70.1, 67.5, 60.9, 60.8, 54.8, 54.3, 21.9, 21.4, 6.8; IR (film) νₘₐₓ 3405, 2986, 2934, 1713, 1609, 1528, 1383, 1250, 1213, 1053, 999, 914, 878, 737 cm⁻¹; HRMS (ESI⁺) m/z: [M+H]⁺ calcd for C₃₄H₃₇NO₁₁, 636.2445. Found, 636.2482.

N-(8-benzyl-7-((2R,3R,4S,5R)-3,4-dihydroxy-5-methoxy-6,6-dimethyltetrahydro-2H-pyran-2-yloxy)-2-oxo-2H-chromen-3-yl)-3′,6-dimethoxybiphenyl-3-carboxamide (26e): Palladium on carbon (10%, 46 mg) was added to 25e (230 mg, 0.38 mmol) in anhydrous THF (2.50 mL) and the solution was placed under an atmosphere of H₂. After 12 hours, the solution was filtered through SiO₂ (1:1 CH₂Cl₂:Acetone) and the eluent was concentrated to afford a yellow solid, which was used without further purification (177 mg, 99%). EDCI (61.5 mg, 0.32 mmol) and 3′,6-dimethoxybiphenyl-3-carboxylic acid (66.3 mg, 0.26 mmol) were added to the amine (60.0 mg, 0.13 mmol) in 30% pyridine/CH₂Cl₂ (3.50 mL). After 12 hours, the solvent was concentrated and the residue purified via column chromatography (SiO₂, 3:1 Hexane:Ether→20:1 CH₂Cl₂:Acetone) to afford a colorless solid, which was used without further purification (12.3 mg, 14%). Triethylamine (150 μL) was added to the carbonate (12.3 mg, 0.017 mmol) in MeOH (1.5 mL) and CH₂Cl₂ (1.5 mL). After 48 hours, the solvent was concentrated and the residue purified via column chromatography (SiO₂, 40:1 CH₂Cl₂:Acetone) to afford 26e as a colorless amorphous solid (6.00 mg, 51%, 7.1% over 3 steps): ¹H NMR (CD₂Cl₂, 400 MHz) [symbol] 8.84 (s, 1H), 8.72 (s, 1H), 7.96 (dd, J=10, 2.4 Hz, 1H), 7.91 (d, J=2.4 Hz, 1H), 7.50 (d, J=8.8 Hz, 1H), 7.39 (t, J=7.9 Hz, 1H), 7.31 (d, J=8.8 Hz, 1H), 7.28-7.25 (m, 5H), 7.21-7.18 (m, 1H), 7.15-7.11 (m, 2H), 6.97-6.94 (m, 1H), 5.54 (d, J=2.7 Hz, 1H), 4.25 (t, J=15.1 Hz, 2H), 4.17-4.11 (m, 1H), 4.05 (d, J=2.6 Hz, 1H), 3.93 (s, 3H), 3.88 (s, 3H), 3.58 (s, 3H), 3.31 (d, J=8.7 Hz, 1H), 2.64 (s, 1H), 2.04 (s, 1H), 1.40 (s, 3H), 1.03 (s, 3H); ¹³C NMR (CDCl₃, 125 MHz) [symbol] 165.5, 159.8, 159.3, 159.3, 156.4, 148.9, 140.0 (2C), 138.6, 131.1, 130.0, 129.2, 128.5, 128.3, 128.2, 127.0, 126.2 (2C), 126.0 (2C), 124.1, 122.2, 122.0, 117.2, 115.2, 114.4, 113.2, 111.7, 111.0, 98.0, 70.6 (2C), 68.6, 61.6, 55.9, 55.4, 29.3, 28.9, 28.3; IR (film) νₘₐₓ 3404, 2930, 2359, 2341, 1713, 1670, 1605, 1526, 1502, 1367, 1244, 1180, 1134, 1076, 1026, 960 cm⁻¹; HRMS (ESI⁺) m/z: [M+H]⁺ calcd for C₃₉H₃₉NO₁₀, 682.2652. Found, 682.2653.

N-(7-((2R,3R,4S,5R)-3,4-dihydroxy-5-methoxy-6,6-dimethyltetrahydro-2H-pyran-2-yloxy)-2-oxo-8-phenyl-2H-chromen-3-yl)-3′,6-dimethoxybiphenyl-3-carboxamide (26f): Palladium on carbon (10%, 14 mg) was added to 25f (68.0 mg, 0.12 mmol) in anhydrous THF (800 μL) and the solution was placed under an atmosphere of H₂. After 12 hours, the solution was filtered through SiO₂ (1:1 CH₂Cl₂:Acetone) and the eluent was concentrated to afford a yellow solid, which was used without further purification (52.0 mg, 99%). EDCI (18.5 mg, 0.096 mmol) and 3′,6-dimethoxybiphenyl-3-carboxylic acid (19.9 mg, 0.077 mmol) were added to the amine (17.5 mg, 0.039 mmol) in 30% pyridine/CH₂Cl₂ (1.10 mL). After 12 hours, the solvent was concentrated and the residue purified via column chromatography (SiO₂, 40:1 CH₂Cl₂:Acetone) to afford a colorless solid, which was used without further purification (14.0 mg, 52%). Triethylamine (150 μL) was added to the carbonate (14.0 mg, 0.020 mmol) in MeOH (1.5 mL) and CH₂Cl₂ (1.5 mL). After 48 hours, the solvent was concentrated and the residue purified via column chromatography (SiO₂, 40:1 CH₂Cl₂:Acetone) to afford 26f as a colorless amorphous solid (5.20 mg, 39%, 20% over 3 steps): ¹H NMR (CD₂Cl₂, 500 MHz) [symbol] 8.85 (s, 1H), 8.65 (s, 1H), 7.92 (d, J=2.4 Hz, 1H), 7.90 (d, J=2.4 Hz, 1H), 7.86 (d, J=2.4 Hz, 1H), 7.57-7.43 (m, 3H), 7.36-7.33 (m, 4H), 7.11-7.06 (m, 3H), 6.92 (d, J=0.8 Hz, 1H), 5.52 (d, J=2.4 Hz, 1H), 4.08 (q, J=7.2, Hz, 1H), 3.89 (s, 3H), 3.83 (s, 3H), 3.74 (dd, J=9.0, 3.5 Hz, 1H), 3.50 (s, 3H), 3.23 (d, J=9.0 Hz, 1H), 2.12 (s, 1H), 2.00 (s, 1H), 1.33 (s, 3H), 1.04 (s, 3H); ¹³C NMR (CD₂Cl₂, 125 MHz) δ 164.5, 159.0 (2C), 158.6, 158.1, 154.4, 147.2, 138.0 (2C), 130.7, 130.1, 129.7, 129.1, 128.7, 128.3, 127.4, 127.2, 127.0, 127.0, 125.2, 122.6, 121.6, 121.1, 118.6, 114.5, 113.8, 112.1, 111.3, 110.2, 97.5, 70.1 (2C), 67.4, 60.8, 55.0, 54.5, 21.9, 21.6; IR (film) νₘₐₓ 3402, 2932, 2359, 2341, 1713, 1603, 1524, 1500, 1367, 1267, 1086, 1040, 964, 750 cm⁻¹; HRMS (ESI⁺) m/z: [M+H]⁺ calcd for C₃₈H₃₇NO₁₀, 668.2496. Found, 668.2485.

N-(7-((2S,3R,4S,5R)-3,4-dihydroxy-5-methoxy-6,6-dimethyltetrahydro-2H-pyran-2-yloxy)-8-methoxy-2-oxo-2H-chromen-3-yl)-3′,6-dimethoxybiphenyl-3-carboxamide (26g, KU-174): Palladium on carbon (10%, 47 mg) was added to 25g (237 mg, 0.44 mmol) in anhydrous THF (2.93 mL) and the solution was placed under an atmosphere of H₂. After 12 hours, the solution was filtered through SiO₂ (1:1 CH₂Cl₂:Acetone) and the eluent was concentrated to afford a yellow solid, which was used without further purification (177 mg, 99%). EDCI (69.4 mg, 0.36 mmol) and 3′,6-dimethoxybiphenyl-3-carboxylic acid (74.8 mg, 0.29 mmol) were added to the amine (59.0 mg, 0.14 mmol) in 30% pyridine/CH₂Cl₂ (4.00 mL). After 12 hours, the solvent was concentrated and the residue purified via column chromatography (SiO₂, 3:1 Hexane:Ether→40:1 CH₂Cl₂:Acetone) to afford a colorless solid, which was used without further purification (26.0 mg, 28%).

Triethylamine (150 μL) was added to the carbonate (26.0 mg, 0.040 mmol) in MeOH (2.0 mL) and CH₂Cl₂ (2.0 mL). After 48 hours, the solvent was concentrated and the residue purified via column chromatography (SiO₂, 40:1 CH₂Cl₂:Acetone) to afford 26g as a colorless amorphous solid (15.7 mg, 63%, 18% over 3 steps): ¹H NMR (CD₂Cl₂, 400 MHz) [symbol] 8.82 (s, 1H), 8.73 (s, 1H), 7.96 (dd, J=8.6, 2.4 Hz, 1H), 7.91 (d, J=2.4 Hz, 1H) 7.39 (t, J=7.9 Hz, 1H), 7.30 (s, 2H), 7.14 (d, J=8.6 Hz, 2H), 7.12 (d, J=2.2 Hz, 1H), 6.96 (dd, J=8.3, 2.5 Hz, 1H), 5.61 (d, J=2.4 Hz, 1H), 4.29 (t, J=4.0 Hz, 1H), 4.27-4.25 (m, 1H), 3.98 (s, 3H), 3.93 (s, 3H), 3.88 (s, 3H), 3.62 (s, 3H), 3.47 (s, 1H), 3.37 (d, J=8.8 Hz, 1H), 2.62 (s, 1H), 1.30 (s, 3H), 1.24 (s, 3H); ¹³C NMR (CDCl₃, 125 MHz) [symbol] 164.5, 158.8, 158.3, 157.8, 150.2 (2C), 142.9, 137.5, 135.6, 130.0, 128.9, 128.2, 127.2, 127.2, 124.9, 122.8, 121.6, 121.0, 114.3, 114.2, 112.3, 112.1, 110.0, 97.7; 70.0 (2C), 67.5, 60.8 (2C), 54.9, 54.3, 28.7, 28.3; IR (film) νₘₐₓ 3402, 2961, 2928, 2853, 1713, 1672, 1607, 1526, 1504, 1462, 1367, 1263, 1248, 1086, 1040, 953, 798, 735, 700 cm⁻¹; HRMS (ESI⁺) m/z: [M+H]⁺ calcd for C₃₃H₃₅NO₁₁, 622.2288. Found, 622.2307.

N-(7-((2R,3R,4S,5R)-3,4-dihydroxy-5-methoxy-6,6-dimethyltetrahydro-2H-pyran-2-yloxy)-8-ethyl-2-oxo-2H-chromen-3-yl)-3′,6-dimethoxybiphenyl-3-carboxamide (26h): Palladium on carbon (10%, 12 mg) was added to 25h (121 mg, 0.22 mmol) in anhydrous THF (5.00 mL) and the solution was placed under an atmosphere of H₂. After 12 hours, the solution was filtered through SiO₂ (1:1 CH₂Cl₂:Acetone) and the eluent was concentrated to afford a yellow solid, which was used without further purification (90.0 mg, 99%). EDCI (46.2 mg, 0.24 mmol) and 3′,6-dimethoxybiphenyl-3-carboxylic acid (43.9 mg, 0.19 mmol) were added to the amine (39.0 mg, 0.096 mmol) in 30% pyridine/CH₂Cl₂ (2.65 mL). After 12 hours, the solvent was concentrated and the residue purified via column chromatography (SiO₂, 40:1 CH₂Cl₂:Acetone) to afford a colorless solid, which was used without further purification (39.1 mg, 66%). Triethylamine (150 μL) was added to the carbonate (13.0 mg, 0.020 mmol) in MeOH (1.5 mL). After 12 hours, the solvent was concentrated and the residue purified via column chromatography (SiO₂, 40:1 CH₂Cl₂:Acetone) to afford 26h as a colorless amorphous solid (4.10 mg, 33%, 22% over 3 steps): ¹H NMR (CD₂Cl₂, 500 MHz) [symbol] 8.70 (s, 1H), 8.61 (s, 1H), 7.83 (dd, J=8.5, 2.5 Hz, 1H), 7.78 (d, J=2.5 Hz, 1H), 7.31 (d, J=9.0 Hz, 1H), 7.27 (t, J=7.8 Hz, 1H), 7.17 (d, J=8.5 Hz, 1H), 7.03-6.99 (m, 3H), 6.85-6.82 (m, 1H), 5.49 (d, J=1.5 Hz, 1H), 4.15 (t, J=8.5 Hz, 1H), 4.14 (d, J=8.5 Hz, 1H), 3.90 (s, 2H), 3.87 (s, 3H), 3.81 (s, 3H), 3.28 (s, 3H), 3.27 (d, J=8.5 Hz, 1H), 2.76 (q, J=4.5 Hz, 2H), 1.29 (s, 3H), 1.09 (t, J=7.4 Hz, 3H), 1.08 (s, 3H); ¹³C NMR (CD₂Cl₂, 125 MHz) [symbol] 164.5, 159.0, 158.6, 158.5, 155.1, 147.9, 138.1, 130.1, 129.1, 128.3, 127.4, 125.4, 125.2, 123.1, 121.4, 121.2, 119.4, 114.5, 113.5, 112.1, 110.8, 110.2, 97.6, 83.4, 77.7, 70.6, 67.8, 61.0, 55.1, 54.5, 28.2, 21.6, 15.6, 12.9; IR (film) νₘₐₓ 3404, 2968, 2934, 2359, 2341, 1715, 1605, 1524, 1504, 1367, 1244, 1101, 1024, 995, 960, 800 cm⁻¹; HRMS (ESI⁺) m/z: [M+H]⁺ calcd for C₃₄H₃₇NO₁₀, 620.2496. Found, 620.2507.

N-(7-((2S,3R,4S,5R)-3,4-dihydroxy-5-methoxy-6,6-dimethyltetrahydro-2H-pyran-2-yloxy)-6-methoxy-8-methyl-2-oxo-2H-chromen-3-yl)-1H-indole-2-carboxamide (26i): Palladium on carbon (10%, 15 mg) was added to 25a (74.0 mg, 0.13 mmol) in anhydrous THF (5.00 mL) and the solution was placed under an atmosphere of H₂. After 12 hours, the solution was filtered through SiO₂ (1:1 CH₂Cl₂:Acetone) and the eluent was concentrated to afford a yellow solid, which was used without further purification (60.0 mg, 99%). EDCI (69.0 mg, 0.36 mmol) and 1H-indole-2-carboxylic acid (46.4 mg, 0.29 mmol) were added to the amine (60.0 mg, 0.14 mmol) in 30% pyridine/CH₂Cl₂ (3.50 mL). After 12 hours, the solvent was concentrated and the residue purified via column chromatography (SiO₂, 3:1 Hexane:Ether→40:1 CH₂Cl₂:Acetone) to afford a colorless solid, which was used without further purification (68.0 mg, 85%). Triethylamine (150 μL) was added to the carbonate (68.0 mg, 0.12 mmol) in MeOH (2.5 mL) and CH₂Cl₂ (2.50 mL). After 12 hours, the solvent was concentrated and the residue purified via column chromatography (SiO₂, 40:1 CH₂Cl₂:Acetone) to afford 26i as a colorless amorphous solid (12.6 mg, 19%, 16% over 3 steps): ¹H NMR (CD₂Cl₂, 400 MHz) [symbol] 8.29 (s, 1H), 7.63 (s, 1H), 7.45-7.39 (m, 3H), 6.92 (s, 1H), 6.85 (s, 1H), 6.19 (s, 1H), 5.09 (d, J=6.5 Hz, 1H), 4.31-4.28 (m, 1H), 4.01-3.97 (m, 1H), 3.94 (s, 3H), 3.62 (s, 1H), 3.56 (s, 3H), 3.15 (d, J=4.9 Hz, 1H), 2.46 (s, 3H), 2.36 (s, 1H), 1.38 (d, J=11.5 Hz, 3H), 1.32 (s, 3H); ¹³C NMR (CDCl₃, 125 MHz) δ 157.6, 152.1, 148.2, 145.4, 142.2, 134.5, 129.9, 127.8, 127.7, 127.5, 127.3, 122.3, 121.2, 120.2, 115.0, 105.3, 105.1, 101.3, 81.7, 69.0, 68.0, 66.5, 59.1, 55.2, 28.7, 24.6, 24.1, 9.1; IR (film) νₘₐₓ 2926, 1707, 1526, 1464, 1391, 1340, 1296, 1231, 1207, 1086, 1024, 943, 739, 700, 623 cm⁻¹; HRMS (ESI⁺) m/z: [M+Na]⁺ calcd for C₂₈H₃₀N₂O₉, 561.1849. Found, 561.1781.

N-(7-((2S,3R,4S,5R)-3,4-dihydroxy-5-methoxy-6,6-dimethyltetrahydro-2H-pyran-2-yloxy)-8-methyl-2-oxo-6-propoxy-2H-chromen-3-yl)-1H-indole-2-carboxamide (26j): Palladium on carbon (10%, 85 mg) was added to 25b (425 mg, 0.729 mmol) in anhydrous THF (4.90 mL) and the solution was placed under an atmosphere of H₂. After 12 hours, the solution was filtered through SiO₂ (1:1 CH₂Cl₂:Acetone) and the eluent was concentrated to afford a yellow solid, which was used without further purification (325 mg, 99%). EDCI (116 mg, 0.6026 mmol) and 1H-indole-2-carboxylic acid (77.7 mg, 0.4821 mmol) were added to the amine (108 mg, 0.2410 mmol) in 30% pyridine/CH₂Cl₂ (6.70 mL). After 12 hours, the solvent was concentrated and the residue purified via column chromatography (SiO₂, 3:1 Hexane:Ether 40:1 CH₂Cl₂:Acetone) to afford a colorless solid, which was used without further purification (91.0 mg, 64%). Triethylamine (150 μL) was added to the carbonate (91.0 mg, 0.1536 mmol) in MeOH (2.5 mL) and CH₂Cl₂ (2.50 mL). After 48 hours, the solvent was concentrated and the residue purified via column chromatography (SiO₂, 40:1 CH₂Cl₂:Acetone) to afford 26j as a colorless amorphous solid (17.5 mg, 20%, 13% over 3 steps): ¹H NMR (CD₂Cl₂, 400 MHz) [symbol] 9.32 (s, 1H), 8.80 (s, 1H), 8.76 (s, 1H), 7.77 (d, J=8.0 Hz, 1H), 7.53 (d, J=7.5 Hz, 1H), 7.40-7.36 (m, 1H), 7.24-7.20 (m, 1H), 6.98 (s, 1H), 6.01 (s, 1H), 5.15 (d, J=6.5 Hz, 1H), 4.32-4.25 (m, 1H), 4.11-4.04, (m, 1H), 3.62-3.59 (m, 2H), 3.53 (s, 3H), 3.18-3.12 (m, 1H), 2.64 (s, 1H), 2.49 (s, 3H), 2.18 (s, 1H), 1.95-1.91 (m, 2H), 1.36 (d, J=9.6 Hz, 3H), 1.29 (d, J=9.8 Hz, 3H), 1.12 (t, J=7.4 Hz, 3H); ¹³C NMR (CDCl₃, 125 MHz) [symbol] 160.1, 159.0, 148.8, 146.9, 143.4, 136.9, 129.8, 127.6, 125.5, 123.5, 123.0, 122.6, 122.3, 121.2, 116.0, 112.0, 107.3, 104.3, 102.2, 82.8, 71.0, 70.1, 69.1, 60.2, 59.7, 25.7, 23.1, 23.4, 10.5, 10.2; IR (film) νₘₐₓ 3630, 3304, 2926, 2854, 2359, 2332, 1713, 1705, 1539, 1387, 1240, 1103, 947, 930, 822, 739 cm⁻¹; HRMS (ESI⁺) m/z: [M+H]⁺ calcd for C₃₀H₃₄N₂O₉, 567.2342. Found, 567.2367.

N-(7-((2S,3R,4S,5R)-3,4-dihydroxy-5-methoxy-6,6-dimethyltetrahydro-2H-pyran-2-yloxy)-6-isopropoxy-8-methyl-2-oxo-2H-chromen-3-yl)-1H-indole-2-carboxamide (26k): Palladium on carbon (10%, 4 mg) was added to 25c (19.0 mg, 0.033 mmol) in anhydrous THF (220 μL) and the solution was placed under an atmosphere of H₂. After 12 hours, the solution was filtered through SiO₂ (1:1 CH₂Cl₂:Acetone) and the eluent was concentrated to afford a yellow solid, which was used without further purification (14.5 mg, 99%). EDCI (15.6 mg, 0.081 mmol) and 1H-indole-2-carboxylic acid (10.5 mg, 0.065 mmol) was added to the amine (14.5 mg, 0.033 mmol) in 30% pyridine/CH₂Cl₂ (1.00 mL). After 12 hours, the solvent was concentrated and the residue purified via column chromatography (SiO₂, 40:1 CH₂Cl₂:Acetone) to afford a colorless solid, which was used without further purification (10.0 mg, 50%). Triethylamine (150 βL) was added to the carbonate (10.0 mg, 0.017 mmol) in MeOH (2.5 mL) and CH₂Cl₂ (2.50 mL). After 12 hours, the solvent was concentrated and the residue purified via column chromatography (SiO₂, 10:1 CH₂Cl₂:Acetone) to afford 26k as a colorless amorphous solid (6.00 mg, 46%, 23% over 3 steps): ¹H NMR (CDCl₃, 500 MHz) [symbol] 8.16 (s, 1H), 7.52 (s, 1H), 7.34-7.30 (m, 5H), 6.75 (s, 1H), 4.93 (d, J=5.0 Hz, 1H), 4.56-4.51 (m, 1H), 4.23 (t, J=4.0 Hz, 1H), 3.98-3.96 (m, 1H), 3.76 (s, 1H), 3.43 (s, 3H), 3.06 (d, J=4.3 Hz, 1H), 2.65 (s, 1H), 2.38 (s, 3H), 1.33 (dd, J=11.2, 6.1 Hz, 6H), 1.29 (s, 3H), 1.27 (s, 3H); ¹³C NMR (CDCl₃, 125 MHz) [symbol] 157.6, 152.1, 146.6, 146.0, 142.1, 134.5, 127.7, 127.5, 127.2, 122.2, 121.3 (2C), 120.2 (2C), 115.0 (2C), 108.1 (2C), 101.2, 81.6, 71.2, 68.9, 68.1, 66.5, 59.0, 24.8, 23.6, 20.8 (2C), 9.1; IR (film) νₘₐₓ cm⁻¹ 3406, 2930, 2375, 1705, 1522, 1394, 1229, 1205, 1111, 1078, 1049, 933, 793, 739, 698; HRMS (ESI⁺) m/z: [M+Na]⁺ calcd for C₃₀H₃₄N₂O₉, 589.2162. Found, 589.2111.

N-(7-((2R,3R,4S,5R)-3,4-dihydroxy-5-methoxy-6,6-dimethyltetrahydro-2H-pyran-2-yloxy)-5-methoxy-8-methyl-2-oxo-2H-chromen-3-yl)-1H-indole-2-carboxamide (261): Palladium on carbon (10%, 40 mg) was added to 25d (200 mg, 0.36 mmol) in anhydrous THF (2.40 mL) and the solution was placed under an atmosphere of H₂. After 12 hours, the solution was filtered through SiO₂ (1:1 CH₂Cl₂:Acetone) and the eluent was concentrated to afford a yellow solid, which was used without further purification (150 mg, 99%). EDCI (57.5 mg, 0.30 mmol) and 1H-indole-2-carboxylic acid (38.7 mg, 0.24 mmol) were added to the amine (50.6 mg, 0.12 mmol) in 30% pyridine/CH₂Cl₂ (3.30 mL). After 12 hours, the solvent was concentrated and the residue purified via column chromatography (SiO₂, 40:1 CH₂Cl₂:Acetone) to afford a colorless solid, which was used without further purification (26.3 mg, 39%). Triethylamine (150 μL) was added to the carbonate (26.3 mg, 0.047 mmol) in MeOH (2.00 mL) and CH₂Cl₂ (2.00 mL). After 48 hours, the solvent was concentrated and the residue purified via column chromatography (SiO₂, 40:1 CH₂Cl₂:Acetone) to afford 261 as a colorless amorphous solid (6.60 mg, 26%, 10% over 3 steps): ¹H NMR (CD₂Cl₂, 400 MHz) [symbol] 9.26 (s, 1H), 8.96 (s, 1H), 8.68 (s, 1H), 7.74 (d, J=8.1 Hz, 1H), 7.52 (d, J=8.3 Hz, 1H), 7.38-7.34 (m, 1H), 7.22-7.16 (m, 1H), 6.84 (s, 1H), 6.00 (s, 1H), 5.65 (d, J=1.7 Hz, 1H), 4.26-4.21 (m, 2H), 3.96 (s, 3H), 3.59 (s, 3H), 3.35 (d, J=8.6 Hz, 1H), 2.25 (s, 3H), 1.53 (s, 3H), 1.20 (s, 3H); ¹³C NMR (CDCl₃, 125 MHz) [symbol] 165.1, 157.4, 156.5, 149.7, 136.7, 134.7, 127.7, 125.3, 122.5, 121.1, 120.0, 111.9, 104.6, 103.8, 97.7, 94.0, 84.3, 84.2, 82.6, 69.6, 69.1, 66.1, 62.2, 62.0, 59.7, 23.1, 22.7, 14.2; IR (film) νₘₐₓ 3389, 2924, 2853, 1697, 1605, 1535, 1460, 1340, 1211, 1101, 1088, 962, 729 cm⁻¹; HRMS (ESI⁺) m/z: [M+H]⁺ calcd for C₂₈H₃₀N₂O₉, 539.2030. Found, 539.2056.

N-(8-benzyl-7-((2R,3R,4S,5R)-3,4-dihydroxy-5-methoxy-6,6-dimethyltetrahydro-2H-pyran-2-yloxy)-2-oxo-2H-chromen-3-yl)-1H-indole-2-carboxamide (26m): Palladium on carbon (10%, 46 mg) was added to 25e (230 mg, 0.38 mmol) in anhydrous THF (2.50 mL) and the solution was placed under an atmosphere of H₂. After 12 hours, the solution was filtered through SiO₂ (1:1 CH₂Cl₂:Acetone) and the eluent was concentrated to afford a yellow solid, which was used without further purification (177 mg, 99%). EDCI (61.5 mg, 0.32 mmol) and 1H-indole-2-carboxylic acid (41.4 mg, 0.26 mmol) were added to the amine (60.0 mg, 0.13 mmol) in 30% pyridine/CH₂Cl₂ (3.50 mL). After 12 hours, the solvent was concentrated and the residue purified via column chromatography (SiO₂, 3:1 Hexane:Ether→40:1 CH₂Cl₂:Acetone) to afford a yellow solid, which was used without further purification (66.2 mg, 85%). Triethylamine (150 μL) was added to the carbonate (66.2 mg, 0.11 mmol) in MeOH (2.50 mL) and CH₂Cl₂ (2.50 mL). After 12 hours, the solvent was concentrated and the residue purified via column chromatography (SiO₂, 40:1 CH₂Cl₂:Acetone) to afford 26m as a colorless amorphous solid (6.50 mg, 10%, 8.4% over 3 steps): ¹H NMR (CD₂Cl₂, 500 MHz) [symbol] 8.68 (s, 1H), 8.62 (s, 1H), 7.63-7.61 (m, 1H), 7.47 (dd, J=5.7, 3.3 Hz, 1H), 7.16-7.10 (m, 4H), 7.10-7.04 (m, 4H), 6.99 (t, J=8.2 Hz, 1H), 6.74 (d, J=8.0 Hz, 1H), 6.43 (dd, J=8.1, 0.7 Hz, 1H), 5.31 (d, J=2.9 Hz, 1H), 4.17 (t, J=6.8 Hz, 1H), 4.01 (dd, J=8.5, 2.9 Hz, 1H), 3.90 (d, J=13.1 Hz, 2H), 3.45 (s, 3H), 3.16 (d, J=8.6 Hz, 1H), 2.43 (s, 1H), 2.21 (s, 1H), 1.25 (s, 3H), 0.95 (s, 3H); ¹³C NMR (CDCl₃, 125 MHz) [symbol] 166.8, 155.4, 153.8, 140.2, 131.6, 130.2, 128.0, 127.6, 127.5 (2C), 127.4 (2C), 126.9, 125.1 (2C), 115.2, 108.3 (2C), 105.8 (2C), 97.1 (2C), 83.3 (2C), 77.2, 70.2, 67.9 (2C), 65.0, 60.6, 21.9, 13.1, 13.0; IR (film) νₘₐₓ 3333, 2961, 2926, 2854, 1717, 1601, 1466, 1261, 1090, 1076, 1041, 800, 750 cm⁻¹; HRMS (ESI⁺) m/z: [M+Na]⁺ calcd for C₃₃H₃₂N₂O₈, 607.2056. Found, 607.2056.

N-(7-((2R,3R,4S,5R)-3,4-dihydroxy-5-methoxy-6,6-dimethyltetrahydro-2H-pyran-2-yloxy)-2-oxo-8-phenyl-2H-chromen-3-yl)-1H-indole-2-carboxamide (26n): Palladium on carbon (10%, 14 mg) was added to 25f (68.0 mg, 0.12 mmol) in anhydrous THF (800 μL) and the solution was placed under an atmosphere of H₂. After 12 hours, the solution was filtered through SiO₂(1:1 CH₂Cl₂:Acetone) and the eluent was concentrated to afford a yellow solid, which was used without further purification (52.0 mg, 99%). EDCI (18.5 mg, 0.096 mmol) and 1H-indole-2-carboxylic acid (12.4 mg, 0.077 mmol) were added to the amine (17.5 mg, 0.039 mmol) in 30% pyridine/CH₂Cl₂ (1.10 mL). After 12 hours, the solvent was concentrated and the residue purified via column chromatography (SiO₂, 40:1 CH₂Cl₂:Acetone) to afford a colorless solid, which was used without further purification (8.20 mg, 36%). Triethylamine (150 μL) was added to the carbonate (8.2 mg, 0.014 mmol) in MeOH (1.00 mL) and CH₂Cl₂ (1.00 mL). After 48 hours, the solvent was concentrated and the residue purified via column chromatography (SiO₂, 40:1 CH₂Cl₂:Acetone) to afford 26n as a colorless amorphous solid (4.00 mg, 51%, 18% over 3 steps): ¹HNMR (CD₂Cl₂, 500 MHz) [symbol] 9.23 (s, 1H), 8.80 (s, 1H), 8.67 (s, 1H), 7.71 (dd, J=8.0, 0.7 Hz, 1H), 7.57 (d, J=8.8 Hz, 1H), 7.51-7.48 (m, 3H), 7.46-7.44 (m, 1H), 7.37-7.32 (m, 4H), 7.19-7.17 (m, 2H), 5.53 (d, J=2.4 Hz, 1H), 3.86 (s, 1H), 3.76-3.73 (m, 2H), 3.51 (s, 3H), 3.23 (d, J=9.1 Hz, 1H), 2.41 (s, 1H), 1.34 (s, 3H), 1.05 (s, 3H); ¹³C NMR (CD₂Cl₂, 125 MHz) δ 159.1, 157.9, 154.5, 147.3, 136.0 (2C), 130.7 (2C), 129.8, 129.2, 127.3, 127.1, 127.0, 126.9, 124.5, 122.8, 121.6, 121.2, 120.3, 113.7, 111.4, 111.1, 103.1 (2C), 97.5, 83.2, 77.7, 70.1, 67.5, 60.8, 21.9, 21.7; IR (film) νₘₐₓ 3427, 2961, 2924, 2853, 2062, 1643, 1614, 1537, 1362, 1236, 1094, 1041, 962, 791, 739, 698 cm⁻¹; HRMS (ESI⁺) m/z: [M+Na]⁺ calcd for C₃₂H₃₀N₂O₈, 593.1900. Found, 593.1890.

N-(7-((2S,3R,4S,5R)-3,4-dihydroxy-5-methoxy-6,6-dimethyltetrahydro-2H-pyran-2-yloxy)-8-methoxy-2-oxo-2H-chromen-3-yl)-1H-indole-2-carboxamide (260): Palladium on carbon (10%, 47 mg) was added to 25g (237 mg, 0.44 mmol) in anhydrous THF (2.93 mL) and the solution was placed under an atmosphere of H₂. After 12 hours, the solution was filtered through SiO₂ (1:1 CH₂Cl₂:Acetone) and the eluent was concentrated to afford a yellow solid, which was used without further purification (177 mg, 99%). EDCI (69.4 mg, 0.36 mmol) and 1H-indole-2-carboxylic acid (46.7 mg, 0.29 mmol) were added to the amine (59.0 mg, 0.14 mmol) in 30% pyridine/CH₂Cl₂ (4.00 mL). After 12 hours, the solvent was concentrated and the residue purified via column chromatography (SiO₂, 3:1 Hexane:Ether→40:1 CH₂Cl₂:Acetone) to afford a colorless solid, which was used without further purification (32.0 mg, 49%). Triethylamine (150 μL) was added to the carbonate (32.0 mg, 0.071 mmol) in MeOH (2.00 mL) and CH₂Cl₂ (2.00 mL). After 48 hours, the solvent was concentrated and the residue purified via column chromatography (SiO₂, 3:1 CH₂Cl₂:Acetone) to afford 26o as a colorless amorphous solid (22.1 mg, 73%, 35% over 3 steps): ¹H NMR (CD₂Cl₂, 400 MHz) [symbol] 9.28 (s, 1H), 8.78 (s, 1H), 7.77 (d, J=8.1 Hz, 1H), 7.53 (dd, J=8.3, 0.8 Hz, 1H), 7.38 (m, 1H), 7.31 (s, 2H), 7.24 (d, J=0.9 Hz, 1H), 7.22-7.20 (m, 1H), 6.02 (s, 1H), 5.62 (d, J=2.3 Hz, 1H), 4.25 (t, J=3.5 Hz, 1H), 3.99 (s, 3H), 3.75 (dd, J=9.0, 3.6 Hz, 1H), 3.62 (s, 3H), 3.13 (d, J=3.6 Hz, 1H), 1.30 (s, 3H), 1.27 (s, 3H); ¹³C NMR (CDCl₃, 125 MHz) [symbol] 163.8, 159.1, 157.7, 150.6, 143.3, 136.0, 135.8, 129.2, 126.9, 124.6, 122.8, 121.8, 121.6, 121.4, 120.3, 114.4, 112.5, 111.2, 103.1, 98.0, 83.2, 77.9, 74.0, 60.9, 58.7, 22.3, 21.8; IR (film) νₘₐₓ 3420, 2957, 2924, 2854, 2359, 1653, 1558, 1541, 1246, 1001, 798 cm⁻¹; HRMS (ESI⁺) m/z: [M+H]⁺ calcd for C₂₇H₂₈N₂O₉, 525.1873. Found, 525.1875.

N-(7-((2R,3R,4S,5R)-3,4-dihydroxy-5-methoxy-6,6-dimethyltetrahydro-2H-pyran-2-yloxy)-8-ethyl-2-oxo-2H-chromen-3-yl)-1H-indole-2-carboxamide (26p): Palladium on carbon (10%, 12 mg) was added to 25h (121 mg, 0.22 mmol) in anhydrous THF (5.00 mL) and the solution was placed under an atmosphere of H₂. After 12 hours, the solution was filtered through SiO₂(1:1 CH₂Cl₂:Acetone) and the eluent was concentrated to afford a yellow solid, which was used without further purification (90.0 mg, 99%). EDCI (28.3 mg, 0.15 mmol) and 1H-indole-2-carboxylic acid (19.0 mg, 0.12 mmol) were added to the amine (24.0 mg, 0.059 mmol) in 30% pyridine/CH₂Cl₂ (1.63 mL). After 12 hours, the solvent was concentrated and the residue purified via column chromatography (SiO₂, 40:1 CH₂Cl₂:Acetone) to afford an colorless foam, which was used without further purification (23.8 mg, 73%). Triethylamine (150 μL) was added to the carbonate (14.1 mg, 0.026 mmol) in MeOH (1.50 mL). After 12 hours, the solvent was concentrated and the residue purified via column chromatography (SiO₂, 40:1 CH₂Cl₂:Acetone) to afford 26p as a yellow amorphous solid (5.00 mg, 37%, 27% over 3 steps): ¹H NMR (CD₂Cl₂, 500 MHz) [symbol] 8.65 (s, 1H), 7.62 (d, J=8.0 Hz, 1H), 7.41 (d, J=8.5 Hz, 1H), 7.33 (d, J=8.5 Hz, 1H), 7.23 (t, J=7.8 Hz, 1H), 7.20 (d, J=8.5 Hz, 2H), 7.13 (s, 2H), 7.07 (t, J=7.5 Hz, 1H), 5.46 (d, J=2.0 Hz, 1H), 4.07 (dd, J=9.3, 3.5 Hz, 1H), 4.04 (t, J=3.5 Hz, 1H), 3.51 (s, 3H), 3.25 (d, J=7.2 Hz, 1H), 2.78 (q, J=7.0 Hz, 2H), 1.27 (s, 3H), 1.10 (t, J=7.5 Hz, 3H), 1.06 (s, 3H); ¹³C NMR (CD₂Cl₂, 125 MHz) [symbol] 158.4, 157.3, 154.2, 146.8, 135.3, 128.1, 125.5, 124.0, 123.0, 123.0, 122.8, 120.1, 119.5, 118.7, 118.2, 112.0, 110.1, 109.7, 102.4, 97.0, 82.1, 76.5, 69.3, 66.4, 26.7, 20.3, 14.3, 11.4; IR (film) νₘₐₓ 3435, 3416, 2974, 2935, 2469, 2359, 2339, 1715, 1651, 1520, 1456, 1435, 1379, 1354, 1259, 1180, 1113, 1088, 1026, 997, 962, 798, 739 cm⁻¹; HRMS (ESI⁺) m/z: [M+Na]⁺ calcd for C₂₈H₃₀N₂O₈, 545.1900. Found, 545.1909.

### Example 14

### Heterocyclic Side Chain Derivatives (Triazoles)

In this example, the novobiocin analogues having modified side chain derivatives can be prepared. In particular, triazole derivatives can be prepared. A preferred triazole derivative the amide is substituted according to

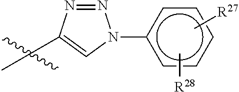

wherein R²⁷ is hydrogen, hydroxyl, alkoxy, or aryloxy; and
wherein R²⁸ is hydrogen, alkoxy, aryloxy, or amino. The coumarin amine can be acylated with popargylic acid to form the corresponding amide, the alkyne of which can be reacted with carefully chosen azides to afford the desired triazole products according to the following Scheme 22. The aromatic azides can consist mainly of two substitution patterns, both of which are meta to the azide and can allow for additional van Der Walls interactions with the binding pockets by the inclusion of alkylated amines and/or ethers.

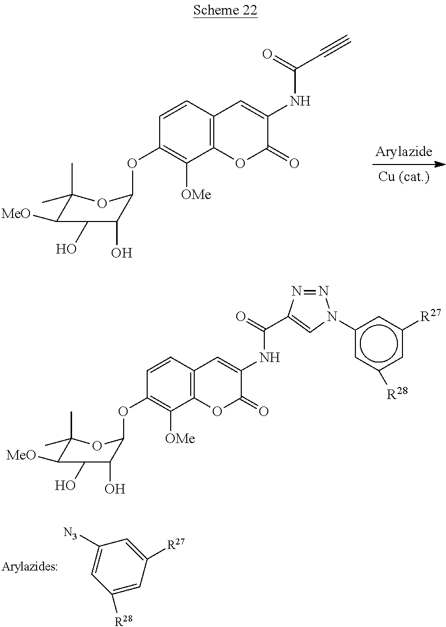

wherein R²⁷ is hydrogen, hydroxyl, alkoxy, or aryloxy (most preferably hydroxyl, methoxy, ethloxy, propoxy, and phenoxy); and wherein R²⁸ is hydrogen, alkoxy, aryloxy, or amino (most preferably hydrogen, methoxy, ethoxy, propoxy, phenoxy, —NH₂, or —N(CH₃)₂).

It will be appreciated that the aforementioned scheme illustrates the preferred location of the R²⁷ and R²⁸ substituents.

### Example 15

### Heterocycle Side Chain Derivatives (Biarylamines and Biaryl Ethers)

In this example, the novobiocin analogues having modified side chain derivatives can be prepared. In particular, biarylamines and biarylethers that target the B subdomain can be prepared. In particular, amides having the following biaryl substitution can be prepared:

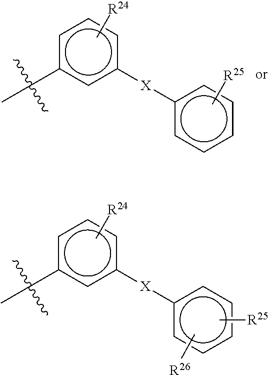

wherein X is ether or amino (most preferably —O—, —NH—, or —NCH₃—);
wherein R²⁴ is alkoxy (most preferably methoxy);
wherein R²⁵ is hydrogen, hydroxyl, alkoxy, or aryloxy alkyl (most preferably hydroxyl, methoxy, propoxy, or phenoxy); and
wherein R26 is hydrogen, alkoxy, aryloxy, or amino (most preferably hydrogen, methoxy, ethoxy, propoxy, phenoxy, —NH₂, or —N(CH₃)₂).

The m-iodo benzamide shown in the Scheme 23 below can be utilized to perform cross coupling reactions with amines and phenols, allowing us to further diversify the second aromatic ring in an effort to achieve greater interactions with the binding pocket. Upon solvolysis of the cyclic carbonate with methanolic triethylamine, the corresponding product can be afforded.

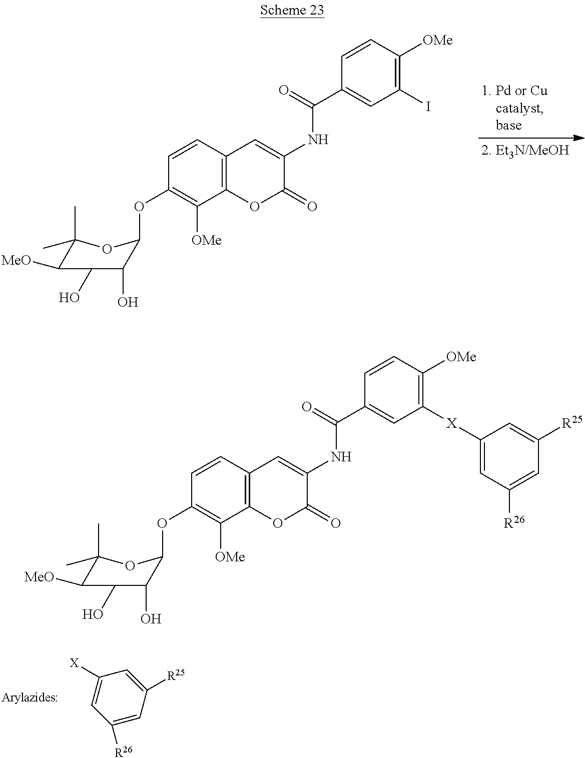

wherein X is ether or amino (most preferably —O—, —NH—, or —NCH₃—);
wherein R²⁵ is hydrogen, hydroxyl, alkoxy, or aryloxy alkyl (most preferably hydroxyl, methoxy, propoxy, or phenoxy); and
wherein R²⁶ is hydrogen, alkoxy, aryloxy, or amino (most preferably hydrogen, methoxy, ethoxy, propoxy, phenoxy, —NH₂, or —N(CH₃)₂).

It will be appreciated that the aforementioned Scheme 23 illustrates the preferred location of the R²⁴, R²⁵, and R²⁶ substituents.

### Example 16

### Heterocycle Side Chain Derivatives (Oxazoles and Pyridines)

In this example, the novobiocin analogues having modified side chain derivatives can be prepared. In particular, benzoxazoles as indole mimics can be prepared. The R²⁹ and R³⁰ substituents of the benzoxazole core project into regions that have been observed as beneficial for Hsp90 inhibition. Thus, in one aspect, the amide side chain is define according to:

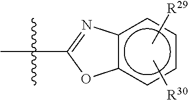

or more preferably

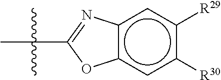

wherein R²⁹ is hydrogen, alkoxy, or amino; and
wherein R³⁰ is hydrogen, alkoxy, or aryloxy.

These side chains can be prepared by coupling a variety of commercially or readily available ortho aminophenols with oxalic acid monomethyl ester to form the requisite amide as set forth in the Scheme 24 below. Simply heating this compound in the presence of acid or treatment with P(O)Cl₃ is known to furnish the benzoxazole ring system. In addition to the molecules drawn, corresponding pyridine analogues, especially the derivative that contains a nitrogen atom in lieu of the C—R functionality, can also be prepared

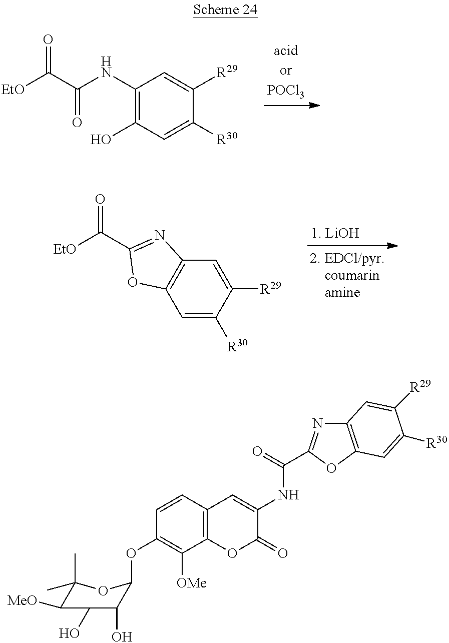

Wherein R²⁹ is hydrogen, alkoxy, amino (most preferably hydrogen, methoxy, ethoxy, propoxy, —NHCH₃, or —N(CH₃)₂); and wherein R³⁰ is hydrogen, alkoxy, aryloxy; (most preferably hydrogen, methoxy, ethoxy, phenoxy; or phenoxy), wherein R³⁰ is hydrogen, alkoxy, or aryloxy.

It will be appreciated that the aforementioned Scheme 24 illustrates the preferred location of the R²⁹ and R³⁰ substituents.

### Example 17

### Design Scaffold for Modified Sugar Analogues of Novobiocin

Modified sugar analogues of the novobiocin scaffold were designed and synthesized to elucidate structure-activity relationships for the noviose sugar of novobiocin. N-heterocycles are found in a wide variety of bioactive compounds, and polyhydroxyl azasugars mimic natural-sugars found in the body and act as potent inhibitors of the enzyme glycosidase. The azasugar analogues, with nitrogen inserted at various positions within the ring structure, sought to probe the hydrogen-bonding interactions with the binding pocket as well as improve solubility. The corresponding cyclohexyl analogues were designed to examine whether it was simply a hydrophobic group that was necessary to fill the sugar binding pocket. Additionally, derivatives having a single methyl group instead of the gem-dimethyl moiety present on the parent compound, as well as an unsubstituted derivative, were prepared. Finally, these analogues aimed to determine whether the diol was a necessary component for Hsp90 inhibition.

The biaryl benzamide side chain was selected were based upon previously obtained SAR for the amide side chain as described in Burlison, J. A., Avila, C., Vielhauer, G., Lubbers, D. J., Holzbeierlein, J., Blagg, B. S. J. J. Org. Chem., 73, 2130-2137 (2008). However, it will be appreciated that other side chains at the 3-position are well within the scope of the present disclosure.

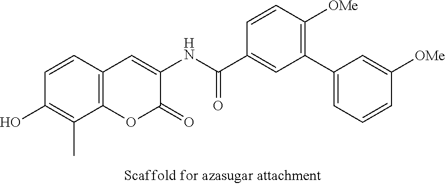

The analogues were assembled in a modular fashion allowing sequential coupling of various sugars and the biaryl acid chloride with the desired scaffold. A Mitsunobu ether coupling reaction between the coumarin phenol 1 and a sugar mimic 2 yielded the desired sugars in good yields.

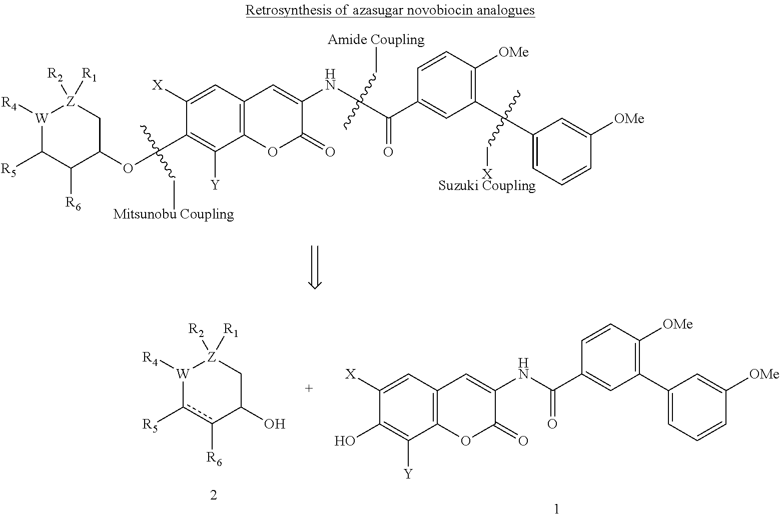

More specifically, as seen in Scheme 25 below, the phenol functionality of 5 was quantitatively protected as the corresponding ester 6 using 30% acetic anhydride in pyridine. The free amine 7 was liberated through hydrogenolysis and coupled with the biaryl acyl chloride 9, which was generated from the corresponding biaryl acid 8. Finally, solvolysis of ester 50 by triethylamine in methanol afforded phenol 1 in good yield. See Burlison, J. A., Avila, C., Vielhauer, G., Lubbers, D. J., Holzbeierlein, J., Blagg, B. S. J. J. Org. Chem., 73, 2130-2137 (2008); Donnelly, A. C., Mays, J. R., Burlison, J. A., Nelson, J. T., Vielhauer, G., Holzbeierlein, J., Blagg, B. S. J. J. Org. Chem., 73, 8901-8920 (2008).

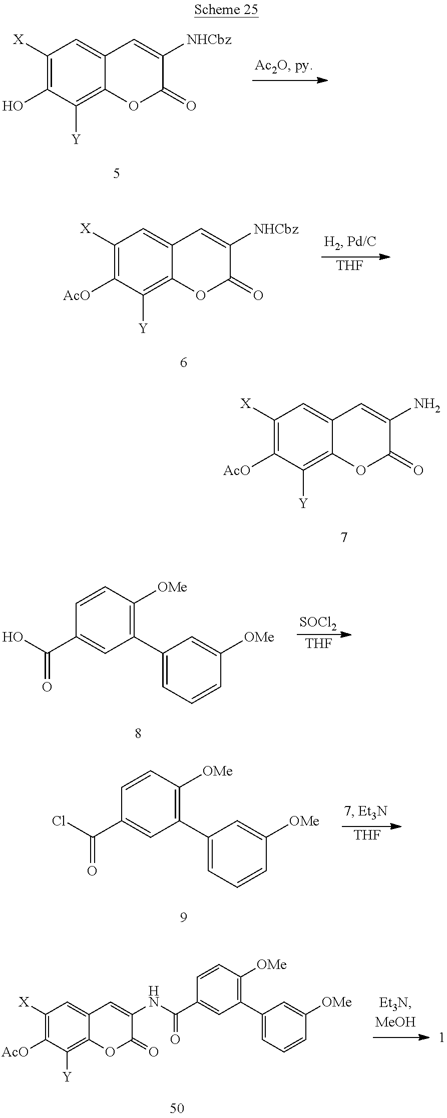

### Synthesis of 1,3-Azasugar Mimics

In order to avoid synthetic complexity, only a single hydroxyl group was installed on the piperidine ring prior to Mitsunobu coupling and the amine was masked by a Boc group (16). As outlined in Scheme 26 below, this compound was synthesized in three steps, starting with the regioselective butadiene monoxide ring opening with allylamine to yield 13 and 14 in a 3:1 ratio. Subsequent protection of the amine with Boc anhydride followed by RCM metathesis in the presence of Grubbs II catalyst afforded compound 16 in good yield.

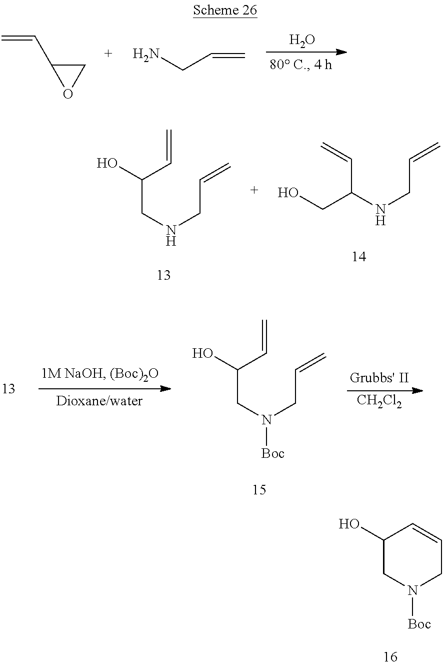

With compound 1 and 16 in hand, compound 17 was synthesized with Mitsunobu conditions as shown in Scheme 27. Deprotection of 17 with either 10% TFA/CH₂Cl₂ or AcCl/MeOH produced amine 18 in quantitative yield. Treatment of 18 with 1 equivalent methyl iodine and excess potassium carbonate gave tertiary amine 19, which was either dihydroxylated using OsO₄/NMO to generate 20 or reduced using 10% Pd/C in THF to furnish 21. Compounds 23 and 25 were synthesized from compound 17 in good yield using the same conditions, and subsequently deprotected by 10% TFA/CH₂Cl₂ to afford compounds 24 and 26, respectively. Acylation of compound 18 with 30% acetic anhydride/pyridine solution yielded 27, which was either reduced to compound 28 or dihydroxylated to compound 29, as discussed previously.

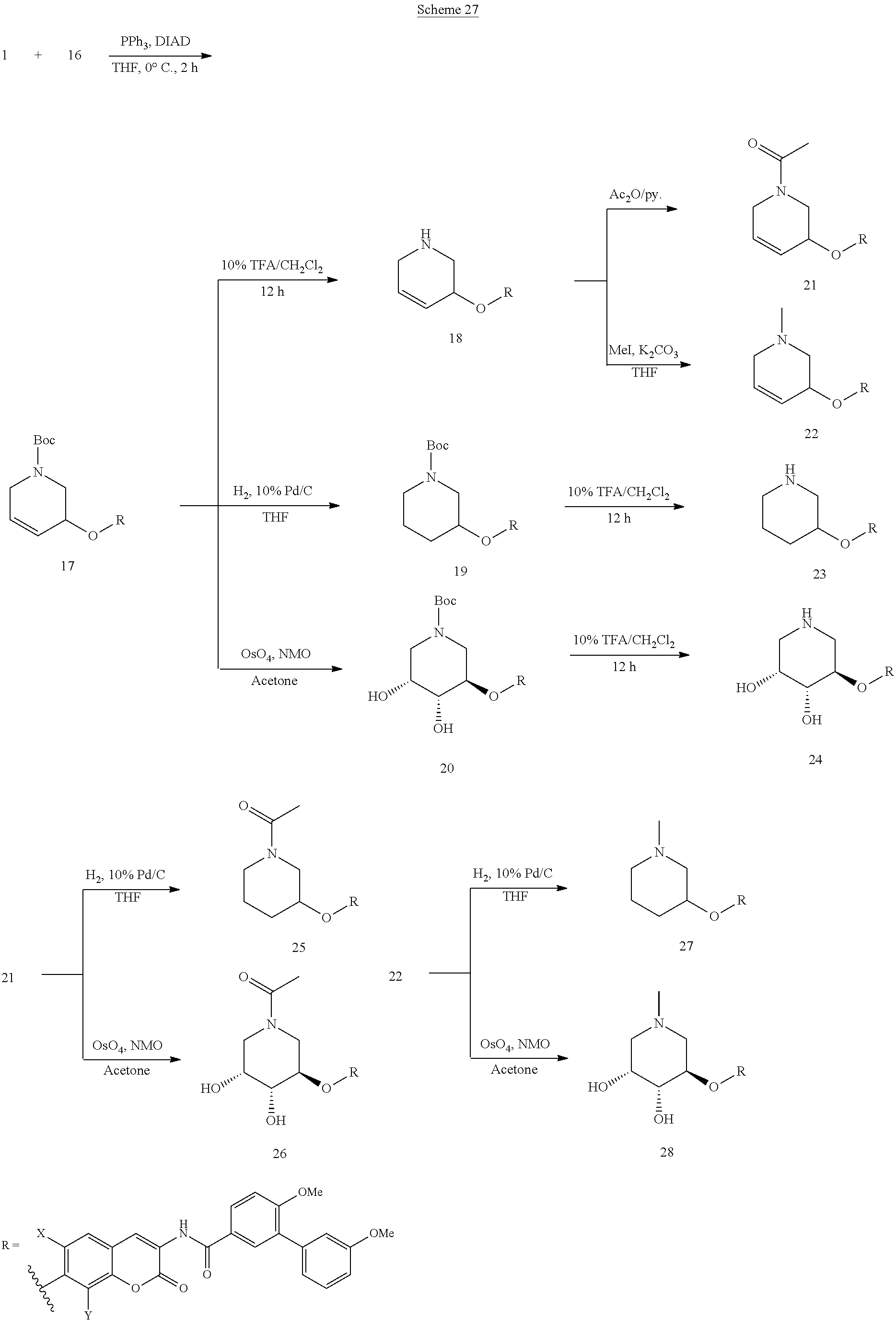

### Synthesis of 1,4-Azasugar Mimics

To elucidate the effect of different substitution patterns on the piperidine ring, a Boc-protected 1,4-azasugar was coupled with phenol 1 to give compound 29. Subsequent deprotection led to compound 30, which was then methylated or acetylated to afford compound 31 and 32 in good yield. The synthesis of these compounds is illustrated in Scheme 28 below.

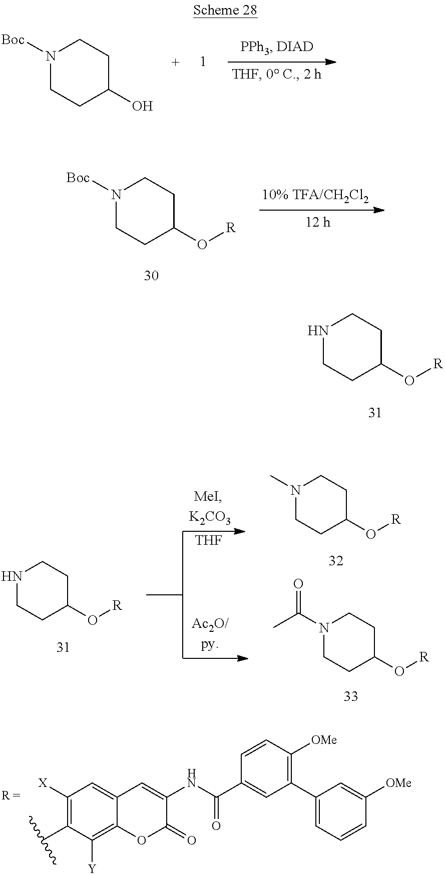

### Cyclohexyl Sugar Analogues

Many of the simplified cyclohexyl sugar mimics were accessible using common procedures (Scheme 29), such as one-pot reductions with lithium aluminum hydride to produce compounds 34 and 35, or via Luche conditions to yield 36. Compounds containing a double bond were designed to be coupled to the scaffold and subsequently dihydroxylated to give the corresponding diols (37-39) as shown in the scheme below.

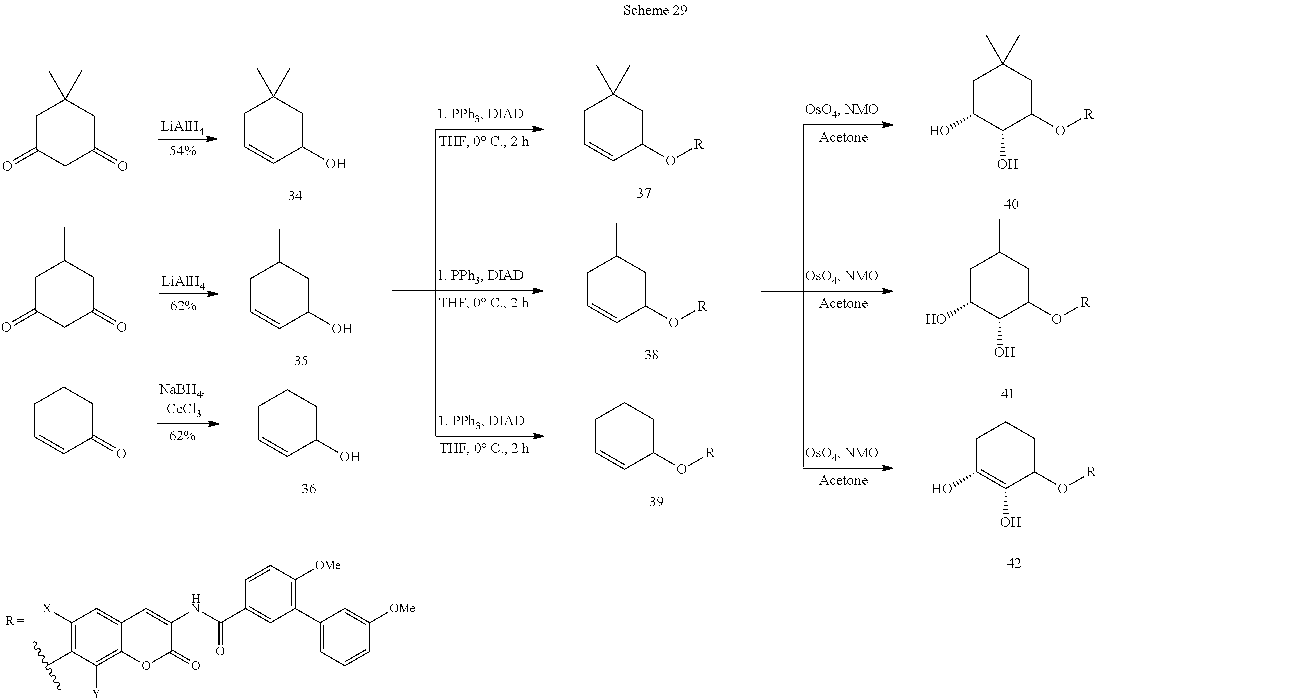

### Straight Chain Analogues

The cyclic azasugar and cyclohexyl sugar analogues allow only a limited range of conformations into which the sugar portion can orient itself. A small series of corresponding aliphatic chain sugar mimics were designed to allow more flexibility to explore the possibility of additional interactions outside of those allowed by the constrained ring structures. An aliphatic amine and dihydroxylated aliphatic chain were appended to the coumarin core 1 through standard Mitsunobu coupling of the Boc-protected amine and addition of allyl bromide followed by subsequent dihydroxylation, respectively (Scheme 30).

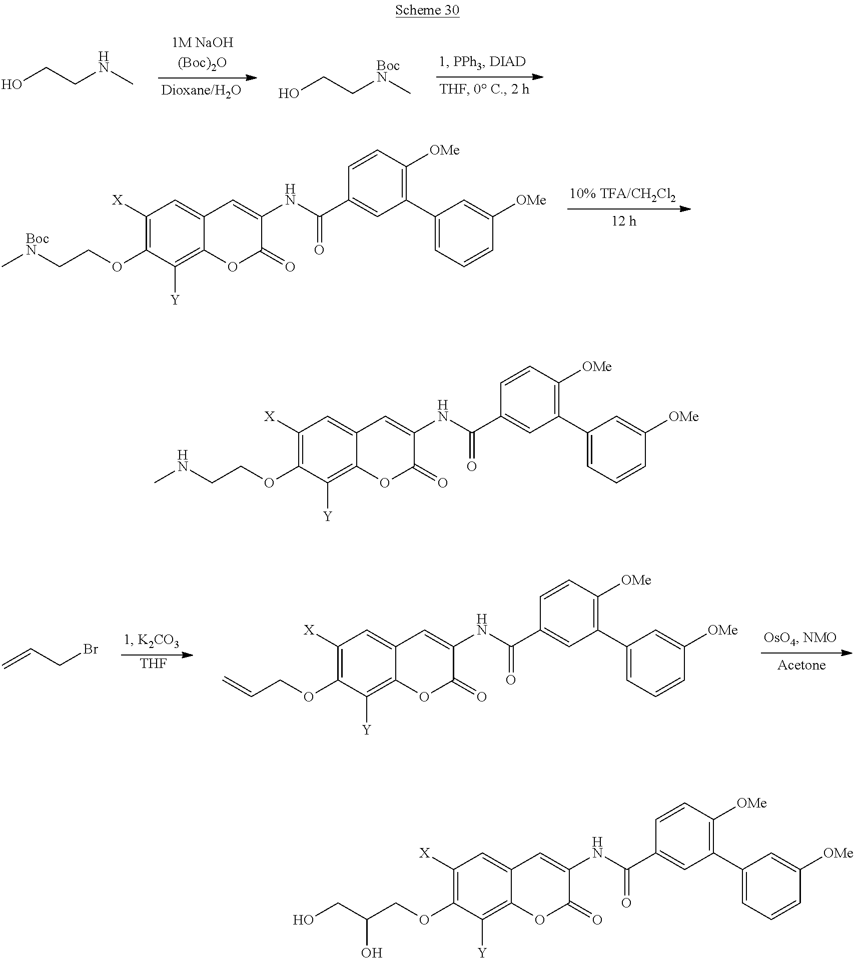

### Furanose, Pyranose, and Oxepanose Derivatives

In addition to these simplified azasugars and corresponding cyclohexyl derivatives, a series of sugars of variable ring size and substitution was synthesized to further probe the binding pocket. Although noviose is a six-membered sugar, the ability of the pocket to better accommodate a sugar of a different size has never been explored or optimized. Thus, 5- and 7-membered sugars were synthesized and coupled to the same aforementioned scaffolds to probe the pocket dimensions and attempt to establish additional favorable interactions. Moreover, although noviose is appended to novobiocin as a single anomer, the β-anomer of alternate sugars may prove more active through properly orienting the sugar in the pocket. The α- and β-anomer of each sugar were separated and tested to identify the most potent in each pair. With these considerations, a set of mono-, di- and trihydroxylated furanoses, pyranoses and oxepanose sugars E-L was designed as shown below. These sugars were synthesized by following the literature protocols previously reported by Blagg and coworkers. See Yu et al., Synthesis of Mono- and dihydroxylated furanoses, pyranoses, and an oxepanose for the Preparation of Natural Product Analogue Libraries, J. Org. Chem. 70, 5599-5605 (2005), which is incorporated by reference in its entirety.

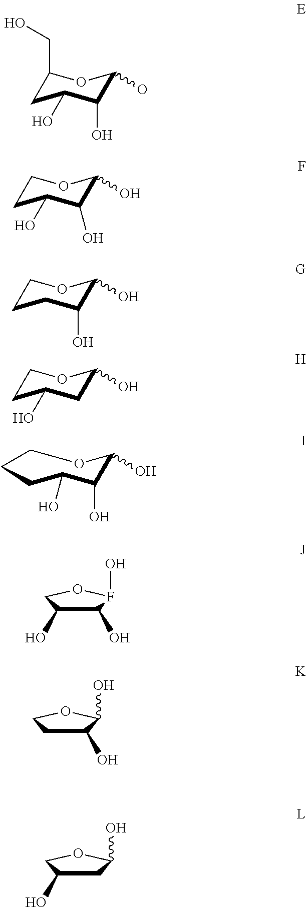

The analogues were assembled as discussed previously, in a modular fashion allowing sequential coupling of various sugars and the biaryl acid chloride with the desired scaffold, as shown in the scheme below. A Mitsunobu ether coupling reaction between the coumarin phenol 1 and sugars E-L yielded the desired analogues in good yields.

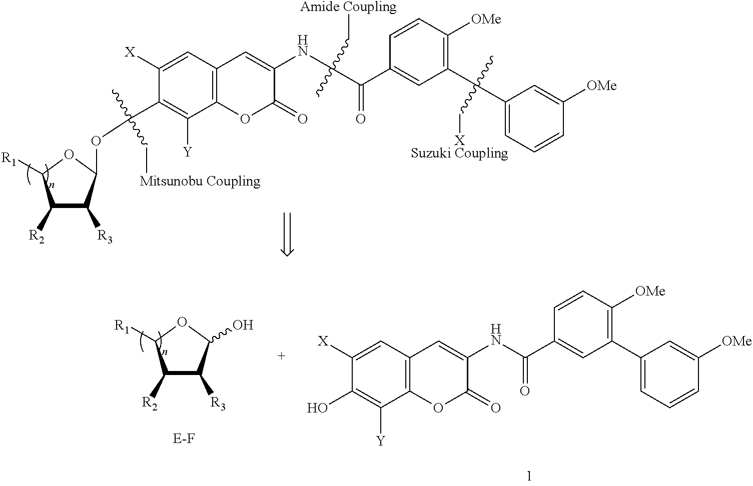

Coumarin phenol 1 and protected sugar F were coupled under Mitsunobu conditions using PPh₃ and diisopropylazadicarboxylate (DIAD) in THF at room temperature for one hour, yielding an inseparable mixture of diastereomers 10a and 10b in a 3:2 ratio (Scheme 31). The cyclic carbonates of the mixture of 10a and 10b were solvolyzed with 5 eq. of lithium hydroxide in MeOH: THF: H₂O (1:3:1) to afford a mixture of diols 11a and 11b in 64% yield. The two diastereomers 11a and 11b were easily separated by flash column chromatography. The assignment of stereochemistry at the anomeric carbon was accomplished by NOESY experiments.

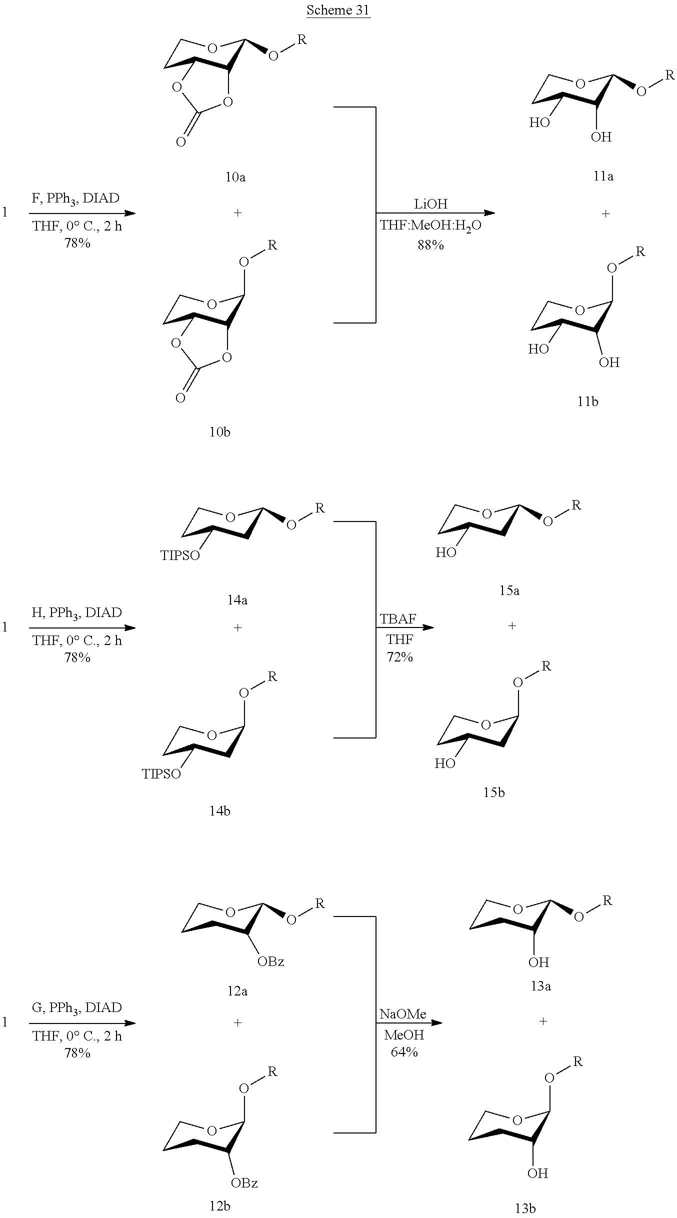

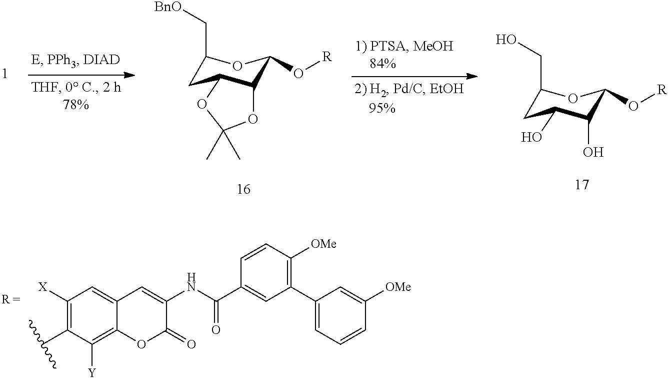

Compounds 12a and 12b and 14a and 14b were synthesized by coupling protected sugars G and H, respectively, with coumarin 1 under standard Mitsunobu conditions. Benzoyl (Bz) deprotection of 12a and 12b was accomplished using sodium methoxide in methanol at 0° C. for 10 minutes to yield 13a and 13b in a 3:2 ratio. TIPS deprotection of 14a and 14b was achieved using TBAF in THF at room temperature for one hour to furnish 15a and 15b in a 3:2 ratio. Interestingly, a single diastereomer 16 was exclusively formed when coumarin 1 and protected sugar E were subjected to standard Mitsunobu conditions. Subsequent deprotection of the acetonide and benzyl protecting groups by treatment with a catalytic amount of PTSA in methanol and hydrogenolysis, respectively, afforded triol 17 in 80% overall yield.

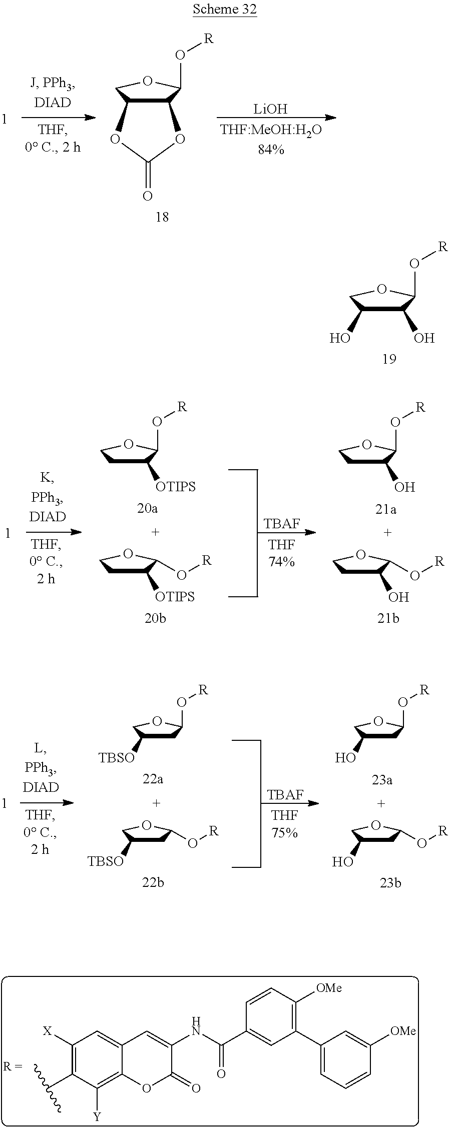

As shown in Scheme 32, protected furanose sugars J-L were coupled to coumarin 1 using Mitsunobu conditions to afford 18, 20a and 20b and 22a and 22b in 54-76% yields. Exclusively diastereomer 18 was formed through the Mitsunobu coupling between protected sugar J and coumarin 1. Hydrolysis of cyclic carbonate in 18 was performed with 5 eq. of lithium hydroxide at room temperature over one hour to obtain diol 19 in 84% yield. The removal of silyl groups in compounds 20a and 20b and 22a and 22b was achieved with TBAF in THF over one hour to furnish compounds 21a and 21b and 23a and 23b, respectively.

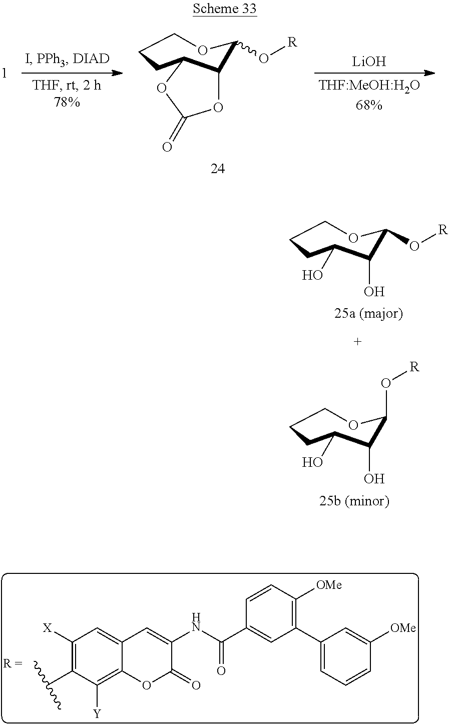

The protected oxepanose (I) was coupled to coumarin 1 using Mitsunobu conditions to afford a diastereomeric mixture of compounds 24. The mixture was hydrolyzed with lithium hydroxide to afford separable diastereomers 25a and 25b in a 3:2 ratio with 68% overall yield as shown in Scheme 33 above.

### Example 18

### Phosphate and Carbamate Sugar Analogues

In addition to the sugar analogues synthesized, several simplified non-sugar molecules were appended in place of the noviose sugar as shown in Scheme 34. The various carbamates were installed through lewis acid catalysis with the corresponding reagents. These two carbamates allow exploration of hydrogen bonding with the nucleotide binding pocket while offering much smaller groups than the bulky noviose sugar. The phosphate ester was introduced through an esterification reaction. This phosphate ester is very different from the noviose sugar, while still offering the ability to interact with the pocket and explore the substituent tolerance of the region in which the noviose sugar resides. Moreover, a phosphate ester can increase the hydrophilicity of the inhibitor, an auspicious trait when considering its potential use as a drug.

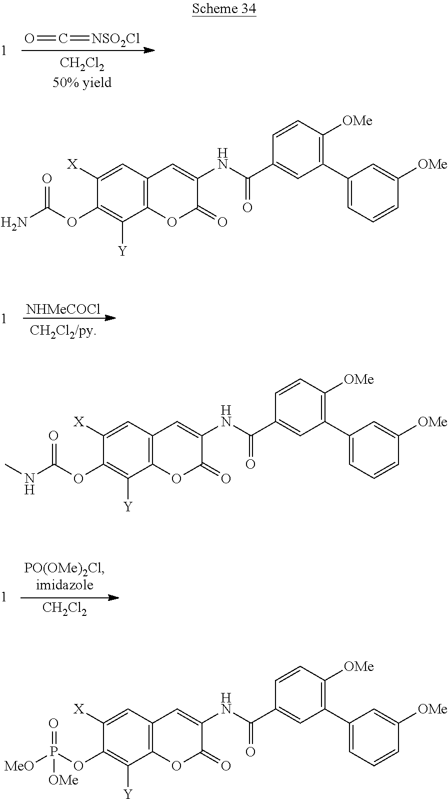

### Mesylate, Tosylate and Dimethyl Carbamate Analogues

As seen in Scheme 35, a series of mesylate, tosylate, and dimethyl carbamate analogues were synthesized starting once again from the various coumarins. Although the tosylate group has been explored by the Renoir group, the coumarin scaffolds are very dissimilar when comparing the analogues. While the mesylate and tosylate explore both the hydrogen bonding network and dimensions of the pocket, the dimethyl carbamate offers contrast with the previously discussed carbamates, specifically with the hydrogen bonding capabilities and space occupied. The desired functional group was installed on the phenol coumarin using the chloride of each corresponding group, in the presence of pyridine. Next, the benzyl carbonate was removed via hydrogenolysis to produce the aminocoumarin, which was readily coupled with the biaryl benzoic acid in the presence of N-(3-dimethylamino-propyl)-N′-ethylcarbodiimide hydrochloride (EDCI) and pyridine.

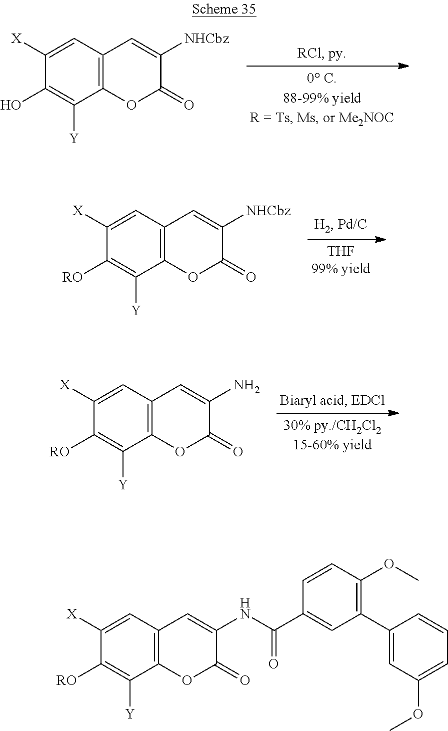

wherein R is tosylate, mesylate, or carbamate.

### Example 19

### Other Analogs

Several additional analogues were synthesized to probe the importance of the 7-phenol of the coumarin ring, specifically. These analogues incorporate similar functionalities, but these groups are attached directly to the ring without the oxygen spacer. In addition to probing the interactions of this oxygen with the binding pocket, these analogues explore the possibility that the sugar or non-sugar side chain is cleaved upon entering the cell, exposing a free phenol to interaction with the target. In order to access these analogues, the benzaldehyde precursor was alkylated in the presence of HCl gas to yield the desired alkyl chloride in modest yield. The desired 3-position regioisomer had to be separated from the 5-position isomer through several purifications. An aqueous treatment of the alkyl chloride with calcium carbonate afforded the desired benzylic alcohol in good yield. Next, the resulting benzaldehyde was converted to its formate ester via Baeyer-Villiger oxidation, and then hydrolyzed to afford the corresponding phenol. The benzylic alcohol was subsequently oxidized to form the desired benzoic acid and then standard coumarin formation procedures yielded the desired coumarin scaffold. Next, the benzyl carbonate was removed via hydrogenolysis to produce the aminocoumarin, which was readily coupled with the biaryl benzoic acid in the presence of N-(3-dimethylamino-propyl)-N-ethylcarbodiimide hydrochloride (EDCI) and pyridine as shown in the Scheme 36 below.

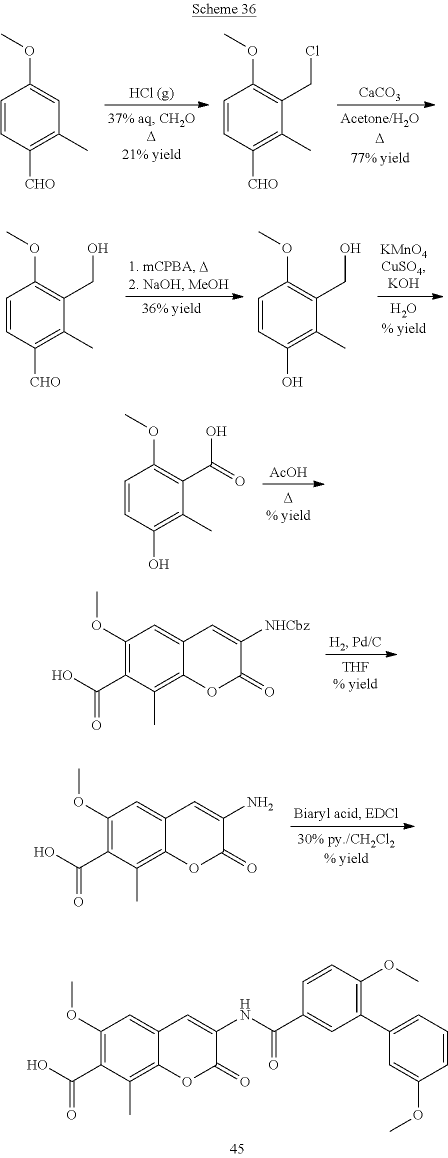

As shown in the Scheme 37 below, the 7-benzoic acid coumarin was converted to the corresponding benzyl alcohol and methyl ether through hydride reduction and esterification, respectively. In addition, a benzamide was installed through a Curtius rearrangement, then subsequently acetylated under standard coupling conditions. Finally, through Sandmeyer conditions, the 7-position benzamide was converted to a 7-position iodide. Standard Suzuki coupling conditions allowed functionalization of this position with 4-pyridine. These analogues allow further probing of key interactions with the sugar binding pocket.

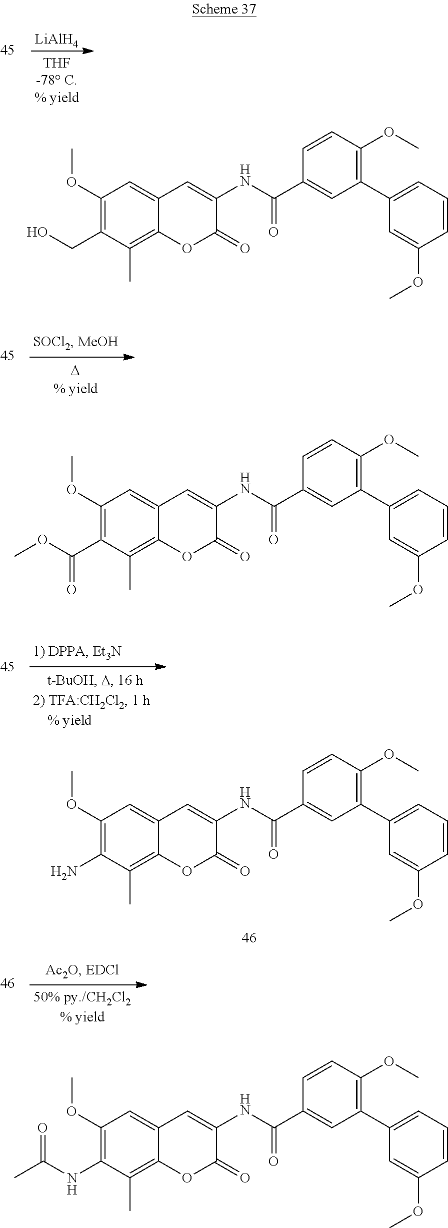

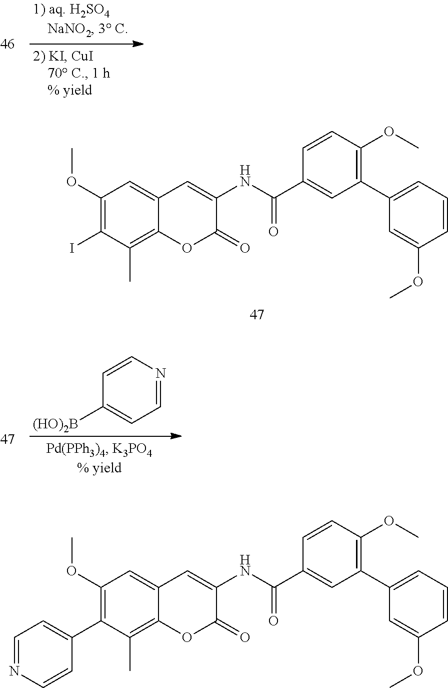

### Example 20

### Novobiocin Analogs with Mono- or Dihydroxylated Furanose or Pyranose Noviose Replacements

The presence of sugar moieties in natural products is known to play a vital role in solubility, activity and bioavailability for these compounds. Furthermore, the ring size can impart significant affinity towards their target protein. With these considerations in mind, a series of mono- and di-hydroxylated furanose and pyranose sugars (1-5 shown below) were synthesized according to previously disclosed procedures²⁶ for incorporation onto the novobiocin scaffold

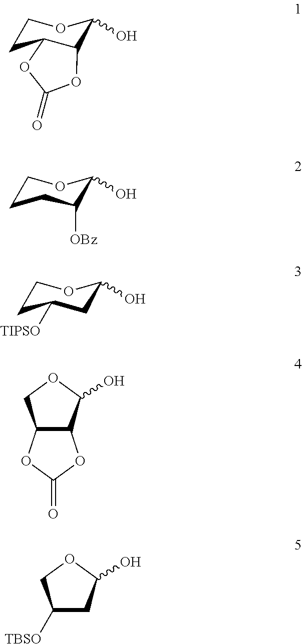

Incorporation of sugars 1-5 was envisioned to occur via a Mitsunobu coupling procedure between protected pyranoses and furanoses with phenol 6, as shown retrosynthetically below.

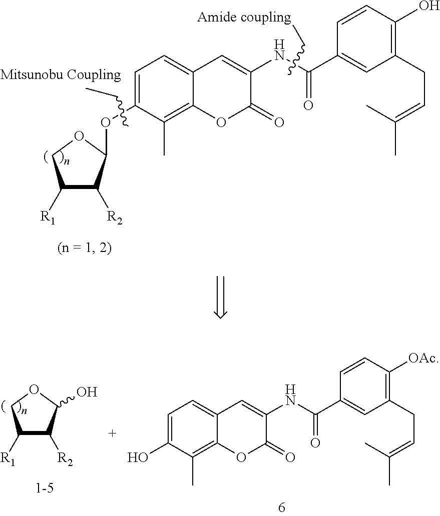

The preparation of intermediate 6 is described and illustrated in Scheme 38 below. The coumarin phenol 7 was converted to the methoxymethyl ether using methoxymethyl chloride and Hunig's base in dimethylformamide. The free aniline, liberated through hydrogenolysis with 10% Pd/C and hydrogen in tetrahydrofuran from 7, was coupled with acid chloride 9 to give benzamide 10. Subsequent cleavage of the methoxymethyl ether with 4N hydrochloride in dioxane provided phenol 6 in high yield.

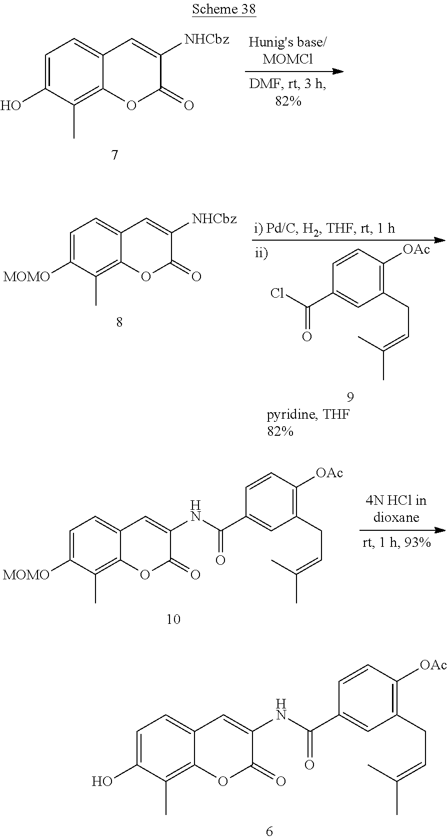

Once prepared, the phenol of 6 was coupled with sugars 1-5 under Mitsunobu conditions to give an inseparable diastereomeric mixture of 11-13 and 15 (Scheme 39). In the case of compound 14, a single diastereomer was formed. Subsequent hydrolysis of the cyclic carbonates and acetyl esters of 11a-b and 14 with lithium hydroxide in THF/MeOH/H₂O (3:2:2, v/v) afforded a diastereomeric mixture of 16a-b and 19, respectively. At this stage, diastereomers 16a and 16b were separated by chromatography. The assignment of stereochemistry at the anomeric center was established through two-dimensional NMR studies utilizing NOESY. In a similar manner, hydrolysis of the benzoyl and acetyl ester of 12 upon treatment with NaOMe/MeOH yielded 17a and 17b, which could be separated by chromatography. The tri-isopropylsilyl of 13 and tert-butyldimethylsilyl groups of 15 were removed by the addition of tetrabutylammonium fluoride to give separable 18a and 18b, and 20a and 20b, respectively. Syntheses of novobiocin analogues containing mono- and di-hydroxylated furanoses and pyranoses are shown below.

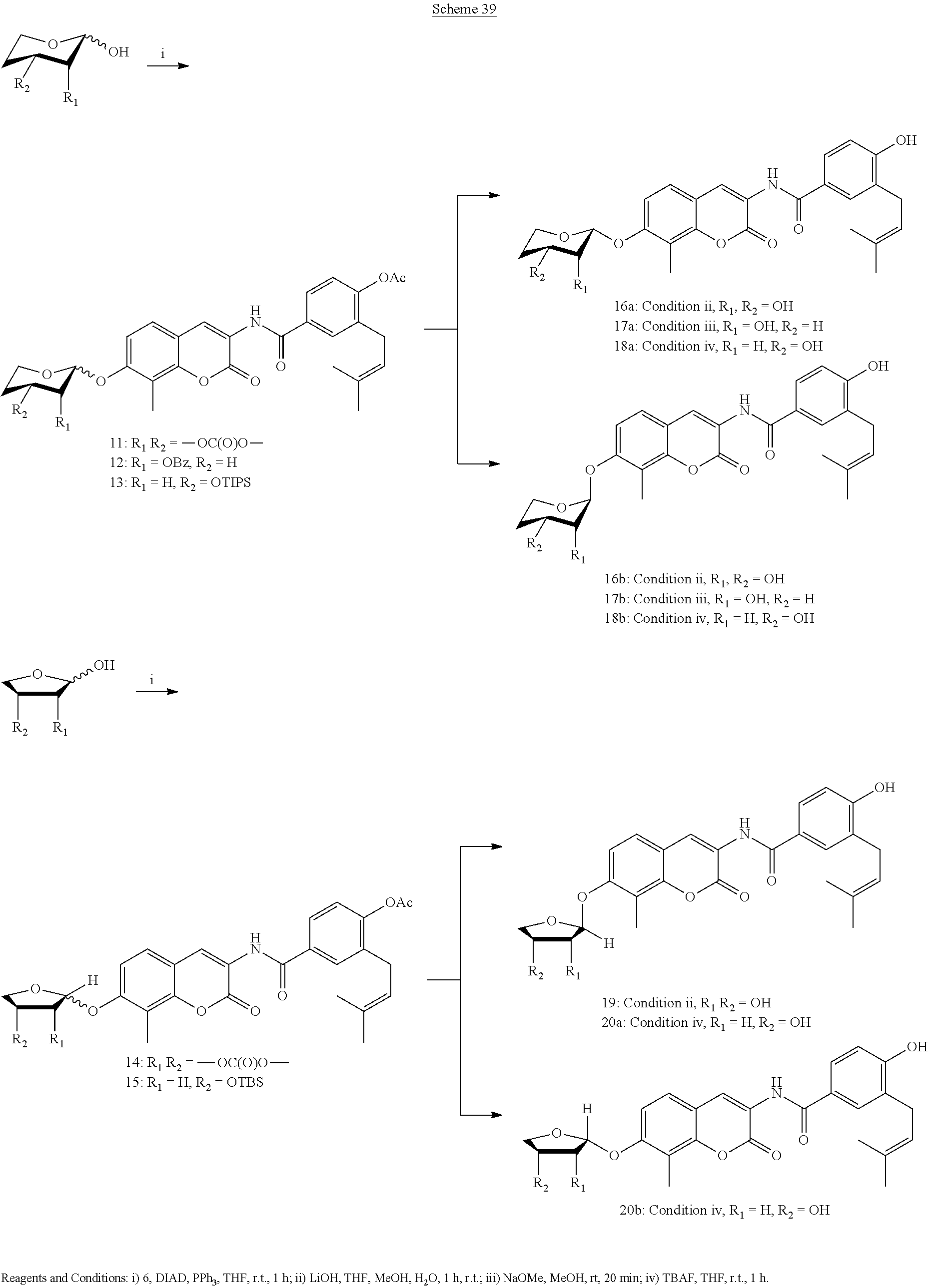

Benzyl 7-(methoxymethoxy)-8-methyl-2-oxo-2H-chromen-3-ylcarbamate (8). To a solution of benzyl 7-hydroxy-8-methyl-2-oxo-2H-chromen-3-ylcarbamate 7. (975 mg, 3.0 mmol) in anhydrous N,N-dimethylformamide (15 mL) was added N,N-diisopropylethylamine (1.01 mL, 7.5 mmol) over 5 minutes at room temperature. The resulting solution was stirred for 30 minutes and cooled to 0° C. To it was added methoxymethylchloride (1.25 mL, 7.5 mmol) dropwise and the resulting mixture was stirred at room temperature for 3 hours. The reaction was quenched by water and the precipitate was filtered, washed with diethyl ether and dried under vacuum to give compound 8 as a white amorphous solid (931 mg, 82%). ¹H NMR (500 MHz, acetone-d₆) δ 8.26 (s, 1H), 8.10 (s, 1H), 7.42 (m, 6H), 7.15 (d, 1H, J=8.7 Hz), 5.34 (s, 2H), 5.26 (s, 2H), 3.49 (s, 3H), 2.29 (s, 3H). ¹³C NMR (100 MHz, acetone-4) δ 158.8, 157.2, 154.2, 149.9, 137.4, 129.3, 129.0, 128.9, 126.2, 123.1, 112.8, 115.0, 114.9, 112.3, 95.6, 67.6, 56.4, 8.3. IR (KBr) νₘₐₓ 3321, 2948, 2931, 28591, 1724, 1693, 1608, 1541, 1367, 1213, 1155, 898 cm⁻¹. HRMS (ESI⁺) m/z [M+Na⁺] calcd for C20H19NNaO6 392.1110. Found 392.1104.

4-(7-(Methoxymethoxy)-8-methyl-2-oxo-2H-chromen-3-ylcarbamoyl)-2-(3-methylbut-2-enyl)phenyl acetate (10). Palladium on carbon (10%, 85.0 mg) was added to a solution of compound 8 (930 mg, 2.52 mmol) in anhydrous THF (10 mL) and the mixture was stirred under an atmosphere of hydrogen for 1 hour and filtered through a celite pad. The filtrate was concentrated and dried under vacuum for 4 hours (573 mg, 90%) before redissolved in THF (3 mL). To it was added a solution of freshly prepared acid chloride 9 (965 mg, 3.63 mmol) in THF (3 mL) followed by pyridine (0.59 mL, 7.26 mmol). The mixture was stirred at room temperature for 12 hours and concentrated to dryness. The residue was purified by column chromatography on silica using hexane and ethyl acetate (3:1) as eluent to afford a white amorphous solid 10 (925 mg, 82%). ¹H NMR (400 MHz, CDCl₃) δ 8.78 (s, 1H), 8.70 (s, 1H), 7.81 (d, 1H, J=2.2 Hz), 7.76 (dd, 1H, J=2.3, 8.4 Hz), 7.32 (d, 1H, J=8.7 Hz), 7.17 (d, 1H, J=8.3), 7.09 (d, 1H, J=8.7 Hz), 5.27 (s, 2H), 5.24 (dd, 1H, J=3.6, 5.0 Hz), 3.50 (s, 3H), 3.32 (d, 2H, J=7.2 Hz), 2.35 (s, 3H), 2.34 (s, 3H), 1.77 (s, 3H), 1.73 (s, 3H). ¹³C NMR (100 MHz, CDCl₃) δ 169.0, 165.5, 159.4, 156.8, 152.1, 149.2, 134.7, 134.4, 131.6, 129.4, 125.9, 125.8, 124.5, 123.0, 121.8, 120.7, 115.0, 114.1, 111.5, 94.7, 56.4, 28.9, 25.8, 21.0, 18.0, 8.4. IR (KBr) νₘₐₓ 3406, 2954, 2914, 1762, 1703, 1666, 1604, 1535, 1369, 1245, 1066, 989 cm'. HRMS (ESI⁺) m/z [M+Na⁺] calcd for C26H27NNaO7 488.1685. Found 488.1704.

-(7-Hydroxy-8-methyl-2-oxo-2H-chromen-3-ylcarbamoyl)-2-(3-methylbut-2-enyl)phenyl acetate (6). To neat compound 10 (900 mg, 1.93 mmol) at room temperature was added 4M HCl in dioxane (2.42 mL, 9.67 mmol) and stirred for 1 hour. The reaction was quenched with cold water (3 mL) and the precipitate was filtered, washed with ether and dried under vacuum to afford compound 6 as a pale yellow amorphous solid (757 mg, 93%). ¹HNMR (500 MHz, CDCl₃) δ 8.73 (s, 1H), 8.61 (s, 1H), 7.76 (d, 1H, J=2.2 Hz), 7.69 (dd, 1H, J=2.3, 8.3 Hz), 7.19 (d, 1H, J=8.4 Hz), 7.17 (s, 1H), 6.72 (d, 1H, J=8.4 Hz), 6.72 (d, 1H, J=8.4 Hz), 6.18 (s, 1H), 5.25 (t, 1H, J=7.2 Hz), 3.32 (d, 2H, J=7.2 Hz), 2.37 (s, 3H), 2.32 (s, 3H), 1.78 (s, 3H), 1.74 (s, 3H). ¹³C NMR (125 MHz, CDCl₃) δ 169.5, 165.4, 159.7, 156.2, 152.2, 149.8, 134.8, 134.5, 131.6, 129.5, 126.0, 125.9, 125.1, 123.0, 121.2, 120.7, 113.3, 113.2, 112.1, 28.9, 25.8, 21.0, 18.1, 8.0. IR (KBr) νₘₐₓ 3386, 3298, 2979, 2931, 1762, 1708, 1535, 1373, 1249, 1215, 1180 cm⁻¹. FIRMS (ESI⁺) m/z [M−H⁻] calcd for C24H22NO6 420.1447. Found 420.1440.

General Procedure I for the Mitsunobu coupling reaction: To a solution of 1 equivalent of phenol 10, 1.2 equivalents of sugar derivatives 1-5 and 2 equivalents of triphenylphosphine in THF (3 mL) was added 2 equivalent of diisopropyl-azadicarboxylate at 0° C. The reaction mixture was stirred at room temperature for 2 hours and then quenched with water and extracted with ethyl acetate (2×10 mL). The combined organic extracts were washed with brine, dried over anhydrous Na₂SO₄ and concentrated to dryness under vacuum. The residues were partially purified by column chromatography on silica to give compounds 11-15 as a mixture of diastereomers except 19 as a single diastereomer (54%-88% yields).

Hydrolysis of compound 11. To a solution of substrate 11 (68 mg, 0.130 mmol) in THF: MeOH: H2O (1.5:1:1 mL) was added lithium hydroxide (27 mg, 0.65 mmol) at room temperature. The reaction mixture was stirred for 1 hour and neutralized with saturated ammonium chloride solution, extracted with ethyl acetate (3×5 mL), washed with brine. The combined organic layers were dried over anhydrous Na₂SO₄ and concentrated under vacuum. The residue was purified by column chromatography on silica using dichloromethane and methanol (96:4) as eluent to give diastereomers 16a (30 mg, 47%) and 16b (20 mg, 31%), both as white amorphous solids.

N-(7-((2R,3R,4R)-3,4-dihydroxytetrahydro-2H-pyran-2-yloxy)-8-methyl-2-oxo-2H-chromen-3-yl)-4-hydroxy-3-(3-methylbut-2-enyl)benzamide (16a). ¹H NMR (500 MHz, CD₂Cl₂) δ 8.70 (s, 1H), 7.65 (d, 1H, J=2.5 Hz), 7.60 (dd, 1H, J=2.5, 8.3 Hz), 7.35 (s, 1H), 7.11 (d, 1H, J=8.7 Hz), 6.85 (d, 1H, J=8.4 Hz), 5.36 (d, 1H, J=Hz), 5.34 (m, 1H), 4.02 (m, 2H), 3.87 (t, 1H, J=3.1 Hz), 3.52 (m, 1H), 3.35 (d, 2H, J=7.2 Hz), 2.37 (s, 3H), 1.90 (m, 2H), 1.75 (s, 3H), 1.73 (s, 3H). ¹³C NMR (125 MHz, CD₂Cl₂) δ 166.7, 159.8, 159.5, 156.8, 149.4, 133.8, 129.3, 129.2, 126.8, 126.0, 124.9, 124.2, 122.6, 122.0, 115.7, 115.1, 115.0, 112.6, 99.2, 69.0, 67.7, 57.5, 30.7, 28.6, 25.7, 17.8, 8.4. IR (KBr) νₘₐₓ 3303, 2977, 2923, 2852, 1708, 1606, 1404, 1282, 1087, 973 cm⁻¹. HRMS (ESI⁺) m/z [M+Na⁺] calcd for C27H29NNaO8 518.1791. Found 518.1786.

N-(7-((2S,3R,4R)-3,4-dihydroxytetrahydro-2H-pyran-2-yloxy)-8-methyl-2-oxo-2H-chromen-3-yl)-4-hydroxy-3-(3-methylbut-2-enyl)benzamide (16b). ¹HNMR (500 MHz, CD₂Cl₂) δ 8.73 (s, 1H), 7.68 (d, 1H, J=2.3 Hz), 7.63 (dd, 1H, J=2.3, 8.3 Hz), 7.40 (d, 1H, J=8.7 Hz), 7.17 (d, 1H, J=8.7 Hz), 6.89 (d, 1H, J=8.4 Hz), 5.57 (d, 1H, J=3.3 Hz), 5.36 (m, 1H), 4.20 (m, 1H), 3.92 (t, 1H, J=3.3 Hz), 3.80 (m, 2H), 3.38 (d, 2H, J=7.2 Hz), 2.36 (s, 3H), 1.99 (m, 2H), 1.79 (s, 3H), 1.76 (s, 3H). ¹³C NMR (125 MHz, CD₂Cl₂) δ 166.8, 159.8, 159.5, 156.3, 149.4, 133.7, 129.2, 129.2, 126.8, 126.0, 124.8, 124.4, 124.4, 122.0, 115.1, 115.1, 114.7, 112.1, 99.4, 69.7, 66.2, 60.3, 29.4, 28.5, 25.7, 17.7, 8.2. IR (KBr) νₘₐₓ 3390, 2958, 2925, 2854, 2520, 1706, 1604, 1404, 1249, 1074, 970 cm⁻¹. HRMS (ESI⁺) m/z [M+Na⁺] calcd for C27H29NNaO8 518.1791. Found 518.1788.

Hydrolysis of compound 12: A solution of the 12 (55 mg, 0.094 mmol) in methanol (3 mL) was treated with sodium methoxide (51 mg, 0.94 mmol) at 0° C. and the resulting yellow solution was stirred for 20 min at room temperature. The reaction mixture was neutralized with saturated ammonium chloride, extracted with ethyl acetate (3×5 mL). The combined organic layers were washed with brine, dried over anhydrous Na₂SO₄, filtered and concentrated under vacuum. The residue was purified by column chromatography on silica using dichloromethane and methanol (98:2) as eluent to give diastereomers 17a (33 mg, 73%) and 17a (8.2 mg, 18%), both as white amorphous solids.

4-Hydroxy-N-(7-((2R,3R)-3-hydroxytetrahydro-2H-pyran-2-yloxy)-8-methyl-2-oxo-2H-chromen-3-yl)-3-(3-methylbut-2-enyl)benzamide (17a). ¹H NMR (400 MHz, CD₃OD) δ 8.72 (s, 1H), 8.70 (s, 1H), 7.65 (d, 1H, J=2.3 Hz), 7.61 (dd, 1H, J=2.3, 8.3 Hz), 7.37 (d, 1H, J=8.6 Hz), 7.16 (d, 1H, J=8.7 Hz), 6.86 (d, 1H, J=8.4 Hz), 5.53 (d, 1H J=3.1 Hz), 5.33 (m, 1H), 3.84 (m, 1H), 3.61 (m, 2H), 3.35 (d, 2H, J=7.2 Hz), 2.40 (s, 3H), 1.95 (m, 1H), 1.76 (m, 1.76 (s, 3H, J=1.6), 1.73 (s, 3H). ¹³C NMR (125 MHz, CDCl₃:MeOH-D4) δ 166.2, 159.5, 158.8, 155.9, 148.9, 134.1, 129.1, 126.5, 125.7, 124.8, 124.0, 122.0, 121.3, 115.0, 114.9, 114.5, 112.1, 97.3, 68.1, 60.3, 29.7, 28.5, 27.4, 25.8, 24.1, 17.8, 8.3. IR (KBr) νₘₐₓ 3405, 2927, 2856, 2524, 1697, 1604, 1504, 1369, 1253, 1120, 983 cm⁻¹. HRMS (ESI⁺) m/z [M+Na⁺] calcd for C27H29NNaO7 502.1842. Found 502.1830.

4-Hydroxy-N-(7-((2S,3R)-3-hydroxytetrahydro-2H-pyran-2-yloxy)-8-methyl-2-oxo-2H-chromen-3-yl)-3-(3-methylbut-2-enyl)benzamide (17b). ¹H NMR (500 MHz, CDCl₃) δ 8.72 (s, 1H), 7.64 (d, 1H, J=2.4 Hz), 7.58 (dd, 1H, J=2.4, 8.3 Hz), 7.30 (d, 1H, J=8.7 Hz), 7.11 (d, 1H, J=8.7 Hz), 6.81 (d, 1H, J=8.4 Hz), 5.49 (d, 1H, J=Hz), 5.31 (m, 1H), 3.80 (m, 1H), 3.58 (m, 3H), 3.33 (d, 2H, J=6.8 Hz), 2.36 (s, 3H), 1.95 (s, 3H), 1.74 (m, 2H), 1.73 (s, 3H), 1.70 (s, 3H). ¹³C NMR (125 MHz, CD₂Cl₂) δ 166.0, 159.7, 159.6, 157.2, 144.9, 133.4, 129.9, 129.3, 127.4, 126.5, 126.0, 124.1, 123.3, 123.0, 115.7, 115.2, 115.0, 112.9, 101.6, 67.7, 63.6, 28.9, 28.9, 25.9, 22.6, 17.9, 8.4. IR (KBr) νₘₐₓ 3407, 2929, 2858, 2522, 1699, 1606, 1520, 1358, 1253, 1109, 983 cm⁻¹. HRMS (ESI⁺) m/z [M+Na⁺] calcd for C27H29NNaO7 502.1842. Found 502.1830.

Deprotection of silyl group and acetate hydrolysis of compound 13: To a solution of compound 13 (98 mg, 0.154 mmol) in THF (4 mL) was added a 1M solution of tetrabutylammonium fluoride in THF (0.31 mL, 0.31 mmol) at 0° C. The resulting mixture was stirred at room temperature for 1 hour and quenched with a saturated ammonium chloride solution (2 mL), extracted with ethyl acetate (3×5 mL). The organic phases were combined, washed with brine, dried over anhydrous Na₂SO₄, filtered and concentrated under vacuum. The residue was purified by column chromatography on silica by using dichloromethane and methanol (98:2) as eluent to give diastereomers 18a (36 mg, 49%) and 18b (24 mg, 33%), both as white amorphous solids.

4-Hydroxy-N-(7-((2S,4R)-4-hydroxytetrahydro-2H-pyran-2-yloxy)-8-methyl-2-oxo-2H-chromen-3-yl)-3-(3-methylbut-2-enyl)benzamide (18a). ¹HNMR (400 MHz, CD₃OD) δ 8.69 (s, 1H), 7.65 (d, 1H, J=2.4 Hz), 7.61 (dd, 1H, J=2.4, 8.3 Hz), 7.37 (d, 1H, J=8.7 Hz), 7.09 (d, 1H, J=8.7 Hz), 6.86 (d, 1H, J=8.4 Hz), 5.35 (m, 1H), 5.23 (dd, 1H, J=2.5, 7.5 Hz), 4.08 (d, 1H, J=12.0 Hz), 3.94 (s, 1H), 3.56 (t, 1H, J=9.7 Hz), 3.35 (d, 2H, J=7.1 Hz), 2.32 (s, 3H), 2.29 (m, 1H), 1.90 (m, 1H), 1.79 (m, 1H), 1.76 (s, 3H), 1.73 (s, 3H), 1.63 (m, 1H). ¹³C NMR (125 MHz, CD₂Cl₂) δ 169.0, 161.9, 161.6, 158.9, 151.4, 135.6, 131.2, 131.2, 128.8, 127.9, 126.8, 126.6, 124.4, 124.1, 117.2, 117.0, 116.7, 114.4, 100.4, 67.5, 63.1, 41.6, 35.9, 30.5, 27.6, 19.6, 10.1. IR (KBr) νₘₐₓ 3396, 2956, 2925, 2864, 2518, 1708, 1604, 1504, 1446, 1367, 1249, 1110, 977 cm⁻¹. HRMS (ESI⁺) m/z [M+H⁺] calcd for C27H30NO7 480.2022. Found 480.1658.

4-Hydroxy-N-(7-((2R,4R)-4-hydroxytetrahydro-2H-pyran-2-yloxy)-8-methyl-2-oxo-2H-chromen-3-yl)-3-(3-methylbut-2-enyl)benzamide (18b), ¹H NMR (500 MHz, acetone-d₆) δ 8.76 (s, 1H), 8.71 (s, 2H), 7.77 (d, 1H, J=2.4 Hz), 7.71 (d, 1H, J=2.4, 8.4 Hz), 7.50 (d, 2H, J=8.6 Hz), 7.21 (d, 2H, J=8.7 Hz), 7.00 (d, 2H, J=8.4 Hz), 5.86 (d, 1H, J=2.9 Hz), 5.39 (m, 1H), 4.28 (m, 1H), 3.99 (br s, OH), 3.77 (m, 2H), 3.40 (d, 3H, J=7.1 Hz), 2.32 (s, 3H), 2.26 (m, 1H), 1.96 (m, 1H), 1.84 (m, 1H), 1.77 (s, 6H), 1.62 (m, 1H). ¹³C NMR. (125 MHz, acetone-d₆) δ 166.0, 159.7, 159.6, 156.9, 149.9, 133.4, 129.9, 129.3, 127.4, 126.5, 126.0, 124.2, 124.1, 123.2, 123.0, 115.7, 114.9, 112.6, 97.7, 63.5, 60.5, 40.3, 35.8, 28.9, 25.9, 17.9, 8.4. IR (KBr) νₘₐₓ 3400, 2960, 2929, 2885, 2522, 1706, 1604, 1502, 1367, 1253, 1085, 970 cm⁻¹. HRMS (ESI⁺) m/z [M+H⁺] calcd for C27H29NO7 480.1658. Found 480.1658.

4-(7-((2S,3S,4S)-3,4-Dihydroxytetrahydrofuran-2-yloxy)-8-methyl-2-oxo-2H-chromen-3-ylcarbamoyl)-2-(3-methylbut-2-enyl)phenyl acetate (19). To a solution of 14 (54 mg, 0.106 mmol) in THF: MeOH: H₂O (1.5:1:1 mL) was added lithium hydroxide (27 mg, 0.53 mmol) at room temperature. The reaction mixture was stirred for 1 hour and neutralized with saturated ammonium chloride solution, extracted with ethyl acetate (3×5 mL), washed with brine. The combined organic layers were dried over anhydrous Na₂SO₄, and concentrated under vacuum. The residue was purified by column chromatography on silica using dichloromethane and methanol (97:3) as eluent to give 19 (35 mg, 68%) as a white amorphous solid. ¹H NMR (500 MHz, acetone-d₆) δ 9.14 (br s, 1H), 8.76 (s, 1H), 8.71 (s, 1H), 7.77 (d, 1H, J=1.9 Hz), 7.71 (dd, 1H, J=1.9, 8.4 Hz), 7.50 (d, 1H, J=8.6 Hz), 7.17 (d, 1H, J=8.7 Hz), 6.99 (d, 1H, J=8.4 Hz), 5.67 (d, 1H, J=1.7 Hz), 5.38 (m, 1H), 4.53 (m, 1H), 4.39 (m, 1H), 4.17 (dd, 1H, J=5.3, 9.3 Hz), 3.89 (dd, 1H, J=3.8, 9.3 Hz), 3.40 (d, 1H, J=7.3 Hz), 2.26 (s, 3H), 1.75 (s, 6H). ¹³C NMR (125 MHz, CDCl₃) δ 166.0, 159.5, 157.2, 149.9, 133.4, 129.9, 126.5, 126.0, 124.1, 124.0, 123.3, 122.9, 115.7, 115.2, 15.1, 113.0, 107.9, 79.2, 77.2, 73.9, 71.2, 28.8, 25.9, 17.9, 8.4. IR (KBr) νₘₐₓ 3396, 2962, 2925, 1701, 1604, 1504, 1369, 1253, 1054, 981 cm⁻¹. HRMS (ESI) m/z. [M−H⁻] calcd for C26H26NO8 480.1658. Found 480.1658.

Silyl group deprotection and acetate hydrolysis of compound 13: To a solution of compound 15 (43 mg, 0.074 mmol) in THF (3 mL) was added a 1M solution of tetrabutylammonium fluoride in THF (0.15 mL, 0.15 mmol) at 0° C. The resulting reaction mixture was stirred at rt for 1 h, quenched with saturated ammonium chloride (2 mL) and extracted with EtOAc (3×4 mL). The organic phases were combined, washed with brine, dried over anhydrous Na₂SO₄, filtered and concentrated under vacuum. The residue was purified by column chromatography on silica using dichloromethane and methanol (98:4) as eluent to give diastereomers 20a (16 mg, 47%) and 20b (11 mg, 31%), both as white amorphous solids.

4-(7-((2S,4R)-4-Hydroxytetrahydrofuran-2-yloxy)-8-methyl-2-oxo-2,4-chromen-3-ylcarbamoyl)-2-(3-methylbut-2-enyl)phenyl acetate (20a). ¹H NMR (400 MHz, acetone-d₆) δ 9.13 (br s, 1H), 8.77 (s, 1H), 8.72 (s, 1H), 7.77 (d, 1H, J=2.2 Hz), 7.71 (dd, 1H, J=2.2, 8.2 Hz), 7.50 (d, 1H, J=8.9 Hz), 7.25 (d, 1H, J=8.6 Hz), 6.99 (d, 1H, J=8.3 Hz), 6.07 (dd, 1H, J=2.7, 5.6 Hz), 5.39 (m, 1H), 4.69 (m, 1H), 4.20 (s, 1H), 4.05 (dd, 1H, J=4.8, 9.6 Hz), 3.86 (d, 1H, J=9.7 Hz), 3.60 (s, 1H), 3.39 (d, 2H, J=7.3 Hz), 2.48 (m, 1H), 2.35 (m, 1H), 2.25 (s, 3H), 1.75 (s, 6H). ¹³C NMR (125 MHz, CDCl₃ and MeOH-d4) δ 166.4, 159.6, 159.0, 156.3, 148.9, 133.7, 128.9, 128.7, 126.4, 125.6, 124.5, 124.1, 121.4, 115.1, 114.8, 114.4, 112.5, 103.3, 76.5, 70.4, 41.7, 29.6, 28.2, 25.7, 17.7, 8.4. IR (KBr) νₘₐₓ 3305, 2960, 2931, 2875, 1708, 1604, 1404, 1367, 1282, 1112, 1058, 974 cm⁻¹. HRMS (ESI) m/z [M−H⁻] calcd for C26H26NO7 464.1709. Found 464.1706.

4-(7-((2R,4R)-4-Hydroxytetrahydrofuran-2-yloxy)-8-methyl-2-oxo-2H-chromen-3-ylcarbamoyl)-2-(3-methylbut-2-enyl)phenyl acetate (20b). ¹H NMR (500 MHz, CDCl₃) δ 8.69 (s, 1H), 8.64 (s, 1H), 7.62 (s, 1H), 7.56 (d, 1H, J=8.3 Hz), 7.30 (d, 1H, J=7.2 Hz), 7.13 (d, 1H, J=8.6 Hz), 6.81 (d, 1H, J=8.4), 5.82 (d, 1H, J=4.8 Hz), 5.30 (m, 1H), 4.48 (m, 1H), 4.09 (dd, 1H, J=5.3, 9.8 Hz), 4.00 (dd, 1H, J=2.9, 9.7 Hz), 3.32 (d, 2H, J=6.4 Hz), 2.33 (m, 2H), 2.27 (s, 3H), 1.72 (s, 3H), 1.69 (s, 3H). ¹³C NMR (125 MHz, CDCl₃: CD₃OD) δ 172.2, 165.3, 164.7, 162.3, 154.6, 139.2, 134.5, 134.4, 132.0, 131.2, 130.0, 127.3, 127.1, 120.5, 120.4, 119.7, 117.9, 108.5, 80.5 76.1, 47.9, 33.7, 31.2, 23.3, 19.0, 13.7. IR (KBr) νₘₐₓ 3305, 2958, 2923, 2854, 2522, 1695, 1604, 1502, 1371, 1261, 1066, 964 cm⁻¹. HRMS (ESI) m/z [M−H⁻] calcd for C26H26NO7 464.1709. Found 464.1702.

### Example 21

### Novobiocin Analogs with Alkylamine and Heterocyclic Noviose Replacements

Although simplified sugar mimics were found to increase the anti-proliferative activity ˜200 times greater than novobiocin, more simplified analogues exhibiting enhanced solubility and activity were desired. N-Heterocycles are found in a variety of biologically active compounds, and in contrast to carbohydrates, are generally ionized at physiological pH.²⁷ Upon review of the first set of studies, we proposed that the noviose appendage was responsible for solubilizing the predominately hydrophobic coumarin core and benzamide side chain. Thus, commercially available amines, 21-27, shown below, were selected as potential replacements for the noviose moiety. These alkylamines and heterocyclic analogues contain an ionizable amine located at various positions within the structure to afford potential hydrogen-bonding interactions while simultaneously enhancing solubility.

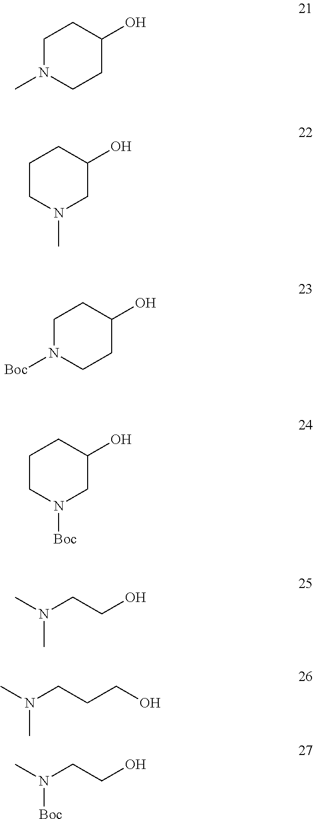

Originally, coupling of these amines with phenol 6 were expected to easily afford the desired analogues. However, the acetyl ester on the benzamide side chain was hydrolyzed under these conditions and resulted in an inseparable mixture of mono- or dialkylated products. To circumvent this issue, the amine was coupled with the coumarin ring and subsequently with the benzamide side chain to afford the desired analogues. The detailed synthesis is described as follows: tertiary amines or Boc-masked secondary amines were reacted with Cbz-protected coumarin in the presence of two equivalents of triphenylphosphine and diisopropylazodicarboxylate in tetrahydrofuran to give amine-derived coumarins, 28a-28f. The Cbz-protecting group was removed by hydrogenolysis to give the free amines, which were then coupled with acid chloride 9 to give compounds 29a-29g in good yield. Removal of the Boc protecting group with trifluoroacetic acid in methylene chloride afforded the secondary amine analogues 30b, 30d and 30g. Hydrolysis of the phenolic ester with 10% triethyl amine in methanol gave denoviosylated analogues, 31a-31g, in good to excellent yield. Synthesis of the amine containing analogues is shown in Scheme 40 below.

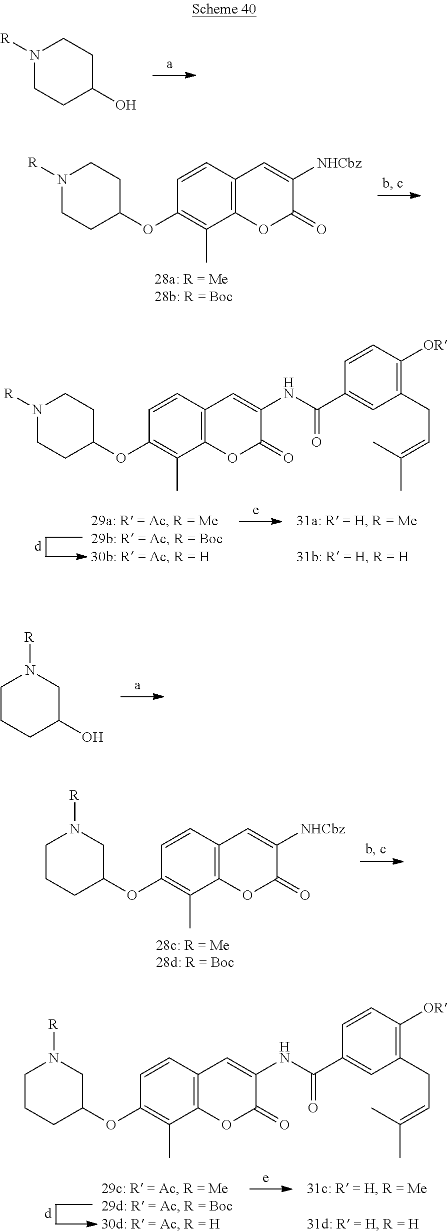

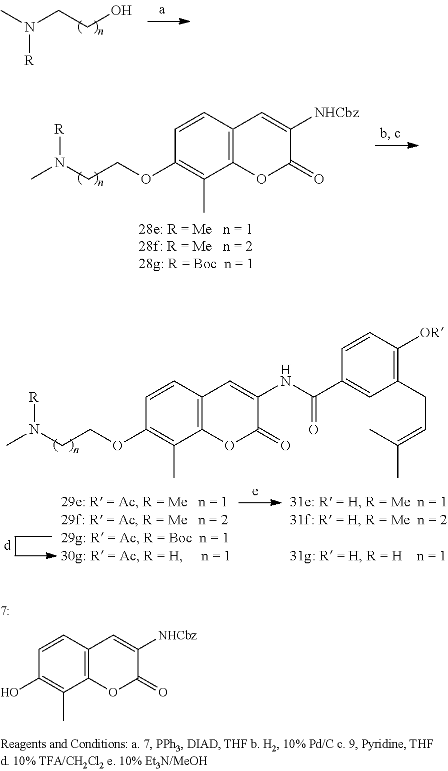

Benzyl 2-(8-methyl-7-(1-methylpiperidin-4-yloxy)-2-oxo-2H-chromen-3-ylamino)-2-oxoacetate (28a). General procedure II for Mitsunobu coupling: To a solution of 21 (0.64 g, 5.59 mmol), 7 (1.74 g, 5.59 mmol) and triphenylphosphine (2.93 g, 11.2 mmol) in THF (50 mL) was added diisopropylazodicarboxylate (2.26 g, 11.2 mmol) dropwise. The reaction mixture was stirred at room temperature for 4 hours and concentrated under vacuum. The residue was purified by column chromatography on silica using methylene chloride and methanol (10:1) as eluent to afford 28a as colorless foam (1.48 g, 63%). ¹H NMR (400 MHz, CDCl₃) δ 8.28 (s, 1H), 7.55 (s, 1H), 7.45˜7.38 (m, 5H), 7.29 (d, J=8.0, 1H), 6.87 (d, J=8.0, 1H), 5.25 (s, 2H), 4.49 (m, 1H), 2.72˜2.68 (m, 2H), 2.52˜2.49 (m, 2H), 2.38 (s. 3H), 2.35 (s, 1H), 2.11˜2.05 (m, 2H), 1.96˜1.92 (m, 2H). ¹³C NMR (100 MHz, CDCl₃) δ 159.1, 156.8, 153.4, 149.4, 135.8, 128.9, 128.7, 128.4, 125.3, 122.5, 121.6, 115.4, 113.5, 110.7, 77.4, 67.6, 52.4, 46.4, 30.8, 8.6. IR (film) νₘₐₓ 3406, 3319, 2939, 2849, 2791, 1711, 1609, 1524, 1366, 1271, 1227, 1204, 1103, 1038, 1024 cm⁻¹. HRMS (ESI⁺) m/z Calc for [M+H⁺] C24H27N2O5 423.1920. Found 423.1920.

Benzyl 2-(8-methyl-7-(1-methylpiperidin-3-yloxy)-2-oxo-2H-chromen-3-ylamino)-2-oxoacetate (28c). Prepared from 22 (81 mg, 0.70 mmol) and 7 (215 mg; 0.70 mmol). 28c was obtained as colorless foam (64 mg, 22%). ¹H NMR (400 MHz, CDCl₃) δ 8.22 (s, 1H), 7.76 (s, 1H), 7.39˜7.33 (m, 5H), 7.22 (d, J=8.0, 1H), 6.88 (d, J=8.0, 1H), 5.21 (s, 2H), 4.39 (m, 1H), 2.97˜2.95 (m, 1H), 2.64˜2.62 (m, 1H), 2.30 (s. 3H), 2.25 (s, 1H), 2.21 (m, 1H), 2.16 (m, 1H), 2.07˜2.05 (m, 2H), 1.85˜1.82 (m, 1H), 1.64˜1.62 (m, 1H), 1.48˜1.46 (m, 1H). ¹³C NMR (100 MHz, CDCl₃) δ 158.8, 157.0, 153.3, 149.1, 135.7, 128.6, 128.4, 128.3, 125.1, 122.5, 121.4, 115.0, 113.3, 110.7, 74.1, 67.3, 59.7, 55.4, 46.3, 29.8, 23.1, 8.3. IR (film) νₘₐₓ 3408, 3302, 2978, 2926, 2853, 1713, 1609, 1522, 1464, 1375, 1290, 1236, 1107 cm⁻¹. HRMS (ESI⁺) m/z Calc for [M+H⁺] C24H27N2O5 423.1920. Found: 423.1920.

Benzyl 7-(2-(dimethylamino)ethoxy)-8-methyl-2-oxo-2H-chromen-3-ylcarbamate

(28e). Prepared from 25 (40 mg, 0.50 mmol) and 7 (138 mg, 0.50 mmol). 28c was obtained as colorless oil (126 mg, 64%). ¹H NMR (400 MHz, CDCl₃) δ 8.24 (s, 1H), 7.58 (s, 1H), 7.40˜7.34 (m, 5H), 7.25 (d, J=8.6, 1H), 6.83 (d, J=8.6, 1H), 5.22 (s, 2H), 4.14 (t, J=5.6, 2H), 2.80 (t, J=5.6, 2H), 2.37 (s, 6H), 2.29 (s, 3H). ¹³C NMR (100 MHz, CDCl₃) δ 159.0, 158.1, 153.3, 149.0, 135.8, 128.8, 128.6, 128.3, 125.3, 122.4, 121.4, 114.2, 113.4, 108.9, 67.6, 67.5, 58.3, 46.3, 21.9, 8.3. IR (film) νₘₐₓ 3406, 3302, 2980, 2939, 2878, 2824, 2773, 1713, 1609, 1524, 1456, 1383, 1227, 1111, 1024 cm⁻¹. HRMS (ESI⁺) m/z Calc for [M+H⁺] C22H25N2O5 397.1763. Found: 397.1759.

Benzyl 7-(3-(dimethylamino)propoxy)-8-methyl-2-oxo-2H-chromen-3-yl carbamate (28f). Prepared from 26 (230 mg, 2.3 mmol) and 7 (703 mg, 2.3 mmol). 28f was obtained as colorless oil (670 mg, 72%). ¹HNMR (400 MHz, CDCl₃): δ 8.11 (s, 1H), 7.45 (s, 1H), 7.29˜7.23 (m, 5H), 7.11 (d, J=8.6, 1H), 6.70 (d, J=8.6, 1H), 5.10 (s, 2H), 3.97 (t, J=5.8, 2H), 2.46 (t, J=5.8, 2H), 2.22 (s, 6H), 2.17 (s, 3H), 1.93 (m, 2H). ¹³C NMR (100 MHz, CDCl₃) δ 159.0, 158.2, 153.3, 149.0, 135.8, 128.8, 128.6, 128.4, 125.3, 122.5, 121.4, 114.0, 113.3, 108.9, 67.5, 66.8, 56.4, 45.4, 27.4, 8.2. IR (film) νₘₐₓ 3404, 3323, 2978, 2943, 2816, 2768, 1713, 1610, 1524, 1381, 1366, 1273, 1227, 1204, 1109, 1022 cm⁻¹. HRMS (ESI⁺) m/z Calc for [M+H⁺] C23H26N2O5 411.1920. Found: 411.1918.

4-(8-Methyl-7-(1-methylpiperidin-4-yloxy)-2-oxo-2H-chromen-3-ylcarbamoyl)-2-(3-methylbut-2-enyl)phenyl acetate (29a). General procedure III for the Preparation of benzamide 29a-g: Palladium on carbon (10%, 10 mg) was added to a solution of 28a (85 mg, 0.2 mmol) in THF. The suspension was stirred overnight under a hydrogen atmosphere before it was filtered. The filtrate was concentrated and dried under vacuum for 4 hours before it was redissolved in THF (5 mL). To it was added freshly prepared acid chloride 9 (80 mg, 0.3 mmol) in THF (5 mL) and dry pyridine (32 mg, 0.4 mmol). The resulting mixture was stirred at room temperature overnight and concentrated to dryness. The residue was purified by column chromatography on silica using methylene chloride and methanol (10:1) as eluent to afford 29a as white amorphous solid (76 mg, 73%). ¹H NMR (500 MHz, CDCl₃), δ 8.79 (s, 1H), 8.71 (s, 1H), 7.81 (d, J=2.1, 1H), 7.77 (dd, J=8.3, 2.1, 1H), 7.34 (d, J=8.6, 1H), 7.18 (d, J=8.3, 1H), 6.89 (d, J=8.6, 1H), 5.25 (m, 1H), 4.52 (m, 1H), 3.33 (d, J=7.2, 2H), 2.76˜2.70 (m, 2H), 2.55˜2.45 (m, 2H), 2.40 (s, 3H), 2.35 (s, 6H), 2.15˜2.09 (m, 2H), 1.97˜4.92 (m, 2H), 1.78 (s, 3H), 1.74 (s, 3H). ¹³C NMR 5(125 MHz, CDCl₃) 169.1, 165.6, 159.6, 157.1, 152.3, 149.7, 134.8, 134.5, 131.8, 129.5, 126.1, 125.8, 124.8, 123.1, 121.7, 120.8, 115.4, 113.5, 110.6, 77.4, 52.3, 46.2, 30.5, 29.0, 25.9, 21.1, 18.1, 8.6. IR (film) νₘₐₓ 3400, 2922, 2851, 1765, 1711, 1672, 1607, 1526, 1493, 1369, 1248, 1202, 1175, 1099, 1040 cm⁻¹. HRMS (ESI⁺) m/z Calc for [M+H⁺] C30H35N2O6 519.2495. Found: 519.2485.

4-(8-Methyl-7-(1-methylpiperidin-3-yloxy)-2-oxo-2H-chromen-3-ylcarbamoyl)-2-(3-methylbut-2-enyl)phenyl acetate (29c). Compound 29c was obtained as a white amorphous solid (72 mg, 92%). ¹H NMR (500 MHz, CDCl₃) δ 8.77 (s, 1H), 8.70 (s, 1H), 7.81 (d, J=2.1, 1H), 7.77 (dd, J=8.3, 2.3, 1H), 7.32 (d, J=8.7, 1H), 7.17 (d, J=8.3, 1H), 6.97 (d, J=8.7, 1H), 5.24 (m, 1H), 4.54 (m, 1H), 3.31 (d, J=7.2, 2H), 3.18˜3.16 (m, 1H), 2.86˜2.83 (m, 1H), 2.41 (s, 3H), 2.34 (s, 3H), 2.31 (s, 3H), 2.29˜2.12 (m, 3H), 1.91˜1.86 (m, 1H), 1.76 (s, 3H), 1.73 (s, 3H), 1.69˜1.65 (m, 1H), 1.54˜1.47 (m, 1H). ¹³C NMR δ (125 MHz, CDCl₃) 169.0, 165.5, 159.6, 157.3, 152.2, 149.6, 134.7, 134.4, 131.8, 129.5, 126.0, 125.9, 124.7, 123.0, 121.6, 120.8, 115.4, 113.6, 110.9, 73.4, 59.2, 55.1, 45.8, 29.6, 28.9, 25.9, 22.7, 21.0, 18.1, 8.5. IR (film) νₘₐₓ 3402, 2939, 2856, 2786, 1763, 1711, 1672, 1607, 1526, 1491, 1367, 1250, 1204, 1173, 1099 cm⁻¹. HRMS (ESI⁺) m/z Calc for [M+H⁺] C30H35N2O6 519.2495. Found: 519.2493.

4-(7-(2-(Dimethylamino)ethoxy)-8-methyl-2-oxo-2H-chromen-3-ylcarbamoyl)-2-(3-methylbut-2-enyl)phenyl acetate (29e). Compound 29e (26 mg, 84%) was obtained as a light yellow, amorphous solid. ¹H NMR (500 MHz, CDCl₃) δ 8.79 (s, 1H), 8.70 (s, 1H), 7.81 (s, 1H), 7.77 (d, J=8.3, 1H), 7.34 (d, J=8.7, 1H), 7.18 (d, J=8.3, 1H), 6.88 (d, J=8.7, 1H), 5.25 (m, 1H), 4.22 (t, J=5.5, 2H), 3.32 (d, J=7.2, 2H), 2.91 (t, J=5.5, 2H), 2.46 (s, 6H), 2.35 (s, 3H), 2.34 (s, 3H), 1.77 (s, 3H), 1.73 (s, 3H). ¹³C NMR (125 MHz, CDCl₃) δ 169.1, 165.6, 159.7, 158.4, 152.3, 149.4, 134.8, 134.5, 131.8, 129.5, 126.1, 126.0, 124.8, 123.1, 121.7, 120.8, 114.4, 113.7, 109.1, 67.2, 58.2, 46.0, 29.0, 26.0, 21.1, 18.1, 8.4. IR (film) νₘₐₓ 3393, 2961, 2859, 1761, 1705, 1664, 1607, 1528, 1491, 1369, 1267, 1246, 1202, 1177, 1109 cm⁻¹. HRMS (ESI⁺) m/z Calc for [M+H⁺] C28H33N2O6 493.2339. Found: 493.2336.

4-(7-(3-(Dimethylamino)propoxy)-8-methyl-2-oxo-2H-chromen-3-ylcarbamoyl)-2-(3-methylbut-2-enyl)phenyl acetate (290. Compound 29f (79 mg, 77%) was obtained as a white, light yellow, amorphous solid. ¹H NMR (500 MHz, CDCl₃) δ 8.79 (s, 1H), 8.70 (s, 1H), 7.81 (s, 1H), 7.77 (d, J=8.3, 1H), 7.34 (d, J=8.6, 1H), 7.18 (d, J=8.3, 1H), 6.88 (d, J=8.6, 1H), 5.25 (m, 1H), 4.15 (t, J=6.0, 2H), 3.32 (d, J=7.2, 2H), 2.77 (t, J=7.4, 2H), 2.49 (s, 6H), 2.35 (s, 3H), 2.33 (s, 3H), 2.17 (m, 2H), 1.77 (s, 3H), 1.73 (s, 3H). ¹³C NMR (125 MHz, CDCl₃) δ 169.1, 165.6, 159.7, 158.4, 152.3, 149.4, 134.8, 134.5, 131.8, 129.6, 126.09, 126.05, 124.9, 123.1, 121.7, 120.9, 114.3, 113.6, 109.1, 66.7, 56.4, 45.0, 29.0, 26.8, 26.0, 21.1, 18.2, 8.4. IR (film) νₘₐₓ 3393, 2964, 2930, 1761, 1705, 1666, 1607, 1549, 1371, 1246, 1202, 1182, 1107 cm⁻¹. HRMS (ESI⁺) m/z Calc for [M+H⁺] C29H35N2O6 507.2495. Found: 507.2501.

4-(8-methyl-2-oxo-7-(piperidin-4-yloxy)-2H-chromen-3-ylcarbamoyl)-2-(3-methylbut-2-enyl)phenyl acetate (30b). General procedure III for preparation of 30b, 30d and 30g through Mitsunobu coupling, benzamide coupling and Boc deprotection: Boc protected amine 23 (99 mg, 0.49 mmol) was coupled with 7 (152 mg, 0.49 mmol) following general procedure II to afford partially purified 28b (160 mg), which went through hydrogenolysis and coupled with acid chloride following the described general procedure III to give compound 29b. Compound 29b was dissolved in 10% trifluoroacetic acid in methylene chloride, stirred at room temperature for 2 hours and concentrated. The residue was purified by column chromatography on silica by using methylene chloride and methanol (10:1) to give 30b as a light brown, amorphous solid (106 mg, 3 steps, 43%). ¹H NMR 8 (400 MHz, DMSOd₆), 9.69 (s, 1H), 8.50 (s, 1H), 7.86 (s, 1H), 7.84 (d, J=8.3, 1H), 7.61 (d, J=8.4, 1H), 7.25 (d, J=8.3, 1H), 7.18 (d, J=8.4, 1H), 5.20 (m, 1H), 4.84 (m, 1H), 3.28 (d, J=7.2, 2H), 3.21 (m, 2H), 3.11 (m, 2H), 2.29 (s, 3H), 2.25 (s, 3H), 2.17˜2.13 (m, 2H), 1.93˜1.91 (m, 2H), 1.70 (s, 3H), 1.69 (s, 3H). ¹³C NMR (125 MHz, DMSOd₆) δ 168.9, 165.2, 158.1, 156.3, 151.5, 149.8, 133.6, 132.8, 131.4, 129.6, 129.4, 126.6, 126.3, 122.9, 121.4, 121.3, 113.5, 112.9, 110.6, 69.7, 40.2, 28.3, 27.1, 25.5, 20.7, 17.7, 8.1. IR (film) νₘₐₓ 3400, 2984, 2854, 1745, 1718, 1678, 1607, 1535, 1442, 1371, 1205, 1142, 1103 cm⁻¹. HRMS (ESI⁺) m/z Calc for [M+H⁺] C29H33N2O6 505.2339. Found: 505.2340.

4-(8-methyl-2-oxo-7-(piperidin-3-yloxy)-2H-chromen-3-ylcarbamoyl)-2-(3-methylbut-2-enyl)phenyl acetate (30d). Compound 30d was obtained as a light brown, amorphous solid (31 mg, 3 steps, 22%). ¹H NMR (500 MHz, CDCl₃-CD₃OD), δ 8.68 (s, 1H), 7.71 (s, 1H), 7.68 (d, J=8.4, 1H), 7.30 (d, J=8.6, 1H), 7.09 (d, J=8.4, 1H), 6.86 (d, J=8.8, 1H), 5.15 (m, 1H), 4.56 (m, 1H), 3.27-3.22 (m, 3H), 3.10-3.06 (m, 1H), 2.99-2.97 (m, 2H), 2.27 (s, 3H), 2.26 (s, 3H), 2.03-1.82 (m, 4H), 1.68 (s, 3H), 1.64 (s, 3H). ¹³C NMR (125 MHz, CDCl₃) δ 169.1, 165.6, 159.3, 155.8, 152.3, 149.5, 134.8, 134.5, 131.6, 129.6, 126.1, 125.9, 124.2, 123.1, 122.1, 120.8, 115.8, 114.4, 110.2, 69.4, 45.0, 43.9, 29.0, 27.6, 25.9, 25.7, 21.1, 18.1, 8.4. IR (film) νₘₐₓ 3402, 2968, 2935, 2860, 1763, 1701, 1676, 1607, 1528, 1493, 1439, 1369, 1252, 1204, 1178, 1140, 1099 cm⁻¹. HRMS (ESI⁺) m/z Calc for [M+H⁺] C29H33N2O6 505.2339. Found: 505.2340.

4-(8-methyl-7-(2-methylamino)ethoxy)-2-oxo-2H-chromen-3-ylcarbamoyl)-2-(3-methylbut-2-enyl)phenyl acetate (30g). Compound 30g was obtained as a light yellow, amorphous solid (53 mg, 3 steps, 34%). ¹H NMR (CDCl₃, 500 MHz), δ 8.63 (s, 1H), 7.66 (s, 1H), 7.63 (dd, J=8.3, 2.1, 1H), 7.28 (d, J=8.6, 1H), 7.05 (d, J=8.3, 1H), 6.82 (d, J=8.6, 1H), 5.10 (m, 1H), 4.20 (t, J=5.0, 2H), 3.26 (t, J=5.0, 2H), 3.18 (d, J=7.2, 2H), 2.62 (s, 3H), 2.21 (s, 6H), 1.62 (s, 3H), 1.59 (s, 3H). ¹³C NMR δ (CDCl₃, 125 MHz) 169.4, 165.9, 159.4, 157.4, 152.1, 149.2, 134.7, 134.3, 131.4, 129.3, 126.0, 125.9, 125.1, 122.9, 121.6, 120.5, 114.3, 114.0, 109.1, 64.7, 49.5, 33.8, 28.7, 25.5, 20.7, 17.7, 7.8. IR (film) νₘₐₓ 3393, 2964, 2918, 2849, 1767, 1710, 1676, 1605, 1528, 1491, 1369, 1252, 1202, 1178, 1136, 1111 cm⁻¹. HRMS (ESI⁺) m/z Calc for [M+H⁺] C27H31N2O6 479.2182. Found: 479.2181.

4-Hydroxy-N-(8-methyl-7-(1-methylpiperidin-4-yloxy)-2-oxo-2H-chromen-3-yl)-3-(3-methylbut-2-enyl)benzamide (31a). General procedure IV for the preparation of compounds 31a-g: Compound 29a (52 mg, 0.1 mmol) was dissolved in 10% triethylamine/methanol (3 mL). The solution was stirred at room temperature overnight and concentrated. The residue was purified by column chromatography on silica by using methylene chloride and methanol (10:1) to give 31a as a white, amorphous solid (37 mg, 73%). ¹H NMR (500 MHz, DMSOd₆) δ 9.22 (s, 1H), 8.47 (s, 1H), 7.68˜7.66 (m, 2H), 7.53 (d, J=8.7, 1H), 7.11 (d, J=8.9, 1H), 6.90 (d, J=8.7, 1H), 5.30 (m, 1H), 4.57 (m, 1H), 3.27 (d, J=7.3, 2H), 2.62˜2.54 (m, 2H), 2.34˜2.28 (m, 2H), 2.22 (s, 3H), 2.21 (s, 3H), 1.96˜1.92 (m, 2H), 1.76˜1.72 (m, 2H), 1.71 (s, 3H), 1.69 (s, 3H). ¹³C NMR (125 MHz, DMSOd₆) δ165.3, 158.8, 158.4, 156.6, 149.5, 131.9, 129.2, 127.8, 127.7, 126.8, 126.0, 124.0, 122.3, 121.3, 114.6, 113.4, 112.7, 110.8, 72.1, 51.8, 45.7, 30.2, 27.9, 25.6, 17.7, 8.2. IR (film) νₘₐₓ 3421, 1703, 1666, 1601, 1528, 1504, 1366, 1248, 1178, 1150, 1094, 1040 cm⁻¹. HRMS (ESI⁺) m/z Calc for [M+H⁺] C28H33N2O5 477.2389. Found: 477.2397.

4-Hydroxy-N-(8-methyl-2-oxo-7-(piperidin-4-yloxy)-2H-chromen-3-yl)-3-(3-methylbut-2-enyl)benzamide (31b). Compound 31b was obtained as a light brown, amorphous solid (13 mg, 56%). ¹H NMR (500 MHz, DMSOd₆) δ 9.21 (s, 1H), 8.47 (s, 1H), 7.67˜7.65 (m, 2H), 7.53 (d, J=8.7, 1H), 7.12 (d, J=8.9, 1H), 6.90 (d, J=8.7, 1H), 5.31 (m, 1H), 4.58 (m, 1H), 3.27 (d, J=7.3, 2H), 2.95˜2.93 (m, 2H), 2.62˜2.58 (m, 2H), 2.23 (s, 3H), 1.92˜1.90 (m, 2H), 1.71 (s, 3H), 1.70 (s, 3H), 1.54˜1.45 (m, 2H). ¹³C NMR (125 MHz, DMSOd₆) δ165.4, 159.1, 158.4, 156.7, 149.5, 131.8, 129.2, 127.9, 127.7, 126.9, 126.0, 123.8, 122.4, 121.3, 114.6, 113.4, 112.6, 110.9, 74.1, 43.3, 32.1, 28.0, 25.6, 17.7, 8.2. IR (film) νₘₐₓ 3401, 2959, 2927, 2872, 2858, 1724, 1693, 1643, 1632, 1605, 1529, 1447, 1367, 1261, 1117, 1072 cm⁻¹. HRMS (ESI⁺) m/z Calc for [M+H⁺] C27H31N2O5 463.2233. Found: 463.2245.

4-Hydroxy-N-(8-methyl-7-(1-methylpiperidin-3-yloxy)-2-oxo-2H-chromen-3-yl)-3-(3-methylbut-2-enyl)benzamide (31c). Compound 31c was obtained as a white, amorphous solid (19 mg, 69%). ¹H NMR (400 MHz, CDCl₃-CD₃OD) δ 8.69 (s, 1H), 7.62 (d, J=2.1, 1H), 7.56 (dd, J=8.4, 2.1, 1H), 7.29 (d, J=8.7, 1H), 6.90 (d, J=8.7, 1H), 6.80 (d, J=8.3, 1H), 5.30 (m, 1H), 4.42 (m, 1H), 3.32 (d, J=7.1, 2H), 3.05˜3.02 (m, 1H), 2.74˜2.71 (m, 1H), 2.32 (s, 3H), 2.27 (s, 3H), 2.25˜2.06 (m, 3H), 1.85˜1.81 (m, 1H), 1.72 (s, 3H), 1.69 (s, 3H), 1.67˜1.63 (m, 1H), 1.50˜1.40 (m, 1H). ¹³C NMR (100 MHz, CD₂Cl₂-CD₃OD) δ167.1, 160.3, 159.9, 157.6, 150.0, 133.9, 129.52, 129.47, 127.1, 126.3, 125.0, 122.8, 122.4, 122.3, 115.7, 115.3, 114.3, 111.3, 73.7, 59.6, 55.6, 46.1, 29.7, 28.8, 25.9, 22.8, 18.0, 8.4. IR (film) νₘₐₓ 3423, 1710, 1663, 1603, 1528, 1375, 1277, 1256, 1204, 1140 cm⁻¹. HRMS (ESI⁺) m/z Calc for [M+H⁺] C28H33N2O5 477.2389. Found: 477.2391.

4-Hydroxy-N-(8-methyl-2-oxo-7-(piperidin-3-yloxy)-2H-chromen-3-yl)-3-(3-methylbut-2-enyl)benzamide (31d). Compound 31d was obtained as a light brown, amorphous solid (21 mg, 72%). ¹H NMR (500 MHz, CDCl₃-CD₃OD), δ 8.43 (s, 1H), 7.38 (d, J=2.2, 1H), 7.34 (dd, J=8.3, 2.2, 1H), 7.11 (d, J=8.6, 1H), 6.71 (d, J=8.6, 1H), 6.60 (d, J=8.3, 1H), 5.07 (m, 1H), 4.42 (m, 1H), 3.08 (d, J=7.2, 2H), 3.02 (m, 1H), 2.77-2.75 (m, 2H), 2.07 (s, 3H), 1.79-1.65 (m, 3H), 1.48 (s, 3H), 1.48-1.45 (m, 1H), 1.45 (s, 3H). ¹³C NMR (125 MHz, CD₂Cl₂-CD₃OD) δ 167.6, 163.2, 162.9, 160.2, 156.9, 150.2, 133.7, 129.7, 127.3, 126.7, 125.0, 122.6, 118.6, 116.3, 115.6, 115.4, 114.9, 111.2, 70.3, 54.0, 47.4, 44.6, 28.8, 27.4, 25.8, 19.3, 17.8, 8.3. IR (film) νₘₐₓ 3402, 2959, 2928, 2872, 2858, 1726, 1668, 1605, 1526, 1502, 1454, 1366, 1259, 1120, 1072 cm⁻¹. HRMS (ESI⁺) m/z Calc for [M+H⁺] C27H31N2O5 463.2233. Found: 463.2226.

N-(7-(2-(Dimethylamino)ethoxy)-8-methyl-2-oxo-2H-chromen-3-yl)-4-hydroxy-3-(3-methylbut-2-enyl)benzamide (31e). Compound 31e was obtained as a white, amorphous foam (8.4 mg, 61%). ¹H NMR (400 MHz, CD₂Cl₂-CD₃OD) δ 8.58 (s, 1H), 7.60 (d, J=2.4, 1H), 7.54 (dd, J=8.4, 2.4, 1H), 7.33 (d, J=8.7, 1H), 6.90 (d, J=8.7, 1H), 6.82 (d, J=8.4, 1H), 5.31 (m, 1H), 4.16 (t, J=6.2, 2H), 3.32 (d, J=7.2, 2H), 2.85 (t, J=6.9, 2H), 2.39 (s, 6H), 2.26 (s, 3H), 1.74 (s, 3H), 1.71 (s, 3H). ¹³C NMR (125 MHz, CD₂Cl₂-MeOD) δ 167.3, 160.3, 160.1, 158.9, 149.9, 133.8, 129.6, 129.5, 127.1, 126.6, 125.4, 125.0, 122.5, 122.2, 115.3, 114.5, 114.3, 109.5, 67.1, 58.4, 45.8, 28.8, 25.8, 17.8, 8.2. IR (film) νₘₐₓ 3408, 2968, 2930, 2883, 2862, 1757, 1705, 1664, 1605, 1501, 1367, 1263, 1178, 1109 cm⁻¹. HRMS (ESI⁺) m/z Calc for [M+H⁺] C26H31N2O5 451.2233. Found: 451.2231.

N-(7-(3-(Dimethylamino)propoxy)-8-methyl-2-oxo-2H-chromen-3-yl)-4-hydroxy-3-(3-methylbut-2-enyl)benzamide (310. Compound 31f was obtained as a white, amorphous solid (22 mg, 69%). ¹H NMR (500 MHz, DMSOd₆) δ 9.24 (s, 1H), 8.47 (s, 1H), 7.68˜7.66 (m, 2H), 7.56 (d, J=8.7, 1H), 7.07 (d, J=8.7, 1H), 6.89 (d, J=8.7, 1H), 5.31 (m, 1H), 4.13 (t, J=6.2, 2H), 3.27 (d, J=7.2, 2H), 2.47 (t, J=6.9, 2H), 2.22 (s, 3H), 2.21 (s, 6H), 1.92 (m, 2H), 1.71 (s, 3H), 1.70 (s, 3H). ¹³C NMR (125 MHz, DMSOd₆) δ 165.3, 158.7, 158.4, 158.1, 149.3, 131.8, 129.2, 128.1, 127.6, 126.8, 126.1, 124.0, 122.3, 121.2, 114.5, 112.7, 112.4, 109.1, 66.6, 55.5, 45.0, 40.1, 27.9, 26.6, 25.5, 17.7, 7.9.1R (film) νₘₐₓ 3408, 2961, 2928, 1709, 1666, 1607, 1529, 1504, 1367, 1256, 1178, 1109 cm⁻¹. HRMS (ESI⁺) m/z Calc for [M+H⁺] C27H33N2O5 465.2389. Found: 465.2388.

4-Hydroxy-N-(8-methyl-7-(2-(methylamino)ethoxy)-2-oxo-2H-chromen-3-yl)-3-(3-methylbut-2-enyl)benzamide (31g). Compound 31g was obtained as a light brown, amorphous solid (11 mg, 46%). NMR (400 MHz, CDCl₃-CD₃OD) δ 8.42 (s, 1H), 7.38 (d, J=2.3, 1H), 7.33 (dd, J=8.4, 2.4, 1H), 7.11 (d, J=8.7, 1H), 6.68 (d, J=8.7, 1H), 6.59 (d, J=8.4, 1H), 5.07 (m, 1H), 3.94 (t, J=5.0, 2H), 3.08 (d, J=7.2, 2H), 2.88 (t, J=5.0, 2H), 2.32 (s, 3H), 2.06 (s, 3H), 1.49 (s, 3H), 1.45 (s, 3H). ¹³C NMR (100 MHz, CDCl₃-MeOD) δ 166.5, 159.4, 159.1, 157.6, 148.8, 133.0, 128.6, 128.5, 126.1, 125.6, 124.5, 124.4, 123.9, 121.3, 114.4, 113.7, 113.5, 108.8, 66.2, 49.3, 34.6, 27.7, 25.1, 17.1, 7.4.1R (film) νₘₐₓ 3398, 2956, 2924, 2854, 1697, 1655, 1605, 1533, 1508, 1373, 1261, 1111 cm⁻¹. HRMS (ESI⁺) m/z Calc for [M+H⁺] C25H29N2O5 437.2076. Found: 437.2076.

### Example 22

### Primary Cultures of Human ADPKD Cells

Primary cultures of human ADPKD cyst-lining epithelial cells were established as described in Yamaguchi, T., et al., Calcium restores a normal proliferation phenotype in human polycystic kidney disease epithelial cells. J Am Soc Nephrol, 2006. 17(1): p. 178-87. Cystic tissue from ADPKD kidneys were minced and digested overnight in DMEM/F12 (1:1) mixture that contained 220 IU/ml type IV collagenase and 100 IU/ml penicillin G and 0.1 mg/ml streptomycin (P/S) (13). Collagenase digestion was stopped by the addition of FBS. Cells were rinsed in medium and propagated in DMEM/F12 supplemented with 5% FBS, 5 μg/ml insulin, 5 μg/ml transferrin, and 5 ng/ml sodium selenite (ITS) and penicillin/streptomycin (P/S). At 70 to 80% confluence, cells were lifted from the plastic and either frozen in medium that contained 10% DMSO for storage in liquid N2 or used directly for experiments.

### Example 23

### Cell Proliferation Assay

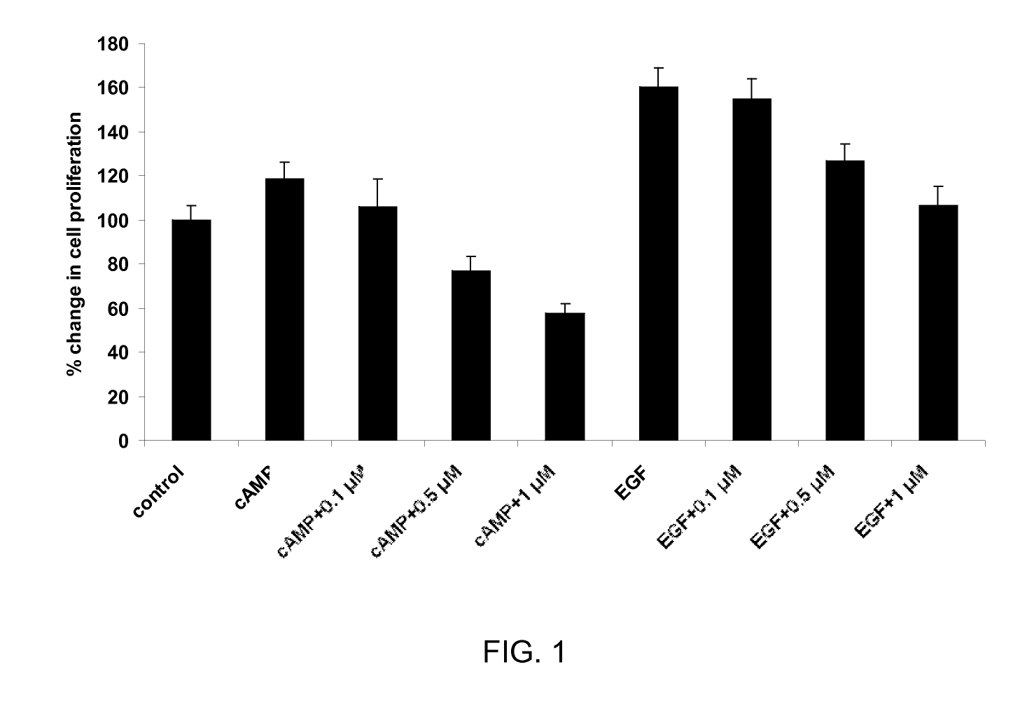

The novobiocin analog KU-174 was studied to determine the effect on cAMP and EGF-stimulated proliferation of ADPKD cyst-lining epithelial cells. Cell proliferation was determined by the Promega Cell Titer 96 MTT assay method as described in Yamaguchi, T., et al., Calcium restores a normal proliferation phenotype in human polycystic kidney disease epithelial cells. J Am Soc Nephrol, 2006. 17(1): p. 178-87. Human ADPKD cyst-lining epithelial cells (4×10³/well) cells were seeded in a 96-well culture plate (n=6 wells per treatment) and incubated for 24 h in DMEM/F12 with 1% FBS, ITS, and P/S. The FBS and ITS were removed, and the cells were incubated for 24 h before the addition of 100 μM cAMP or 25 ng/ml EGF to stimulate cell proliferation. Increasing concentrations of KU-174 (0.1 uM, 0.5 uM, 1 uM KU-174) were added to the medium. After 72 hours of treatment, cell proliferation was measured by adding the MTT assay reagent and stopping the reaction four hours later. Absorbance was read on a spectrophotometer at 570 nm. Optical densities were measured using a spectrophotometer. In each group, results were expressed as a percentage of the control group (not treated with KU-174). FIG. 1 shows dose-dependent inhibition of p-ERK in EGF stimulated ADPKD cells treated with KU-174 by Western blot. MTT assays showed that KU-174 significantly inhibited both cAMP- and EGF-induced proliferation of PKD (e.g., ADPKD) cells in a dose-dependent manner (0.1 uM, 0.5 uM, 1 uM KU-174 for 72 h).

### Example 24

### Inhibition of p-ERK in EGF Stimulated ADPKD Cells Treated with KU-174 by Western Blot Assay

Human ADPKD cells were plated at a density of 1×10⁵ cells per well in medium containing 1% FBS in a 6-well plate. Cells were serum-starved for 24 hours and treated with 25 ng/ml EGF to stimulate cell proliferation, then treated with increasing concentrations of KU-174. As a control for inhibition of the mTOR pathway, ADPKD cells were stimulated by EGF and treated with Rapamycin or LY294002. After 24 or 48 hours, cytoplasmic protein extracts were prepared by lysing cells in ice-cold lysis buffer (10 mM Tris-Cl pH 7.5, 150 mM NaCl, 2 mM EDTA pH 8.0, 1% Triton X-100, 0.5% NP-40, 25 mM glycerol 2-phosphate, 1 mM sodium orthovanadate, 1 mM phenylmethylsulfonyl fluoride, and 0.1% v/v Sigma protease inhibitor cocktail). Nuclei and other Triton-insoluble components were removed by high speed centrifugation. Protein concentration was measured using the Pierce BCA assay kit. 20 μg total protein was boiled with SDS sample buffer and fractionated on 7.5, 10 or 12.5% SDS-PAGE gels. Proteins were transferred to PVDF membranes and non-specific binding blocked with 5% powdered milk in TBS-T (10 mM Tris-Cl pH 7.5, 150 mM NaCl, and 0.1% Tween 20) for 1 h at room temperature. Blocked membranes were incubated with primary antibodies in 5% powdered milk or 5% BSA (for phospho-proteins) in TBS-T overnight at 4° C. Membranes were then washed three times with TBS-T and incubated with alkaline phosphatase-conjugated secondary antibodies in 5% milk in TBS-T for 30 min at room temperature. The membranes were washed three times with TBS-T, and protein bands were visualized using the CDP-star detection reagent (GE healthcare). Intensity was detected and quantitatively analyzed by the Fluor-S MAX multi-imager system (Bio-Rad).

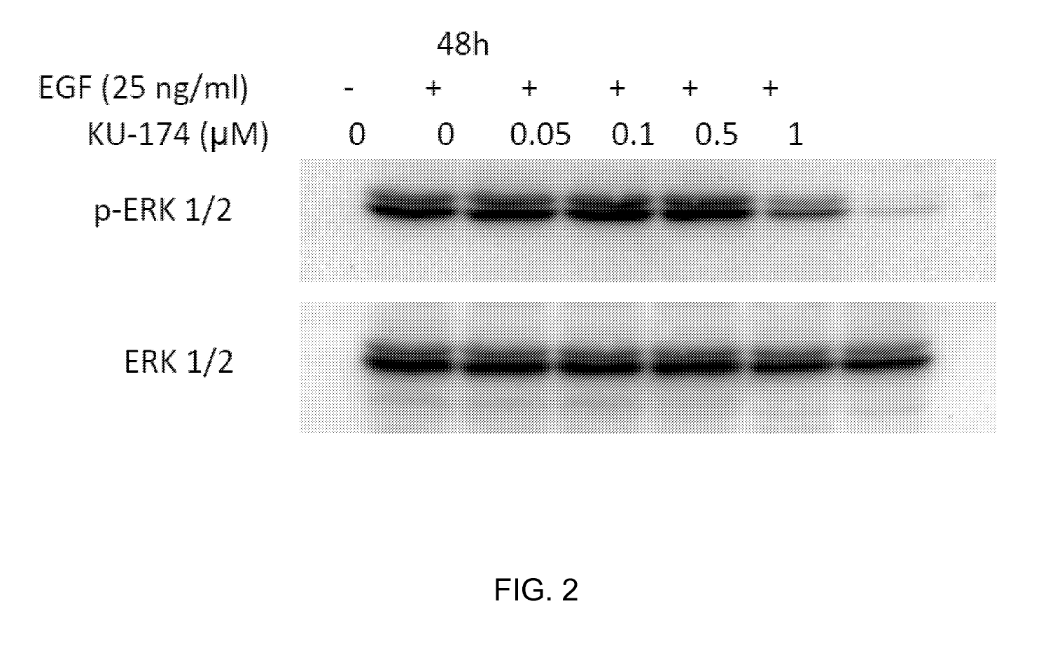

Treatment of cells with 0.5 μM and 1 μM KU-174 treatment strongly decreased the phosphorylation of ERK 1/2 (70% and 80% decreases respectively; n=3) whereas total ERK levels remained virtually unchanged. FIG. 2 shows dose-dependent inhibition of p-ERK in EGF stimulated ADPKD cells treated with KU-174 by Western blot. Inhibition of p-ERK correlates with the decrease in cell proliferation seen in FIG. 1.

### Example 25

### Inhibition of mTOR Pathway in EGF Stimulated ADPKD Cells Treated with KU-174 by Western Blot Assay

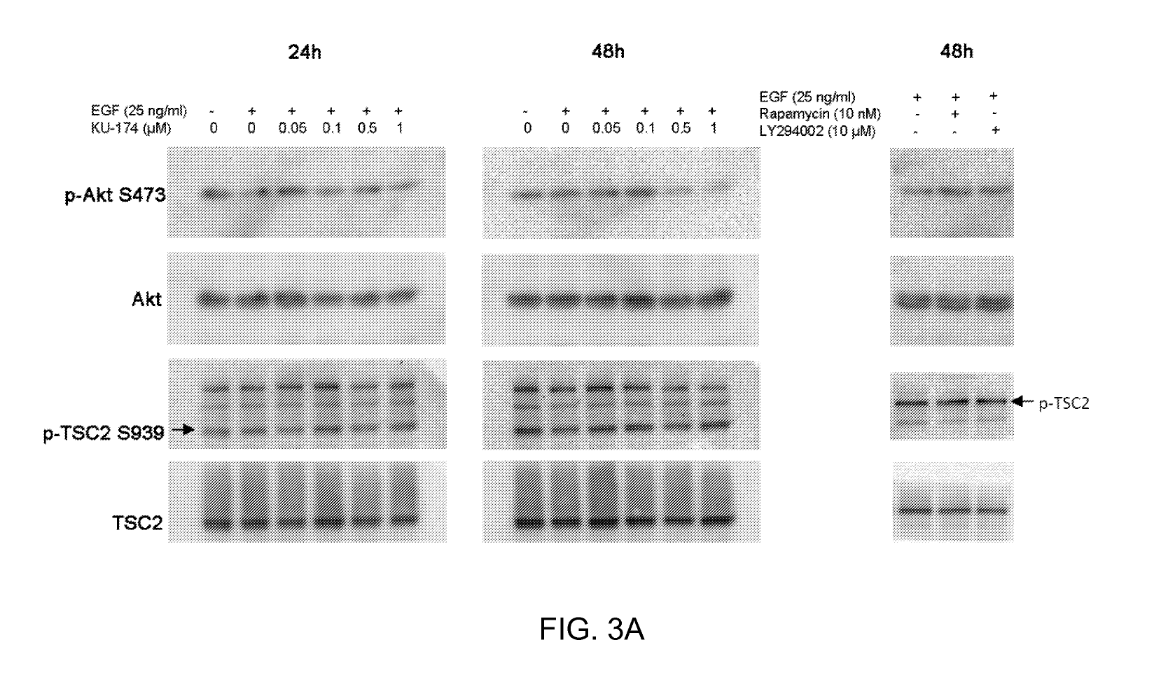

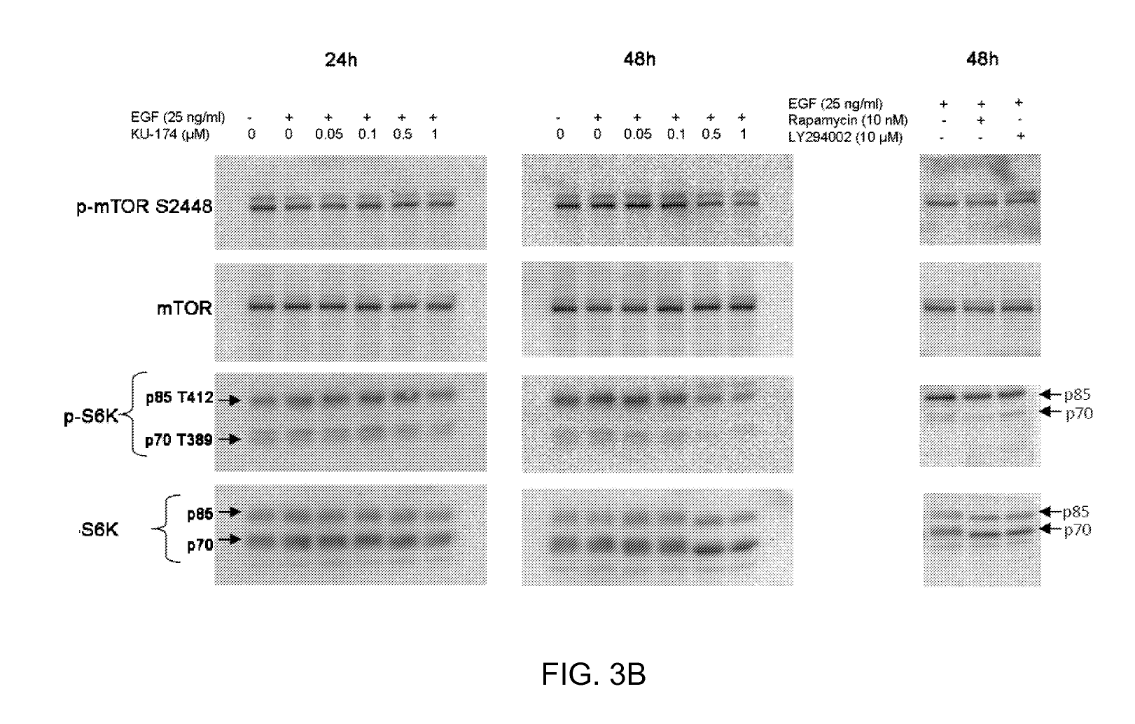

Human ADPKD cells were plated at a density of 1×10⁵ cells per well in medium containing 1% FBS in a 6-well plate. Cells were serum-starved for 24 hours and treated with 25 ng/ml EGF to stimulate cell proliferation, then treated with increasing concentrations of KU-174. As a control for inhibition of the mTOR pathway, ADPKD cells were stimulated by EGF and treated with Rapamycin or LY294002, (an inhibitor of phosphoinositide-3-kinases, PI3Ks). After 24 or 48 hours, cells were lysed and 20 μg total cell lysate analyzed by Western blotting. Blots were probed with antibodies against p-Akt (S473), Akt, p-TSC2 (S939), TSC2, p-mTOR (S2448), mTOR, p-S6K (T412 and T389; phospho-p70 S6 Kinase phosphorylated at T389 or T412) and S6K (p70 S6 kinase; p70S6K1). FIG. 3A shows Western blot of mTOR pathway mediators p-Akt, Akt, p-TSC2, and TSC2 from EGF-stimulated ADPKD cells at 24 and 48 hours post-stimulation. FIG. 3B shows Western blot of mTOR pathway mediators p-mTOR, mTOR, p-S6K and S6K EGF-stimulated cells at 24 and 48 hours. After 24 h of treatment, there was no change in any of the phospho-proteins, except for a slight decrease in p-S6K at the 1 μM KU-174 concentration. By 48 h, both 0.5 and 1 μM KU-174 were effective in decreasing the phosphoproteins. In the 1 μM KU-174 treated cell lysates, p-Akt was decreased by 64%, p-mTOR by 56%, and p-S6K isoforms p85 and p70 by 75% compared with control EGF treated cells and normalized with respect to the corresponding total protein (n=3). There was no change in p-TSC2 levels. Even though Akt is a well-known Hsp90 client protein, KU-174 did not appear to decrease total Akt levels at the concentrations examined. The figures represent results from independent experiments using primary human ADPKD cells from three different kidneys.

### Example 26

### KU-174 Treatment Decreases Hsp90 Client Protein Levels in EGF Stimulated Human ADPKD Cells by Western Blot

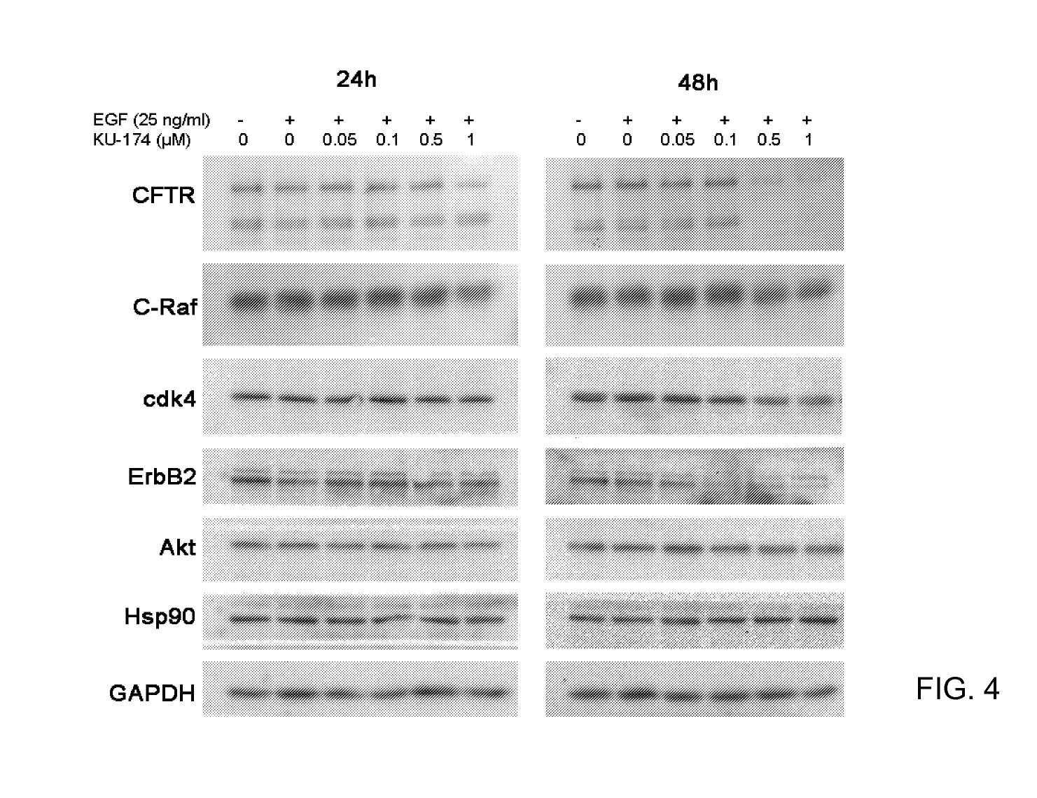

Proteins from 20 μg total cell lysate were analyzed by Western blotting. Blots were probed with antibodies against Hsp90 and client proteins CFTR, C-Raf, cdk4, ErbB2 and Akt. GAPDH served as the internal control. As shown in FIG. 4, at 24 h, 1 μM KU-174 decreased the total levels of CFTR and C-Raf in ADPKD cells, while at 48h, both 0.5 and 1 μM were effective in decreasing all the client proteins examined, except Akt.

### Example 27

### Microcyst Assay

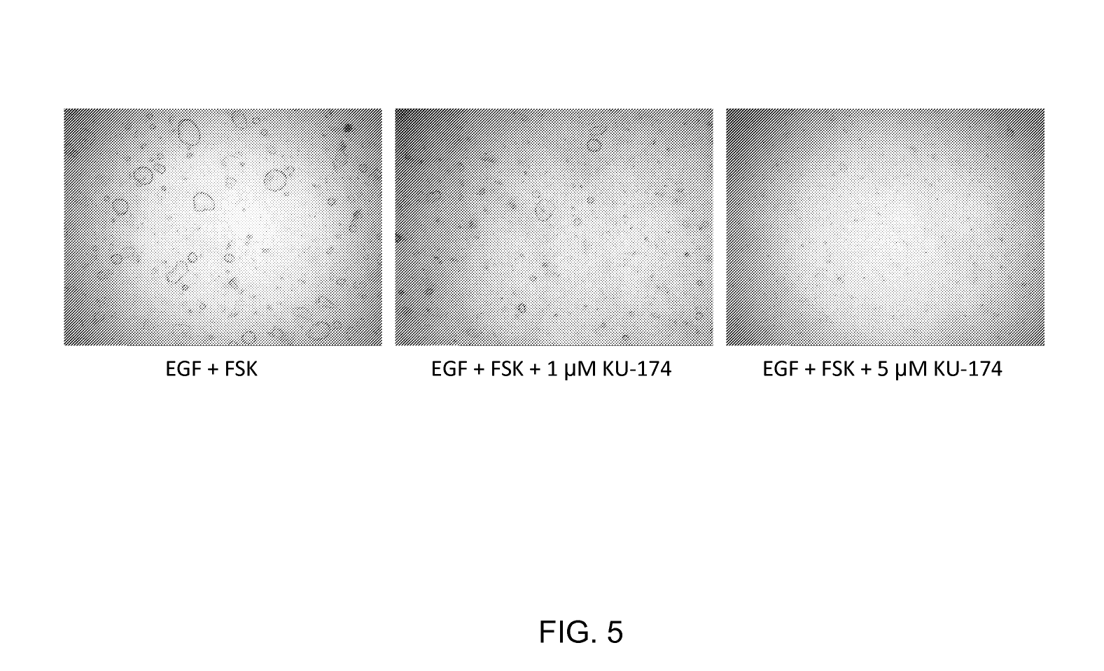

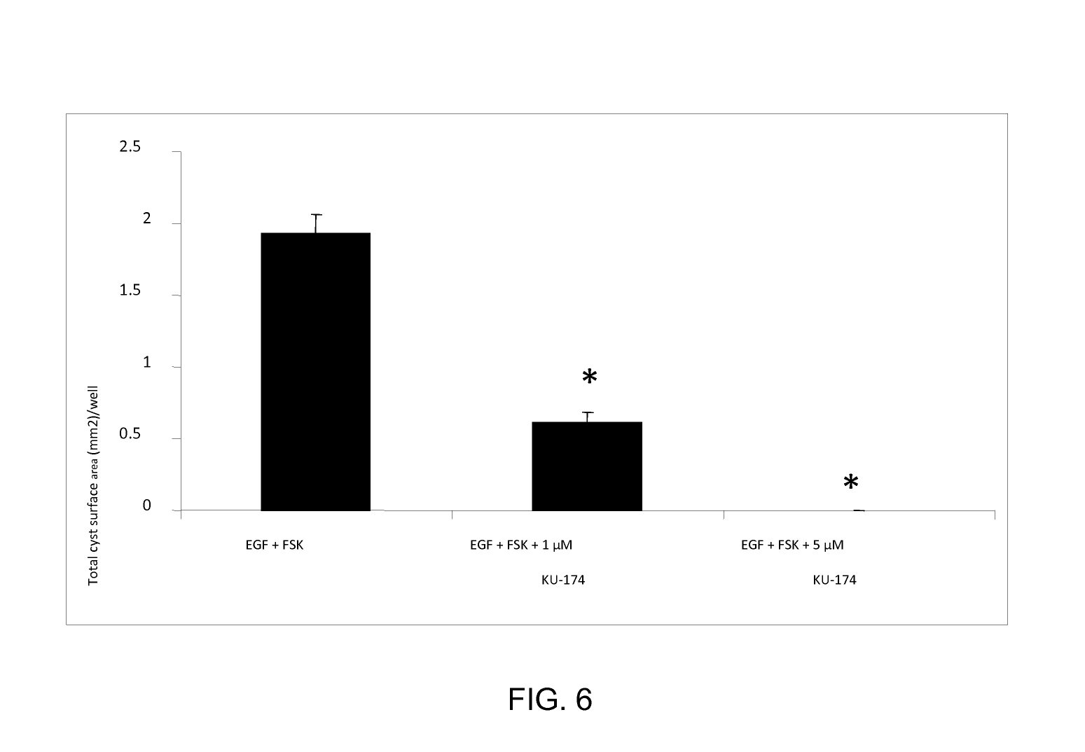

Microcyst assay was performed as described previously by Yamaguchi, T., et al., Calcium restores a normal proliferation phenotype in human polycystic kidney disease epithelial cells. J Am Soc Nephrol, 2006. 17(1): p. 178-87. Primary cultures of human ADPKD cells (4×10³ cells/well) were dispersed within an ice-cold type I collagen matrix (Vitrogen; Collagen Corp., Palo Alto, Calif.) in wells of a 96-well culture plate. Warming the plate to 37° C. caused polymerization of the collagen, trapping the cells within the gel. A defined medium (DMEM/F12 with ITS, 5×10⁻⁸M hydrocortisone, and 5×10⁻⁵ M triiodothyronine) supplemented with 5 μM forskolin and 5 ng/ml EGF was added for 3 days to initiate cyst growth. Forskolin (FSK) is a small molecule agonist of adenylyl cyclase, the enzyme that makes cAMP. Thus, FSK treatment of cells increases the levels of intracellular cAMP. Fresh media containing EGF and forskolin plus 1 μM or 5 μM KU-174 were added for an additional 5-7 days. FIG. 5 shows the collagen gel matrix containing cysts under various conditions. Gels containing cysts were fixed in 1% formaldehyde and cyst diameters were measured using a morphometric analysis system. Outer diameters of cross-sectional images of spherical cysts with distinct lumens were measured using an inverted microscope with a digital camera and video analysis software. The total surface area (SA) of the cysts within each well was calculated from the individual cyst diameters (cysts≧100 μm). KU-174 inhibits microcyst formation by ADPKD cells in a collagen gel matrix. As shown in FIG. 6, 1 μM KU-174 caused a significant inhibition in cyst growth, and 5 μM KU-174 completely eliminated cyst formation (*p<0.01).

### Example 28

### Metanephric Organ Culture

Pkd1^{m1Bei} mice were obtained from the Mutant Mouse Regional Resource Center (University of North Carolina, Chapel Hill, N.C.) and were stabilized onto a C57BL/6 background (>10 backcrosses). This mouse has a point mutation (T to G at 9248 bp) that causes an M to R substitution that affects the first transmembrane domain of polycystin-1 as described by Herron, B. J., et al., Efficient generation and mapping of recessive developmental mutations using ENU mutagenesis. Nat Genet, 2002. 30(2): p. 185-9.

Mouse metanephric kidneys were cultured according to methods described in Magenheimer, B. S., et al., Early embryonic renal tubules of wild-type and polycystic kidney disease kidneys respond to cAMP stimulation with cystic fibrosis transmembrane conductance regulator/Na(+),K(+),2Cl(−) Co-transporter-dependent cystic dilation. J Am Soc Nephrol, 2006. 17(12): p. 3424-37. Metanephroi were dissected from Pkd1+/− embryonic mice and placed on transparent Falcon 0.4-mm cell culture inserts. DMEM/F12-defined culture medium (supplemented with 2 mM L-glutamine, 10 mM HEPES, 5 μg/ml insulin, 5 μg/ml transferrin, 2.8 nM selenium, 25 ng/ml prostaglandin E, 32 pg/ml T3, 250 U/ml penicillin, and 250 μg/ml streptomycin) was added under the culture inserts, and organ cultures were maintained in a 37° C. humidified CO₂ incubator for up to 5 days. Kidneys were treated with 100 μM cAMP along with 10 μM KU-174 or captisol (vehicle) for four days. Upon culturing (day 0) and approximately 24 h later (day 1) and each day after (days 2 to 4), kidneys were photographed using a 2× or 4× objective, and the images were acquired and analyzed using the analySIS imaging program (Soft Imaging System, Munster, Germany).

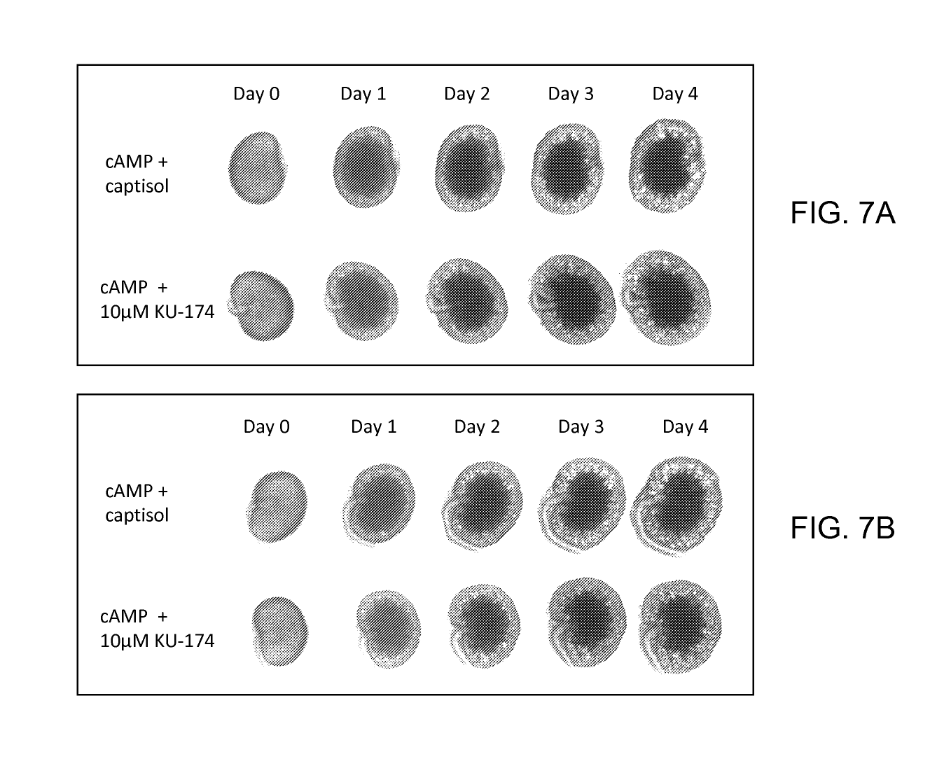

To study the effects of the novobiocin analogue, KU-174, on cyst formation and enlargement in kidney organ culture, a mouse model of PKD, the Pkd1m1bei mouse, was utilized. The gene is orthologous to the gene involved in ˜85% of human ADPKD cases. To determine whether KU-174 alters the cyst-forming process in response to cAMP, embryonic day 15.5 mouse kidneys from Pkd1+/− mice were placed in metanephric culture on Transwell membranes and treated with 100 uM cAMP with or without KU-174 (10 uM) or Captisol (vehicle) for 4 days. Treatment with KU-174 reduced cyst formation. FIGS. 7A and 7B show two different Pkd1+/− metanephric kidneys treated with KU-174. KU-174 treatment reduces the cyst burden in cAMP-treated metanephric kidneys.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.
(1) Powers, M. V., Workman, P. In Endocrine-Related Cancer 2006; Vol. 13, p S125-S135.
(2) Pratt, W. B., Toft, D. O. Exp. Biol. Med. 2003, 228, 111-133.
(3) Issacs, J. S., Xu, W., Neckers, L. Cancer Cell 2003, 3, 213-217.
(4) Terasawa, K., Minami, M., Minami, Y. J. Biochem. 2005, 137, 443-447.
(5) Buchner, J. Trends Biochem. Sci. 1999, 24, 136-141.
(6) Picard, D. Cell. Mol. Life. Sci. 2002, 59, 1640-1648.
(7) Yonehara, M., Minami, Y., Kawata, Y. Nagai, J., Yahara, I. J. Biol. Chem. 1996, 271, 2641-2645.
(8) Xiao, L., Lu, X., Ruden, D. M. Mini Rev. Med. Chem. 2006, 6, 1137-1143.
(9) Zhao, R., Houry, W. A. Biochem. Cell Biol. 2005, 83, 703.
(10) Neckers, L., Ivy, S. P. Curr. Opin. Oncol. 2003, 15, 419-424.
(11) Blagg, B. S. J., Kerr, T. D. Med. Res. Rev. 2006, 26, 310-338.
(12) Yu, X. M., Shen, G., Neckers, L., Blake, H., Holzbeierlein, J., Cronk, B., Blagg, B. S. J. J. Am. Chem. Soc. 2005, 127, 12778-12779.
(13) Zhang, H., Burrows, F. J. Mol. Med. 2004, 82, 488.
(14) Chiosis, G., Vilenchik, M., Kim, J., Solit, D. Drug Discuss. Today 2004, 9, 881.
(15) Burlison, J. A., Neckers, L., Smith, A. B., Maxwell, A., Blagg, B. S. J. J. Am. Chem. Soc. 2006, 128, 15529-15536.
(16) Toft, D. O. Trends Endocrin. Metab. 1998, 9, 238-243.
(17) Walter, S., Buchner, J. J. Agnew. Chem., Int. Ed. 2002, 41, 1098-1113.
(18) Maloney, A., Workman, P. Expert Opin. Biol. Ther. 2002, 2, 3-24.
(19) Workman, P. Cancer Letters 2004, 206, 149-157.
(20) Sreedhar, A. S., Kalmar, E., Csermely, P., Shen, Y. F. FEBS Letters 2004, 562, 11-15.
(21) Lewis, R. J., Tsai, F. T., Wigley, D. B. BioEssays 1996, 18, 661-671.
(22) Reece, R. J., Maxwell, A. Crit. Rev. Biochem. Mol. Biol. 1991, 26, 335-375.
(23) Laurin, P., Ferroud, D., Schio, L., Klich, M., Dupuis-Hamelin, C., Mauvais, P., Lassaigne, P., Bonnefoy, A., Musicki, B. Bioorg. Med. Chem. Lett. 1999, 9, 2875-2880.
(24) Ali, J. A., Jackson, A. P., Howells, A. J., Maxwell, A. Biochemistry 1993, 32.
(25) Holdgate, G. A., Tunnicliffe, A., Ward, W. H. J., Weston, S. A., Rosenbrock, G., Barth, P. T, Taylor, I. W. F., Paupit, R. A., Timms, D. Biochemistry 1997, 36, 9663-9673.
(26) Lewis, R. J., Singh, O. M. P., Smith, C. V., Skarzyknski, T., Maxwell, A., Wonacott, A. J., Wigley, D. B. EMBO J. 1996, 15, 1412-1420.
(27) Tsai, F. T. F., Singh, O. M. P., Skarzynski, T., Wonacott, A. J., Weston, S., Tucker, A., Pauptit, R. A., Breeze, A. L., Poyser, J. P., O'Brien, R., Ladbury, J. E., Wigley, D. B. Proteins: Struct., Fund., Genet. 1997, 28, 41-52.
(28) Roe, S. M., Prodromou, C., O'Brien, R., Ladbury, J. E., Piper, P. W., Pearl, L. H. J. Med. Chem. 1999, 42, 260-266.
(29) Gobernado, M., Canton, E., Santos, M. J. Clin. Microbiol. 1984, 3, 371.
(30) Schwartz, G. N., Teicher, B. A., Eder, J. P., Jr., Korbut, T., Holden, S. A., Ara, G., Herman, T. S. Cancer Chemother. Pharmacol. 1993, 32, 455-462.
(31) Nordenberg, J., Albukrek, D., hadar, T., Fux, A., Wasserman, L., Novogrodsky, A., Sidid, Y. Br. J. Cancer 1992, 65, 183-188.
(32) Hombrouck, C., Capmau, M., Moreau, N. Cell Mol. Biol. 1999, 45, 347-352.
(33) Marcu, M. G., Schulte, T. W., Neckers, L. J. Natl. Cancer Inst. 2000, 92, 242-248.
(34) Marcu, M. G., Chadli, A., Bohouche, I., Catelli, B., Neckers, L. M. J. Biol. Chem. 2001, 276, 37181-37186.
(35) Allan, R. K., Mok, D., Ward, B. K., Ratajczak, T. J. Biol. Chem. 2006, 281, 7161-7171.
(36) Burlison, J. A., Blagg, B. S. J. Org. Lett. 2006, 8, 4855-4858.
(37) Burlison, J. A., Avila, C., Vielhauer, G., Lubbers, D. J., Holzbeierlein, J., Blagg, B. S. J. J. Org. Chem. 2008, 73, 2130-2137.
(38) Huang, Y.-T., Blagg, B. S. J. J. Org. Chem.: 2007, 72, 3609-3613.
(39) Donnelly, A. C., Mays, J. R., Burlison, J. A., Nelson, J. T., Vielhauer, G., Holzbeierlein, J., Blagg, B. S. J. J. Org. Chem. 2008, 73, 8901-8920.
(40) Le Bras, G., Radanyi, C., Peyrat, J.-F., Brio, J.-D., Alami, M., Marsaud, V., Stella, B., Renoir, J.-M. J. Med. Chem. 2007, 50, 6189-6200.
(41) Radanyi, C., Le Bras, G., Messaoudi, S., Bouclier, C., Peyrat, J.-F. Brion, J.-D., Marsaud, V., Renoir, J.-M., Alami, M. Bioorg. Med. Chem. Lett. 2008, 18, 2495-2498.
(42) Wiley, K. P. Hum. Reprod. Update 1999, 5, 330.
(43) Larchen, H. G., Von dem Brunch, K. J. J. Prakt. Chem. (Weinhaim Ger) 2000, 342, 753.
(44) Yu, Y. M., Han, H., Blagg, B. S. J.\ J. Org. Chem. 2005, 70, 5599.
(45) Andrew, J. R., Olga, V. S., Andrei, V. N. Carbohydrate Research 2006, 341, 1954.
(46) Soti, C., Vermes, A., Haystead, T. A. J., Csermely, P. Eur. J. Biochem. 2003, 270, 2421-2428.

From the foregoing it will be seen that this disclosure is one well adapted to attain all ends and objectives herein-above set forth, together with the other advantages which are obvious and which are inherent to the disclosure. Since many possible embodiments may be made of the disclosure without departing from the scope thereof, it is to be understood that all matters herein set forth or shown in the accompanying drawings are to be interpreted as illustrative, and not in a limiting sense. While specific embodiments have been shown and discussed, various modifications may of course be made, and the disclosure is not limited to the specific forms or arrangement of parts and steps described herein, except insofar as such limitations are included in the following claims. Further, it will be understood that certain features and subcombinations are of utility and may be employed without reference to other features and subcombinations. This is contemplated by and is within the scope of the claims.

We claim:

1. A method for treating, inhibiting, and/or preventing one or more symptoms of polycystic kidney disease (PKD) in a subject in need thereof, the method comprising administering to the subject a therapeutically effective amount of a compound according to Formula I: 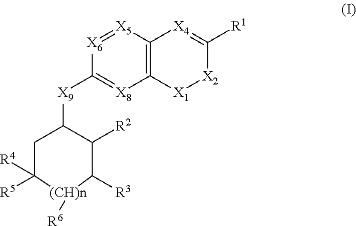
wherein R¹ is an amido which is NR′COR″, and R′ is hydrogen and R″ is C₁-C₄ alkyl, aryl or heterocycle, each optionally substituted with one or more hydroxy, nitro, amino, alkyl, alkenyl, aryl, alkoxy or halo groups;
wherein R² is hydrogen, hydroxy, or —R⁸—OR⁹, wherein R⁸ is a covalent bond or alkyl, and R⁹ is C-amido or acyl; or R² together with R³ and the atoms to which they are attached form a heterocyclic ring having 4 to 8 ring members with at least one heteroatom selected from oxygen or nitrogen;
wherein R³ is hydrogen, hydroxy, or —R¹⁰—O—R¹¹, wherein R¹⁰ is a covalent bond or alkyl, and R¹¹ is C-amido or acyl; or R³ together with R² and the atoms to which they are attached form a heterocyclic ring having 4 to 8 ring members with at least one heteroatom selected from oxygen or nitrogen;
wherein R⁴ is hydrogen, hydroxy, alkyl, carboxyl, —R¹²—O—R¹³, or —R¹²-R¹⁴, and wherein R¹² is a covalent bond or alkyl, and R¹³ is C-amido or acyl, and R¹⁴ is N-amido, —POR¹⁵R¹⁶, —SO₂R¹⁷, or sulfonamido and wherein R¹⁵, R¹⁶, R¹⁷ are independently alkoxy;
wherein R⁵ is hydrogen, alkyl, alkenyl, alkynyl, aryl, or aralkyl;
wherein R⁶ is hydrogen, alkyl, alkenyl, alkynyl, aryl, aralkyl, alkoxy, aryloxy, or aralkoxy;
wherein X₁ is —O—;
wherein X₂ is —CO—;
wherein X₄ is —CR²⁰—, wherein R²⁰ is hydrogen, alkyl, alkenyl, or alkynyl;
wherein X₅ is —CR²¹, wherein R²¹ is hydrogen, alky, alkenyl, alkynyl, or alkoxy;
wherein X₆, is —CR²² wherein R²² is hydrogen, alkyl, alkenyl, alkynyl, alkoxy, aryl, aralkyl, halogen, or nitro;
wherein X₈, is —CR²³, wherein R²³ is hydrogen, alkyl, alkenyl, alkynyl, aryl, or alkoxy;
wherein X₉ is alkyl, alkenyl, alkynyl, ether, secondary or tertiary amino, or sulfanyl; and
wherein n is 1; or a pharmaceutically acceptable salt thereof.

2. The method according to claim 1 comprising administering to the subject a therapeutically effective amount of a compound according to Formula I: 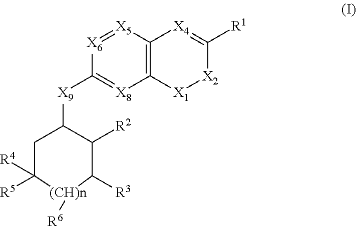
wherein R¹ is an amido which is NR′COR″, and R′ is hydrogen and R″ is aryl or heterocycle;
wherein R² is hydrogen or hydroxy;
wherein R³ is hydrogen or hydroxy;
wherein R⁴ is hydrogen or methyl;
wherein R⁵ is hydrogen, or alkyl;
wherein R⁶ is alkoxy;
wherein X₄ is —CR²⁰—, wherein R²⁰ is hydrogen;
wherein X₅, is —CR²¹, wherein R²¹ is hydrogen;
wherein X₆, is —CR²², wherein R²² is hydrogen, alkyl, or alkoxy;
wherein X₈, is —CR²³, wherein R²³ is hydrogen, alkyl, or alkoxy; and
wherein X₉ is ether.

3. The method according to claim 1 wherein R¹ is an amido which is NR′COR″, and R′ is hydrogen and R″ is aryl according to: 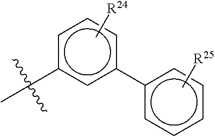
wherein R²⁴ and R²⁵ are independently hydrogen, alkyl, amino, halo, hydroxy, or alkoxy.

4. The method according to claim 1 wherein R¹ is an amido which is NR′COR″, and R′ is hydrogen and R″ is aryl according to: 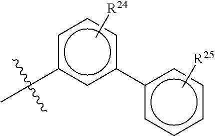
wherein R²⁴ and R²⁵ are hydrogen, alkyl or alkoxy.

5. The method according to claim 1 wherein R¹ is an amido which is NR′COR″, and wherein R′ is hydrogen and R″ is aryl according to: 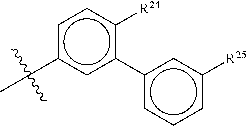
wherein R²⁴ and R²⁵ are alkoxy.

6. The method according to claim 1 wherein R¹ is an amido which is NR′COR″, and wherein R′ is hydrogen and R″ is aryl selected from the group consisting of:
N-(7-((2R,3R,4S,5R)-3,4-dihydroxy-5-methoxy-6,6-dimethyltetrahydro-2H-pyran-2-yloxy)-8-methyl-2-oxo-2H-chromen-3-yl)-6-methoxybiphenyl-3-carboxamide (28);
N-(7-((2R,3R,4S,5R)-3,4-dihydroxy-5-methoxy-6,6-dimethyltetrahydro-2H-pyran-2-yloxy)-8-methyl-2-oxo-2H-chromen-3-yl)-6-methoxy-2′-methylbiphenyl-3-carboxamide (29);
N-(7-((2R,3R,4S,5R)-3,4-dihydroxy-5-methoxy-6,6-dimethyltetrahydro-2H-pyran-2-yloxy)-8-methyl-2-oxo-2H-chromen-3-yl)-6-methoxy-3′-methylbiphenyl-3-carboxamide (30);
N-(7-((2R,3R,4S,5R)-3,4-dihydroxy-5-methoxy-6,6-dimethyltetrahydro-2H-pyran-2-yloxy)-8-methyl-2-oxo-2H-chromen-3-yl)-6-methoxy-4′-methylbiphenyl-3-carboxamide (31);
N-(7-((2R,3R,4S,5R)-3,4-dihydroxy-5-methoxy-6,6-dimethyltetrahydro-2H-pyran-2-yloxy)-8-methyl-2-oxo-2H-chromen-3-yl)-2′,6-dimethoxybiphenyl-3′-carboxamide (32);
N-(7-((2R,3R,4S,5R)-3,4-dihydroxy-5-methoxy-6,6-dimethyltetrahydro-2H-pyran-2-yloxy)-8-methyl-2-oxo-2H-chromen-3-yl)-3′,6-dimethoxybiphenyl-3-carboxamide (33);
N-(7-((2R,3R,4S,5R)-3,4-dihydroxy-5-methoxy-6,6-dimethyltetrahydro-2H-pyran-2-yloxy)-8-methyl-2-oxo-2H-chromen-3-yl)-4′,6-dimethoxybiphenyl-3′-carboxamide (34);
N-(7-((2R,3R,4S,5R)-3,4-dihydroxy-5-methoxy-6,6-dimethyltetrahydro-2H-pyran-2-yloxy)-8-methyl-2-oxo-2H-chromen-3-yl)-2′-hydroxy-6-methoxybiphenyl-3-carboxamide (35);
N-(7-((2R,3R,4S,5R)-3,4-dihydroxy-5-methoxy-6,6-dimethyltetrahydro-2H-pyran-2-yloxy)-8-methyl-2-oxo-2H-chromen-3-yl)-3′-hydroxy-6-methoxybiphenyl-3-carboxamide (36); and
N-(7-((2R,3R,4S,5R)-3,4-dihydroxy-5-methoxy-6,6-dimethyltetrahydro-2H-pyran-2-yloxy)-8-methyl-2-oxo-2H-chromen-3-yl)-4′-hydroxy-6-methoxybiphenyl-3-carboxamide (37).

7. The method according to claim 1 wherein R¹ is an amido which is NR′COR″, and wherein R′ is hydrogen and R″ is aryl according to: 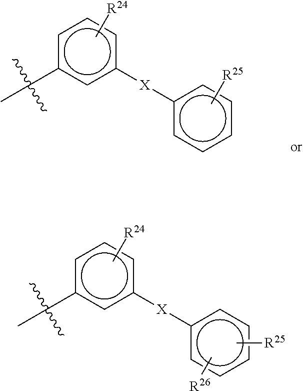
wherein X is ether or amino;
wherein R²⁴ is alkoxy;
wherein R²⁵ is hydrogen, hydroxy, alkoxy, or aryloxy; and
wherein R²⁶ is hydrogen, alkoxy, aryloxy, or amino.

8. The method according to claim 1 wherein R¹ is an amido which is NR′COR″, and wherein R′ is hydrogen and R″ is an indole according to: 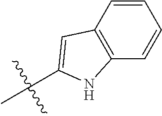
or a R″ is a pendant aryl according to:

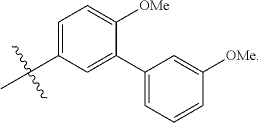

9. The method according to claim 1 wherein selected from the group consisting of:
N-(7-((2S,3R,4S,5R)-3,4-dihydroxy-5-methoxy-6,6-dimethyltetrahydro-2H-pyran-2-yloxy)-6-methoxy-8-methyl-2-oxo-2H-chromen-3-yl)-3′,6-dimethoxybiphenyl-3-carboxamide (26a);
N-(7-((2S,3R,4S,5R)-3,4-dihydroxy-5-methoxy-6,6-dimethyltetrahydro-2H-pyran-2-yloxy)-8-methyl-2-oxo-6-propoxy-2H-chromen-3-yl)-3′,6-dimethoxybiphenyl-3-carboxamide (26b);
N-(7-((2S,3R,4S,5R)-3,4-dihydroxy-5-methoxy-6,6-dimethyltetrahydro-2H-pyran-2-yloxy)-6-isopropoxy-8-methyl-2-oxo-2H-chromen-3-yl)-3′,6-dimeth-oxybiphenyl-3-carboxamide (26c);
N-(7-((2R,3R,4S,5R)-3,4-dihydroxy-5-methoxy-6,6-dimethyltetrahydro-2H-pyran-2-yloxy)-5-methoxy-8-methyl-2-oxo-2H-chromen-3-yl)-3′,6-dimethoxybiphenyl-3-carboxamide (26d);
N-(8-benzyl-7-((2R,3R,4S,5R)-3,4-dihydroxy-5-methoxy-6,6-dimethyltetrahydro-2H-pyran-2-yloxy)-2-oxo-2H-chromen-3-yl)-3′,6-dimethoxybiphenyl-3-carboxamide (26e);
N-(7-((2R,3R,4S,5R)-3,4-dihydroxy-5-methoxy-6,6-dimethyltetrahydro-2H-pyran-2-yloxy)-2-oxo-8-phenyl-2H-chromen-3-yl)-3′,6-dimethoxybiphenyl-3-carboxamide (26f);
N-(7-((2S,3R,4S,5R)-3,4-dihydroxy-5-methoxy-6,6-dimethyltetrahydro-2H-pyran-2-yloxy)-8-methoxy-2-oxo-2H-chromen-3-yl)-3′,6-dimethoxybiphenyl-3-carboxamide (26g, KU-174);
N-(7-((2R,3R,4S,5R)-3,4-dihydroxy-5-methoxy-6,6-dimethyltetrahydro-2H-pyran-2-yloxy)-8-ethyl-2-oxo-2H-chromen-3-yl)-3′,6-dimethoxybiphenyl-3-carboxamide (26h).

10. The method of claim 9 wherein the compound is N-(7-((2S,3R,4S,5R)-3,4-dihydroxy-5-methoxy-6,6-dimethyltetrahydro-2H-pyran-2-yloxy)-8-methoxy-2-oxo-2H-chromen-3-yl)-3′,6-dimethoxybiphenyl-3-carboxamide (26g, KU-174).

11. The method according to claim 1 wherein the compound is selected from the group consisting of:
N-(7-((2S,3R,4S,5R)-3,4-dihydroxy-5-methoxy-6,6-dimethyltetrahydro-2H-pyran-2-yloxy)-6-methoxy-8-methyl-2-oxo-2H-chromen-3-yl)-1H-indole-2-carboxamide (26i);
N-(7-((2S,3R,4S,5R)-3,4-dihydroxy-5-methoxy-6,6-dimethyltetrahydro-2H-pyran-2-yloxy)-8-methyl-2-oxo-6-propoxy-2H-chromen-3-yl)-1H-indole-2-carboxamide (26j);
N-(7-((2S,3R,4S,5R)-3,4-dihydroxy-5-methoxy-6,6-dimethyltetrahydro-2H-pyran-2-yloxy)-6-isopropoxy-8-methyl-2-oxo-2H-chromen-3-yl)-1H-indole-2-carboxamide (26k);
N-(7-((2R,3R,4S,5R)-3,4-dihydroxy-5-methoxy-6,6-dimethyltetrahydro-2H-pyran-2-yloxy)-5-methoxy-8-methyl-2-oxo-2H-chromen-3-yl)-1H-indole-2-carboxamide (26l);
N-(8-benzyl-7-((2R,3R,4S,5R)-3,4-dihydroxy-5-methoxy-6,6-dimethyltetrahydro-2H-pyran-2-yloxy)-2-oxo-2H-chromen-3-yl)-1H-indole-2-carboxamide (26m);
N-(7-((2R,3R,4S,5R)-3,4-dihydroxy-5-methoxy-6,6-dimethyltetrahydro-2H-pyran-2-yloxy)-2-oxo-8-phenyl-2H-chromen-3-y 1)-1H-indole-2-carboxamide (26n);
N-(7-((2S,3R,4S,5R)-3,4-dihydroxy-5-methoxy-6,6-dimethyltetrahydro-2H-pyran-2-yloxy)-8-methoxy-2-oxo-2H-chromen-3-yl)-1H-indole-2-carboxamide (26o); and
N-(7-((2R,3R,4S,5R)-3,4-dihydroxy-5-methoxy-6,6-dimethyltetrahydro-2H-pyran-2-yloxy)-8-ethyl-2-oxo-2H-chromen-3-yl)-1H-indo le-2-carboxamide (26p).

12. The method according to claim 1 wherein R¹ is an amido which is NR′COR″, and R′ is hydrogen and R″ is a heterocycle selected from the group consisting of pyridine, benzofuran, indole, and oxazole.

13. The method according to claim 1 wherein R¹ is an amido which is NR′COR″, and R′ is hydrogen and R″ is aryl or heterocycle according to: 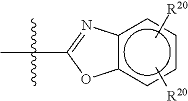
wherein R²⁹ is hydrogen, alkoxy, or amino; and
wherein R³⁰ is hydrogen, alkoxy, or aryloxy.

14. The method according to claim 1 wherein R¹ is an amido which is NR′COR″, and R′ is hydrogen and R″ is a heterocycle according to: 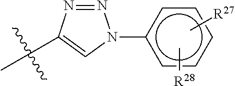
wherein R²⁷ is hydrogen, hydroxy, alkoxy, or aryloxy; and
wherein R²⁸ is hydrogen, alkoxy, aryloxy, or amino.

15. The method according to claim 1 wherein R¹ is an amido which is NR′COR″, and R′ is hydrogen and R″ is a heterocyle according to: 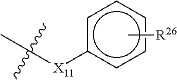
wherein X₁₁ is a covalent bond, alkyl, alkenyl, alkynyl, or —OCH₂—
wherein R²⁶ is hydrogen, aryl, amino, or hydroxy.

16. The method according to claim 1 wherein R¹ is an amido which is NR′COR″, and R′ is hydrogen and R″ is aryl according to one of the following:

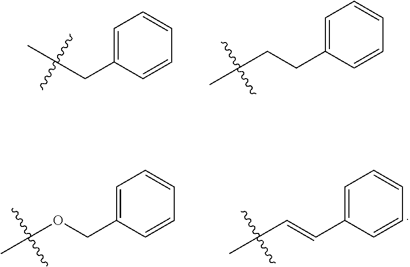

17. The method according to claim 1 wherein the compound is selected from the group consisting of:
N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)-2-phenylacetamide (22);
N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)-3-phenylpropanamide (23);
Benzyl 7-((2R,3R,4S,5R)-3,4-dihydroxy-5-methoxy-6,6-dimethyltetrahydro-2H-pyran-2-yloxy)-8-methyl-2-oxo-2H-chromen-3-ylcarbamate (24); and
N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)cinnamamide (25).

18. The method according to claim 1 wherein R¹ is an amido which is NR′COR″, and R′ is hydrogen and R″ is a heterocycle according to one of the following:

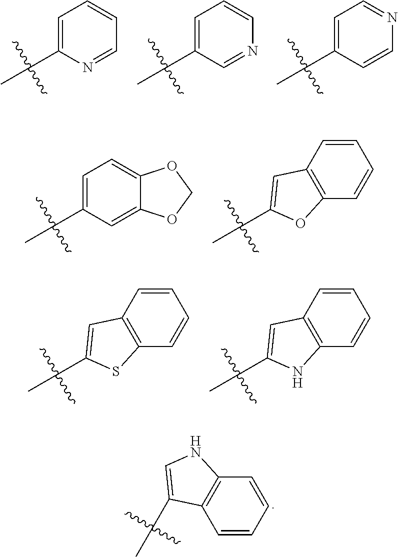

19. The method according to claim 1 wherein the compound is selected from the group, consisting of:
N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)picolinamide (40);
N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)nicotinamide (41);
N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)isonicotinamide (42);
N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)benzofuran-2-carboxamide (43);
N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)-1H-indole-2-carboxamide (46); and
N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)-1H-indole-3-carboxamide (47).

20. The method according to claim 19 wherein the compound is:
N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)-1H-indole-2-carboxamide (46).

21. The method of claim 1 wherein the compound is selected from the group consisting of:
N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)benzamide (8);
N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)biphenyl-2-carboxamide (12);
N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)biphenyl-3-carboxamide (13);
2-Amino-N-(7-((2R,3R,4S,5R)-3,4-dihydroxy-5-methoxy-6,6-dimethyltetrahydro-2H-pyran-2-yloxy)-8-methyl-2-oxo-2H-chromen-3-yl)benzamide (18);
3-Amino-N-(7-((2R,3R,4S,5R)-3,4-dihydroxy-5-methoxy-6,6-dimethyltetrahydro-2H-pyran-2-yloxy)-8-methyl-2-oxo-2H-chromen-3-yl)benzamide (19);
4-Amino-N-(7-((2R,3R,4S,5R)-3,4-dihydroxy-5-methoxy-6,6-dimethyltetrahydro-2H-pyran-2-yloxy)-8-methyl-2-oxo-2H-chromen-3-yl)benzamide (20);
N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)-2-methoxybenzamide (9);
N-(7-((2R,3R,4S,5R)-3,4-Dihydroxy-5-methoxy-6,6-dimethyltetrahydro-2H-pyran-2-yloxy)-8-methyl-2-oxo-2H-chromen-3-yl)-3-methoxybenzamide (10); and
N-(7-((2R,3R4S,5R)-3,4-Dihydroxy-5-methoxy-6,6-dimethyltetrahydro-2H-pyran-2-yloxy)-8-methyl-2-oxo-2H-chromen-3-yl)-4-methoxybenzamide (11).

22. The method of claim 1 wherein the PKD is autosomal dominant polycystic kidney disease (ADPKD).

23. The method of claim 1 wherein the one or more symptoms are selected from the group consisting of cyst formation in the kidneys, increase in cyst size in the kidneys, increase in number of cysts in the kidneys, increase in kidney size, and end stage renal disease.

24. A method for treating, inhibiting, and/or preventing one or more symptoms of polycystic kidney disease (PKD) in a subject in need thereof, the method comprising administering to the subject a therapeutically effective amount of a compound according to Formula II: 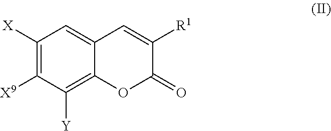
wherein:
R¹ is —NHCOR″, where R″ is a C₁-C₄ alkyl, aryl or heterocyclic group, each optionally substituted with one or more hydroxy, nitro, amino, alkyl, alkenyl, aryl, alkoxy or halo groups;
X⁹ is —O-alkyl, —O-alkylamino, —O-cycloalkyl, —O—(CO)-alkyl, —O—(CO)-cycloalkyl, —O—(CH₂)ₙ-pyridinyl, —O—(CH₂)ₙ-piperidinyl, —O—(CH₂)ₙ-pyrrolino, or —O—(CH₂)ₙ-pyrrolidinyl, each substituted with one or more amino, amido, alkyl, alkoxy, halo, pyrrolidinyl, or hydroxyl groups; and where n is 0, 1 2 or 3; or —O-mono-hydroxylated furanose, —O-dihydroxylated furanose,
—O-mono-hydroxylated pyranose, —O-dihydroxylated pyranose, —O-trihydroxylated pyranose, —O-mono-hydroxylatedoxepinose, —O-dihydroxylated oxepinose, —O-azasugar, —O-acyl, ester, amino, amido, carbamate, phosphate ester, tosylate, mesylate or —OH;
X is H, nitrile, halo, amino, amido, C₁-C₄ alkyl or alkoxy; and
Y is H, amido, ester, amino, C₁-C₄ alkyl or alkoxy; or
a pharmaceutically acceptable salt thereof.

25. The method of claim 24 wherein X₉ is —O-alkyl, —O-alkylamino, —O-cycloalkyl, —O—(CO)-alkyl, —O—(CO)-cycloalkyl, —O—(CH₂)ₙ-pyridinyl, —O—(CH₂)ₙ-piperidinyl, —O—(CH₂)ₙ-pyrrolino, or —O—(CH₂)ₙ-pyrrolidinyl, each optionally substituted with one or more amino, amido alkyl, halo, alkoxy, or hydroxyl groups.

26. The method of claim 24 wherein X₉ is

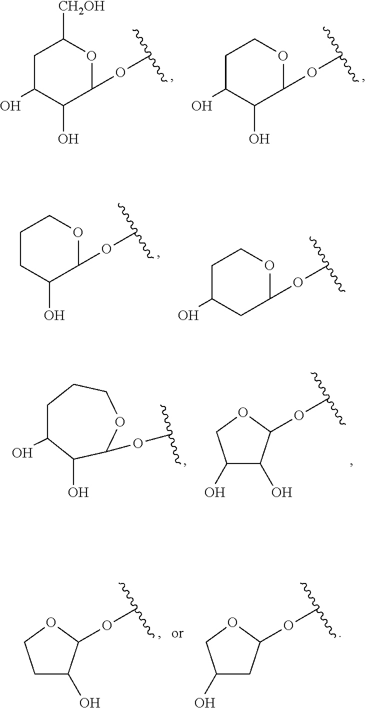

27. The method of claim 24 wherein R¹ is —NHCOCH₃.

28. The method of claim 24 wherein R″ is an aryl group selected from: 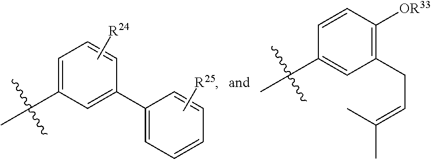
wherein R²⁴ and R²⁵ are independently H, C₁-C₄ alkyl, hydroxy or alkoxy; and
R³³ is H, C₁-C₄ alkyl, C₁-C₄ alkylamino, —(CO)—C₁-C₄ alkyl, or
piperidinyl, each optionally substituted with C₁-C₄ alkyl; or
R″ is a heterocyclic group: 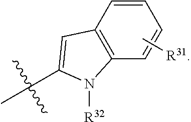
wherein R³¹ is H, halo, C₁-C₄ alkyl, hydroxy or alkoxy; and R³² is H or C₁-C₄ alkyl.

29. The method of claim 28 wherein R″ is an aryl group:

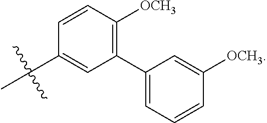

30. The method of claim 29 in which the compound is selected from the group consisting of:

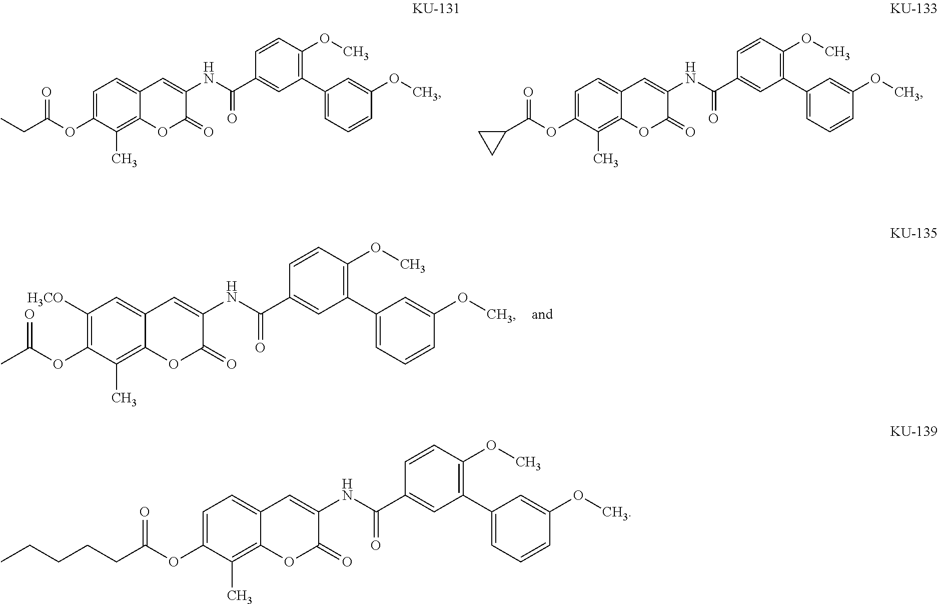

31. The compound of claim 28 wherein R″ is aryl according to: 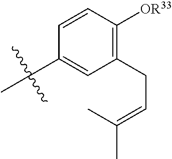
wherein R³³ is H, —CH₃, —COCH₃, —CH₂CH₂N(CH₃)₂, —CH₂CH₂CH₂N(CH₃)₂, or

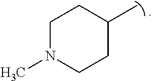

32. The method of claim 31 wherein the compound is selected from the group consisting of
4-(8-Methyl-7-(1-methylpiperidin-4-yloxy)-2-oxo-2H-chromen-3-ylcarbamoyl)-2-(3-methylbut-2-enyl)phenyl acetate (29a, KU-397);
4-(8-Methyl-7-(1-methylpiperidin-3-yloxy)-2-oxo-2H-chromen-3-ylcarbamoyl)-2-(3-methylbut-2-enyl)phenyl acetate (29c, KU-417);
4-(7-(2-(Dimethylamino)ethoxy)-8-methyl-2-oxo-2H-chromen-3-ylcarbamoyl)-2-(3-methylbut-2-enyl)phenyl acetate (29e, KU-421);
4-(7-(3-(Dimethylamino)propoxy)-8-methyl-2-oxo-2H-chromen-3-ylcarbamoyl)-2-(3-methylbut-2-enyl)phenyl acetate (29f, KU-406);
4-(8-methyl-2-oxo-7-(piperidin-4-yloxy)-2H-chromen-3-ylcarbamoyl)-2-(3-methylbut-2-enyl)phenyl acetate (30b, KU-415);
4-(8-methyl-2-oxo-7-(piperidin-3-yloxy)-2H-chromen-3-ylcarbamoyl)-2-(3-methylbut-2-enyl)phenyl acetate (30d, KU-419);
4-(8-methyl-7-(2-(methylamino)ethoxy)-2-oxo-2H-chromen-3-ylcarbamoyl)-2-(3-methylbut-2-enyl)phenyl acetate (30g, KU-423);
4-Hydroxy-N-(8-methyl-7-(1-methylpiperidin-4-yloxy)-2-oxo-2H-chromen-3-yl)-3-(3-methylbut-2-enyl)benzamide (31a, KU-398);
4-Hydroxy-N-(8-methyl-2-oxo-7-(piperidin-4-yloxy)-2H-chromen-3-yl)-3-(3-methylbut-2-enyl)benzamide (31b, KU-416);
4-Hydroxy-N-(8-methyl-7-(1-methylpiperidin-3-yloxy)-2-oxo-2H-chromen-3-yl)-3-(3-methylbut-2-enyl)benzamide (31c, KU-418);
4-Hydroxy-N-(8-methyl-2-oxo-7-(piperidin-3-yloxy)-2H-chromen-3-yl)-3-(3-methylbut-2-enyl)benzamide (31d, KU-420);
N-(7-(2-(Dimethylamino)ethoxy)-8-methyl-2-oxo-2H-chromen-3-yl)-4-hydroxy-3-(3-methylbut-2-enyl)benzamide (31e, KU-422);
N-(7-(3-(Dimethylamino)propoxy)-8-methyl-2-oxo-2H-chromen-3-yl)-4-hydroxy-3-(3-methylbut-2-enyl)benzamide (31f, KU-407);
4-Hydroxy-N-(8-methyl-7-(2-(methylamino)ethoxy)-2-oxo-2H-chromen-3-yl)-3-(3-methylbut-2-enyl)benzamide (31g, KU-424);
N-(7-((2R,3R,4R)-3,4-dihydroxytetrahydro-2H-pyran-2-yloxy)-8-methyl-2-oxo-2H-chromen-3-yl)-4-hydroxy-3-(3-methylbut-2-enyl)benzamide (16a, KU-425);
N-(7-((2S,3R,4R)-3,4-dihydroxytetrahydro-2H-pyran-2-yloxy)-8-methyl-2-oxo-2H-chromen-3-yl)-4-hydroxy-3-(3-methylbut-2-enyl)benzamide (16b, KU-426);
4-Hydroxy-N-(7-((2R,3R)-3-hydroxytetrahydro-2H-pyran-2-yloxy)-8-methyl-2-oxo-2H-chromen-3-yl)-3-(3-methylbut-2-enyl)benzamide (17a, KU-247);
4-Hydroxy-N-(7-((2S,3R)-3-hydroxytetrahydro-2H-pyran-2-yloxy)-8-methyl-2-oxo-2H-chromen-3-yl)-3-(3-methylbut-2-enyl)benzamide (17b, KU-428);
4-Hydroxy-N-(7-((2S,4R)-4-hydroxytetrahydro-2H-pyran-2-yloxy)-8-methyl-2-oxo-2H-chromen-3-yl)-3-(3-methylbut-2-enyl)benzamide (18a, KU-429);
4-Hydroxy-N-(7-((2R,4R)-4-hydroxytetrahydro-2H-pyran-2-yloxy)-8-methyl-2-oxo-2H-chromen-3-yl)-3-(3-methylbut-2-enyl)benzamide (18b, KU-430);
4-(7-((2S,3S,4S)-3,4-Dihydroxytetrahydrofuran-2-yloxy)-8-methyl-2-oxo-2H-chromen-3-ylcarbamoyl)-2-(3-methylbut-2-enyl)phenyl acetate (19, KU-431);
4-(7-((2S,4R)-4-Hydroxytetrahydrothran-2-yloxy)-8-methyl-2-oxo-2H-chromen-3-ylcarbamoyl)-2-(3-methylbut-2-enyl)phenyl acetate (20a, KU-432); and
4-(7-((2R,4R)-4-Hydroxytetrahydrofuran-2-yloxy)-8-methyl-2-oxo-2H-chromen-3-ylcarbamoyl)-2-(3-methylbut-2-enyl)phenyl acetate (20b, KU-433).

33. The method of claim 31 wherein the compound is:

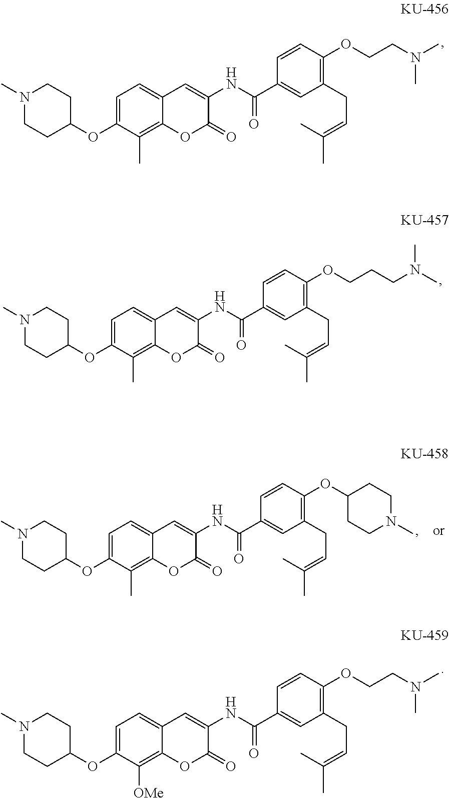

34. The method of claim 28 wherein R″ is a heterocyclic group according to: 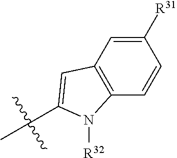
wherein R³¹ is H, halo, or alkoxy; and R³² is H or alkyl.

35. The method of claim 34 wherein the compound is according to Formula III: 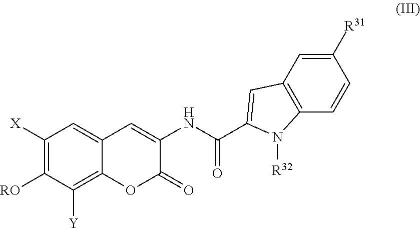
wherein
X is H or —OCH₃,
Y is —CH₃ or —OCH₃;
R³¹ is H, Cl, —CH₂CH₂N(CH₃)₂, —CH₂CH₂CH₂N(CH₃)₂, —OCH₃, or 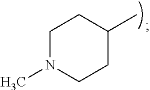
H₃C—N
R³² is H or —CH₃; and
R is selected from the group consisting of:

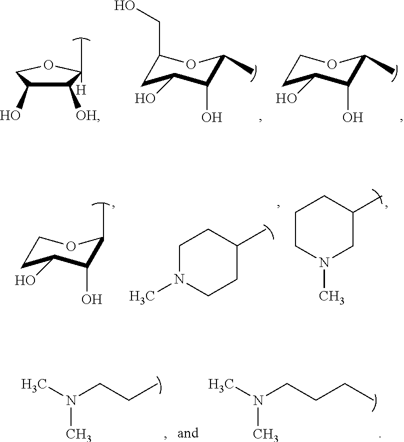

36. The method of claim 35 wherein the compound is:

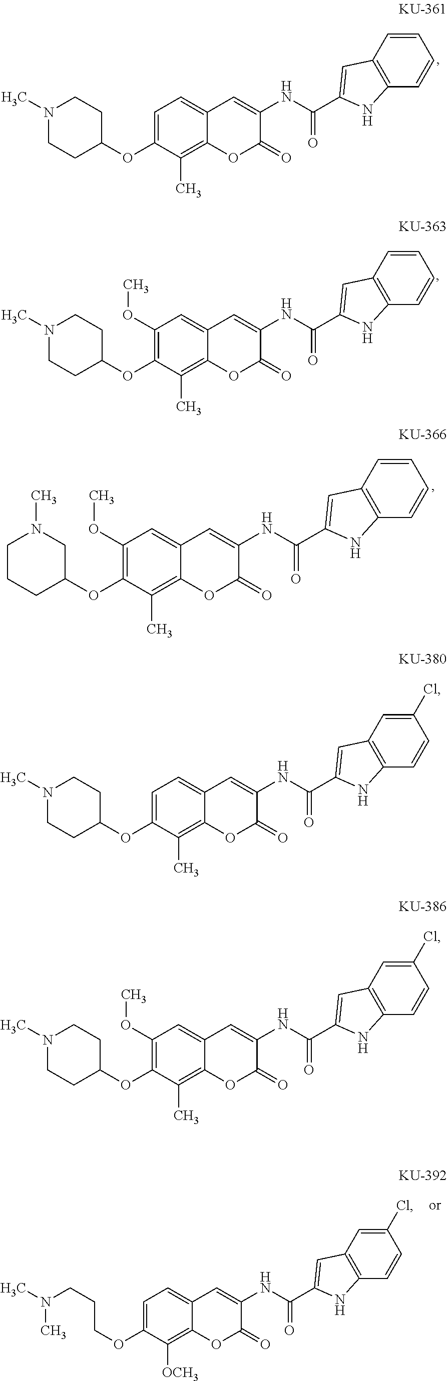

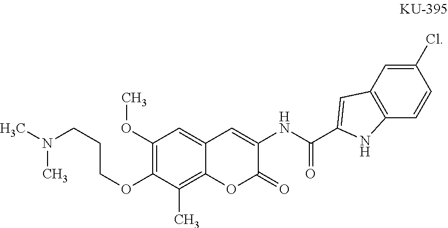

37. The method of claim 29 wherein the compound is according to Formula IV: 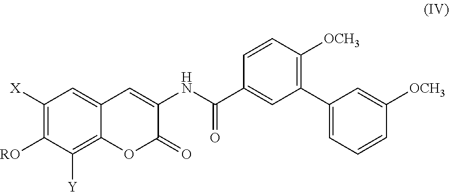
wherein
X is H or —OCH₃;
Y is —CH₃ or —OCH₃; and
R is selected from the group consisting of: H, —COCH₃, mesylate, tosylate, —CONH₂,
—CONHCH₃, —CON(CH₃)₂, —PO(OCH₃)₂, —COCH₃,

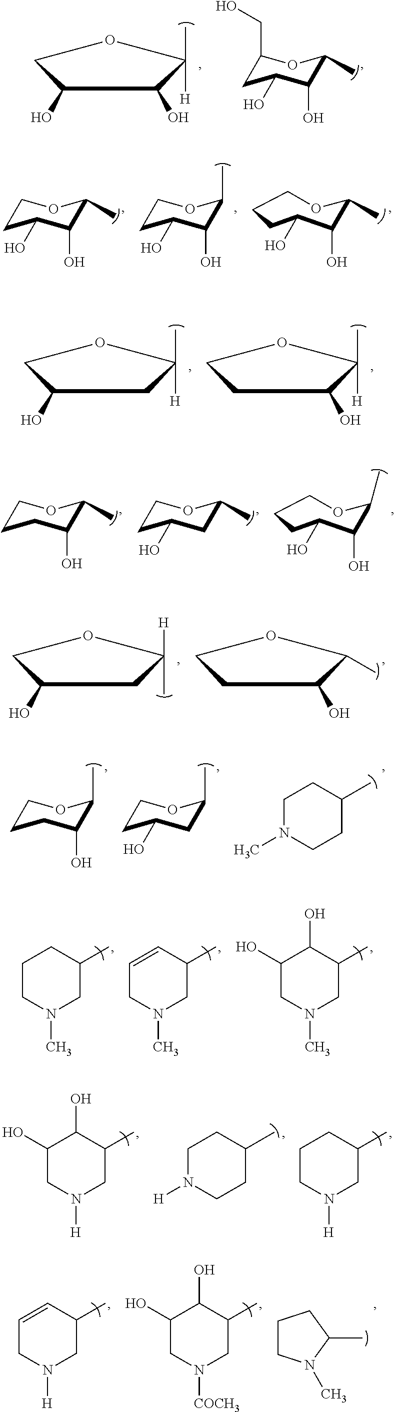

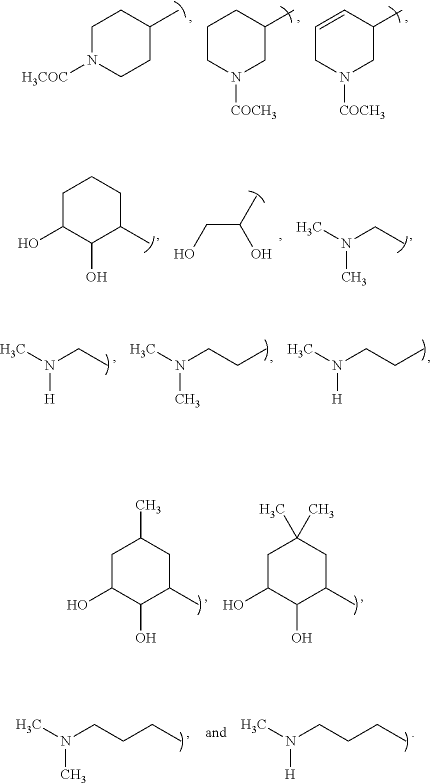

38. The method of claim 37 wherein the compound is:

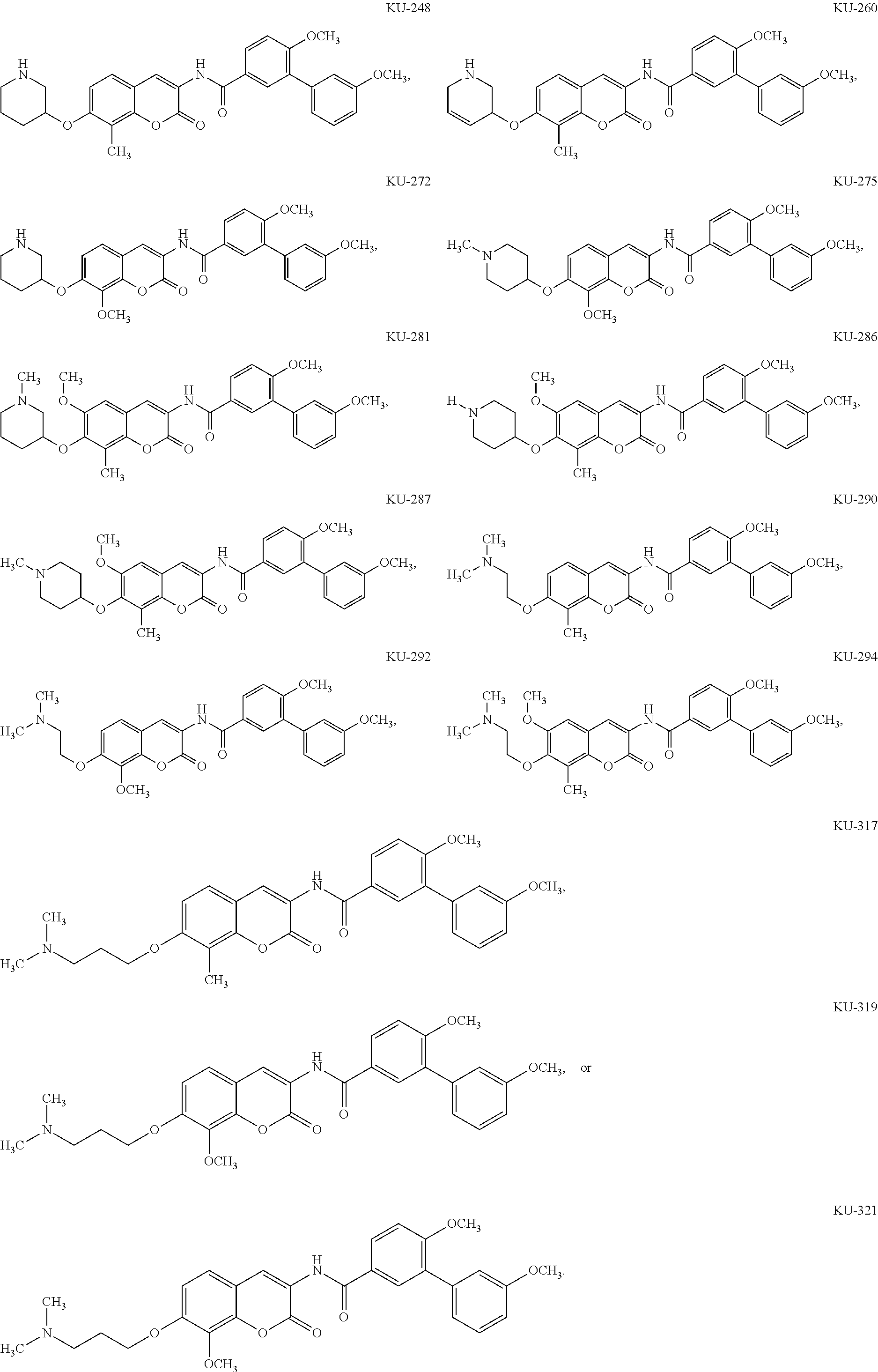

39. The method of claim 24 wherein the PKD is autosomal dominant polycystic kidney disease (ADPKD).

40. The method of claim 24 wherein the one or more symptoms are selected from the group consisting of cyst formation in the kidneys, increase in cyst size in the kidneys, increase in number of cysts in the kidneys, increase in kidney size, and end stage renal disease.

41. The method of claim 1 wherein the compound is KU-32 [chemical structure]
